(19) United States
(12) Patent Application Publication
(10) Pub. No.: US 2005/0069877 A1
(43) Pub. Date: Mar. 31, 2005

(54) Protein modification and maintenance molecules

(76) Inventors: Ameena R. Gandhi, San Francisco, CA (US); Amy E Kable, Silver Spring, MD (US); Anita Swarnakar, San Francisco, CA (US); April J A Hafalia, Daly City, CA (US); Bao Tran, Santa Clara, CA (US); Brendan M Duggan, Sunnyvale, CA (US)

Correspondence Address: FOLEY AND LARDNER;SUITE 500, 3000 K STREET NW, WASHINGTON, DC, 20007, US

(21) Appl. No.: 10/480,988

(22) PCT Filed: Jun. 18, 2002
(86) PCT No.: PCT/US02/19360

Related U.S. Application Data
(60) Provisional application No. 60/300,508, filed on Jun. 22, 2001.

(51) Int. Cl. C12Q 1/68; C07H 21/04; C12P 21/06; C12N 9/64

(52) U.S. Cl. 435/6; 435/69.1; 435/226; 435/320.1; 435/325; 536/23.2

(57) ABSTRACT

Various embodiments of the invention provide human proteinmodification and maintenance molecules (PMOD) and polynucleotides which identify and encode PMOD. Embodiments of the invention also provide expression vectors, host cells, antibodies, agonists, andantagonists. Other embodiments provide methods for diagnosing, eating, or preventing disorders associated with aberrant expression of PMOD.

### TECHNICAL FIELD

The invention relates to novel nucleic acids, protein modification and maintenance molecules encoded by these nucleic acids, and to the use of these nucleic acids and proteins in the diagnosis, treatment, and prevention of gastrointestinal, cardiovascular, autoimmune/inflammatory, cell proliferative, developmental, epithelial, neurological, and reproductive disorders. The invention also relates to the assessment of the effects of exogenous compounds on the expression of nucleic acids and protein modification and maintenance molecules.

### BACKGROUND OF THE INVENTION

The cellular processes regulating modification and maintenance of protein molecules coordinate their function, conformation, stabilization, and degradation. Each of these processes is mediated by key enzymes or proteins such as kinases, phosphatases, proteases, protease inhibitors, isomerases, transferases, and molecular chaperones.

Kinases

Kinases catalyze the transfer of high-energy phosphate groups from adenosine triphosphate (ATP) to target proteins on the hydroxyamino acid residues serine, threonine, or tyrosine. Addition of a phosphate group alters the local charge on the acceptor molecule, causing internal conformational changes and potentially influencing intermolecular contacts. Reversible protein phosphorylation is the ubiquitous strategy used to control many of the intracellular events in eukaryotic cells. It is estimated that more than ten percent of proteins active in a typical mammalian cell are phosphorylated. Extracellular signals including hormones, neurotransmitters, and growth and differentiation factor can activate kinases, which can occur as cell surface receptors or as the activator of the final effector protein, but can also occur along the signal transduction pathway. Kinases are involved in all aspects of a cell's function, from basic metabolic processes, such as glycolysis, to cell-cycle regulation, differentiation, and communication with the extracellular environment through signal transduction cascades. Inappropriate phosphorylation of proteins in cells has been linked to changes in cell cycle progression and cell differentiation. Changes in the cell cycle have been linked to induction of apoptosis or cancer. Changes in cell differentiation have been linked to diseases and disorders of the reproductive system, immune system, and skeletal muscle.

There are two classes of protein kinases. One class, protein tyrosine kinases (PTKs), phosphorylates tyrosine residues, and the other class, protein serine/threonine kinases (STKs), phosphorylates serine and threonine residues. Some PTKs and STKs possess structural characteristics of both families and have dual specificity for both tyrosine and serine/threonine residues. Almost all kinases contain a conserved 250-300 amino acid catalytic domain containing specific residues and sequence motifs characteristic of the kinase family. (Reviewed in Hardie, G. and Hanks, S. (1995) The Protein Kinase Facts Book. Vol I p.p. 17-20 Academic Press, San Diego, Calif.).

Phosphatases

Phosphatases hydrolytically remove phosphate groups from proteins. Phosphatases are essential in determining the extent of phosphorylation in the cell and, together with kinases, regulate key cellular processes such as metabolic enzyme activity, proliferation, cell growth and differentiation, cell adhesion, and cell cycle progression. Protein phosphatases are characterized as either serine/threonine- or tyrosine-specific based on their preferred phospho-amino acid substrate. Some phosphatases (DSPs, for dual specificity phosphatases) can act on phosphorylated tyrosine, serine, or threonine residues. The protein serine/threonine phosphatases (PSPs) are important regulators of many cAMP-mediated hormone responses in cells. Protein tyrosine phosphatases (PTPs) play a significant role in cell cycle and cell signaling processes.

Proteases

Proteases cleave proteins and peptides at the peptide bond that forms the backbone of the protein or peptide chain. Proteolysis is one of the most important and frequent enzymatic reactions that occurs both within and outside of cells. Proteolysis is responsible for the activation and maturation of nascent polypeptides, the degradation of misfolded and damaged proteins, and the controlled turnover of peptides within the cell. Proteases participate in digestion, endocrine function, and tissue remodeling during embryonic development, wound healing, and normal growth. Proteases can play a role in regulatory processes by affecting the half life of regulatory proteins. Proteases are involved in the etiology or progression of disease states such as inflammation, angiogenesis, tumor dispersion and metastasis, cardiovascular disease, neurological disease, and bacterial, parasitic, and viral infections.

Proteases can be categorized on the basis of where they cleave their substrates. Exopeptidases, which include aminopeptidases, dipeptidyl peptidases, tripeptidases, carboxypeptidases, peptidyl-di-peptidases, dipeptidases, and omega peptidases, cleave residues at the termini of their substrates. Endopeptidases, including serine proteases, cysteine proteases, and metalloproteases, cleave at residues within the peptide. Four principal categories of mammalian proteases have been identified based on active site structure, mechanism of action, and overall three-dimensional structure. (See Beynon, R. J. and J. S. Bond (1994) Proteolytic Enzymes: A Practical Approach, Oxford University Press, New York, N.Y., pp. 1-5.)

Serine Proteases

The serine proteases (SPs) are a large, widespread family of proteolytic enzymes that include the digestive enzymes trypsin and chymotrypsin, components of the complement and blood-clotting cascades, and enzymes that control the degradation and turnover of macromolecules within the cell and in the extracellular matrix. Most of the more than 20 subfamilies can be grouped into six clans, each with a common ancestor. These six clans are hypothesized to have descended from at least four evolutionarily distinct ancestors. SPs are named for the presence of a serine residue found in the active catalytic site of most families. The active site is defined by the catalytic triad, a set of conserved asparagine, histidine, and serine residues critical for catalysis. These residues form a charge relay network that facilitates substrate binding. Other residues outside the active site form an oxyanion hole that stabilizes the tetrahedral transition intermediate formed during catalysis. SPs have a wide range of substrates and can be subdivided into subfamilies on the basis of their substrate specificity. The main subfamilies are named for the residue(s) after which they cleave: trypases (after arginine or lysine), aspases (after aspartate), chymases (after phenylalanine or leucine), metases (methionine), and serases (after serine) (Rawlings, N. D. and A. J. Barrett (1994) Meth. Enz. 244:19-61).

Most mammalian serine proteases are synthesized as zymogens, inactive precursors that are activated by proteolysis. For example, trypsinogen is converted to its active form, trypsin, by enteropeptidase. Enteropeptidase is an intestinal protease that removes an N-terminal fragment from trypsinogen. The remaining active fragment is trypsin, which in turn activates the precursors of the other pancreatic enzymes. Likewise, proteolysis of prothrombin, the precursor of thrombin, generates three separate polypeptide fragments. The N-terminal fragment is released while the other two fragments, which comprise active thrombin, remain associated through disulfide bonds.

The two largest SP subfamilies are the chymotrypsin (S1) and subtilisin (S8) families. Some members of the chymotrypsin family contain two structural domains unique to this family. Kringle domains are triple-looped, disulfide cross-linked domains found in varying copy number. Kringles are thought to play a role in binding mediators such as membranes, other proteins or phospholipids, and in the regulation of proteolytic activity (PROSITE PDOC00020). Apple domains are 90 amino-acid repeated domains, each containing six conserved cysteines. Three disulfide bonds link the first and sixth, second and fifth, and third and fourth cysteines (PROSITE PDOC00376). Apple domains are involved in protein-protein interactions. S1 family members include trypsin, chymotrypsin, coagulation factors IX-XII, complement factors B, C, and D, granzymes, kallikrein, and tissue- and urokinase-plasminogen activators. The subtilisin family has members found in the eubacteria, archaebacteria, eukaryotes, and viruses. Subtilisins include the proprotein-processing endopeptidases kexin and furin and the pituitary prohormone convertases PC1, PC2, PC3, PC6, and PACE4 (Rawlings and Barrett, supra).

SPs have functions in many normal processes and some have been implicated in the etiology or treatment of disease. Enterokinase, the initiator of intestinal digestion, is found in the intestinal brush border, where it cleaves the acidic propeptide from trypsinogen to yield active trypsin (Kitamoto, Y. et al. (1994) Proc. Natl. Acad. Sci. USA 91: 7588-7592). Prolylcarboxypeptidase, a lysosomal serine peptidase that cleaves peptides such as angiotensin II and III and [des-Arg9] bradykinin, shares sequence homology with members of both the serine carboxypeptidase and prolylendopeptidase families (Tan, F. et al. (1993) J. Biol. Chem. 268:16631-16638). The protease neuropsin may influence synapse formation and neuronal connectivity in the hippocampus in response to neural signaling (Chen, Z.-L. et al. (1995) J Neurosci 15:5088-5097). Tissue plasminogen activator is useful for acute management of stroke (Zivin, J. A. (1999) Neurology 53:14-9) and myocardial infarction (Ross, A. M. (1999) Clin Cardiol 22:165-71). Some receptors (PAR, for proteinase-activated receptor), highly expressed throughout the digestive tract, are activated by proteolytic cleavage of an extracellular domain. The major agonists for PARs, thrombin, trypsin, and mast cell tryptase, are released in allergy and inflammatory conditions. Control of PAR activation by proteases has been suggested as a promising therapeutic target (Vergnolle, N. (2000) Aliment. Pharmacol. Ther. 14:257-266; Rice, K. D. et al. (1998) Curr. Pharm. Des. 4:381-396). Prostate-specific antigen (PSA) is a kallikrein-like serine protease synthesized and secreted exclusively by epithelial cells in the prostate gland. Serum PSA is elevated in prostate cancer and is the most sensitive physiological marker for monitoring cancer progression and response to therapy. PSA can also identify the prostate as the origin of a metastatic tumor. (Brawer, M. K. and Lange, P. H. (1989) Urology 33:11-16).

The kallikreins are a subfamily of serine proteases. KLK14 is a kallikrein gene located within the human kallikrein locus at 19q13.4. KLK14 is approximately 5.4 kb in length and transcribes two alternative transcripts present only in prostate and skeletal muscle. In prostate, KLK14 is expressed by both benign and malignant glandular epithelial cells, thus exhibiting an expression pattern similar to that of two other prostatic kallikreins, KLK2 and KLK3, which encode K2 and prostate-specific antigen, respectively (Hooper, J. D. et al. (2001) Genomics 73:117-122).

The signal peptidase is a specialized class of SP found in all prokaryotic and eukaryotic cell types that serves in the processing of signal peptides from certain proteins. Signal peptides are amino-terminal domains of a protein which direct the protein from its ribosomal assembly site to a particular cellular or extracellular location. Once the protein has been exported, removal of the signal sequence by a signal peptidase and posttranslational processing, e.g., glycosylation or phosphorylation, activate the protein. Signal peptidases exist as multi-subunit complexes in both yeast and mammals. The canine signal peptidase complex is composed of five subunits, all associated with the microsomal membrane and containing hydrophobic regions that span the membrane one or more times (Shelness, G. S. and G. Blobel (1990) J. Biol. Chem. 265:9512-9519). Some of these subunits serve to fix the complex in its proper position on the membrane while others contain the actual catalytic activity.

Another family of proteases which have a serine in their active site are dependent on the hydrolysis of ATP for their activity. These proteases contain proteolytic core domains and regulatory ATPase domains which can be identified by the presence of the P-loop, an ATP/GTP-binding motif (PROSITE PDOC00803). Members of this family include the eukaryotic mitochondrial matrix proteases, Clp protease and the proteasome. Clp protease was originally found in plant chloroplasts but is believed to be widespread in both prokaryotic and eukaryotic cells. The gene for early-onset torsion dystonia encodes a protein related to Clp protease (Ozelius, L. J. et al. (1998) Adv. Neurol. 78:93-105).

The proteasome is an intracellular protease complex found in some bacteria and in all eukaryotic cells, and plays an important role in cellular physiology. Proteasomes are associated with the ubiquitin conjugation system (UCS), a major pathway for the degradation of cellular proteins of all types, including proteins that function to activate or repress cellular processes such as transcription and cell cycle progression (Ciechanover, A. (1994) Cell 79:13-21). In the UCS pathway, proteins targeted for degradation are conjugated to ubiquitin, a small heat stable protein. The ubiquitinated protein is then recognized and degraded by the proteasome. The resultant ubiquitin-peptide complex is hydrolyzed by a ubiquitin carboxyl terminal hydrolase, and free ubiquitin is released for reutilization by the UCS. Ubiquitin-proteasome systems are implicated in the degradation of mitotic cyclic kinases, oncoproteins, tumor suppressor genes (p53), cell surface receptors associated with signal transduction, transcriptional regulators, and mutated or damaged proteins (Ciechanover, supra). This pathway has been implicated in a number of diseases, including cystic fibrosis, Angelman's syndrome, and Liddle syndrome (reviewed in Schwartz, A. L. and A. Ciechanover (1999) Ann. Rev. Med. 50:57-74). A murine proto-oncogene, Unp, encodes a nuclear ubiquitin protease whose overexpression leads to oncogenic transformation of NIH3T3 cells. The human homologue of this gene is consistently elevated in small cell tumors and adenocarcinomas of the lung (Gray, D. A. (1995) Oncogene 10:2179-2183). Ubiquitin carboxyl terminal hydrolase is involved in the differentiation of a lymphoblastic leukemia cell line to a non-dividing mature state (Maki, A. et al. (1996) Differentiation 60:59-66). In neurons, ubiquitin carboxyl terminal hydrolase (PGP 9.5) expression is strong in the abnormal structures that occur in human neurodegenerative diseases (Lowe, J. et al. (1990) J. Pathol. 161:153-160). The proteasome is a large (˜2000 kDa) multisubunit complex composed of a central catalytic core containing a variety of proteases arranged in four seven-membered rings with the active sites facing inwards into the central cavity, and terminal ATPase subunits covering the outer port of the cavity and regulating substrate entry (for review, see Schmidt, M. et al. (1999) Curr. Op. Chem. Biol. 3:584-591).

Cysteine Proteases

Cysteine proteases (CPs) are involved in diverse cellular processes ranging from the processing of precursor proteins to intracellular degradation. Nearly half of the CPs known are present only in viruses. CPs have a cysteine as the major catalytic residue at the active site where catalysis proceeds via a thioester intermediate and is facilitated by nearby histidine and asparagine residues. A glutamine residue is also important, as it helps to form an oxyanion hole. Two important CP families include the papain-like enzymes (C1) and the calpains (C2). Papain-like family members are generally lysosomal or secreted and therefore are synthesized with signal peptides as well as propeptides. Most members bear a conserved motif in the propeptide that may have structural significance (Karrer, K. M. et al. (1993) Proc. Natl. Acad. Sci. USA 90:3063-3067). Three-dimensional structures of papain family members show a bilobed molecule with the catalytic site located between the two lobes. Papains include cathepsins B, C, H, L, and S, certain plant allergens and dipeptidyl peptidase (for a review, see Rawlings, N. D. and A. J. Barrett (1994) Meth. Enz. 244:461-486).

Some CPs are expressed ubiquitously, while others are produced only by cells of the immune system. Of particular note, CPs are produced by monocytes, macrophages and other cells which migrate to sites of inflammation and secrete molecules involved in tissue repair. Overabundance of these repair molecules plays a role in certain disorders. In autoimmune diseases such as rheumatoid arthritis, secretion of the cysteine peptidase cathepsin C degrades collagen, laminin, elastin and other structural proteins found in the extracellular matrix of bones. Bone weakened by such degradation is also more susceptible to tumor invasion and metastasis. Cathepsin L expression may also contribute to the influx of mononuclear cells which exacerbates the destruction of the rheumatoid synovium (Keyszer, G. M. (1995) Arthritis Rheum. 38:976-984).

Calpains are calcium-dependent cytosolic endopeptidases which contain both an N-terminal catalytic domain and a C-terminal calcium-binding domain. Calpain is expressed as a proenzyme heterodimer consisting of a catalytic subunit unique to each isoform and a regulatory subunit common to different isoforms. Each subunit bears a calcium-binding EF-hand domain. The regulatory subunit also contains a hydrophobic glycine-rich domain that allows the enzyme to associate with cell membranes. Calpains are activated by increased intracellular calcium concentration, which induces a change in conformation and limited autolysis. The resultant active molecule requires a lower calcium concentration for its activity (Chan S. L. and Mattson M. P. (1999) J. Neurosci. Res. 58:167-190). Calpain expression is predominantly neuronal, although it is present in other tissues. Several chronic neurodegenerative disorders, including ALS, Parkinson's disease and Alzheimer's disease are associated with increased calpain expression (Chan and Mattson, supra). Calpain-mediated breakdown of the cytoskeleton has been proposed to contribute to brain damage resulting from head injury (McCracken E. et al. (1999) J. Neurotrauma 16:749-61). Calpain-3 is predominantly expressed in skeletal muscle, and is responsible for limb-girdle muscular dystrophy type 2A (Minami, N. et al. (1999) J. Neurol. Sci. 171:31-37).

Another family of thiol proteases is the caspases, which are involved in the initiation and execution phases of apoptosis. A pro-apoptotic signal can activate initiator caspases that trigger a proteolytic caspase cascade, leading to the hydrolysis of target proteins and the classic apoptotic death of the cell. Two active site residues, a cysteine and a histidine, have been implicated in the catalytic mechanism. Caspases are among the most specific endopeptidases, cleaving after aspartate residues. Caspases are synthesized as inactive zymogens consisting of one large (p20) and one small (p10) subunit separated by a small spacer region, and a variable N-terminal prodomain. This prodomain interacts with cofactors that can positively or negatively affect apoptosis. An activating signal causes autoproteolytic cleavage of a specific aspartate residue (D297 in the caspase-1 numbering convention) and removal of the spacer and prodomain, leaving a p10/p20 heterodimer. Two of these heterodimers interact via their small subunits to form the catalytically active tetramer. The long prodomains of some caspase family members have been shown to promote dimerization and auto-processing of procaspases. Some caspases contain a “death effector domain” in their prodomain by which they can be recruited into self-activating complexes with other caspases and FADD protein associated death receptors or the TNF receptor complex. In addition, two dimers from different caspase family members can associate, changing the substrate specificity of the resultant tetramer. Endogenous caspase inhibitors (inhibitor of apoptosis proteins, or IAPs) also exist. All these interactions have clear effects on the control of apoptosis (reviewed in Chan and Mattson, supra; Salveson, G. S. and V. M. Dixit (1999) Proc. Nat. Acad. Sci. USA 96:10964-10967).

Caspases have been implicated in a number of diseases. Mice lacking some caspases have severe nervous system defects due to failed apoptosis in the neuroepithelium and suffer early lethality. Others show severe defects in the inflammatory response, as caspases are responsible for processing IL-1b and possibly other inflammatory cytokines (Chan and Mattson, supra). Cowpox virus and baculoviruses target caspases to avoid the death of their host cell and promote successful infection. In addition, increases in inappropriate apoptosis have been reported in AIDS, neurodegenerative diseases and ischemic injury, while a decrease in cell death is associated with cancer (Salveson and Dixit, supra; Thompson, C. B. (1995) Science 267:1456-1462).

Aspartyl Proteases

Aspartyl proteases (APs) include the lysosomal proteases cathepsins D and E, as well as chymosin, renin, and the gastric pepsins. Most retroviruses encode an AP, usually as part of the pol polyprotein. APs, also called acid proteases, are monomeric enzymes consisting of two domains, each domain containing one half of the active site with its own catalytic aspartic acid residue. APs are most active in the range of pH 2-3, at which one of the aspartate residues is ionized and the other neutral. The pepsin family of APs contains many secreted enzymes, and all are likely to be synthesized with signal peptides and propeptides. Most family members have three disulfide loops, the first 5 residue loop following the first aspartate, the second 5-6 residue loop preceding the second aspartate, and the third and largest loop occurring toward the C terminus. Retropepsins, on the other hand, are analogous to a single domain of pepsin, and become active as homodimers with each retropepsin monomer contributing one half of the active site. Retropepsins are required for processing the viral polyproteins.

APs have roles in various tissues, and some have been associated with disease. Renin mediates the first step in processing the hormone angiotensin, which is responsible for regulating electrolyte balance and blood pressure (reviewed in Crews, D. E. and S. R. Williams (1999) Hum. Biol. 71:475-503). Abnormal regulation and expression of cathepsins are evident in various inflammatory disease states. Expression of cathepsin D is elevated in synovial tissues from patients with rheumatoid arthritis and osteoarthritis. The increased expression and differential regulation of the cathepsins are linked to the metastatic potential of a variety of cancers (Chambers, A. F. et al. (1993) Crit. Rev. Oncol. 4:95-114).

Metalloproteases

Metalloproteases require a metal ion for activity, usually manganese or zinc. Most zinc-dependent metalloproteases share a common sequence in the zinc-binding domain. The active site is made up of two histidines which act as zinc ligands and a catalytic glutamic acid C-terminal to the first histidine. Proteins containing this signature sequence are known as the metzincins and include aminopeptidase N, angiotensin-converting enzyme, neurolysin, the matrix metalloproteases and the adamalysins (ADAMS). An alternate sequence is found in the zinc carboxypeptidases, in which all three conserved residues—two histidines and a glutamic acid—are involved in zinc binding.

A number of the neutral metalloendopeptidases, including angiotensin converting enzyme and the aminopeptidases, are involved in the metabolism of peptide hormones. High aminopeptidase B activity, for example, is found in the adrenal glands and neurohypophyses of hypertensive rats (Prieto, I. Et al. (1998) Horm. Metab. Res. 30:246-248). Oligopeptidase M/neurolysin can hydrolyze bradykinin as well as neurotensin (Serizawa, A. et al. (1995) J. Biol. Chem 270:2092-2098). Neurotensin is a vasoactive peptide that can act as a neurotransmitter in the brain, where it has been implicated in limiting food intake (Tritos, N. A. et al. (1999) Neuropeptides 33:339-349).

The matrix metalloproteases (MMPs) are a family of at least 23 enzymes that can degrade components of the extracellular matrix (ECM). They are Zn⁺² endopeptidases with an N-terminal catalytic domain. Nearly all members of the family have a hinge peptide and C-terminal domain which can bind to substrate molecules in the ECM or to inhibitors produced by the tissue (TIMPs, for tissue inhibitor of metalloprotease; Campbell, I. L. et al. (1999) Trends Neurosci. 22:285). The presence of fibronectin-like repeats, transmembrane domains, or C-terminal hemopexinase-like domains can be used to separate MMPs into collagenase, gelatinase, stromelysin and membrane-type MMP subfamilies. In the inactive form, the Zn⁺² ion in the active site interacts with a cysteine in the pro-sequence. Activating factors disrupt the Zn⁺²-cysteine interaction, or “cysteine switch,” exposing the active site. This partially activates the enzyme, which then cleaves off its propeptide and becomes fully active. MMPs are often activated by the serine proteases plasmin and furin. MMPs are often regulated by stoichiometric, noncovalent interactions with inhibitors; the balance of protease to inhibitor, then, is very important in tissue homeostasis (reviewed in Yong, V. W. et al. (1998) Trends Neurosci. 21:75).

MMPs are implicated in a number of diseases including osteoarthritis (Mitchell, P. et al. (1996) J. Clin. Inv. 97:761), atherosclerotic plaque rupture (Sukhova, G. K. et al. (1999) Circulation 99:2503), aortic aneurysm (Schneiderman, J. et al. (1998) Am. J. Path. 152:703), non-healing wounds (Saarialho-Kere, U.K. et al. (1994) J. Clin. Inv. 94:79), bone resorption (Blavier, L. and J. M. Delaisse (1995) J. Cell Sci. 108:3649), age-related macular degeneration (Steen, B. et al. (1998) Invest. Ophthalmol. Vis. Sci. 39:2194), emphysema (Finlay, G. A. et al. (1997) Thorax 52:502), myocardial infarction (Rohde, L. E. et al. (1999) Circulation 99:3063) and dilated cardiomyopathy (Thomas, C. V. et al. (1998) Circulation 97:1708). MMP inhibitors prevent metastasis of mammary carcinoma and experimental tumors in rat, and Lewis lung carcinoma, hemangioma, and human ovarian carcinoma xenografts in mice (Eccles S. A. et al. (1996) Cancer Res. 56:2815; Anderson et al. (1996) Cancer Res. 56:715-718; Volpert, O. V. et al. (1996) J. Clin. Invest. 98:671; Taraboletti, G. et al. (1995) JNCI 87:293; Davies, B. et al. (1993) Cancer Res. 53:2087). MMPs may be active in Alzheimer's disease. A number of MMPs are implicated in multiple sclerosis, and administration of MMP inhibitors can relieve some of its symptoms (reviewed in Yong, supra).

Another family of metalloproteases is the ADAMs, for A Disintegrin and Metalloprotease Domain, which they share with their close relatives the adamalysins, snake venom metalloproteases (SVMPs). ADAMs combine features of both cell surface adhesion molecules and proteases, containing a prodomain, a protease domain, a disintegrin domain, a cysteine rich domain, an epidermal growth factor repeat, a transmembrane domain, and a cytoplasmic tail. The first three domains listed above are also found in the SVMPs. The ADAMs possess four potential functions: proteolysis, adhesion, signaling and fusion. The ADAMs share the metzincin zinc binding sequence and are inhibited by some MMP antagonists such as TIMP-1.

ADAMs are implicated in such processes as sperm-egg binding and fusion, myoblast fusion, and protein-ectodomain processing or shedding of cytokines, cytokine receptors, adhesion proteins and other extracellular protein domains (Schlöndorff, J. and C. P. Blobel (1999) J. Cell. Sci. 112:3603-3617). The Kuzbanian protein cleaves a substrate in the NOTCH pathway (possibly NOTCH itself), activating the program for lateral inhibition in Drosophila neural development. Two ADAMs, TACE (ADAM 17) and ADAM 10, are proposed to have analogous roles in the processing of amyloid precursor protein in the brain (Schlöndorff and Blobel, supra). TACE has also been identified as the TNF activating enzyme (Black, R. A. et al. (1997) Nature 385:729). TNF is a pleiotropic cytokine that is important in mobilizing host defenses in response to infection or trauma, but can cause severe damage in excess and is often overproduced in autoimmune disease. TACE cleaves membrane-bound pro-TNF to release a soluble form. Other ADAMs may be involved in a similar type of processing of other membrane-bound molecules.

The ADAMTS sub-family has all of the features of ADAM family metalloproteases and contain an additional thrombospondin domain (TS). The prototypic ADAMTS was identified in mouse, found to be expressed in heart and kidney and upregulated by proinflammatory stimuli (Kuno, K. et al. (1997) J. Biol. Chem. 272:556). To date eleven members are recognized by the Human Genome Organization (HUGO; http://www.gene.ucl.ac.uk/users/hester/adamts.html#Approved). Members of this family have the ability to degrade aggrecan, a high molecular weight proteoglycan which provides cartilage with important mechanical properties including compressibility, and which is lost during the development of arthritis. Enzymes which degrade aggrecan are thus considered attractive targets to prevent and slow the degradation of articular cartilage (See, e.g., Tortorella, M. D. (1999) Science 284:1664; Abbaszade, I. (1999) J. Biol. Chem. 274:23443). Other members are reported to have antiangiogenic potential (Kuno et al., supra) and/or procollagen processing (Colige, A. et al. (1997) Proc.Natl. Acad. Sci. USA 94:2374).

All members of the MDC family of integral membrane proteins contain a metalloproteinase-like domain, a disintegrin-like domain and a cysteine-rich domain. They have been identified in a wide range of mammalian tissues and many are abundantly expressed in the male reproductive tract. A number of MDC proteins (fertilin alpha, fertilin beta, tMDC I, tMDC II and tMDC III) are localized to spermatogenic cells and processed as spermatozoa pass through the epididymis, yielding proteins that retain their disintegrin domain on mature spermatozoa. Fertilin beta and tMDC I have been implicated in egg recognition, mediated by a disintegrin-integrin interaction (Frayne, J. et al. (1998) J. Reprod. Fertil. Suppl. 53:149-155).

Examples of manganese metalloenzymes include aminopeptidase P and human proline dipeptidase (PEPD). Aminopeptidase P can degrade bradykinin, a nonapeptide activated in a variety of inflammatory responses. Aminopeptidase P has been implicated in coronary ischemia/reperfusion injury. Administration of aminopeptidase P inhibitors has been shown to have a cardioprotective effect in rats (Ersahin, C. et al (1999) J. Cardiovasc. Pharmacol. 34:604-611).

Protease Inhibitors

Protease inhibitors and other regulators of protease activity control the activity and effects of proteases. Protease inhibitors have been shown to control pathogenesis in animal models of proteolytic disorders (Murphy, G. (1991) Agents Actions Suppl. 35:69-76). In patients with HIV disease protease inhibitors have been shown to be effective in preventing disease progression and reducing mortality (Barry, M. et al. (1997) Clin. Pharmacokinet. 32:194-209). Low levels of the cystatins, low molecular weight inhibitors of the cysteine proteases, correlate with malignant progression of tumors. (Calkins, C. et al. (1995) Biol. Biochem. Hoppe Seyler 376:71-80). The cystatin superfamily of protease inhibitors is characterized by a particular pattern of linearly arranged and tandemly repeated disulfide loops (Kellermann, J. et al. (1989) J. Biol. Chem. 264:14121-14128). An example of a representative of a structural prototype of a novel family among the cystatin superfamily is human alpha 2-HS glycoprotein (AHSG), a plasma protein synthesized in liver and selectively concentrated in bone matrix, dentine, and other mineralized tissues (Triffitt, J. T. (1976) Calcif. Tissue Res. 22:27-33), which is also classified as belonging to the fetuin family. Fetuins are characterized by the presence of 2 N-terminally located cystatin-like repeats and a unique C-terminal domain which is not present in other proteins of the cystatin superfamily (PROSITE PDOC00966). AHSG has been reported to be involved in bone formation and resorption as well as immune responses (Yang, F. et al. (1992) 1130:149-156; Lee, C. C. et al. (1987) PNAS USA 84:4403-4407; Nakamura, O. et al. (1999) Biosci. Biotechnol. Biochem. 63:1383-1391). Additionally, AHSG has been implicated in infertility associated with endometriosis (Mathur, S. P. (2000) Am. J. Reprod. Immunol. 44:89-95; Mathur, S. P. et al. (1999) Autoimmunity 29:121-127) and inhibition of osteogenesis (Binkert, C. et al, (1999) J. Biol Chem. 274:28514-28520). Decreased serum levels of AHSG have been detected in patients with acute leukemias, chronic granulocyte and myelomonocyte leukemias, lymphomas, myelofibrosis, multiple myeloma, metastatizing solid tumors, systemic lupus erythematosus, rheumatoid arthritis, acute alcoholic hepatitis, fatty liver, chronic active hepatitis, liver cirrhosis, acute and chronic pancreatitis, and Crohn's disease (Kalabay, L. et al. (1992) Orv. Hetil. 133:1553-1554; 1559-1560). Serpins are inhibitors of mammalian plasma serine proteases. Many serpins serve to regulate the blood clotting cascade and/or the complement cascade in mammals. Sp32 is a positive regulator of the mammalian acrosomal protease, acrosin, that binds the proenzyme, proacrosin, and thereby aides in packaging the enzyme into the acrosomal matrix (T. Baba et al. (1994) J. Biol. Chem. 269:10133-10140). The Kunitz family of serine protease inhibitors are characterized by one or more “Kunitz domains” containing a series of cysteine residues that are regularly spaced over approximately 50 amino acid residues and form three intrachain disulfide bonds. Members of this family include aprotinin, tissue factor pathway inhibitor (TFPI-1 and TFPI-2), inter-α-trypsin inhibitor (ITI), and bikunin. (Marlor, C. W. et al. (1997) J. Biol. Chem. 272:12202-12208.) Members of this family are potent inhibitors (in the nanomolar range) against serine proteases such as kallikrein and plasmin. has clinical utility in reduction of perioperative blood loss. ITI has been found to inactivate human trypsin, chymotrypsin, neutrophil elastase and cathepsin G (Morii, M. et al. (1985) Biol. Chem. Hoppe Seyler 366:19-21); and is suspected of playing a key role in the biology of the extracellular matrix and in the pathophysiology of chronic bronchopulmonary diseases or lung cancer progression (Cuvelier, A. et al. (2000) Rev. Mal. Respir. 17:437-446).

Eppin (Epididymal protease inhibitor) is a family of protease inhibitors expressed in the epididymis and testis. Two eppin isoforms contain both Kunitz-type and WAP-type four disulfide core protease inhibitor consensus sequences. Eppin-1 is expressed only in the testis and epididymis; Eppin-2 is expressed only in the epididymis and Eppin-3 only in the testis (Richardson, R. T. et al. (2001) Gene 270:93-102).

Human cystatin C is a potent inihibitor of cysteine proteases. Further, it has amyloidogenic properties. It refolds to produce very tight two-fold symmetric dimers while retaining the secondary structure of the monomeric form. The structure suggests a mechanism for its aggregation in the brain arteries of elderly people with amyloid angiopathy. A more severe ‘conformational disease’ is associated with the L68Q mutant of human cystatin C, which causes massive amyloidosis, cerebral hemorrhage, and death in young adults (Janowski, R. et al. (2001) Nat. Struct. Biol. 8(4):316-20).

A major portion of all proteins synthesized in eukaryotic cells are synthesized on the cytosolic surface of the endoplasmic reticulum (ER). Before these immature proteins are distributed to other organelles in the cell or are secreted, they must be transported into the interior lumen of the ER where post-translational modifications are performed. These modifications include protein folding and the formation of disulfide bonds, and N-linked glycosylations.

Protein Isomerases

Protein folding in the ER is aided by two principal types of protein isomerases, protein disulfide isomerase (PDI), and peptidyl-prolyl isomerase (PPI). PDI catalyzes the oxidation of free sulfhydryl groups in cysteine residues to form intramolecular disulfide bonds in proteins. PPI, an enzyme that catalyzes the isomerization of certain proline imidic bonds in oligopeptides and proteins, is considered to govern one of the rate limiting steps in the folding of many proteins to their final functional conformation. The cyclophilins represent a major class of PPI that was originally identified as the major receptor for the immunosuppressive drug cyclosporin A (Handschumacher, R. E. et al. (1984) Science 226: 544-547).

Protein Glycosylation

The glycosylation of most soluble secreted and membrane-bound proteins by oligosaccharides linked to asparagine residues in proteins is also performed in the ER. This reaction is catalyzed by a membrane-bound enzyme, oligosaccharyl transferase. Although the exact purpose of this “N-linked” glycosylation is unknown, the presence of oligosaccharides tends to make a glycoprotein resistant to protease digestion. In addition, oligosaccharides attached to cell-surface proteins called selectins are known to function in cell-cell adhesion processes (Alberts, B. et al. (1994) Molecular Biology of the Cell Garland Publishing Co., New York, N.Y. p.608). “O-linked” glycosylation of proteins also occurs in the ER by the addition of N-acetylgalactosamine to the hydroxyl group of a serine or threonine residue followed by the sequential addition of other sugar residues to the first. This process is catalyzed by a series of glycosyltransferases each specific for a particular donor sugar nucleotide and acceptor molecule (Lodish, H. et al. (1995) Molecular Cell Biology, W. H. Freeman and Co., New York, N.Y. pp.700-708). In many cases, both − and O-linked oligosaccharides appear to be required for the secretion of proteins or the movement of plasma membrane glycoproteins to the cell surface. For example, one of the glycosyltransferases in the dolichol pathway, dolichol phosphate mannose synthase, is required in N:-glycosylation, O-mannosylation, and glycosylphosphatidylinositol membrane anchoring of protein (Tomita, S. et al. (1998) J. Biol. Chem. 9249-9254). Thus, in many cases, both N- and O-linked oligosaccharides appear to be required for the secretion of proteins or the movement of plasma membrane glycoproteins to the cell surface.

An additional glycosylation mechanism operates in the ER specifically to target lysosomal enzymes to lysosomes and prevent their secretion. Lysosomal enzymes in the ER receive an N-linked oligosaccharide, like plasma membrane and secreted proteins, but are then phosphorylated on one or two mannose residues. The phosphorylation of mannose residues occurs in two steps, the first step being the addition of an N-acetylglucosamine phosphate residue by N-acetylglucosamine phosphotransferase, and the second the removal of the N-acetylglucosamine group by phosphodiesterase. The phosphorylated mannose residue then targets the lysosomal enzyme to a mannose 6-phosphate receptor which transports it to a lysosome vesicle (Lodish et al. supra, pp. 708-711).

Chaperones

Molecular chaperones are proteins that aid in the proper folding of immature proteins and refolding of improperly folded ones, the assembly of protein subunits, and in the transport of unfolded proteins across membranes. Chaperones are also called heat-shock proteins (hsp) because of their tendency to be expressed in dramatically increased amounts following brief exposure of cells to elevated temperatures. This latter property most likely reflects their need in the refolding of proteins that have become denatured by the high temperatures. Chaperones may be divided into several classes according to their location, function, and molecular weight, and include hsp60, TCP1, hsp70, hsp40 (also called DnaJ), and hsp90. For example, hsp90 binds to steroid hormone receptors, represses transcription in the absence of the ligand, and provides proper folding of the ligand-binding domain of the receptor in the presence of the hormone (Burston, S. G. and A. R. Clarke (1995) Essays Biochem. 29:125-136). Hsp60 and hsp70 chaperones aid in the transport and folding of newly synthesized proteins. Hsp70 acts early in protein folding, binding a newly synthesized protein before it leaves the ribosome and transporting the protein to the mitochondria or ER before releasing the folded protein. Hsp60, along with hsp10, binds misfolded proteins and gives them the opportunity to refold correctly. All chaperones share an affinity for hydrophobic patches on incompletely folded proteins and the ability to hydrolyze ATP. The energy of ATP hydrolysis is used to release the hsp-bound protein in its properly folded state (Alberts, B. et al. supra, pp 214, 571-572).

Dipeptidyl-peptidase I, a lysosomal cysteine proteinase, is important in intracellular degradation of proteins and appears to be a central coordinator for activation of many serine proteinases in immune/inflammatory cells. The gene has been mapped to chromosomal region 11q14.1-q14.3. Dipeptidyl-peptidase I is expressed at high levels in lung, kidney, and placenta, and also at high levels in polymorphonuclear leukocytes and alveolar macrophages and their precursor cells (Rao, N. V. et al. (1997) J. Biol. Chem. 272:10260-10265).

IAP is a protein family that has baculovirus IAP repeat (BIR) domains and inhibits apoptosis. A human IAP family gene, Apollon, encodes a 530 kDa protein that contains a single BIR domain and a ubiquitin-conjugating enzyme domain. Apollon has been observed to protect cells from undergoing apoptosis and implicated in tumorigenesis and drug resistance (Chen, Z. et al. (1999) Biochem. Biophys. Res. Commun. 264:847-854).

The RTVL-H family is a medium repetitive family of endogenous retrovirus-like sequences found in the genomes of humans and other primates. Different subfamilies of RTVL-H elements are designated Type I, Type Ia, and Type II (Goodchild, N. L. (1993) Virology 196:778-788).

Lysyl Hydroxylases

Lysyl hydroxylase is an enzyme involved in collagen biosynthesis. Collagens are a family of fibrous structural proteins that are found in essentially all tissues. Collagens are the most abundant proteins in mammals, and are essential for the formation of connective tissue such as skin, bone, tendon, cartilage, blood vessels and teeth. Members of the collagen family can be distinguished from one another by the degree of cross-linking between collagen fibers and by the number of carbohydrate units (e.g., galactose or glucosylgalactose) attached to the collagen fibers. Hydroxylated lysine residues (hydroxylysine) are essential for stability of cross-linking and as attachment points for carbohydrate units.

The enzyme lysyl hydroxylase catalyzes the hydroxylation of lysine residues to form hydroxylysine. Lysyl hydroxylase targets the lysine residue of the sequence, X-lys-gly (lys=lysine, gly=glycine, and X=any amino acid residue). Three isoforms of lysyl hydroxylase have been characterized, termed LH1 (or PLOD; procollagen-lysine, 2-oxoglutarate 5-dioxygenase), LH2 (or PLOD2), and LH3. The three enzymes share 60% sequence identity overall, with even higher similarity in the C-terminal region. In addition, there are regions in the middle of the molecule that have an identity of more than 80% (Valtavaara, M. et al. (1998) J. Biol. Chem. 273:12881-12886).

Diminished lysyl hydroxylase activity is involved in certain connective tissue disorders. In particular mutations, including a truncation and duplications within the coding region of the gene for PLOD, have been described in patients with type VI Ehlers-Danos syndrome (Hyland, J. et al. (1992) Nature Genet. 2:228-31; Hautala, T. et al. (1993) Genomics 15:399-404).

Ubiquitin-Associated Proteins

The ubiquitin conjugation system (UCS), is a major pathway for the degradation of cellular proteins of all types, including proteins that function to activate or repress cellular processes such as transcription, cell cycle progression, and immune recognition (Ciechanover, A. (1994) Cell 79:13-21). The process of ubiquitin conjugation and protein degradation involves several steps (Jentsch, S. (1992) Annu. Rev. Genet. 26:179-207). First ubiquitin (Ub), a small, heat stable protein is activated by a ubiquitin-activating enzyme (E1) in an ATP dependent reaction which binds the C-terminus of Ub to the thiol group of an internal cysteine residue in E1. Activated Ub is then transferred to one of several Ub-conjugating enzymes (E2). Different ubiquitin-dependent proteolytic pathways employ structurally similar, but distinct ubiquitin-conjugating enzymes that are associated with recognition subunits which direct them to proteins carrying a particular degradation signal. E2 then transfers the Ub molecule through its C-terminal glycine to a member of the ubiquitin-protein ligase family, E3. Next, E3 transfers the Ub molecule to the target protein. Additional Ub molecules may be added to the target protein forming a multi-Ub chain structure. The ubiquitinated protein is then recognized and degraded by the proteasome, an intracellular protease complex found in some bacteria and in all eukaryotic cells. The resultant ubiquitin-peptide complex is hydrolyzed by a ubiquitin carboxyl terminal hydrolase, and free ubiquitin is released for reutilization by the UCS.

Ubiquitin-proteasome systems are implicated in the degradation of mitotic cyclic kinases, oncoproteins, tumor suppressor genes (p53), cell surface receptors associated with signal transduction, transcriptional regulators, and mutated or damaged proteins (Ciechanover, supra). This pathway has been implicated in a number of diseases, including cystic fibrosis, Angelman's syndrome, and Liddle syndrome (reviewed in Schwartz, A. L. and A. Ciechanover (1999) Annu. Rev. Med. 50:57-74). A murine proto-oncogene, Unp, encodes a nuclear ubiquitin protease whose overexpression leads to oncogenic transformation of NIH3T3 cells. The human homologue of this gene is consistently elevated in small cell tumors and adenocarcinomas of the lung (Gray, D. A. (1995) Oncogene 10:2179-2183). Ubiquitin carboxyl terminal hydrolase is involved in the differentiation of a lymphoblastic leukemia cell line to a non-dividing mature state (Maki, A. et al. (1996) Differentiation 60:59-66). In neurons, ubiquitin carboxyl terminal hydrolase (PGP 9.5) expression is strong in the abnormal structures that occur in human neurodegenerative diseases (Lowe, J. et al. (1990) J. Pathol. 161:153-160).

Additional ubiquitin-like proteins which also possess the ability to covalently modify other cellular proteins have been identified in recent years. (For review, see Yeh, E. T. H. et al. (2000) Gene 248:1-14; and Jentsch, S. and Pyrowolakis, G. (2000) Trends Cell Biol. 10:335-342.) These ubiquitin-like protein modifiers include the sentrins (also known as SUMO proteins), NEDD8, and Apg12. The conjugation pathways for these proteins closely resemble that for ubiquitin. For example, conjugation of sentrin requires the E1 heterodimer AOS1/UBA2, and a single E2 enzyme, UBC9. The recently discovered protein S3 may function as a sentrin ligase. The yeast protein Ulp1 is a sentrin hydrolase. Inactivation of Ulp1 in yeast results in severe cell cycle defects. In humans, seven sentrin specific proteases (SENP) have been identified, which range in size from 238 to 1112 amino acid residues (Yeh, supra). All human SENPs share a conserved C-terminal domain. The N-terminal regions may regulate cellular location and substrate specificity.

Sentrinization does not promote protein degradation as does ubiquitin. In some cases sentrinization appears to be important for stable localization of target proteins in nuclear bodies. Substrates for sentrinization include PML, a RING finger protein with tumor suppressor activity, HIPK2, a co-repressor for homeodomain transcription factors, and the tumor suppressor p53. IκBα, a cytosolic inhibitor of NFκB, a transcription factor involved in induction of inflammation associated proteins, is also a substrate for sentrinization. Sentrinized IκBα cannot be ubiquitinated and is resistant to proteasomal degradation, suggesting links between the ubiquitin and sentrin pathways. Jentsch, supra).

Expression Profiling

Microarrays are analytical tools used in bioanalysis. A microarray has a plurality of molecules spatially distributed over, and stably associated with, the surface of a solid support. Microarrays of polypeptides, polynucleotides, and/or antibodies have been developed and find use in a variety of applications, such as gene sequencing, monitoring gene expression, gene mapping, bacterial identification, drug discovery, and combinatorial chemistry.

One area in particular in which microarrays find use is in gene expression analysis. Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

Steroids Affecting Protein Modification

Steroids are a class of lipid-soluble molecules, including cholesterol, bile acids, vitamin D, and hormones, that share a common four-ring structure based on cyclopentanoperhydrophenanthrene and that carrry out a wide variety of functions. Cholesterol, for example, is a component of cell membranes that controls membrane fluidity. It is also a precursor for bile acids which solubilize lipids and facilitate absorption in the small intestine during digestion. Vitamin D regulates the absorption of calcium in the small intestine and controls the concentration of calcium in plasma. Steroid hormones, produced by the adrenal cortex, ovaries, and testes, include glucocorticoids, mineralocorticoids, androgens, and estrogens. They control various biological processes by binding to intracellular receptors that regulate transcription of specific genes in the nucleus. Glucocorticoids, for example, increase blood glucose concentrations by regulation of gluconeogenesis in the liver, increase blood concentrations of fatty acids by promoting lipolysis in adipose tissues, modulate sensitivity to catcholamines in the central nervous system, and reduce inflammation. The principal mineralocorticoid, aldosterone, is produced by the adrenal cortex and acts on cells of the distal tubules of the kidney to enhance sodium ion reabsorption. Androgens, produced by the interstitial cells of Leydig in the testis, include the male sex hormone testosterone, which triggers changes at puberty, the production of sperm and maintenance of secondary sexual characteristics. Female sex hormones, estrogen and progesterone, are produced by the ovaries and also by the placenta and adrenal cortex of the fetus during pregnancy. Estrogen regulates female reproductive processes and secondary sexual characteristics. Progesterone regulates changes in the endometrium during the menstrual cycle and pregnancy.

Steroid hormones are widely used for fertility control and in anti-inflammatory treatments for physical injuries and diseases such as arthritis, asthma, and auto-immune disorders. Progesterone, a naturally occurring progestin, is primarily used to treat amenorrhea, abnormal uterine bleeding, or as a contraceptive. Endogenous progesterone is responsible for inducing secretory activity in the endometrium of the estrogen-primed uterus in preparation for the implantation of a fertilized egg and for the maintenance of pregnancy. It is secreted from the corpus luteum in response to luteinizing hormone (LH). The primary contraceptive effect of exogenous progestins involves the suppression of the midcycle surge of LH. At the cellular level, progestins diffuse freely into target cells and bind to the progesterone receptor. Target cells include the female reproductive tract, the mammary gland, the hypothalamus, and the pituitary. Once bound to the receptor, progestins slow the frequency of release of gonadotropin releasing hormone from the hypothalamus and blunt the pre-ovulatory LH surge, thereby preventing follicular maturation and ovulation. Progesterone has minimal estrogenic and androgenic activity. Progesterone is metabolized hepatically to pregnanediol and conjugated with glucuronic acid.

Medroxyprogesterone (MAH), also known as 6α-methyl-17-hydroxyprogesterone, is a synthetic progestin with a pharmacological activity about 15 times greater than progesterone. MAH is used for the treatment of renal and endometrial carcinomas, amenorrhea, abnormal uterine bleeding, and endometriosis associated with hormonal imbalance. MAH has a stimulatory effect on respiratory centers and has been used in cases of low blood oxygenation caused by sleep apnea, chronic obstructive pulmonary disease, or hypercapnia.

Mifepristone, also known as RU-486, is an antiprogesterone drug that blocks receptors of progesterone. It counteracts the effects of progesterone, which is needed to sustain pregnancy. Mifepristone induces spontaneous abortion when administered in early pregnancy followed by treatment with the prostaglandin, misoprostol. Further, studies show that mifepristone at a substantially lower dose can be highly effective as a postcoital contraceptive when administered within five days after unprotected intercourse, thus providing women with a “morning-after pill” in case of contraceptive failure or sexual assault. Mifepristone also has potential uses in the treatment of breast and ovarian cancers in cases in which tumors are progesterone-dependent. It interferes with steroid-dependent growth of brain meningiomas, and may be useful in treatment of endometriosis where it blocks the estrogen-dependent growth of endometrial tissues. It may also be useful in treatment of uterine fibroid tumors and Cushing's Syndrome. Mifepristone binds to glucocorticoid receptors and interferes with cortisol binding. Mifepristone also may act as an anti-glucocorticoid and be effective for treating conditions where cortisol levels are elevated such as AIDS, anorexia nervosa, ulcers, diabetes, Parkinson's disease, multiple sclerosis, and Alzheimer's disease.

Danazol is a synthetic steroid derived from ethinyl testosterone. Danazol indirectly reduces estrogen production by lowering pituitary synthesis of follicle-stimulating hormone and LH. Danazol also binds to sex hormone receptors in target tissues, thereby exhibiting anabolic, antiestrognic, and weakly androgenic activity. Danazol does not possess any progestogenic activity, and does not suppress normal pituitary release of corticotropin or release of cortisol by the adrenal glands. Danazol is used in the treatment of endometriosis to relieve pain and inhibit endometrial cell growth. It is also used to treat fibrocystic breast disease and hereditary angioedema.

Corticosteroids are used to relieve inflammation and to suppress the immune response. They inhibit eosinophil, basophil, and airway epithelial cell function by regulation of cytokines that mediate the inflammatory response. They inhibit leukocyte infiltration at the site of inflammation, interfere in the function of mediators of the inflammatory response, and suppress the humoral immune response. Corticosteroids are used to treat allergies, asthma, arthritis, and skin conditions. Beclomethasone is a synthetic glucocorticoid that is used to treat steroid-dependent asthma, to relieve symptoms associated with allergic or nonallergic (vasomotor) rhinitis, or to prevent recurrent nasal polyps following surgical removal. The anti-inflammatory and vasoconstrictive effects of intranasal beclomethasone are 5000 times greater than those produced by hydrocortisone. Budesonide is a corticosteroid used to control symptoms associated with allergic rhinitis or asthma. Budesonide has high topical anti-inflammatory activity but low systemic activity. Dexamethasone is a synthetic glucocorticoid used in anti-inflammatory or immunosuppressive compositions. It is also used in inhalants to prevent symptoms of asthma. Due to its greater ability to reach the central nervous system, dexamethasone is usually the treatment of choice to control cerebral edema. Dexamethasone is approximately 20-30 times more potent than hydrocortisone and 5-7 times more potent than prednisone. Prednisone is metabolized in the liver to its active form, prednisolone, a glucocorticoid with anti-inflammatory properties. Prednisone is approximately 4 times more potent than hydrocortisone and the duration of action of prednisone is intermediate between hydrocortisone and dexamethasone. Prednisone is used to treat allograft rejection, asthma, systemic lupus erythematosus, arthritis, ulcerative colitis, and other inflammatory conditions. Betamethasone is a synthetic glucocorticoid with antiinflammatory and immunosuppressive activity and is used to treat psoriasis and fungal infections, such as athlete's foot and ringworm.

The anti-inflammatory actions of corticosteroids are thought to involve phospholipase A₂ inhibitory proteins, collectively called lipocortins. Lipocortins, in turn, control the biosynthesis of potent mediators of inflammation such as prostaglandins and leukotrienes by inhibiting the release of the precursor molecule arachidonic acid. Proposed mechanisms of action include decreased IgE synthesis, increased number of β-adrenergic receptors on leukocytes, and decreased arachidonic acid metabolism. During an immediate allergic reaction, such as in chronic bronchial asthma, allergens bridge the IgE antibodies on the surface of mast cells, which triggers these cells to release chemotactic substances. Mast cell influx and activation, therefore, is partially responsible for the inflammation and hyperirritability of the oral mucosa in asthmatic patients. This inflammation can be retarded by administration of corticosteroids.

Toxicology Testing:

Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for examining which genes are tissue specific, carry out housekeeping functions, are parts of a signaling cascade, or are specifically related to a particular genetic predisposition, condition, disease, or disorder.

The potential application of gene expression profiling is particularly relevant to improving diagnosis, prognosis, and treatment of disease. For example, both the levels and sequences expressed in tissues from subjects with hyperlipidemia may be compared with the levels and sequences expressed in normal tissue.

Toxicity testing is a mandatory and time-consuming part of drug development programs in the pharmaceutical industry. A more rapid screen to determine the effects upon metabolism and to detect toxicity of lead drug candidates may be the use of gene expression microarrays. For example, microarrays of various kinds may be produced using full length genes or gene fragments. These arrays can then be used to test samples treated with the drug candidates to elucidate the gene expression pattern associated with drug treatment. This gene pattern can be compared with gene expression patterns associated with compounds which produce known metabolic and toxicological responses.

The human C3A cell line is a clonal derivative of HepG2/C3 (hepatoma cell line, isolated from a 15-year-old male with liver tumor), which was selected for strong contact inhibition of growth. The use of a clonal population enhances the reproducibility of the cells. C3A cells have many characteristics of primary human hepatocytes in culture: i) expression of insulin receptor and insulin-like growth factor II receptor; ii) secretion of a high ratio of serum albumin compared with α-fetoprotein iii) convertion of ammonia to urea and glutamine; iv) metabolism of aromatic amino acids; and v) ability to proliferate in glucose-free and insulin-free medium. The C3A cell line is now well established as an in vitro model of the mature human liver (Mickelson et al. (1995) Hepatology 22:866-875; Nagendra et al. (1997) Am. J. Physiol. 272:G408-416).

Clofibrate is an hypolidemic drug which lowers elevated levels of serum triglycerides. In rodents, chronic treatment produces hepatomegaly and an increase in hepatic peroxisomes (peroxisome proliferation). Peroxisome proliferators (PPs) are a class of drugs which activate the PP-activated receptor in rodent liver, leading to enzyme induction, stimulation of S-phase, and a suppression of apoptosis (Hasmall and Roberts (1999) Pharmacol. Ther. 82:63-70). PPs include the fibrate class of hypolidemic drugs, phenobarbitone, thiazolidinediones, certain non-steroidal anti-inflammatory drugs, and naturally-occuring fatty acid-derived molecules (Gelman et al. (1999) Cell. Mol. Life Sci. 55:932-943). Clofibrate has been shown to increase levels of cytochrome P450 4A. It is also involved in transcription of β-oxidation genes as well as induction of PP-activated receptors (Kawashima et al. (1997) Arch. Biochem. Biophys. 347:148-154). Peroxisome proliferation that is induced by both clofibrate and the chemically-related compound fenofibrate is mediated by a common inhibitory effect on mitochondrial membrane depolarization (Zhou and Wallace (1999) Toxicol. Sci. 48:82-89).

Dexamethasone and its derivatives, dexamethasone sodium phosphate and dexamethasone acetate, are synthetic glucocorticoids used as anti-inflammatory or immunosuppressive agents. Dexamethasone has little to no mineralocorticoid activity and is usually selected for management of cerebral edema because of its superior ability to penetrate the central nervous sytem. Glucocorticoids are naturally occurring hormones that prevent or suppress inflammation and immune responses when administered at pharmacological doses. Responses can include inhibition of leukocyte infiltration at the site of inflammation, interference in the function of mediators of inflammatory response, and suppression of humoral immune responses. The anti-inflammatory actions of corticosteroids are thought to involve phospholipase A₂ inhibitory proteins, collectively called lipocortins. The numerous adverse effects related to corticosteroid use usually depend on the dose administered and the duration of therapy. Proposed mechanisms of action include decreased IgE synthesis, increased number of β-adrenergic receptors on leukocytes, and decreased arachidonic acid metabolism. During an immediate allergic reaction, such as in chronic bronchial asthma, allergens bridge the IgE antibodies on the surface of mast cells, which triggers these cells to release chemotactic substances. Mast cell influx and activation, therefore, is partially responsible for the inflammation and hyperirritability of the oral mucosa in asthmatic patients. This inflammation can be retarded by administration of adrenocorticoids. As with other corticosteroids, the effects upon liver metabolism and hormone clearance mechanisms are important to understand the pharmacodynamics of a drug.

Cancer

Prostate cancer develops through a multistage progression ultimately resulting in an aggressive tumor phenotype. The initial step in tumor progression involves the hyperproliferation of normal luminal and/or basal epithelial cells. Androgen responsive cells become hyperplastic and evolve into early-stage tumors. Although early-stage tumors are often androgen sensitive and respond to androgen ablation, a population of androgen independent cells evolve from the hyperplastic population. These cells represent a more advanced form of prostate tumor that may become invasive and potentially become metastatic to the bone, brain, or lung.

Breast cancer develops through a multi-step process in which pre-malignant mammary epithelial cells undergo a relatively defined sequence of events leading to tumor formation. An early event in tumor development is ductal hyperplasia. Cells undergoing rapid neoplastic growth gradually progress to invasive carcinoma and become metastatic to the lung, bone, and potentially other organs. Several variables that may influence the process of tumor progression and malignant transformation include genetic factors, environmental factors, growth factors, and hormones. Based on the complexity of this process, it is critical to study a population of human mammary epithelial cells undergoing the process of malignant transformation, and to associate specific stages of progression with phenotypic and molecular characteristics.

Immune Response Proteins

Interleukin 12 (IL-12) is a pleiotropic cytokine produced by macrophages and B lymphocytes that can have multiple effects on T cells and natural killer (NK) cells. Effects include inducing production of IFN-γ and TNF by resting and activated T and NK cells; enhancing the cytotoxic activity of resting NK and T cells, inducing and synergizing with IL-2 in the generation of lymphokine-activated killer (LAK) cells; acting as a comitogen to stimulate proliferation of resting T cells; and inducing proliferation of activated T and NK cells. Current evidence indicates that IL-12, produced by macrophages in response to infectious agents, is a central mediator of the cell-mediated immune response by its actions on the development, proliferation, and activities of TH1 cells. As the initiator of cell-mediated immunity, IL-12 may stimulate cell-mediated immune responses to microbial pathogens, metastatic cancers, and viral infections such as AIDS.

There is a need in the art for new compositions, including nucleic acids and proteins, for the diagnosis, prevention, and treatment of gastrointestinal, cardiovascular, autoimmune/inflammatory, cell proliferative, developmental, epithelial, neurological, and reproductive disorders.

### SUMMARY OF THE INVENTION

Various embodiments of the invention provide purified polypeptides, protein modification and maintenance molecules, referred to collectively as “PMOD” and individually as “PMOD-1,” “PMOD-2,” “PMOD-3,” “PMOD-4,” “PMOD-5,” “PMOD-6,” “PMOD-7,” “PMOD-8,” “PMOD-9,” “PMOD-10,” “PMOD-11,” “PMOD-12,” “PMOD-13,” “PMOD-14,” “PMOD-15,” “PMOD-16,” “PMOD-17,” “PMOD-18,” “PMOD-19,” “PMOD-20,” “PMOD-21,” “PMOD-22,” “PMOD-23,” “PMOD-24,” “PMOD-25,” “PMOD-26,” “PMOD-27,” and “PMOD-28,” and methods for using these proteins and their encoding polynucleotides for the detection, diagnosis, and treatment of diseases and medical conditions. Embodiments also provide methods for utilizing the purified protein modification and maintenance molecules and/or their encoding polynucleotides for facilitating the drug discovery process, including determination of efficacy, dosage, toxicity, and pharmacology. Related embodiments provide methods for utilizing the purified protein modification and maintenance molecules and/or their encoding polynucleotides for investigating the pathogenesis of diseases and medical conditions.

An embodiment provides an isolated polypeptide selected from the group consisting of a) a polypeptide comprising an amino acid sequence selected from the group consisting of SEQ ID NO:1-28, b) a polypeptide comprising a naturally occurring amino acid sequence at least 90% identical or at least about 90% identical to an amino acid sequence selected from the group consisting of SEQ ID NO:1-28, c) a biologically active fragment of a polypeptide having an amino acid sequence selected from the group consisting of SEQ ID NO:1-28, and d) an immunogenic fragment of a polypeptide having an amino acid sequence selected from the group consisting of SEQ ID NO:1-28. Another embodiment provides an isolated polypeptide comprising an amino acid sequence of SEQ ID NO:1-28.

Still another embodiment provides an isolated polynucleotide encoding a polypeptide selected from the group consisting of a) a polypeptide comprising an amino acid sequence selected from the group consisting of SEQ ID NO:1-28, b) a polypeptide comprising a naturally occurring amino acid sequence at least 90% identical or at least about 90% identical to an amino acid sequence selected from the group consisting of SEQ ID NO:1-28, c) a biologically active fragment of a polypeptide having an amino acid sequence selected from the group consisting of SEQ ID NO:1-28, and d) an immunogenic fragment of a polypeptide having an amino acid sequence selected from the group consisting of SEQ ID NO:1-28. In another embodiment, the polynucleotide encodes a polypeptide selected from the group consisting of SEQ ID NO:1-28. In an alternative embodiment, the polynucleotide is selected from the group consisting of SEQ ID NO:29-56.

Still another embodiment provides a recombinant polynucleotide comprising a promoter sequence operably linked to a polynucleotide encoding a polypeptide selected from the group consisting of a) a polypeptide comprising an amino acid sequence selected from the group consisting of SEQ ID NO:1-28, b) a polypeptide comprising a naturally occurring amino acid sequence at least 90% identical or at least about 90% identical to an amino acid sequence selected from the group consisting of SEQ ID NO:1-28, c) a biologically active fragment of a polypeptide having an amino acid sequence selected from the group consisting of SEQ ID NO:1-28, and d) an immunogenic fragment of a polypeptide having an amino acid sequence selected from the group consisting of SEQ ID NO:1-28. Another embodiment provides a cell transformed with the recombinant polynucleotide. Yet another embodiment provides a transgenic organism comprising the recombinant polynucleotide.

Another embodiment provides a method for producing a polypeptide selected from the group consisting of a) a polypeptide comprising an amino acid sequence selected from the group consisting of SEQ ID NO:1-28, b) a polypeptide comprising a naturally occurring amino acid sequence at least 90% identical or at least about 90% identical to an amino acid sequence selected from the group consisting of SEQ ID NO:1-28, c) a biologically active fragment of a polypeptide having an amino acid sequence selected from the group consisting of SEQ ID NO:1-28, and d) an immunogenic fragment of a polypeptide having an amino acid sequence selected from the group consisting of SEQ ID NO:1-28. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Yet another embodiment provides an isolated antibody which specifically binds to a polypeptide selected from the group consisting of a) a polypeptide comprising an amino acid sequence selected from the group consisting of SEQ ID NO:1-28, b) a polypeptide comprising a naturally occurring amino acid sequence at least 90% identical or at least about 90% identical to an amino acid sequence selected from the group consisting of SEQ ID NO:1-28, c) a biologically active fragment of a polypeptide having an amino acid sequence selected from the group consisting of SEQ ID NO:1-28, and d) an immunogenic fragment of a polypeptide having an amino acid sequence selected from the group consisting of SEQ ID NO:1-28.

Still yet another embodiment provides an isolated polynucleotide selected from the group consisting of a) a polynucleotide comprising a polynucleotide sequence selected from the group consisting of SEQ ID NO:29-56, b) a polynucleotide comprising a naturally occurring polynucleotide sequence at least 90% identical or at least about 90% identical to a polynucleotide sequence selected from the group consisting of SEQ ID NO:29-56, c) a polynucleotide complementary to the polynucleotide of a), d) a polynucleotide complementary to the polynucleotide of b), and e) an RNA equivalent of a)-d). In other embodiments, the polynucleotide can comprise at least about 20, 30, 40, 60, 80, or 100 contiguous nucleotides.

Yet another embodiment provides a method for detecting a target polynucleotide in a sample, said target polynucleotide being selected from the group consisting of a) a polynucleotide comprising a polynucleotide sequence selected from the group consisting of SEQ ID NO:29-56, b) a polynucleotide comprising a naturally occurring polynucleotide sequence at least 90% identical or at least about 90% identical to a polynucleotide sequence selected from the group consisting of SEQ ID NO:29-56, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex. In a related embodiment, the method can include detecting the amount of the hybridization complex. In still other embodiments, the probe can comprise at least about 20, 30, 40, 60, 80, or 100 contiguous nucleotides.

Still yet another embodiment provides a method for detecting a target polynucleotide in a sample, said target polynucleotide being selected from the group consisting of a) a polynucleotide comprising a polynucleotide sequence selected from the group consisting of SEQ ID NO:29-56, b) a polynucleotide comprising a naturally occurring polynucleotide sequence at least 90% identical or at least about 90% identical to a polynucleotide sequence selected from the group consisting of SEQ ID NO:29-56, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof. In a related embodiment, the method can include detecting the amount of the amplified target polynucleotide or fragment thereof.

Another embodiment provides a composition comprising an effective amount of a polypeptide selected from the group consisting of a) a polypeptide comprising an amino acid sequence selected from the group consisting of SEQ ID NO:1-28, b) a polypeptide comprising a naturally occurring amino acid sequence at least 90% identical or at least about 90% identical to an amino acid sequence selected from the group consisting of SEQ ID NO:1-28, c) a biologically active fragment of a polypeptide having an amino acid sequence selected from the group consisting of SEQ ID NO:1-28, and d) an immunogenic fragment of a polypeptide having an amino acid sequence selected from the group consisting of SEQ ID NO:1-28, and a pharmaceutically acceptable excipient. In one embodiment, the composition can comprise an amino acid sequence selected from the group consisting of SEQ ID NO:1-28. Other embodiments provide a method of treating a disease or condition associated with decreased or abnormal expression of functional PMOD, comprising administering to a patient in need of such treatment the composition.

Yet another embodiment provides a method for screening a compound for effectiveness as an agonist of a polypeptide selected from the group consisting of a) a polypeptide comprising an amino acid sequence selected from the group consisting of SEQ ID NO:1-28, b) a polypeptide comprising a naturally occurring amino acid sequence at least 90% identical or at least about 90% identical to an amino acid sequence selected from the group consisting of SEQ ID NO:1-28, c) a biologically active fragment of a polypeptide having an amino acid sequence selected from the group consisting of SEQ ID NO:1-28, and d) an immunogenic fragment of a polypeptide having an amino acid sequence selected from the group consisting of SEQ ID NO:1-28. The method comprises a) exposing a sample comprising the polypeptide to a compound, and b) detecting agonist activity in the sample. Another embodiment provides a composition comprising an agonist compound identified by the method and a pharmaceutically acceptable excipient. Yet another embodiment provides a method of treating a disease or condition associated with decreased expression of functional PMOD, comprising administering to a patient in need of such treatment the composition.

Still yet another embodiment provides a method for screening a compound for effectiveness as an antagonist of a polypeptide selected from the group consisting of a) a polypeptide comprising an amino acid sequence selected from the group consisting of SEQ ID NO:1-28, b) a polypeptide comprising a naturally occurring amino acid sequence at least 90% identical or at least about 90% identical to an amino acid sequence selected from the group consisting of SEQ ID NO:1-28, c) a biologically active fragment of a polypeptide having an amino acid sequence selected from the group consisting of SEQ ID NO:1-28, and d) an immunogenic fragment of a polypeptide having an amino acid sequence selected from the group consisting of SEQ ID NO:1-28. The method comprises a) exposing a sample comprising the polypeptide to a compound, and b) detecting antagonist activity in the sample. Another embodiment provides a composition comprising an antagonist compound identified by the method and a pharmaceutically acceptable excipient. Yet another embodiment provides a method of treating a disease or condition associated with overexpression of functional PMOD, comprising administering to a patient in need of such treatment the composition.

Another embodiment provides a method of screening for a compound that specifically binds to a polypeptide selected from the group consisting of a) a polypeptide comprising an amino acid sequence selected from the group consisting of SEQ ID NO:1-28, b) a polypeptide comprising a naturally occurring amino acid sequence at least 90% identical or at least about 90% identical to an amino acid sequence selected from the group consisting of SEQ ID NO:1-28, c) a biologically active fragment of a polypeptide having an amino acid sequence selected from the group consisting of SEQ ID NO:1-28, and d) an immunogenic fragment of a polypeptide having an amino acid sequence selected from the group consisting of SEQ ID NO:1-28. The method comprises a) combining the polypeptide with at least one test compound under suitable conditions, and b) detecting binding of the polypeptide to the test compound, thereby identifying a compound that specifically binds to the polypeptide.

Yet another embodiment provides a method of screening for a compound that modulates the activity of a polypeptide selected from the group consisting of a) a polypeptide comprising an amino acid sequence selected from the group consisting of SEQ ID NO:1-28, b) a polypeptide comprising a naturally occurring amino acid sequence at least 90% identical or at least about 90% identical to an amino acid sequence selected from the group consisting of SEQ ID NO:1-28, c) a biologically active fragment of a polypeptide having an amino acid sequence selected from the group consisting of SEQ ID NO:1-28, and d) an immunogenic fragment of a polypeptide having an amino acid sequence selected from the group consisting of SEQ ID NO:1-28. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

Still yet another embodiment provides a method for screening a compound for effectiveness in altering expression of a target polynucleotide, wherein said target polynucleotide comprises a polynucleotide sequence selected from the group consisting of SEQ ID NO:29-56, the method comprising a) exposing a sample comprising the target polynucleotide to a compound, b) detecting altered expression of the target polynucleotide, and c) comparing the expression of the target polynucleotide in the presence of varying amounts of the compound and in the absence of the compound.

Another embodiment provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:29-56, ii) a polynucleotide comprising a naturally occurring polynucleotide sequence at least 90% identical or at least about 90% identical to a polynucleotide sequence selected from the group consisting of SEQ ID NO:29-56, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO:29-56, ii) a polynucleotide comprising a naturally occurring polynucleotide sequence at least 90% identical or at least about 90% identical to a polynucleotide sequence selected from the group consisting of SEQ ID NO:29-56, iii) a polynucleotide complementary to the polynucleotide of i), iv) a polynucleotide complementary to the polynucleotide of ii), and v) an RNA equivalent of i)-iv). Alternatively, the target polynucleotide can comprise a fragment of a polynucleotide selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for full length polynucleotide and polypeptide embodiments of the invention.

Table 2 shows the GenBank identification number and annotation of the nearest GenBank homolog, and the PROTEOME database identification numbers and annotations of PROTEOME database homologs, for polypeptide embodiments of the invention. The probability scores for the matches between each polypeptide and its homolog(s) are also shown.

Table 3 shows structural features of polypeptide embodiments, including predicted motifs and domains, along with the methods, algorithms, and searchable databases used for analysis of the polypeptides.

Table 4 lists the cDNA and/or genomic DNA fragments which were used to assemble polynucleotide embodiments, along with selected fragments of the polynucleotides.

Table 5 shows representative cDNA libraries for polynucleotide embodiments.

Table 6 provides an appendix which describes the tissues and vectors used for construction of the cDNA libraries shown in Table 5.

Table 7 shows the tools, programs, and algorithms used to analyze polynucleotides and polypeptides, along with applicable descriptions, references, and threshold parameters.

Table 8 shows single nucleotide polymorphisms found in polynucleotide embodiments, along with allele frequencies in different human populations.

### DESCRIPTION OF THE INVENTION

Before the present proteins, nucleic acids, and methods are described, it is understood that embodiments of the invention are not limited to the particular machines, instruments, materials, and methods described, as these may vary. It is also to be understood that the terminology used herein is for the purpose of describing particular embodiments only, and is not intended to limit the scope of the invention.

As used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with various embodiments of the invention. Nothing herein is to be construed as an admission that the invention is not entitled to antedate such disclosure by virtue of prior invention.

### DEFINITIONS

“PMOD” refers to the amino acid sequences of substantially purified PMOD obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of PMOD. Agonists may include proteins, nucleic acids, carbohydrates, small molecules, or any other compound or composition which modulates the activity of PMOD either by directly interacting with PMOD or by acting on components of the biological pathway in which PMOD participates.

An “allelic variant” is an alternative form of the gene encoding PMOD. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding PMOD include those sequences with deletions, insertions, or substitutions of different nucleotides, resulting in a polypeptide the same as PMOD or a polypeptide with at least one functional characteristic of PMOD. Included within this definition are polymorphisms which may or may not be readily detectable using a particular oligonucleotide probe of the polynucleotide encoding PMOD, and improper or unexpected hybridization to allelic variants, with a locus other than the normal chromosomal locus for the polynucleotide encoding PMOD. The encoded protein may also be “altered,” and may contain deletions, insertions, or substitutions of amino acid residues which produce a silent change and result in a functionally equivalent PMOD. Deliberate amino acid substitutions may be made on the basis of one or more similarities in polarity, charge, solubility, hydrophobicity, hydrophilicity, and/or the amphipathic nature of the residues, as long as the biological or immunological activity of PMOD is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” can refer to an oligopeptide, a peptide, a polypeptide, or a protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid. Amplification may be carried out using polymerase chain reaction (PCR) technologies or other nucleic acid amplification technologies well known in the art.

The term “antagonist” refers to a molecule which inhibits or attenuates the biological activity of PMOD. Antagonists may include proteins such as antibodies, anticalins, nucleic acids, carbohydrates, small molecules, or any other compound or composition which modulates the activity of PMOD either by directly interacting with PMOD or by acting on components of the biological pathway in which PMOD participates.

The term “antibody” refers to intact immunoglobulin molecules as well as to fragments thereof, such as Fab, F(ab′)₂, and Fv fragments, which are capable of binding an epitopic determinant. Antibodies that bind PMOD polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), described in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide may be replaced by 2′-F or 2′-NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers may be specifically cross-linked to their cognate ligands, e.g., by photo-activation of a cross-linker. (See, e.g., Brody, E. N. and L. Gold (2000) J. Biotechnol. 74:5-13.)

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

The term “antisense” refers to any composition capable of base-pairing with the “sense” (coding) strand of a polynucleotide having a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic PMOD, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide” and a “composition comprising a given polypeptide” can refer to any composition containing the given polynucleotide or polypeptide. The composition may comprise a dry formulation or an aqueous solution. Compositions comprising polynucleotides encoding PMOD or fragments of PMOD may be employed as hybridization probes. The probes may be stored in freeze-dried form and may be associated with a stabilizing agent such as a carbohydrate. In hybridizations, the probe may be deployed in an aqueous solution containing salts (e.g., NaCl, detergents (e.g., sodium dodecyl sulfate; SDS), and other components (e.g., Denhardt's solution, dry milk, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (GCG, Madison Wis.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalently or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

“Exon shuffling” refers to the recombination of different coding regions (exons). Since an exon may represent a structural or functional domain of the encoded protein, new proteins may be assembled through the novel reassortment of stable substructures, thus allowing acceleration of the evolution of new protein functions.

A “fragment” is a unique portion of PMOD or a polynucleotide encoding PMOD which can be identical in sequence to, but shorter in length than, the parent sequence. A fragment may comprise up to the entire length of the defined sequence, minus one nucleotide/amino acid residue. For example, a fragment may comprise from about 5 to about 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO:29-56 can comprise a region of unique polynucleotide sequence that specifically identifies SEQ ID NO:29-56, for example, as distinct from any other sequence in the genome from which the fragment was obtained. A fragment of SEQ ID NO:29-56 can be employed in one or more embodiments of methods of the invention, for example, in hybridization and amplification technologies and in analogous methods that distinguish SEQ ID NO:29-56 from related polynucleotides. The precise length of a fragment of SEQ ID NO:29-56 and the region of SEQ ID NO:29-56 to which the fragment corresponds are routinely determinable by one of ordinary skill in the art based on the intended purpose for the fragment.

A fragment of SEQ ID NO:1-28 is encoded by a fragment of SEQ ID NO:29-56. A fragment of SEQ ID NO:1-28 can comprise a region of unique amino acid sequence that specifically identifies SEQ ID NO:1-28. For example, a fragment of SEQ ID NO:1-28 can be used as an immunogenic peptide for the development of antibodies that specifically recognize SEQ ID NO:1-28. The precise length of a fragment of SEQ ID NO:1-28 and the region of SEQ ID NO:1-28 to which the fragment corresponds can be determined based on the intended purpose for the fragment using one or more analytical methods described herein or otherwise known in the art.

A “full length” polynucleotide is one containing at least a translation initiation codon (e.g., methionine) followed by an open reading frame and a translation termination codon. A “full length” polynucleotide sequence encodes a “full length” polypeptide sequence.

“Homology” refers to sequence similarity or, interchangeably, sequence identity, between two or more polynucleotide sequences or two or more polypeptide sequences.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using one or more computer algorithms or programs known in the art or described herein. For example, percent identity can be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989) CABIOS 5:151-153 and in Higgins, D. G. et al. (1992) CABIOS 8:189-191. For pairwise alignments of polynucleotide sequences, the default parameters are set as follows: Ktuple=2, gap penalty=5, window=4, and “diagonals saved”=4. The “weighted” residue weight table is selected as the default. Percent identity is reported by CLUSTAL V as the “percent similarity” between aligned polynucleotide sequences.

Alternatively, a suite of commonly used and freely available sequence comparison algorithms which can be used is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nih.gov/gorf/bl2.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (Apr. 21, 2000) set at default parameters. Such default parameters may be, for example:
Matrix: BLOSUM62
Rewardfor match: 1
Penalty for mismatch: −-2
Open Gap: 5 and Extension Gap: 2 penalties
Gap×drop-off: 50
Expect: 10
Word Size: 11
Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.12 (Apr. 21, 2000) with blastp set at default parameters. Such default parameters may be, for example:
Matrix: BLOSUM62
Open Gap: 11 and Extension Gap: 1 penalties
Gap×drop-off: 50
Expect: 10
Word Size: 3
Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. et al. (1989) Molecular Cloning: A Laboratory Manual, 2^{nd} ed., vol. 1-3, Cold Spring Harbor Press, Plainview N.Y.; specifically see volume 2, chapter 9.

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acids by virtue of the formation of hydrogen bonds between complementary bases. A hybridization complex may be formed in solution (e.g., C₀t or R₀t analysis) or formed between one nucleic acid present in solution and another nucleic acid immobilized on a solid support (e.g., paper, membranes, filters, chips, pins or glass slides, or any other appropriate substrate to which cells or their nucleic acids have been fixed).

The words “insertion” and “addition” refer to changes in an amino acid or polynucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of PMOD which is capable of eliciting an immune response when introduced into a living organism, for example, a mammal. The term “immunogenic fragment” also includes any polypeptide or oligopeptide fragment of PMOD which is useful in any of the antibody production methods disclosed herein or known in the art.

The term “microarray” refers to an arrangement of a plurality of polynucleotides, polypeptides, antibodies, or other chemical compounds on a substrate.

The terms “element” and “array element” refer to a polynucleotide, polypeptide, antibody, or other chemical compound having a unique and defined position on a microarray.

The term “modulate” refers to a change in the activity of PMOD. For example, modulation may cause an increase or a decrease in protein activity, binding characteristics, or any other biological, functional, or immunological properties of PMOD.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an PMOD may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of PMOD.

“Probe” refers to nucleic acids encoding PMOD, their complements, or fragments thereof, which are used to detect identical, allelic or related nucleic acids. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme.

Primer pairs can be used for amplification (and identification) of a nucleic acid, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in the references, for example Sambrook, J. et al. (1989) Molecular Cloning: A Laboratory Manual, 2^{nd} ed., vol. 1-3, Cold Spring Harbor Press, Plainview N.Y.; Ausubel, F. M. et al. (1987) Current Protocols in Molecular Biology, Greene Publ. Assoc. & Wiley-Intersciences, New York N.Y.; Innis, M. et al. (1990) PCR Protocols, A Guide to Methods and Applications, Academic Press, San Diego Calif. PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a nucleic acid that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, supra. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA molecule, is composed of the same linear sequence of nucleotides as the reference DNA molecule with the exception that all occurrences of the nitrogenous base thymine are replaced with uracil, and the sugar backbone is composed of ribose instead of deoxyribose.

The term “sample” is used in its broadest sense. A sample suspected of containing PMOD, nucleic acids encoding PMOD, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least about 60% free, preferably at least about 75% free, and most preferably at least about 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” or “expression profile” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. In another embodiment, the nucleic acid can be introduced by infection with a recombinant viral vector, such as a lentiviral vector (Lois, C. et al. (2002) Science 295:868-872). The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook et al. (1989), supra.

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May 7, 1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotides that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity to the particular polypeptide sequence over a certain length of one of the polypeptide sequences using blastp with the “BLAST 2 Sequences” tool Version 2.0.9 (May 7, 1999) set at default parameters. Such a pair of polypeptides may show, for example, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides.

### THE INVENTION

Various embodiments of the invention include new human protein modification and maintenance molecules (PMOD), the polynucleotides encoding PMOD, and the use of these compositions for the diagnosis, treatment, or prevention of gastrointestinal, cardiovascular, autoimmune/inflammatory, cell proliferative, developmental, epithelial, neurological, and reproductive disorders.

Table 1 summarizes the nomenclature for the full length polynucleotide and polypeptide embodiments of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown. Column 6 shows the Incyte ID numbers of physical, full length clones corresponding to polypeptide and polynucleotide embodiments. The full length clones encode polypeptides which have at least 95% sequence identity to the polypeptides shown in column 3.

Table 2 shows sequences with homology to the polypeptides of the invention as identified by BLAST analysis against the GenBank protein (genpept) database and the PROTEOME database. Columns 1 and 2 show the polypeptide sequence identification number (Polypeptide SEQ ID NO:) and the corresponding Incyte polypeptide sequence number (Incyte Polypeptide ID) for polypeptides of the invention. Column 3 shows the GenBank identification number (GenBank ID) NO:) of the nearest GenBank homolog and the PROTEOME database identification numbers (PROTEOME ID NO:) of the nearest PROTEOME database homologs. Column 4 shows the probability scores for the matches between each polypeptide and its homolog(s). Column 5 shows the annotation of the GenBank and PROTEOME database homolog(s) along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows potential phosphorylation sites, and column 5 shows potential glycosylation sites, as determined by the MOTIFS program of the GCG sequence analysis software package (Genetics Computer Group, Madison Wis.). Column 6 shows amino acid residues comprising signature sequences, domains, and motifs. Column 7 shows analytical methods for protein structure/function analysis and in some cases, searchable databases to which the analytical methods were applied.

Together, Tables 2 and 3 summarize the properties of polypeptides of the invention, and these properties establish that the claimed polypeptides are protein modification and maintenance molecules. For example, SEQ ID NO:1 is 43% identical, from residue K223 to residue A774, to Arabidopsis thaliana ubiquitin-protein ligase 1 (GenBank ID g7108521) as determined by the Basic Local Alignment Search Tool (BLAST). (See Table 2.) The BLAST probability score is 1.3e-85, which indicates the probability of obtaining the observed polypeptide sequence alignment by chance. SEQ ID NO:1 also contains a HECT (ubiquitin-transferase) domain as determined by searching for statistically significant matches in the hidden Markov model (HMM)-based PFAM database of conserved protein family domains. (See Table 3.) Data from BLIMPS and BLAST analyses provide further corroborative evidence that SEQ ID NO:1 is a ubiquitin-protein ligase.

As another example, SEQ ID NO:5 is 38% identical, from residue E22 to residue K368, to Arabidopsis thaliana ubiquitin-specific protease 26 (GenBank ID g11993492) as determined by the Basic Local Alignment Search Tool (BLAST). (See Table 2.) The BLAST probability score is 1.1e-79, which indicates the probability of obtaining the observed polypeptide sequence alignment by chance. SEQ ID NO:5 also contains a ubiquitin carboxl-terminal hydrolases 1 domain and a ubiquitin carboxl-terminal hydrolases 2 domain as determined by searching for statistically significant matches in the hidden Markov model (HMM)-based PFAM database of conserved protein family domains. (See Table 3.) Data from BLIMPS, MOTIFS, and additional BLAST analyses provide further corroborative evidence that SEQ ID NO:5 is a ubiquitin carboxyl terminal hydrolase.

As another example, SEQ ID NO:7 is 91% identical, from residue P23 to residue S531, to a human carboxypeptidase N (GenBank ID g179936) as determined by the Basic Local Alignment Search Tool (BLAST). (See Table 2.) The BLAST probability score is 1.7e-235, which indicates the probability of obtaining the observed polypeptide sequence alignment by chance. SEQ ID NO:7 also contains leucine-rich repeat domains as determined by searching for statistically significant matches in the hidden Markov model (HMM)-based PFAM database of conserved protein family domains. (See Table 3.) Data from MOTIFS analysis provides further corroborative evidence that SEQ ID NO:7 is a carboxypeptidase.

As another example, SEQ ID NO:10 is 46% identical, from residue R8 to residue S143, to mouse testatin, which is related to the cysteine protease inhibitors, cystatins (GenBank ID g3928491) as determined by the Basic Local Alignment Search Tool (BLAST). (See Table 2.) The BLAST probability score is 1.4e-27, which indicates the probability of obtaining the observed polypeptide sequence alignment by chance. SEQ ID NO:10 also contains a cystatin domain as determined by searching for statistically significant matches in the hidden Markov model (HMM)-based PFAM database of conserved protein family domains. (See Table 3.) Data from further BLAST analysis provides corroborative evidence that SEQ ID NO:10 is a cysteine protease inhibitor.

As another example, SEQ ID NO:20 is 99% identical, from residue M17 to residue K267, to human kallikrein 14 (GenBank ID g13897995) as determined by the Basic Local Alignment Search Tool (BLAST). (See Table 2.) The BLAST probability score is 2.1e-136, which indicates the probability of obtaining the observed polypeptide sequence alignment by chance. SEQ ID NO:20 also contains a trypsin domain as determined by searching for statistically significant matches in the hidden Markov model (HMM)-based PFAM database of conserved protein family domains. (See Table 3.) Data from BLIMPS, MOTIFS, and PROFILESCAN analyses provide further corroborative evidence that SEQ ID NO:20 is a serine protease.

As another example, SEQ ID NO:27 is 96% identical, from residue M1 to residue T242, to human putative mast cell mMCP-7-like II tryptase (GenBank ID g4336577) as determined by the Basic Local Alignment Search Tool (BLAST). (See Table 2.) The BLAST probability score is 1.8e-130, which indicates the probability of obtaining the observed polypeptide sequence alignment by chance. SEQ ID NO:27 also contains a trypsin domain as determined by searching for statistically significant matches in the hidden Markov model (HMM)-based PFAM database of conserved protein family domains. (See Table 3.) Data from BLIMPS, MOTIFS, and PROFILESCAN analyses provide further corroborative evidence that SEQ ID NO:27 is a trypsin-like serine protease.

SEQ ID NO:2-4, SEQ ID NO:6, SEQ ID NO:8-9, SEQ ID NO:11-19, SEQ ID NO:21-26 and SEQ ID NO:28 were analyzed and annotated in a similar manner. The algorithms and parameters for the analysis of SEQ ID NO:1-28 are described in Table 7.

As shown in Table 4, the full length polynucleotide embodiments were assembled using cDNA sequences or coding (exon) sequences derived from genomic DNA, or any combination of these two types of sequences. Column 1 lists the polynucleotide sequence identification number (Polynucleotide SEQ ID NO:), the corresponding Incyte polynucleotide consensus sequence number (Incyte ID) for each polynucleotide of the invention, and the length of each polynucleotide sequence in basepairs. Column 2 shows the nucleotide start (5′) and stop (3′) positions of the cDNA and/or genomic sequences used to assemble the full length polynucleotide embodiments, and of fragments of the polynucleotides which are useful, for example, in hybridization or amplification technologies that identify SEQ ID NO:29-56 or that distinguish between SEQ ID NO:29-56 and related polynucleotides.

The polynucleotide fragments described in Column 2 of Table 4 may refer specifically, for example, to Incyte cDNAs derived from tissue-specific cDNA libraries or from pooled cDNA libraries. Alternatively, the polynucleotide fragments described in column 2 may refer to GenBank cDNAs or ESTs which contributed to the assembly of the full length polynucleotides. In addition, the polynucleotide fragments described in column 2 may identify sequences derived from the ENSEMBL (The Sanger Centre, Cambridge, UK) database (i.e., those sequences including the designation “ENST”). Alternatively, the polynucleotide fragments described in column 2 may be derived from the NCBI RefSeq Nucleotide Sequence Records Database (i.e., those sequences including the designation “NM” or “NT”) or the NCBI RefSeq Protein Sequence Records (i.e., those sequences including the designation “NP”). Alternatively, the polynucleotide fragments described in column 2 may refer to assemblages of both cDNA and Genscan-predicted exons brought together by an “exon stitching” algorithm. For example, a polynucleotide sequence identified as FL_XXXXXX_N_{1—}N_{2—}YYYYY_N_{3—}N₄ represents a “stitched” sequence in which XXXXXX is the identification number of the cluster of sequences to which the algorithm was applied, and YYYYY is the number of the prediction generated by the algorithm, and N_{1,2,3 . . .} , if present, represent specific exons that may have been manually edited during analysis (See Example V). Alternatively, the polynucleotide fragments in column 2 may refer to assemblages of exons brought together by an “exon-stretching” algorithm. For example, a polynucleotide sequence identified as FLXXXXXX_gAAAAA_gBBBBB_{—}1_N is a “stretched” sequence, with XXXXXX being the Incyte project identification number, gAAAAA being the GenBank identification number of the human genomic sequence to which the “exon-stretching” algorithm was applied, gBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithm, a RefSeq identifier (denoted by “NM,” “NP,” or “NT”) may be used in place of the GenBank identifier (i.e., gBBBBB).

Alternatively, a prefix identifies component sequences that were hand-edited, predicted from genomic DNA sequences, or derived from a combination of sequence analysis methods. The following Table lists examples of component sequence prefixes and corresponding sequence analysis methods associated with the prefixes (see Example IV and Example V).

| | Type of analysis and/ |
|---|---|
| Prefix | or examples of programs |
| GNN, GFG, | Exon prediction from genomic |
| ENST | sequences using, for example, |
| | GENSCAN (Stanford University, |
| | CA, USA) or FGENES (Computer |
| | Genomics Group, The Sanger |
| | Centre, Cambridge, UK). |
| GBI | Hand-edited analysis of |
| | genomic sequences. |
| FL | Stitched or stretched genomic |
| | sequences (see Example V). |
| INCY | Full length transcript and exon |
| | prediction from mapping of EST |
| | sequences to the genome. Genomic |
| | location and EST composition |
| | data are combined to predict |
| | the exons and resulting transcript. |

In some cases, Incyte cDNA coverage redundant with the sequence coverage shown in Table 4 was obtained to confirm the final consensus polynucleotide sequence, but the relevant Incyte cDNA identification numbers are not shown.

Table 5 shows the representative cDNA libraries for those full length polynucleotides which were assembled using Incyte cDNA sequences. The representative cDNA library is the Incyte cDNA library which is most frequently represented by the Incyte cDNA sequences which were used to assemble and confirm the above polynucleotides. The tissues and vectors which were used to construct the cDNA libraries shown in Table 5 are described in Table 6.

Table 8 shows single nucleotide polymorphisms (SNPs) found in polynucleotide embodiments, along with allele frequencies in different human populations. Columns 1 and 2 show the polynucleotide sequence identification number (SEQ ID NO:) and the corresponding Incyte project identification number (PID) for polynucleotides of the invention. Column 3 shows the Incyte identification number for the EST in which the SNP was detected (EST ID), and column 4 shows the identification number for the SNP (SNP ID). Column 5 shows the position within the EST sequence at which the SNP is located (EST SNP), and column 6 shows the position of the SNP within the full-length polynucleotide sequence (CB1 SNP). Column 7 shows the allele found in the EST sequence. Columns 8 and 9 show the two alleles found at the SNP site. Column 10 shows the amino acid encoded by the codon including the SNP site, based upon the allele found in the EST. Columns 11-14 show the frequency of allele 1 in four different human populations. An entry of n/d (not detected) indicates that the frequency of allele 1 in the population was too low to be detected, while n/a (not available) indicates that the allele frequency was not determined for the population.

The invention also encompasses PMOD variants. A preferred PMOD variant is one which has at least about 80%, or alternatively at least about 90%, or even at least about 95% amino acid sequence identity to the PMOD amino acid sequence, and which contains at least one functional or structural characteristic of PMOD.

Various embodiments also encompass polynucleotides which encode PMOD. In a particular embodiment, the invention encompasses a polynucleotide sequence comprising a sequence selected from the group consisting of SEQ ID NO:29-56, which encodes PMOD. The polynucleotide sequences of SEQ ID NO:29-56, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses variants of a polynucleotide encoding PMOD. In particular, such a variant polynucleotide will have at least about 70%, or alternatively at least about 85%, or even at least about 95% polynucleotide sequence identity to a polynucleotide encoding PMOD. A particular aspect of the invention encompasses a variant of a polynucleotide comprising a sequence selected from the group consisting of SEQ ID NO:29-56 which has at least about 70%, or alternatively at least about 85%, or even at least about 95% polynucleotide sequence identity to a nucleic acid sequence selected from the group consisting of SEQ ID NO:29-56. Any one of the polynucleotide variants described above can encode a polypeptide which contains at least one functional or structural characteristic of PMOD.

In addition, or in the alternative, a polynucleotide variant of the invention is a splice variant of a polynucleotide encoding PMOD. A splice variant may have portions which have significant sequence identity to a polynucleotide encoding PMOD, but will generally have a greater or lesser number of polynucleotides due to additions or deletions of blocks of sequence arising from alternate splicing of exons during mRNA processing. A splice variant may have less than about 70%, or alternatively less than about 60%, or alternatively less than about 50% polynucleotide sequence identity to a polynucleotide encoding PMOD over its entire length; however, portions of the splice variant will have at least about 70%, or alternatively at least about 85%, or alternatively at least about 95%, or alternatively 100% polynucleotide sequence identity to portions of the polynucleotide encoding PMOD. For example, a polynucleotide comprising a sequence of SEQ ID NO:31 is a splice variant of a polynucleotide comprising a sequence of SEQ ID NO:34, and a polynucleotide comprising a sequence of SEQ ID NO:44 is a splice variant of a polynucleotide comprising a sequence of SEQ ID NO:56. Any one of the splice variants described above can encode a polypeptide which contains at least one functional or structural characteristic of PMOD.

It will be appreciated by those skilled in the art that as a result of the degeneracy of the genetic code, a multitude of polynucleotide sequences encoding PMOD, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring PMOD, and all such variations are to be considered as being specifically disclosed.

Although polynucleotides which encode PMOD and its variants are generally capable of hybridizing to polynucleotides encoding naturally occurring PMOD under appropriately selected conditions of stringency, it may be advantageous to produce polynucleotides encoding PMOD or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding PMOD and its derivatives without altering the encoded amino acid sequences include the production of RNA transcripts having more desirable properties, such as a greater half-life, than transcripts produced from the naturally occurring sequence.

The invention also encompasses production of polynucleotides which encode PMOD and PMOD derivatives, or fragments thereof, entirely by synthetic chemistry. After production, the synthetic polynucleotide may be inserted into any of the many available expression vectors and cell systems using reagents well known in the art. Moreover, synthetic chemistry may be used to introduce mutations into a polynucleotide encoding PMOD or any fragment thereof.

Embodiments of the invention can also include polynucleotides that are capable of hybridizing to the claimed polynucleotides, and, in particular, to those having the sequences shown in SEQ ID NO:29-56 and fragments thereof, under various conditions of stringency. (See, e.g., Wahl, G. M. and S. L. Berger (1987) Methods Enzymol. 152:399-407; Kimmel, A. R. (1987) Methods Enzymol. 152:507-511.) Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase I, SEQUENASE (US Biochemical, Cleveland Ohio), Taq polymerase (Applied Biosystems), thermostable T7 polymerase (Amersham Biosciences, Piscataway N.J.), or combinations of polymerases and proofreading exonucleases such as those found in the ELONGASE amplification system (Invitrogen, Carlsbad Calif.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Amersham Biosciences), or other systems known in the art. The resulting sequences are analyzed using a variety of algorithms which are well known in the art. (See, e.g., Ausubel, F. M. (1997) Short Protocols in Molecular Biology, John Wiley & Sons, New York N.Y., unit 7.7; Meyers, R. A. (1995) Molecular Biology and Biotechnology, Wiley VCH, New York N.Y., pp. 856-853.)

The nucleic acids encoding PMOD may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector. (See, e.g., Sarkar, G. (1993) PCR Methods Applic. 2:318-322.) Another method, inverse PCR, uses primers that extend in divergent directions to amplify unknown sequence from a circularized template. The template is derived from restriction fragments comprising a known genomic locus and surrounding sequences. (See, e.g., Triglia, T. et al. (1988) Nucleic Acids Res. 16:8186.) A third method, capture PCR, involves PCR amplification of DNA fragments adjacent to known sequences in human and yeast artificial chromosome DNA. (See, e.g., Lagerstrom, M. et al. (1991) PCR Methods Applic. 1:111-119.) In this method, multiple restriction enzyme digestions and ligations may be used to insert an engineered double-stranded sequence into a region of unknown sequence before performing PCR. Other methods which may be used to retrieve unknown sequences are known in the art. (See, e.g., Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotides or fragments thereof which encode PMOD may be cloned in recombinant DNA molecules that direct expression of PMOD, or fragments or functional equivalents thereof, in appropriate host cells. Due to the inherent degeneracy of the genetic code, other polynucleotides which encode substantially the same or a functionally equivalent polypeptides may be produced and used to express PMOD.

The polynucleotides of the invention can be engineered using methods generally known in the art in order to alter PMOD-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C.-C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of PMOD, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, polynucleotides encoding PMOD may be synthesized, in whole or in part, using one or more chemical methods well known in the art. (See, e.g., Caruthers, M. H. et al. (1980) Nucleic Acids Symp. Ser. 7:215-223; and Horn, T. et al. (1980) Nucleic Acids Symp. Ser. 7:225-232.) Alternatively, PMOD itself or a fragment thereof may be synthesized using chemical methods known in the art. For example, peptide synthesis can be performed using various solution-phase or solid-phase techniques. (See, e.g., Creighton, T. (1984) Proteins, Structures and Molecular Properties, WH Freeman, New York N.Y., pp. 55-60; and Roberge, J. Y. et al. (1995) Science 269:202-204.) Automated synthesis may be achieved using the ABI 431A peptide synthesizer (Applied Biosystems). Additionally, the amino acid sequence of PMOD, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography. (See, e.g., Chiez, R. M. and F. Z. Regnier (1990) Methods Enzymol. 182:392-421.) The composition of the synthetic peptides may be confirmed by amino acid analysis or by sequencing. (See, e.g., Creighton, supra, pp. 28-53.)

In order to express a biologically active PMOD, the polynucleotides encoding PMOD or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotides encoding PMOD. Such elements may vary in their strength and specificity. Specific initiation signals may also be used to achieve more efficient translation of polynucleotides encoding PMOD. Such signals include the ATG initiation codon and adjacent sequences, e.g. the Kozak sequence. In cases where a polynucleotide sequence encoding PMOD and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used. (See, e.g., Scharf, D. et al. (1994) Results Probl. Cell Differ. 20:125-162.)

Methods which are well known to those skilled in the art may be used to construct expression vectors containing polynucleotides encoding PMOD and appropriate transcriptional and translational control elements. These methods include in vitro recombinant DNA techniques, synthetic techniques, and in vivo genetic recombination. (See, e.g., Sambrook, J. et al. (1989) Molecular Cloning. A Laboratory Manual, Cold Spring Harbor Press, Plainview N.Y., ch. 4, 8, and 16-17; Ausubel, F. M. et al. (1995) Current Protocols in Molecular Biology, John Wiley & Sons, New York N.Y., ch. 9, 13, and 16.)

A variety of expression vector/host systems may be utilized to contain and express polynucleotides encoding PMOD. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems. (See, e.g., Sambrook, supra; Ausubel, supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; and Harrington, J. J. et al. (1997) Nat. Genet. 15:345-355.) Expression vectors derived from retroviruses, adenoviruses, or herpes or vaccinia viruses, or from various bacterial plasmids, may be used for delivery of polynucleotides to the targeted organ, tissue, or cell population. (See, e.g., Di Nicola, M. et al. (1998) Cancer Gen. Ther. 5(6):350-356; Yu, M. et al. (1993) Proc. Natl. Acad. Sci. USA 90(13):6340-6344; Buller, R. M. et al. (1985) Nature 317(6040):813-815; McGregor, D. P. et al. (1994) Mol. Immunol. 31(3):219-226; and Verma, I. M. and N. Somia (1997) Nature 389:239-242.) The invention is not limited by the host cell employed.

In bacterial systems, a number of cloning and expression vectors may be selected depending upon the use intended for polynucleotides encoding PMOD. For example, routine cloning, subcloning, and propagation of polynucleotides encoding PMOD can be achieved using a multifunctional E. coli vector such as PBLUESCRIPT (Stratagene, La Jolla Calif.) or PSPORT1 plasmid (Invitrogen). Ligation of polynucleotides encoding PMOD into the vector's multiple cloning site disrupts the lacZ gene, allowing a colorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence. (See, e.g., Van Heeke, G. and S. M. Schuster (1989) J. Biol. Chem. 264:5503-5509.) When large quantities of PMOD are needed, e.g. for the production of antibodies, vectors which direct high level expression of PMOD may be used. For example, vectors containing the strong, inducible SP6 or T7 bacteriophage promoter may be used.

Yeast expression systems may be used for production of PMOD. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign polynucleotide sequences into the host genome for stable propagation. (See, e.g., Ausubel, 1995, supra; Bitter, G. A. et al. (1987) Methods Enzymol. 153:516-544; and Scorer, C. A. et al. (1994) Bio/Technology 12:181-184.)

Plant systems may also be used for expression of PMOD. Transcription of polynucleotides encoding PMOD may be driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used. (See, e.g., Coruzzi, G. et al. (1984) EMBO J. 3:1671-1680; Broglie, R. et al. (1984) Science 224:838-843; and Winter, J. et al. (1991) Results Probl. Cell Differ. 17:85-105.) These constructs can be introduced into plant cells by direct DNA transformation or pathogen-mediated transfection. (See, e.g., The McGraw Hill Yearbook of Science and Technology (1992) McGraw Hill, New York N.Y., pp. 191-196.)

In mammalian cells, a number of viral-based expression systems may be utilized. In cases where an adenovirus is used as an expression vector, polynucleotides encoding PMOD may be ligated into an adenovirus transcription/translation complex consisting of the late promoter and tripartite leader sequence. Insertion in a non-essential E1 or E3 region of the viral genome may be used to obtain infective virus which expresses PMOD in host cells. (See, e.g., Logan, J. and T. Shenk (1984) Proc. Natl. Acad. Sci. USA 81:3655-3659.) In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes. (See, e.g., Harrington, J. J. et al. (1997) Nat. Genet. 15:345-355.)

For long term production of recombinant proteins in mammalian systems, stable expression of PMOD in cell lines is preferred. For example, polynucleotides encoding PMOD can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk⁻ and apr⁻ cells, respectively. (See, e.g., Wigler, M. et al. (1977) Cell 11:223-232; Lowy, I. et al. (1980) Cell 22:817-823.) Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulfuron and phosphinotricin acetyltransferase, respectively. (See, e.g., Wigler, M. et al. (1980) Proc. Natl. Acad. Sci. USA 77:3567-3570; Colbere-Garapin, F. et al. (1981) J. Mol. Biol. 150:1-14.) Additional selectable genes have been described, e.g., trpB and hisD, which alter cellular requirements for metabolites. (See, e.g., Hartman, S. C. and R. C. Mulligan (1988) Proc. Natl. Acad. Sci. USA 85:8047-8051.) Visible markers, e.g., anthocyanins, green fluorescent proteins (GFP; Clontech), β glucuronidase and its substrate β-glucuronide, or luciferase and its substrate luciferin may be used. These markers can be used not only to identify transformants, but also to quantify the amount of transient or stable protein expression attributable to a specific vector system. (See, e.g., Rhodes, C. A. (1995) Methods Mol. Biol. 55:121-131.)

Although the presence/absence of marker gene expression suggests that the gene of interest is also present, the presence and expression of the gene may need to be confirmed. For example, if the sequence encoding PMOD is inserted within a marker gene sequence, transformed cells containing polynucleotides encoding PMOD can be identified by the absence of marker gene function. Alternatively, a marker gene can be placed in tandem with a sequence encoding PMOD under the control of a single promoter. Expression of the marker gene in response to induction or selection usually indicates expression of the tandem gene as well.

In general, host cells that contain the polynucleotide encoding PMOD and that express PMOD may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of PMOD using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on PMOD is preferred, but a competitive binding assay may be employed. These and other assays are well known in the art. (See, e.g., Hampton, R. et al. (1990) Serological Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; and Pound, J. D. (1998) Immunochemical Protocols, Humana Press, Totowa N.J.)

A wide variety of labels and conjugation techniques are known by those skilled in the art and may be used in various nucleic acid and amino acid assays. Means for producing labeled hybridization or PCR probes for detecting sequences related to polynucleotides encoding PMOD include oligolabeling, nick translation, end-labeling, or PCR amplification using a labeled nucleotide. Alternatively, polynucleotides encoding PMOD, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Biosciences, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with polynucleotides encoding PMOD may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode PMOD may be designed to contain signal sequences which direct secretion of PMOD through a prokaryotic or eukaryotic cell membrane.

In addition, a host cell strain may be chosen for its ability to modulate expression of the inserted polynucleotides or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and WI38) are available from the American Type Culture Collection (ATCC, Manassas Va.) and may be chosen to ensure the correct modification and processing of the foreign protein.

In another embodiment of the invention, natural, modified, or recombinant polynucleotides encoding PMOD may be ligated to a heterologous sequence resulting in translation of a fusion protein in any of the aforementioned host systems. For example, a chimeric PMOD protein containing a heterologous moiety that can be recognized by a commercially available antibody may facilitate the screening of peptide libraries for inhibitors of PMOD activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the PMOD encoding sequence and the heterologous protein sequence, so that PMOD may be cleaved away from the heterologous moiety following purification. Methods for fusion protein expression and purification are discussed in Ausubel (1995, supra, ch. 10). A variety of commercially available kits may also be used to facilitate expression and purification of fusion proteins.

In another embodiment, synthesis of radiolabeled PMOD may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

PMOD, fragments of PMOD, or variants of PMOD may be used to screen for compounds that specifically bind to PMOD. One or more test compounds may be screened for specific binding to PMOD. In various embodiments, 1, 2, 3, 4, 5, 10, 20, 50, 100, or 200 test compounds can be screened for specific binding to PMOD. Examples of test compounds can include antibodies, anticalins, oligonucleotides, proteins (e.g., ligands or receptors), or small molecules.

In related embodiments, variants of PMOD can be used to screen for binding of test compounds, such as antibodies, to PMOD, a variant of PMOD, or a combination of PMOD and/or one or more variants PMOD. In an embodiment, a variant of PMOD can be used to screen for compounds that bind to a variant of PMOD, but not to PMOD having the exact sequence of a sequence of SEQ ID NO:1-28. PMOD variants used to perform such screening can have a range of about 50% to about 99% sequence identity to PMOD, with various embodiments having 60%, 70%, 75%, 80%, 85%, 90%, and 95% sequence identity.

In an embodiment, a compound identified in a screen for specific binding to PMOD can be closely related to the natural ligand of PMOD, e.g., a ligand or fragment thereof, a natural substrate, a structural or functional mimetic, or a natural binding partner. (See, e.g., Coligan, J. E. et al. (1991) Current Protocols in Immunology 1(2):Chapter 5.) In another embodiment, the compound thus identified can be a natural ligand of a receptor PMOD. (See, e.g., Howard, A. D. et al. (2001) Trends Pharmacol. Sci.22: 132-140; Wise, A. et al. (2002) Drug Discovery Today 7:235-246.)

In other embodiments, a compound identified in a screen for specific binding to PMOD can be closely related to the natural receptor to which PMOD binds, at least a fragment of the receptor, or a fragment of the receptor including all or a portion of the ligand binding site or binding pocket. For example, the compound may be a receptor for PMOD which is capable of propagating a signal, or a decoy receptor for PMOD which is not capable of propagating a signal (Ashkenazi, A. and V. M. Divit (1999) Curr. Opin. Cell Biol. 11:255-260; Mantovani, A. et al. (2001) Trends Immunol. 22:328-336). The compound can be rationally designed using known techniques. Examples of such techniques include those used to construct the compound etanercept (ENBREL; Immunex Corp., Seattle Wash.), which is efficacious for treating rheumatoid arthritis in humans. Etanercept is an engineered p75 tumor necrosis factor (TNF) receptor dimer linked to the Fc portion of human IgG₁ (Taylor, P. C. et al. (2001) Curr. Opin. Immunol. 13:611-616).

In one embodiment, two or more antibodies having similar or, alternatively, different specificities can be screened for specific binding to PMOD, fragments of PMOD, or variants of PMOD. The binding specificity of the antibodies thus screened can thereby be selected to identify particular fragments or variants of PMOD. In one embodiment, an antibody can be selected such that its binding specificity allows for preferential identification of specific fragments or variants of PMOD. In another embodiment, an antibody can be selected such that its binding specificity allows for preferential diagnosis of a specific disease or condition having increased, decreased, or otherwise abnormal production of PMOD.

In an embodiment, anticalins can be screened for specific binding to PMOD, fragments of PMOD, or variants of PMOD. Anticalins are ligand-binding proteins that have been constructed based on a lipocalin scaffold (Weiss, G. A. and H. B. Lowman (2000) Chem. Biol. 7:R177-R184; Skerra, A. (2001) J. Biotechnol. 74:257-275). The protein architecture of lipocalins can include a beta-barrel having eight antiparallel beta-strands, which supports four loops at its open end. These loops form the natural ligand-binding site of the lipocalins, a site which can be re-engineered in vitro by amino acid substitutions to impart novel binding specificities. The amino acid substitutions can be made using methods known in the art or described herein, and can include conservative substitutions (e.g., substitutions that do not alter binding specificity) or substitutions that modestly, moderately, or significantly alter binding specificity.

In one embodiment, screening for compounds which specifically bind to, stimulate, or inhibit PMOD involves producing appropriate cells which express PMOD, either as a secreted protein or on the cell membrane. Preferred cells include cells from mammals, yeast, Drosophila, or E. coli. Cells expressing PMOD or cell membrane fractions which contain PMOD are then contacted with a test compound and binding, stimulation, or inhibition of activity of either PMOD or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with PMOD, either in solution or affixed to a solid support, and detecting the binding of PMOD to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

An assay can be used to assess the ability of a compound to bind to its natural ligand and/or to inhibit the binding of its natural ligand to its natural receptors. Examples of such assays include radio-labeling assays such as those described in U.S. Pat. No. 5,914,236 and U.S. Pat. No. 6,372,724. In a related embodiment, one or more amino acid substitutions can be introduced into a polypeptide compound (such as a receptor) to improve or alter its ability to bind to its natural ligands. (See, e.g., Matthews, D. J. and J. A. Wells. (1994) Chem. Biol. 1:25-30.) In another related embodiment, one or more amino acid substitutions can be introduced into a polypeptide compound (such as a ligand) to improve or alter its ability to bind to its natural receptors. (See, e.g., Cunningham, B. C. and J. A. Wells (1991) Proc. Natl. Acad. Sci. USA 88:3407-3411; Lowman, H. B. et al. (1991) J. Biol. Chem. 266:10982-10988.)

PMOD, fragments of PMOD, or variants of PMOD may be used to screen for compounds that modulate the activity of PMOD. Such compounds may include agonists, antagonists, or partial or inverse agonists. In one embodiment, an assay is performed under conditions permissive for PMOD activity, wherein PMOD is combined with at least one test compound, and the activity of PMOD in the presence of a test compound is compared with the activity of PMOD in the absence of the test compound. A change in the activity of PMOD in the presence of the test compound is indicative of a compound that modulates the activity of PMOD. Alternatively, a test compound is combined with an in vitro or cell-free system comprising PMOD under conditions suitable for PMOD activity, and the assay is performed. In either of these assays, a test compound which modulates the activity of PMOD may do so indirectly and need not come in direct contact with the test compound. At least one and up to a plurality of test compounds may be screened.

In another embodiment, polynucleotides encoding PMOD or their mammalian homologs may be “knocked out” in an animal model system using homologous recombination in embryonic stem (ES) cells. Such techniques are well known in the art and are useful for the generation of animal models of human disease. (See, e.g., U.S. Pat. No. 5,175,383 and U.S. Pat. No. 5,767,337.) For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding PMOD may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding PMOD can also be used to create “knockin” humanized animals (pigs) or transgenic animals (mice or rats) to model human disease. With knockin technology, a region of a polynucleotide encoding PMOD is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress PMOD, e.g., by secreting PMOD in its milk, may also serve as a convenient source of that protein (Janne, J. et al. (1998) Biotechnol. Annu. Rev. 4:55-74).

### THERAPEUTICS

Chemical and structural similarity, e.g., in the context of sequences and motifs, exists between regions of PMOD and protein modification and maintenance molecules. In addition, the expression of PMOD is closely associated with epithilial, brain, brain tumor, ileum, lymph node, liver, ovarian, placental, prostate, cerebellum, pituitary gland, small intestine, and testis tissues and promonocyte cells. Further examples of tissues expressing PMOD can be found in Table 6 and can also be found in Example XI. Therefore, PMOD appears to play a role in gastrointestinal, cardiovascular, autoimmune/inflammatory, cell proliferative, developmental, epithelial, neurological, and reproductive disorders. In the treatment of disorders associated with increased PMOD expression or activity, it is desirable to decrease the expression or activity of PMOD. In the treatment of disorders associated with decreased PMOD expression or activity, it is desirable to increase the expression or activity of PMOD.

Therefore, in one embodiment, PMOD or a fragment or derivative thereof may be administered to a subject to treat or prevent a disorder associated with decreased expression or activity of PMOD. Examples of such disorders include, but are not limited to, a gastrointestinal disorder, such as dysphagia, peptic esophagitis, esophageal spasm, esophageal stricture, esophageal carcinoma, dyspepsia, indigestion, gastritis, gastric carcinoma, anorexia, nausea, emesis, gastroparesis, antral or pyloric edema, abdominal angina, pyrosis, gastroenteritis, intestinal obstruction, infections of the intestinal tract, peptic ulcer, cholelithiasis, cholecystitis, cholestasis, pancreatitis, pancreatic carcinoma, biliary tract disease, hepatitis, hyperbilirubinemia, cirrhosis, passive congestion of the liver, hepatoma, infectious colitis, ulcerative colitis, ulcerative proctitis, Crohn's disease, Whipple's disease, Mallory-Weiss syndrome, colonic carcinoma, colonic obstruction, irritable bowel syndrome, short bowel syndrome, diarrhea, constipation, gastrointestinal hemorrhage, acquired immunodeficiency syndrome (AIDS) enteropathy, jaundice, hepatic encephalopathy, hepatorenal syndrome, hepatic steatosis, hemochromatosis, Wilson's disease, alpha₁-antitrypsin deficiency, Reye's syndrome, primary sclerosing cholangitis, liver infarction, portal vein obstruction and thrombosis, centrilobular necrosis, peliosis hepatis, hepatic vein thrombosis, veno-occlusive disease, preeclampsia, eclampsia, acute fatty liver of pregnancy, intrahepatic cholestasis of pregnancy, and hepatic tumors including nodular hyperplasias, adenomas, and carcinomas; a cardiovascular disorder, such as arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery, congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, and complications of cardiac transplantation; an autoimmune/inflammatory disease, such as acquired immunodeficiency syndrome (AIDS), Addison's disease, adult respiratory distress syndrome, allergies, ankylosing spondylitis, amyloidosis, anemia, asthma, atherosclerosis, atherosclerotic plaque rupture, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, degradation of articular cartilage, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; a developmental disorder, such as renal tubular acidosis, anemia, Cushing's syndrome, achondroplastic dwarfism, Duchenne and Becker muscular dystrophy, bone resorption, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, age-related macular degeneration, and sensorineural hearing loss; an epithelial disorder, such as dyshidrotic eczema, allergic contact dermatitis, keratosis pilaris, melasma, vitiligo, actinic keratosis, basal cell carcinoma, squamous cell carcinoma, seborrheic keratosis, folliculitis, herpes simplex, herpes zoster, varicella, candidiasis, dermatophytosis, scabies, insect bites, cherry angioma, keloid, dermatofibroma, acrochordons, urticaria, transient acantholytic dermatosis, xerosis, eczema, atopic dermatitis, contact dermatitis, hand eczema, nummular eczema, lichen simplex chronicus, asteatotic eczema, stasis denratitis and stasis ulceration, seborrheic dermatitis, psoriasis, lichen planus, pityriasis rosea, impetigo, ecthyma, dermatophytosis, tinea versicolor, warts, acne vulgaris, acne rosacea, pemphigus vulgaris, pemphigus foliaceus, paraneoplastic pemphigus, bullous pemphigoid, herpes gestationis, dermatitis herpetiformis, linear IgA disease, epidermolysis bullosa acquisita, dermatomyositis, lupus erythematosus, scleroderma and morphea, erythroderma, alopecia, figurate skin lesions, telangiectasias, hypopigmentation, hyperpigmentation, vesicles/bullae, exanthems, cutaneous drug reactions, papulonodular skin lesions, chronic non-healing wounds, photosensitivity diseases, epidermolysis bullosa simplex, epidermolytic hyperkeratosis, epidermolytic and nonepidermolytic palmoplantar keratoderma, ichthyosis bullosa of Siemens, ichthyosis exfoliativa, keratosis palmaris et plantaris, keratosis palmoplantaris, palmoplantar keratoderma, keratosis punctata, Meesmann's corneal dystrophy, pachyonychia congenita, white sponge nevus, steatocystoma multiplex, epidermal nevi/epidermolytic hyperkeratosis type, monilethrix, trichothiodystrophy, chronic hepatitis/cryptogenic cirrhosis, and colorectal hyperplasia; a neurological disorder, such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; and a reproductive disorder, such as infertility, including tubal disease, ovulatory defects, and endometriosis, a disorder of prolactin production, a disruption of the estrous cycle, a disruption of the menstrual cycle, polycystic ovary syndrome, ovarian hyperstimulation syndrome, an endometrial or ovarian tumor, a uterine fibroid, autoimmune disorders, an ectopic pregnancy, and teratogenesis; cancer of the breast, fibrocystic breast disease, and galactorrhea; a disruption of spermatogenesis, abnormal sperm physiology, cancer of the testis, cancer of the prostate, benign prostatic hyperplasia, prostatitis, Peyronie's disease, impotence, carcinoma of the male breast, and gynecomastia.

In another embodiment, a vector capable of expressing PMOD or a fragment or derivative thereof may be administered to a subject to treat or prevent a disorder associated with decreased expression or activity of PMOD including, but not limited to, those described above.

In a further embodiment, a composition comprising a substantially purified PMOD in conjunction with a suitable pharmaceutical carrier may be administered to a subject to treat or prevent a disorder associated with decreased expression or activity of PMOD including, but not limited to, those provided above.

In still another embodiment, an agonist which modulates the activity of PMOD may be administered to a subject to treat or prevent a disorder associated with decreased expression or activity of PMOD including, but not limited to, those listed above.

In a further embodiment, an antagonist of PMOD may be administered to a subject to treat or prevent a disorder associated with increased expression or activity of PMOD. Examples of such disorders include, but are not limited to, those gastrointestinal, cardiovascular, autoimmune/inflammatory, cell proliferative, developmental, epithelial, neurological, and reproductive disorders described above. In one aspect, an antibody which specifically binds PMOD may be used directly as an antagonist or indirectly as a targeting or delivery mechanism for bringing a pharmaceutical agent to cells or tissues which express PMOD.

In an additional embodiment, a vector expressing the complement of the polynucleotide encoding PMOD may be administered to a subject to treat or prevent a disorder associated with increased expression or activity of PMOD including, but not limited to, those described above.

In other embodiments, any protein, agonist, antagonist, antibody, complementary sequence, or vector embodiments may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of PMOD may be produced using methods which are generally known in the art. In particular, purified PMOD may be used to produce antibodies or to screen libraries of pharmaceutical agents to identify those which specifically bind PMOD. Antibodies to PMOD may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are generally preferred for therapeutic use. Single chain antibodies (e.g., from camels or llamas) may be potent enzyme inhibitors and may have advantages in the design of peptide mimetics, and in the development of immuno-adsorbents and biosensors (Muyldermans, S. (2001) J. Biotechnol. 74:277-302).

For the production of antibodies, various hosts including goats, rabbits, rats, mice, camels, dromedaries, llamas, humans, and others may be immunized by injection with PMOD or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to PMOD have an amino acid sequence consisting of at least about 5 amino acids, and generally will consist of at least about 10 amino acids. It is also preferable that these oligopeptides, peptides, or fragments are identical to a portion of the amino acid sequence of the natural protein. Short stretches of PMOD amino acids may be fused with those of another protein, such as KLH, and antibodies to the chimeric molecule may be produced.

Monoclonal antibodies to PMOD may be prepared using any technique which provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; and Cole, S. P. et al. (1984) Mol. Cell Biol. 62:109-120.)

In addition, techniques developed for the production of “chimeric antibodies,” such as the splicing of mouse antibody genes to human antibody genes to obtain a molecule with appropriate antigen specificity and biological activity, can be used. (See, e.g., Morrison, S. L. et al. (1984) Proc. Natl. Acad. Sci. USA 81:6851-6855; Neuberger, M. S. et al. (1984) Nature 312:604-608; and Takeda, S. et al. (1985) Nature 314:452-454.) Alternatively, techniques described for the production of single chain antibodies may be adapted, using methods known in the art, to produce PMOD-specific single chain antibodies. Antibodies with related specificity, but of distinct idiotypic composition, may be generated by chain shuffling from random combinatorial immunoglobulin libraries. (See, e.g., Burton, D. R. (1991) Proc. Natl. Acad. Sci. USA 88:10134-10137.) Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. USA 86:3833-3837; Winter, G. et al. (1991) Nature 349:293-299.)

Antibody fragments which contain specific binding sites for PMOD may also be generated. For example, such fragments include, but are not limited to, F(ab′)₂ fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity. (See, e.g., Huse, W. D. et al. (1989) Science 246:1275-1281.)

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between PMOD and its specific antibody. A two-site, monoclonal-based immunoassay utilizing monoclonal antibodies reactive to two non-interfering PMOD epitopes is generally used, but a competitive binding assay may also be employed (Pound, supra).

Various methods such as Scatchard analysis in conjunction with radioimmunoassay techniques may be used to assess the affinity of antibodies for PMOD. Affinity is expressed as an association constant, Kₐ, which is defined as the molar concentration of PMOD-antibody complex divided by the molar concentrations of free antigen and free antibody under equilibrium conditions. The Kₐ determined for a preparation of polyclonal antibodies, which are heterogeneous in their affinities for multiple PMOD epitopes, represents the average affinity, or avidity, of the antibodies for PMOD. The Kₐ determined for a preparation of monoclonal antibodies, which are monospecific for a particular PMOD epitope, represents a true measure of affinity. High-affinity antibody preparations with Kₐ ranging from about 10⁹ to 10¹² L/mole are preferred for use in immunoassays in which the PMOD-antibody complex must withstand rigorous manipulations. Low-affinity antibody preparations with Kₐ ranging from about 10⁶ to 10⁷ L/mole are preferred for use in immunopurification and similar procedures which ultimately require dissociation of PMOD, preferably in active form, from the antibody (Catty, D. (1988) Antibodies, Volume I: A Practical Approach, IRL Press, Washington D.C.; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of PMOD-antibody complexes. Procedures for evaluating antibody specificity, titer, and avidity, and guidelines for antibody quality and usage in various applications, are generally available. (See, e.g., Catty, supra, and Coligan et al. supra.)

In another embodiment of the invention, polynucleotides encoding PMOD, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding PMOD. Such technology is well known in the art, and antisense oligonucleotides or larger fragments can be designed from various locations along the coding or control regions of sequences encoding PMOD. (See, e.g., Agrawal, S., ed. (1996) Antisense Therapeutics, Humana Press Inc., Totawa N.J.)

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein. (See, e.g., Slater, J. E. et al. (1998) J. Allergy Clin. Immunol. 102(3):469-475; and Scanlon, K. J. et al. (1995) 9(13): 1288-1296.) Antisense sequences can also be introduced intracellularly through the use of viral vectors, such as retrovirus and adeno-associated virus vectors. (See, e.g., Miller, A. D. (1990) Blood 76:271; Ausubel, supra; Uckert, W. and W. Walther (1994) Pharmacol. Ther. 63(3):323-347.) Other gene delivery mechanisms include liposome-derived systems, artificial viral envelopes, and other systems known in the art. (See, e.g., Rossi, J. J. (1995) Br. Med. Bull. 51(1):217-225; Boado, R. J. et al. (1998) J. Pharm. Sci. 87(11):1308-1315; and Morris, M. C. et al. (1997) Nucleic Acids Res. 25(14):2730-2736.)

In another embodiment of the invention, polynucleotides encoding PMOD may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475-480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassamias, familial hypercholesterolemia, and hemophilia resulting from Factor VIII or Factor IX deficiencies (Crystal, R. G. (1995) Science 270:404-410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA 93:11395-11399), hepatitis B or C virus (HBV, HCV); fungal parasites, such as Candida albicans and Paracoccidioides brasiliensis; and protozoan parasites such as Plasmodium falciparum and Trypanosoma cruzi). In the case where a genetic deficiency in PMOD expression or regulation causes disease, the expression of PMOD from an appropriate population of transduced cells may alleviate the clinical manifestations caused by the genetic deficiency.

In a further embodiment of the invention, diseases or disorders caused by deficiencies in PMOD are treated by constructing mammalian expression vectors encoding PMOD and introducing these vectors by mechanical means into PMOD-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J-L. and H. Récipon (1998) Curr. Opin. Biotechnol. 9:445-450).

Expression vectors that may be effective for the expression of PMOD include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (Clontech, Palo Alto Calif.). PMOD may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451-456), commercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, F. M. V. and H. M. Blau, supra)), or (iii) a tissue-specific promoter or the native promoter of the endogenous gene encoding PMOD from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to PMOD expression are treated by constructing a retrovirus vector consisting of (i) the polynucleotide encoding PMOD under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. USA 95:1201-1206; Su, L. (1997) Blood 89:2283-2290).

In an embodiment, an adenovirus-based gene therapy delivery system is used to deliver polynucleotides encoding PMOD to cells which have one or more genetic abnormalities with respect to the expression of PMOD. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999) Annu. Rev. Nutr. 19:511-544 and Verma, I. M. and N. Somia (1997) Nature 18:389:239-242, both incorporated by reference herein.

In another embodiment, a herpes-based, gene therapy delivery system is used to deliver polynucleotides encoding PMOD to target cells which have one or more genetic abnormalities with respect to the expression of PMOD. The use of herpes simplex virus (HSV)-based vectors may be especially valuable for introducing PMOD to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999) J. Virol. 73:519-532 and Xu, H. et al. (1994) Dev. Biol. 163:152-161, hereby incorporated by reference. The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another embodiment, an alphavirus (positive, single-stranded RNA virus) vector is used to deliver polynucleotides encoding PMOD to target cells. The biology of the prototypic alphavirus, Semliki Forest Virus (SFV), has been studied extensively and gene transfer vectors have been based on the SFV genome (Garoff, H. and K.-J. Li (1998) Curr. Opin. Biotechnol. 9:464-469). During alphavirus RNA replication, a subgenomic RNA is generated that normally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for PMOD into the alphavirus genome in place of the capsid-coding region results in the production of a large number of PMOD-coding RNAs and the synthesis of high levels of PMOD in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of PMOD into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee, J. E. et al. (1994) in Huber, B. E. and B. I. Carr, Molecular and Immunologic Approaches, Futura Publishing, Mt. Kisco N.Y., pp. 163-177.) A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of RNA molecules encoding PMOD.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA molecules encoding PMOD. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

An additional embodiment of the invention encompasses a method for screening for a compound which is effective in altering expression of a polynucleotide encoding PMOD. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased PMOD expression or activity, a compound which specifically inhibits expression of the polynucleotide encoding PMOD may be therapeutically useful, and in the treatment of disorders associated with decreased PMOD expression or activity, a compound which specifically promotes expression of the polynucleotide encoding PMOD may be therapeutically useful.

At least one, and up to a plurality, of test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding PMOD is exposed to at least one test compound thus obtained. The sample may comprise, for example, an intact or permeabilized cell, or an in vitro cell-free or reconstituted biochemical system. Alterations in the expression of a polynucleotide encoding PMOD are assayed by any method commonly known in the art. Typically, the expression of a specific nucleotide is detected by hybridization with a probe having a nucleotide sequence complementary to the sequence of the polynucleotide encoding PMOD. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art. (See, e.g., Goldman, C. K. et al. (1997) Nat. Biotechnol. 15:462-466.)

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of PMOD, antibodies to PMOD, and mimetics, agonists, antagonists, or inhibitors of PMOD.

The compositions utilized in this invention may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery has the advantage of administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising PMOD or fragments thereof. For example, liposome preparations containing a cell-impermeable macromolecule may promote cell fusion and intracellular delivery of the macromolecule. Alternatively, PMOD or a fragment thereof may be joined to a short cationic N-terminal portion from the HIV Tat-1 protein. Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example PMOD or fragments thereof, antibodies of PMOD, and agonists, antagonists or inhibitors of PMOD, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

### DIAGNOSTICS

In another embodiment, antibodies which specifically bind PMOD may be used for the diagnosis of disorders characterized by expression of PMOD, or in assays to monitor patients being treated with PMOD or agonists, antagonists, or inhibitors of PMOD. Antibodies useful for diagnostic purposes may be prepared in the same manner as described above for therapeutics. Diagnostic assays for PMOD include methods which utilize the antibody and a label to detect PMOD in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring PMOD, including ELISAs, RIAs, and FACS, are known in the art and provide a basis for diagnosing altered or abnormal levels of PMOD expression. Normal or standard values for PMOD expression are established by combining body fluids or cell extracts taken from normal mammalian subjects, for example, human subjects, with antibodies to PMOD under conditions suitable for complex formation. The amount of standard complex formation may be quantitated by various methods, such as photometric means. Quantities of PMOD expressed in subject, control, and disease samples from biopsied tissues are compared with the standard values. Deviation between standard and subject values establishes the parameters for diagnosing disease.

In another embodiment of the invention, polynucleotides encoding PMOD may be used for diagnostic purposes. The polynucleotides which may be used include oligonucleotides, complementary RNA and DNA molecules, and PNAs. The polynucleotides may be used to detect and quantify gene expression in biopsied tissues in which expression of PMOD may be correlated with disease. The diagnostic assay may be used to determine absence, presence, and excess expression of PMOD, and to monitor regulation of PMOD levels during therapeutic intervention.

In one aspect, hybridization with PCR probes which are capable of detecting polynucleotides, including genomic sequences, encoding PMOD or closely related molecules may be used to identify nucleic acid sequences which encode PMOD. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding PMOD, allelic variants, or related sequences.

Probes may also be used for the detection of related sequences, and may have at least 50% sequence identity to any of the PMOD encoding sequences. The hybridization probes of the subject invention may be DNA or RNA and may be derived from the sequence of SEQ ID NO:29-56 or from genomic sequences including promoters, enhancers, and introns of the PMOD gene.

Means for producing specific hybridization probes for polynucleotides encoding PMOD include the cloning of polynucleotides encoding PMOD or PMOD derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotides encoding PMOD may be used for the diagnosis of disorders associated with expression of PMOD. Examples of such disorders include, but are not limited to, a gastrointestinal disorder, such as dysphagia, peptic esophagitis, esophageal spasm, esophageal stricture, esophageal carcinoma, dyspepsia, indigestion, gastritis, gastric carcinoma, anorexia, nausea, emesis, gastroparesis, antral or pyloric edema, abdominal angina, pyrosis, gastroenteritis, intestinal obstruction, infections of the intestinal tract, peptic ulcer, cholelithiasis, cholecystitis, cholestasis, pancreatitis, pancreatic carcinoma, biliary tract disease, hepatitis, hyperbilirubinemia, cirrhosis, passive congestion of the liver, hepatoma, infectious colitis, ulcerative colitis, ulcerative proctitis, Crohn's disease, Whipple's disease, Mallory-Weiss syndrome, colonic carcinoma, colonic obstruction, irritable bowel syndrome, short bowel syndrome, diarrhea, constipation, gastrointestinal hemorrhage, acquired immunodeficiency syndrome (AIDS) enteropathy, jaundice, hepatic encephalopathy, hepatorenal syndrome, hepatic steatosis, hemochromatosis, Wilson's disease, alpha₁-antitrypsin deficiency, Reye's syndrome, primary sclerosing cholangitis, liver infarction, portal vein obstruction and thrombosis, centrilobular necrosis, peliosis hepatis, hepatic vein thrombosis, veno-occlusive disease, preeclampsia, eclampsia, acute fatty liver of pregnancy, intrahepatic cholestasis of pregnancy, and hepatic tumors including nodular hyperplasias, adenomas, and carcinomas; a cardiovascular disorder, such as arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery, congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, and complications of cardiac transplantation; an autoimmune/inflammatory disease, such as acquired immunodeficiency syndrome (AIDS), Addison's disease, adult respiratory distress syndrome, allergies, ankylosing spondylitis, amyloidosis, anemia, asthma, atherosclerosis, atherosclerotic plaque rupture, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, degradation of articular cartilage, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; a developmental disorder, such as renal tubular acidosis, anemia, Cushing's syndrome, achondroplastic dwarfism, Duchenne and Becker muscular dystrophy, bone resorption, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, age-related macular degeneration, and sensorineural hearing loss; an epithelial disorder, such as dyshidrotic eczema, allergic contact dermatitis, keratosis pilaris, melasma, vitiligo, actinic keratosis, basal cell carcinoma, squamous cell carcinoma, seborrheic keratosis, folliculitis, herpes simplex, herpes zoster, varicella, candidiasis, dermatophytosis, scabies, insect bites, cherry angioma, keloid, dermatofibroma, acrochordons, urticaria, transient acantholytic dermatosis, xerosis, eczema, atopic dermatitis, contact dermatitis, hand eczema, nummular eczema, lichen simplex chronicus, asteatotic eczema, stasis dermatitis and stasis ulceration, seborrheic dermatitis, psoriasis, lichen planus, pityriasis rosea, impetigo, ecthyma, dermatophytosis, tinea versicolor, warts, acne vulgaris, acne rosacea, pemphigus vulgaris, pemphigus foliaceus, paraneoplastic pemphigus, bullous pemphigoid, herpes gestationis, dermatitis herpetiformis, linear IgA disease, epidermolysis bullosa acquisita, dermatomyositis, lupus erythematosus, scleroderma and morphea, erythroderma, alopecia, figurate skin lesions, telangiectasias, hypopigmentation, hyperpigmentation, vesicles/bullae, exanthems, cutaneous drug reactions, papulonodular skin lesions, chronic non-healing wounds, photosensitivity diseases, epidermolysis bullosa simplex, epidermolytic hyperkeratosis, epidermolytic and nonepidermolytic palmoplantar keratoderma, ichthyosis bullosa of Siemens, ichthyosis exfoliativa, keratosis palmaris et plantaris, keratosis palmoplantaris, palmoplantar keratoderma, keratosis punctata, Meesmann's corneal dystrophy, pachyonychia congenita, white sponge nevus, steatocystoma multiplex, epidermal nevi/epidermolytic hyperkeratosis type, monilethrix, trichothiodystrophy, chronic hepatitis/cryptogenic cirrhosis, and colorectal hyperplasia; a neurological disorder, such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; and a reproductive disorder, such as infertility, including tubal disease, ovulatory defects, and endometriosis, a disorder of prolactin production, a disruption of the estrous cycle, a disruption of the menstrual cycle, polycystic ovary syndrome, ovarian hyperstimulation syndrome, an endometrial or ovarian tumor, a uterine fibroid, autoimmune disorders, an ectopic pregnancy, and teratogenesis; cancer of the breast, fibrocystic breast disease, and galactorrhea; a disruption of spermatogenesis, abnormal sperm physiology, cancer of the testis, cancer of the prostate, benign prostatic hyperplasia, prostatitis, Peyronie's disease, impotence, carcinoma of the male breast, and gynecomastia. Polynucleotides encoding PMOD may be used in Southern or northern analysis, dot blot, or other membrane-based technologies; in PCR technologies; in dipstick, pin, and multiformat ELISA-like assays; and in microarrays utilizing fluids or tissues from patients to detect altered PMOD expression. Such qualitative or quantitative methods are well known in the art.

In a particular aspect, polynucleotides encoding PMOD may be used in assays that detect the presence of associated disorders, particularly those mentioned above. Polynucleotides complementary to sequences encoding PMOD may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of polynucleotides encoding PMOD in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of PMOD, a normal or standard profile for expression is established. This may be accomplished by combining body fluids or cell extracts taken from normal subjects, either animal or human, with a sequence, or a fragment thereof, encoding PMOD, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the cancer.

Additional diagnostic uses for oligonucleotides designed from the sequences encoding PMOD may involve the use of PCR. These oligomers may be chemically synthesized, generated enzymatically, or produced in vitro. Oligomers will preferably contain a fragment of a polynucleotide encoding PMOD, or a fragment of a polynucleotide complementary to the polynucleotide encoding PMOD, and will be employed under optimized conditions for identification of a specific gene or condition. Oligomers may also be employed under less stringent conditions for detection or quantification of closely related DNA or RNA sequences.

In a particular aspect, oligonucleotide primers derived from polynucleotides encoding PMOD may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from polynucleotides encoding PMOD are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (isSNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

SNPs may be used to study the genetic basis of human disease. For example, at least 16 common SNPs have been associated with non-insulin-dependent diabetes mellitus. SNPs are also useful for examining differences in disease outcomes in monogenic disorders, such as cystic fibrosis, sickle cell anemia, or chronic granulomatous disease. For example, variants in the mannose-binding lectin, MBL2, have been shown to be correlated with deleterious pulmonary outcomes in cystic fibrosis. SNPs also have utility in pharmacogenomics, the identification of genetic variants that influence a patient's response to a drug, such as life-threatening toxicity. For example, a variation in N-acetyl transferase is associated with a high incidence of peripheral neuropathy in response to the anti-tuberculosis drug isoniazid, while a variation in the core promoter of the ALOX5 gene results in diminished clinical response to treatment with an anti-asthma drug that targets the 5-lipoxygenase pathway. Analysis of the distribution of SNPs in different populations is useful for investigating genetic drift, mutation, recombination, and selection, as well as for tracing the origins of populations and their migrations. (Taylor, J. G. et al. (2001) Trends Mol. Med. 7:507-512; Kwok, P.-Y. and Z. Gu (1999) Mol. Med. Today 5:538-543; Nowotny, P. et al. (2001) Curr. Opin. Neurobiol. 11:637-641.)

Methods which may also be used to quantify the expression of PMOD include radiolabeling or biotinylating nucleotides, coamplification of a control nucleic acid, and interpolating results from standard curves. (See, e.g., Melby, P. C. et al. (1993) J. Immunol. Methods 159:235-244; Duplaa, C. et al. (1993) Anal. Biochem. 212:229-236.) The speed of quantitation of multiple samples may be accelerated by running the assay in a high-throughput format where the oligomer or polynucleotide of interest is presented in various dilutions and a spectrophotometric or colorimetric response gives rapid quantitation.

In further embodiments, oligonucleotides or longer fragments derived from any of the polynucleotides described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, PMOD, fragments of PMOD, or antibodies specific for PMOD may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time. (See Seilhamer et al., “Comparative Gene Transcript Analysis,” U.S. Pat. No. 5,840,484, expressly incorporated by reference herein.) Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity. (See, for example, Press Release 00-02 from the National Institute of Environmental Health Sciences, released Feb. 29, 2000, available at http://www.niehs.nih.gov/oc/news/toxchip.htm.) Therefore, it is important and desirable in toxicological screening using toxicant signatures to include all expressed gene sequences.

In an embodiment, the toxicity of a test compound can be assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another embodiment relates to the use of the polypeptides disclosed herein to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of interest. In some cases, further sequence data may be obtained for definitive protein identification.

A proteomic profile may also be generated using antibodies specific for PMOD to quantify the levels of PMOD expression. In one embodiment, the antibodies are used as elements on a microarray, and protein expression levels are quantified by exposing the microarray to the sample and detecting the levels of protein bound to each array element (Lueking, A. et al. (1999) Anal. Biochem. 270:103-111; Mendoze, L. G. et al. (1999) Biotechniques 27:778-788). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art. (See, e.g., Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; and Heller, M. J. et al. (1997) U.S. Pat. No. 5,605,662.) Various types of microarrays are well known and thoroughly described in DNA Microarrays: A Practical Approach, M. Schena, ed. (1999) Oxford University Press, London.

In another embodiment of the invention, nucleic acid sequences encoding PMOD may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries. (See, e.g., Harrington, J. J. et al. (1997) Nat. Genet. 15:345-355; Price, C. M. (1993) Blood Rev. 7:127-134; and Trask, B. J. (1991) Trends Genet. 7:149-154.) Once mapped, the nucleic acid sequences may be used to develop genetic linkage maps, for example, which correlate the inheritance of a disease state with the inheritance of a particular chromosome region or restriction fragment length polymorphism (RFLP). (See, for example, Lander, E. S. and D. Botstein (1986) Proc. Natl. Acad. Sci. USA 83:7353-7357.)

Fluorescent in situ hybridization (FISH) may be correlated with other physical and genetic map data. (See, e.g., Heinz-Ulrich, et al. (1995) in Meyers, supra, pp. 965-968.) Examples of genetic map data can be found in various scientific journals or at the Online Mendelian Inheritance in Man (OMIM) World Wide Web site. Correlation between the location of the gene encoding PMOD on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation. (See, e.g., Gatti, R. A. et al. (1988) Nature 336:577-580.) The nucleotide sequence of the instant invention may also be used to detect differences in the chromosomal location due to translocation, inversion, etc., among normal, carrier, or affected individuals.

In another embodiment of the invention, PMOD, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between PMOD and the agent being tested may be measured.

Another technique for drug screening provides for high throughput screening of compounds having suitable binding affinity to the protein of interest. (See, e.g., Geysen, et al. (1984) PCT application WO84/03564.) In this method, large numbers of different small test compounds are synthesized on a solid substrate. The test compounds are reacted with PMOD, or fragments thereof, and washed. Bound PMOD is then detected by methods well known in the art. Purified PMOD can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding PMOD specifically compete with a test compound for binding PMOD. In this manner, antibodies can be used to detect the presence of any peptide which shares one or more antigenic determinants with PMOD.

In additional embodiments, the nucleotide sequences which encode PMOD may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

The disclosures of all patents, applications, and publications mentioned above and below, including U.S. Ser. No. 60/300,508, U.S. Ser. No. 60/303,445, U.S. Ser. No. 60/305,405, U.S. Ser. No. 60/311,442, U.S. Ser. No. 60/314,821, U.S. Ser. No. 60/315,992, and U.S. Ser. No. 60/378/205, are hereby expressly incorporated by reference.

### EXAMPLES

I. Construction of cDNA Libraries

Incyte cDNAs were derived from cDNA libraries described in the LIFESEQ GOLD database (Incyte Genomics, Palo Alto Calif.). Some tissues were homogenized and lysed in guanidinium isothiocyanate, while others were homogenized and lysed in phenol or in a suitable mixture of denaturants, such as TRIZOL (Invitrogen), a monophasic solution of phenol and guanidine isothiocyanate. The resulting lysates were centrifuged over CsCl cushions or extracted with chloroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A)+ RNA was isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Invitrogen), using the recommended procedures or similar methods known in the art. (See, e.g., Ausubel, 1997, supra, units 5.1-6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Biosciences) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Invitrogen), PCDNA2.1 plasmid (Invitrogen, Carlsbad Calif.), PBK-CMV plasmid (Stratagene), PCR2-TOPOTA plasmid (Invitrogen), PCMV-ICIS plasmid (Stratagene), pIGEN (Incyte Genomics, Palo Alto Calif.), pRARE (Incyte Genomics), or pINCY (Incyte Genomics), or derivatives thereof. Recombinant plasmids were transformed into competent E. coli cells including XL1-Blue, XL1-BlueMRF, or SOLR from Stratagene or DH5α, DH10B, or ElectroMAX DH10B from Invitrogen.

II. Isolation of cDNA Clones

Plasmids obtained as described in Example I were recovered from host cells by in vivo excision using the UNIZAP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a FLUOROSKAN II fluorescence scanner (Labsystems Oy, Helsinki, Finland).

III. Sequencing and Analysis

Incyte cDNA recovered in plasmids as described in Example II were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions were prepared using reagents provided by Amersham Biosciences or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Amersham Biosciences); the ABI PRISM 373 or 377 sequencing system (Applied Biosystems) in conjunction with standard ABI protocols and base calling software; or other sequence analysis systems known in the art. Reading frames within the cDNA sequences were identified using standard methods (reviewed in Ausubel, 1997, supra, unit 7.7). Some of the cDNA sequences were selected for extension using the techniques disclosed in Example VIII.

The polynucleotide sequences derived from Incyte cDNAs were validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof were then queried against a selection of public databases such as the GenBank primate, rodent, mammalian, vertebrate, and eukaryote databases, and BLOCKS, PRINTS, DOMO, PRODOM; PROTEOME databases with sequences from Homo sapiens, Ratuus norvegicus, Mus musculus, Caenorhabditis elegans, Saccharomyces cerevisiae, Schizosaccharomyces pombe, and Candida albicans (Incyte Genomics, Palo Alto Calif.); hidden Markov model (HMM)-based protein family databases such as PFAM, INCY, and TIGRFAM (Haft, D. H. et al. (2001) Nucleic Acids Res. 29:41-43); and HMM-based protein domain databases such as SMART (Schultz et al. (1998) Proc. Natl. Acad. Sci. USA 95:5857-5864; Letunic, I. et al. (2002) Nucleic Acids Res. 30:242-244). (HMM is a probabilistic approach which analyzes consensus primary structures of gene families. See, for example, Eddy, S. R. (1996) Curr. Opin. Struct. Biol. 6:361-365.) The queries were performed using programs based on BLAST, FASTA, BLIMPS, and HMMER. The Incyte cDNA sequences were assembled to produce full length polynucleotide sequences. Alternatively, GenBank cDNAs, GenBank ESTs, stitched sequences, stretched sequences, or Genscan-predicted coding sequences (see Examples IV and V) were used to extend Incyte cDNA assemblages to full length. Assembly was performed using programs based on Phred, Phrap, and Consed, and cDNA assemblages were screened for open reading frames using programs based on GeneMark, BLAST, and FASTA. The full length polynucleotide sequences were translated to derive the corresponding full length polypeptide sequences. Alternatively, a polypeptide may begin at any of the methionine residues of the full length translated polypeptide. Full length polypeptide sequences were subsequently analyzed by querying against databases such as the GenBank protein databases (genpept), SwissProt, the PROTEOME databases, BLOCKS, PRINTS, DOMO, PRODOM, Prosite, hidden Markov model (HMM)-based protein family databases such as PFAM, INCY, and TIGRFAM; and HMM-based protein domain databases such as SMART. Full length polynucleotide sequences are also analyzed using MACDNASIS PRO software (Hitachi Software Engineering, South San Francisco Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 summarizes the tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences were also used to identify polynucleotide sequence fragments from SEQ ID NO:29-56. Fragments from about 20 to about 4000 nucleotides which are useful in hybridization and amplification technologies are described in Table 4, column 2.

IV. Identification and Editing of Coding Sequences from Genomic DNA

Putative protein modification and maintenance molecules were initially identified by running the Genscan gene identification program against public genomic sequence databases (e.g., gbpri and gbhtg). Genscan is a general-purpose gene identification program which analyzes genomic DNA sequences from a variety of organisms (See Burge, C. and S. Karlin (1997) J. Mol. Biol. 268:78-94, and Burge, C. and S. Karlin (1998) Curr. Opin. Struct. Biol. 8:346-354). The program concatenates predicted exons to form an assembled cDNA sequence extending from a methionine to a stop codon. The output of Genscan is a FASTA database of polynucleotide and polypeptide sequences. The maximum range of sequence for Genscan to analyze at once was set to 30 kb. To determine which of these Genscan predicted cDNA sequences encode protein modification and maintenance molecules, the encoded polypeptides were analyzed by querying against PFAM models for protein modification and maintenance molecules. Potential protein modification and maintenance molecules were also identified by homology to Incyte cDNA sequences that had been annotated as protein modification and maintenance molecules. These selected Genscan-predicted sequences were then compared by BLAST analysis to the genpept and gbpri public databases. Where necessary, the Genscan-predicted sequences were then edited by comparison to the top BLAST hit from genpept to correct errors in the sequence predicted by Genscan, such as extra or omitted exons. BLAST analysis was also used to find any Incyte cDNA or public cDNA coverage of the Genscan-predicted sequences, thus providing evidence for transcription. When Incyte cDNA coverage was available, this information was used to correct or confirm the Genscan predicted sequence. Full length polynucleotide sequences were obtained by assembling Genscan-predicted coding sequences with Incyte cDNA sequences and/or public cDNA sequences using the assembly process described in Example III. Alternatively, full length polynucleotide sequences were derived entirely from edited or unedited Genscan-predicted coding sequences.

V. Assembly of Genomic Sequence Data with cDNA Sequence Data

“Stitched” Sequences

Partial cDNA sequences were extended with exons predicted by the Genscan gene identification program described in Example IV. Partial cDNAs assembled as described in Example III were mapped to genomic DNA and parsed into clusters containing related cDNAs and Genscan exon predictions from one or more genomic sequences. Each cluster was analyzed using an algorithm based on graph theory and dynamic programming to integrate cDNA and genomic information, generating possible splice variants that were subsequently confirmed, edited, or extended to create a full length sequence. Sequence intervals in which the entire length of the interval was present on more than one sequence in the cluster were identified, and intervals thus identified were considered to be equivalent by transitivity. For example, if an interval was present on a cDNA and two genomic sequences, then all three intervals were considered to be equivalent. This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genomic sequence). The resultant stitched sequences were translated and compared by BLAST analysis to the genpept and gbpri public databases. Incorrect exons predicted by Genscan were corrected by comparison to the top BLAST hit from genpept. Sequences were further extended with additional cDNA sequences, or by inspection of genomic DNA, when necessary.

“Stretched” Sequences

Partial DNA sequences were extended to full length with an algorithm based on BLAST analysis. First, partial cDNAs assembled as described in Example III were queried against public databases such as the GenBank primate, rodent, mammalian, vertebrate, and eukaryote databases using the BLAST program. The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

VI. Chromosomal Mapping of PMOD Encoding Polynucleotides

The sequences which were used to assemble SEQ ID NO:29-56 were compared with sequences from the Incyte LIFESEQ database and public domain databases using BLAST and other implementations of the Smith-Waterman algorithm. Sequences from these databases that matched SEQ ID NO:29-56 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Généthon were used to determine if any of the clustered sequences had been previously mapped. Inclusion of a mapped sequence in a cluster resulted in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Généthon which provide boundaries for radiation hybrid markers whose sequences were included in each of the clusters. Human genome maps and other resources available to the public, such as the NCBI “GeneMap'99” World Wide Web site (http://www.ncbi.nlm.nih.gov/genemap/), can be employed to determine if previously identified disease genes map within or in proximity to the intervals indicated above.

VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound. (See, e.g., Sambrook, supra, ch. 7; Ausubel (1995) supra, ch. 4 and 16.)

Analogous computer techniques applying BLAST were used to search for identical or related molecules in cDNA databases such as GenBank or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch. Two sequences may share more than one HSP (separated by gaps). If there is more than one HSP, then the pair with the highest BLAST score is used to calculate the product score. The product score represents a balance between fractional overlap and quality in a. BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotides encoding PMOD are analyzed with respect to the tissue sources from which they were derived. For example, some full length sequences are assembled, at least in part, with overlapping Incyte cDNA sequences (see Example III). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding PMOD. cDNA sequences and cDNA library/tissue information are found in the LIFESEQ GOLD database (Incyte Genomics, Palo Alto Calif.).

VIII. Extension of PMOD Encoding Polynucleotides

Full length polynucleotides are produced by extension of an appropriate fragment of the full length molecule using oligonucleotide primers designed from this fragment. One primer was synthesized to initiate 5′ extension of the known fragment, and the other primer was synthesized to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄, and 2-mercaptoethanol, Taq DNA polymerase (Amersham Biosciences), ELONGASE enzyme (Invitrogen), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene Oreg.) dissolved in 1×TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Biosciences). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Biosciences), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2× carb liquid media.

The cells were lysed, and DNA was amplified by PCR using Taq DNA polymerase (Amersham Biosciences) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Biosciences) or the ABI PRISM BIGDYE Terminator cycle sequencing ready reaction kit (Applied Biosystems).

In like manner, full length polynucleotides are verified using the above procedure or are used to obtain 5′ regulatory sequences using the above procedure along with oligonucleotides designed for such extension, and an appropriate genomic library.

IX. Identification of Single Nucleotide Polymorphisms in PMOD Encoding Polynucleotides

Common DNA sequence variants known as single nucleotide polymorphisms (SNPs) were identified in SEQ ID NO:29-56 using the LIFESEQ database (Incyte Genomics). Sequences from the same gene were clustered together and assembled as described in Example III, allowing the identification of all sequence variants in the gene. An algorithm consisting of a series of filters was used to distinguish SNPs from other sequence variants. Preliminary filters removed the majority of basecall errors by requiring a minimum Phred quality score of 15, and removed sequence alignment errors and errors resulting from improper trimming of vector sequences, chimeras, and splice variants. An automated procedure of advanced chromosome analysis analysed the original chromatogram files in the vicinity of the putative SNP. Clone error filters used statistically generated algorithms to identify errors introduced during laboratory processing, such as those caused by reverse transcriptase, polymerase, or somatic mutation. Clustering error filters used statistically generated algorithms to identify errors resulting from clustering of close homologs or pseudogenes, or due to contamination by non-human sequences. A final set of filters removed duplicates and SNPs found in immunoglobulins or T-cell receptors.

Certain SNPs were selected for further characterization by mass spectrometry using the high throughput MASSARRAY system (Sequenom, Inc.) to analyze allele frequencies at the SNP sites in four different human populations. The Caucasian population comprised 92 individuals (46 male, 46 female), including 83 from Utah, four French, three Venezualan, and two Amish individuals. The African population comprised 194 individuals (97 male, 97 female), all African Americans. The Hispanic population comprised 324 individuals (162 male, 162 female), all Mexican Hispanic. The Asian population comprised 126 individuals (64 male, 62 female) with a reported parental breakdown of 43% Chinese, 31% Japanese, 13% Korean, 5% Vietnamese, and 8% other Asian. Allele frequencies were first analyzed in the Caucasian population; in some cases those SNPs which showed no allelic variance in this population were not further tested in the other three populations.

X. Labeling and Use of Individual Hybridization Probes

Hybridization probes derived from SEQ ID NO:29-56 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Biosciences), and T4 polynucleotide kinase (DuPont NEN, Boston Mass.). The labeled oligonucleotides are substantially purified using a SEPHADEX G-25 superfine size exclusion dextran bead column (Amersham Biosciences). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under conditions of up to, for example, 0.1×saline sodium citrate and 0.5% sodium dodecyl sulfate. Hybridization patterns are visualized using autoradiography or an alternative imaging means and compared.

XI. Microarrays

The linkage or synthesis of array elements upon a microarray can be achieved utilizing photolithography, piezoelectric printing (ink-jet printing, See, e.g., Baldeschweiler, supra.), mechanical microspotting technologies, and derivatives thereof. The substrate in each of the aforementioned technologies should be uniform and solid with a non-porous surface (Schena (1999), supra). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements. (See, e.g., Schena, M. et al. (1995) Science 270:467-470; Shalon, D. et al. (1996) Genome Res. 6:639-645; Marshall, A. and J. Hodgson (1998) Nat. Biotechnol. 16:27-31.)

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray may be assessed. In one embodiment, microarray preparation and usage is described in detail below.

Tissue or Cell Sample Preparation

Total RNA is isolated from tissue samples using the guanidinium thiocyanate method and poly(A)⁺ RNA is purified using the oligo-(dT) cellulose method. Each poly(A)⁺ RNA sample is reverse transcribed using MMLV reverse-transcriptase, 0.05 pg/μl oligo-(dT) primer (21 mer), 1× first strand buffer, 0.03 units/μl RNase inhibitor, 500 μM dATP, 500 μM dGTP, 500 μM dTTP, 40 μM dCTP, 40 μM dCTP-Cy3 (BDS) or dCTP-Cy5 (Amersham Biosciences). The reverse transcription reaction is performed in a 25 ml volume containing 200 ng poly(A)⁺ RNA with GEMBRIGHT kits (Incyte). Specific control poly(A)⁺ RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (CLONTECH Laboratories, Inc. (CLONTECH), Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Biosciences).

Purified array elements are immobilized on polymer-coated glass slides. Glass microscope slides (Coming) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Microarrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 μl of sample mixture consisting of 0.2 μg each of Cy3 and Cy5 labeled cDNA synthesis products in 5×SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20× microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte). Array elements that exhibited at least about a two-fold change in expression, a signal-to-background ratio of at least 2.5, and an element spot size of at least 40% were identified as differentially expressed using the GEMTOOLS program (Incyte Genomics).

Expression

The human C3A cell line is a clonal derivative of HepG2/C3 (hepatoma cell line, isolated from a 15-year-old male with liver tumor), which was selected for strong contact inhibition of growth. The use of a clonal population enhances the reproducibility of the cells. C3A cells have many characteristics of primary human hepatocytes in culture: i) expression of insulin receptor and insulin-like growth factor II receptor; ii) secretion of a high ratio of serum albumin compared with α-fetoprotein; iii) conversion of ammonia to urea and glutamine; iv) metabolism of aromatic amino acids; and v) proliferation in glucose-free and insulin-free medium. The C3A cell line is now well established as an in vitro model of the mature human liver (Mickelson et al. (1995) Hepatology 22:866-875; Nagendra et al. (1997) Am. J. Physiol. 272:G408-G416). The expression of SEQ ID NO:29 was altered by a factor of 2 or more in cells treated with a variety of steroids including prednisone, dexamethasone, medroxyprogesterone, budesonide, and beclomthasone. In addition, the expression of SEQ ID NO:29 was was altered by a factor of two or more in C3A cells. Therefore, SEQ ID NO:29 can be used in assays related to treatment for cell proliferative disorders.

For example, SEQ ID NO:31 and SEQ ID NO:34 showed differential expression in breast tumor cell lines versus normal breast epithelial cells as determined by microarray analysis. The expression of SEQ ID NO:31 and SEQ ID NO:34 was decreased by at least two fold in breast tumor cell lines that were harvested from donors with both early and late stages of tumor progression and malignant transformation. Therefore, SEQ ID NO:31 and SEQ ID NO:34 can be used in diagnostic assays for breast cancer.

In another example, SEQ ID NO:33 showed differential expression in response to several compounds which produce known metabolic and toxicological responses. The expression of SEQ ID NO:33 was reduced by at least two fold in the human C3A liver cell line incubated for varying lengths of time with compounds including fenofibrate, clofibrate, dexamethasone, beclomethasone, medroxyprogesterone, budesonide, and betamethasone. Therefore, SEQ ID NO:33 can be used in toxicology testing.

SEQ ID NO:35 showed differential expression in human peripheral blood mononuclear cells (PBMCs) following exposure to 5 and 25 μM prednisone for 24 hours. Prednisone is a corticosteroid that is metabolized in the liver to its active form, prednisolone. Prednisone is approximately four times more potent as a glucocorticoid than hydrocortisone. Glucocorticoids are naturally occurring hormones that prevent or suppress inflammation and immune responses when administered at pharmacologic doses. At the molecular level, unbound glucocorticoids readily cross cell membranes and bind with high affinity to specific cytoplasmic receptors. Subsequent to binding, transcription and, ultimately, protein synthesis are affected. The result can include inhibition of leukocyte infiltration at the site of inflammation, interference in the function of mediators of the inflammatory response, and suppression of humoral immune responses. PBMCs can be classified into discrete cellular populations representing the major cellular components of the immune system. PBMCs contain about 52% lymphocytes (12% B lymphocytes, 40% T lymphocytes {25% CD4+ and 15% CD8+}), 20% NK cells, 25% monocytes, and 3% various cells that include dendritic cells and progenitor cells. These cells were pooled from the blood of 6 healthy volunteer donors. The expression of SEQ ID NO:35 was decreased by at least two-fold in prednisone-treated (5 and 25 μM) cells as compared to untreated controls.

SEQ ID NO:44 showed differential expression in normal tissue versus tissue affected by prostate carcinoma by microarray analysis. Expression of SEQ ID NO:44 in a primary prostate epithelial cell line (PrEC) isolated from a normal donor was compared to expression of SEQ ID NO:44 in a prostate carcinoma cell line isolated (LNCaP) from a lymph node biopsy of a 50-year-old male with metastatic prostate carcinoma. Expression of SEQ ID NO:44 was decreased by at least two-fold in the cell line affected by prostate carcinoma. In addition, expression of SEQ ID NO:44 in peripheral blood mononuclear cells isolated from a pool of healthy donors was decreased by at least two-fold by treatment with 1 ng/ml IL12 for 24 hours. The expression of SEQ ID NO:44 was also shown to be differentially expressed in a comparison of breast cells lines by microarray analysis. In four of the seven breast cancer cell lines tested, expression of SEQ ID NO:44 was shown to be decreased by at least two-fold when compared to normal mammary epithelial cells (HMEC), indicating the use of SEQ ID NO:44 as a diagnostic marker, for disease staging, and as a therapeutic target for protease-associated diseases including prostate and breast cancer.

For example, SEQ ID NO:51 showed differential expression in toxicology studies as determined by microarray analysis. The expression of SEQ ID NO:51 was decreased by at least two fold in a human C3A liver cell line treated with various drugs (e.g., steroids, steroid hormones) relative to untreated C3A cells. The human C3A cell line is a clonal derivative of HepG2/C3 (hepatoma cell line, isolated from a 15-year-old male with liver tumor), which was selected for strong contact inhibition of growth. The C3A cell line is well established as an in vitro model of the mature human liver (Mickelson et al. (1995) Hepatology 22:866-875; Nagendra et al. (1997) Am J Physiol 272:G408-G416). Effects upon liver metabolism are important to understanding the pharmacodynamics of a drug. Therefore, SEQ ID NO:51 can be used for understanding the pharmacodynamics of a drug.

In another example, SEQ ID NO:55 showed differential expression in lung adenocarcinoma versus normal lung tissues as determined by microarray analysis. The expression of SEQ ID NO:55 was decreased by at least two fold in lung adenocarcinoma relative to grossly uninvolved normal lung tissue from the same donor. Therefore, SEQ ID NO:55 can be used as a diagnostic marker for disease staging or as a potential therapeutic target for lung cancer.

As another example, SEQ ID NO:56 is downregulated in breast cancer cell lines versus nonmalignant mammary epithelial cells, as determined by microarray analysis. In one experiment, gene expression profiles of nonmalignant mammary epithelial cells were compared to gene expression profiles of various breast carcinoma lines at different stages of tumor progression. The cells were grown in defined serum-free H14 medium to 70-80% confluence prior to RNA harvest. Cell lines compared included: a) HMEC, a primary breast epithelial cell line isolated from a normal donor, b) MCF-10A, a breast mammary gland cell line isolated from a 36-year-old woman with fibrocystic breast disease, c) MCF7, a nonmalignant breast adenocarcinoma cell line isolated from the pleural effusion of a 69-year-old female, d) T-47D, a breast carcinoma cell line isolated from a pleural effusion obtained from a 54-year-old female with an infiltrating ductal carcinoma of the breast, e) Sk-BR-3, a breast adenocarcinoma cell line isolated from a malignant pleural effusion of a 43-year-old female, f) BT-20, a breast carcinoma cell line derived in vitro from cells emigrating out of thin slices of the tumor mass isolated from a 74-year-old female, g) MDA-mb-231, a breast tumor cell line isolated from the pleural effusion of a 51-year-old female, and h) MDA-mb-435S, a spindle-shaped strain that evolved from the parent line (435) isolated by R. Cailleau from pleural effusion of a 31-year-old female with metastatic, ductal adenocarcinoma of the breast. Therefore, SEQ ID NO:56 can be used in monitoring treatment of, and diagnostic assays for, breast cancer.

As another example, SEQ ID NO:56 is downregulated in prostate carcinomas versus primary prostate epithelial cells, as determined by microarray analysis. Primary prostate epithelial cells were compared with prostate carcinomas representative of the different stages of tumor progression. Cell lines compared included: a) PrEC, a primary prostate epithelial cell line isolated from a normal donor, b) DU 145, a prostate carcinoma cell line isolated from a metastatic site in the brain of 69-year old male with widespread metastatic prostate carcinoma, c) LNCaP, a prostate carcinoma cell line isolated from a lymph node biopsy of a 50-year-old male with metastatic prostate carcinoma, and d) PC-3, a prostate adenocarcinoma cell line isolated from a metastatic site in the bone of a 62-year-old male with grade IV prostate adenocarcinoma. In one experiment, cells were grown in basal media in the absence of growth factors and hormones. In a second experiment, cells were grown under optimal growth conditions, in the presence of growth factors and nutrients. Cells grown under restrictive conditions were compared to normal PrECs grown under restrictive conditions. Therefore, SEQ ID NO:56 can be used in monitoring treatment of, and diagnostic assays for, prostate cancer.

XII. Complementary Polynucleotides

Sequences complementary to the PMOD-encoding sequences, or any parts thereof, are used to detect, decrease, or inhibit expression of naturally occurring PMOD. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of PMOD. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the PMOD-encoding transcript.

XIII. Expression of PMOD

Expression and purification of PMOD is achieved using bacterial or virus-based expression systems. For expression of PMOD in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express PMOD upon induction with isopropyl beta-D-thiogalactopyranoside (IPTG). Expression of PMOD in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding PMOD by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frupiperda (Sf9) insect cells in most cases, or human hepatocytes, in some cases. Infection of the latter requires additional genetic modifications to baculovirus. (See Engelhard, E. K. et al. (1994) Proc. Natl. Acad. Sci. USA 91:3224-3227; Sandig, V. et al. (1996) Hum. Gene Ther. 7:1937-1945.)

In most expression systems, PMOD is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Biosciences). Following purification, the GST moiety can be proteolytically cleaved from PMOD at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel (1995, supra, ch. 10 and 16). Purified PMOD obtained by these methods can be used directly in the assays shown in Examples XVII, XVIII, and XIX, where applicable.

XIV. Functional Assays

PMOD function is assessed by expressing the sequences encoding PMOD at physiologically elevated levels in mammalian cell culture systems. cDNA is subcloned into a mammalian expression vector containing a strong promoter that drives high levels of cDNA expression. Vectors of choice include PCMV SPORT plasmid (Invitrogen, Carlsbad Calif.) and PCR3.1 plasmid (Invitrogen), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometry, Oxford, New York N.Y.

The influence of PMOD on gene expression can be assessed using highly purified populations of cells transfected with sequences encoding PMOD and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding PMOD and other genes of interest can be analyzed by northern analysis or microarray techniques.

XV. Production of PMOD Specific Antibodies

PMOD substantially purified using polyacrylamide gel electrophoresis (PAGE; see, e.g., Harrington, M. G. (1990) Methods Enzymol. 182:488-495), or other purification techniques, is used to immunize animals (e.g., rabbits, mice, etc.) and to produce antibodies using standard protocols.

Alternatively, the PMOD amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art. (See, e.g., Ausubel, 1995, supra, ch. 11.)

Typically, oligopeptides of about 15 residues in length are synthesized using an ABI 431A peptide synthesizer (Applied Biosystems) using FMOC chemistry and coupled to KLH (Sigma-Aldrich, St. Louis Mo.) by reaction with N-maleimidobenzoyl-N-hydroxysuccinimide ester (MBS) to increase immunogenicity. (See, e.g., Ausubel, 1995, supra.) Rabbits are immunized with the oligopeptide-KLH complex in complete Freund's adjuvant. Resulting antisera are tested for antipeptide and anti-PMOD activity by, for example, binding the peptide or PMOD to a substrate, blocking with 1% BSA, reacting with rabbit antisera, washing, and reacting with radio-iodinated goat anti-rabbit IgG.

XVI. Purification of Naturally Occurring PMOD Using Specific Antibodies

Naturally occurring or recombinant PMOD is substantially purified by immunoaffinity chromatography using antibodies specific for PMOD. An immunoaffinity column is constructed by covalently coupling anti-PMOD antibody to an activated chromatographic resin, such as CNBr-activated SEPHAROSE (Amersham Biosciences). After the coupling, the resin is blocked and washed according to the manufacturer's instructions.

Media containing PMOD are passed over the immunoaffinity column, and the column is washed under conditions that allow the preferential absorbance of PMOD (e.g., high ionic strength buffers in the presence of detergent). The column is eluted under conditions that disrupt antibody/PMOD binding (e.g., a buffer of pH 2 to pH 3, or a high concentration of a chaotrope, such as urea or thiocyanate ion), and PMOD is collected.

XVII. Identification of Molecules which Interact with PMOD

PMOD, or biologically active fragments thereof, are labeled with ¹²⁵I Bolton-Hunter reagent. (See, e.g., Bolton, A. E. and W. M. Hunter (1973) Biochem. J. 133:529-539.) Candidate molecules previously arrayed in the wells of a multi-well plate are incubated with the labeled PMOD, washed, and any wells with labeled PMOD complex are assayed. Data obtained using different concentrations of PMOD are used to calculate values for the number, affinity, and association of PMOD with the candidate molecules.

Alternatively, molecules interacting with PMOD are analyzed using the yeast two-hybrid system as described in Fields, S. and O. Song (1989) Nature 340:245-246, or using commercially available kits based on the two-hybrid system, such as the MATCHMAKER system (Clontech).

PMOD may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

XVII. Demonstration of PMOD Activity

PMOD activity can be demonstrated using a generic immunoblotting strategy or through a variety of specific activity assays, some of which are outlined below. As a general approach, cell lines or tissues transformed with a vector containing PMOD coding sequences can be assayed for PMOD activity by immunoblotting. Transformed cells are denatured in SDS in the presence of β-mercaptoethanol, nucleic acids are removed by ethanol precipitation, and proteins are purified by acetone precipitation. Pellets are resuspended in 20 mM Tris buffer at pH 7.5 and incubated with Protein G-Sepharose pre-coated with an antibody specific for PMOD. After washing, the Sepharose beads are boiled in electrophoresis sample buffer, and the eluted proteins subjected to SDS-PAGE. The SDS-PAGE is transferred to a membrane for immunoblotting, and the PMOD activity is assessed by visualizing and quantifying bands on the blot using the antibody specific for PMOD as the primary antibody and ¹²⁵I-labeled IgG specific for the primary antibody as the secondary antibody.

PMOD kinase activity is measured by quantifying the phosphorylation of a protein substrate by PMOD in the presence of gamma-labeled ³²P-ATP. PMOD is incubated with the protein substrate, ³²P-ATP, and an appropriate kinase buffer. The ³²P incorporated into the substrate is separated from free ³²P-ATP by electrophoresis and the incorporated ³²P is counted using a radioisotope counter. The amount of incorporated ³²P is proportional to the activity of PMOD. A determination of the specific amino acid residue phosphorylated is made by phosphoamino acid analysis of the hydrolyzed protein.

PMOD phosphatase activity is measured by the hydrolysis of P-nitrophenyl phosphate (PNPP). PMOD is incubated together with PNPP in HEPES buffer pH 7.5, in the presence of 0.1% β-mercaptoethanol at 37° C. for 60 min. The reaction is stopped by the addition of 6 ml of 10 N NaOH and the increase in light absorbance at 410 nm resulting from the hydrolysis of PNPP is measured using a spectrophotometer. The increase in light absorbance is proportional to the activity of PMOD in the assay (Diamond, R. H. et al. (1994) Mol. Cell. Biol. 14:3752-62).

The assay for SEQ ID NO:10 is carried out as described above for PMOD using a cysteine protease, such as papain, assayed in the absence and in the presence of various concentrations of SEQ ID NO:10. Inhibition of papain protease activity is proportional to the activity of SEQ ID NO:10 in the assay.

The assay for SEQ ID NO:11 is carried out as described above for PMOD using matrix metalloproteinase assayed in the absence and in the presence of various concentrations of SEQ ID NO:11. Inhibition of matrix metalloproteinase activity is proportional to the activity of SEQ ID NO:11 in the assay.

In the alternative, PMOD phosphatase activity is determined by measuring the amount of phosphate removed from a phosphorylated protein substrate. Reactions are performed with 2 or 4 nM enzyme in a final volume of 30 μl containing 60 mM Tris, pH 7.6, 1 mM EDTA, 1 mM EGTA, 0.1% 2-mercaptoethanol and 10 μM substrate, ³²P-labeled on serine/threonine or tyrosine, as appropriate. Reactions are initiated with substrate and incubated at 30° C. for 10-15 min. Reactions are quenched with 450 μl of 4% (w/v) activated charcoal in 0.6 M HCl, 90 mM Na₄P₂O₇, and 2 mM NaH₂PO₄, then centrifuged at 12,000×g for 5 min. Acid-soluble ³²Pi is quantified by liquid scintillation counting (Sinclair, C. et al. (1999) J. Biol. Chem. 274:23666-23672).

PMOD protease activity is measured by the hydrolysis of appropriate synthetic peptide substrates conjugated with various chromogenic molecules in which the degree of hydrolysis is quantified by spectrophotometric (or fluorometric) absorption of the released chromophore (Beynon, R. J. and J. S. Bond (1994) Proteolytic Enzymes: A Practical Approach. Oxford University Press, New York, N.Y., pp.25-55). Peptide substrates are designed according to the category of protease activity as endopeptidase (serine, cysteine, aspartic proteases, or metalloproteases), aminopeptidase (leucine aminopeptidase), or carboxypeptidase (carboxypeptidases A and B, procollagen C-proteinase). Commonly used chromogens are 2-naphthylamine, 4-nitroaniline, and furylacrylic acid. Assays are performed at ambient temperature and contain an aliquot of the enzyme and the appropriate substrate in a suitable buffer. Reactions are carried out in an optical cuvette, and the increase/decrease in absorbance of the chromogen released during hydrolysis of the peptide substrate is measured. The change in absorbance is proportional to the enzyme activity in the assay.

In the alternative, an assay for PMOD protease activity takes advantage of fluorescence resonance energy transfer (FRET) that occurs when one donor and one acceptor fluorophore with an appropriate spectral overlap are in close proximity. A flexible peptide linker containing a cleavage site specific for PMOD is fused between a red-shifted variant (RSGFP4) and a blue variant (BFP5) of Green Fluorescent Protein. This fusion protein has spectral properties that suggest energy transfer is occurring from BFP5 to RSGFP4. When the fusion protein is incubated with PMOD, the substrate is cleaved, and the two fluorescent proteins dissociate. This is accompanied by a marked decrease in energy transfer which is quantified by comparing the emission spectra before and after the addition of PMOD (Mitra, R. D. et al (1996) Gene 173:13-17). This assay can also be performed in living cells. In this case the fluorescent substrate protein is expressed constitutively in cells and PMOD is introduced on an inducible vector so that FRET can be monitored in the presence and absence of PMOD (Sagot, I. et al (1999) FEBS Letters 447:53-57).

An assay for ubiquitin hydrolase activity measures the hydrolysis of a ubiquitin precursor. The assay is performed at ambient temperature and contains an aliquot of PMOD and the appropriate substrate in a suitable buffer. Chemically synthesized human ubiquitin-valine may be used as substrate. Cleavage of the C-terminal valine residue from the substrate is monitored by capillary electrophoresis (Franklin, K. et al. (1997) Anal. Biochem. 247:305-309).

PMOD protease inhibitor activity for alpha 2-HS-glycoprotein (AHSG) can be measured as a decrease in osteogenic activity in dexamethasone-treated rat bone marrow cell cultures (dex-RBMC). Assays are carried out in 96-well culture plates containing minimal essential medium supplemented with 15% fetal bovine serum, ascorbic acid (50 μg/ml), antibiotics (100 μg/ml penicillin G, 50 μg/ml gentamicin, 0.3 μg/ml fungizone), 10 mM B-glycerophosphate, dexamethasone (10⁻⁸ M) and various concentrations of PMOD for 12-14 days. Mineralized tissue formation in the cultures is quantified by measuring the absorbance at 525 nm using a 96-well plate reader (Binkert, C. et al. supra).

PMOD protease inhibitor activity for inter-alpha-trypsin inhibitor (ITI) can be measured by a continuous spectrophotometric rate determination of trypsin activity. The assay is performed at ambient temperature in a quartz cuvette in pH 7.6 assay buffer containing 63 mM sodium phosphate, 0.23 mM N α-benzoyle-L-arginine ethyl ester, 0.06 mM hydrochloric acid, 100 units trypsin, and various concentrations of PMOD. Immediately after mixing by inversion, the increase in A_{253 nm} is recorded for approximately 5 minutes and the enzyme activity is calculated (Bergmeyer, H. U. et al. (1974) Meth. Enzym. Anal. 1:515-516)

PMOD isomerase activity such as peptidyl prolyl cis/trans isomerase activity can be assayed by an enzyme assay described by Rahfeld, J. U., et al. (1994) (FEBS Lett. 352: 180-184). The assay is performed at 10° C. in 35 mM HEPES buffer, pH 7.8, containing chymotrypsin (0.5 mg/ml) and PMOD at a variety of concentrations. Under these assay conditions, the substrate, Suc-Ala-Xaa-Pro-Phe-4-NA, is in equilibrium with respect to the prolyl bond, with 80-95% in trans and 5-20% in cis conformation. An aliquot (2 ul) of the substrate dissolved in dimethyl sulfoxide (10 mg/ml) is added to the reaction mixture described above. Only the cis isomer of the substrate is a substrate for cleavage by chymotrypsin. Thus, as the substrate is isomerized by PMOD, the product is cleaved by chymotrypsin to produce 4-nitroanilide, which is detected by it's absorbance at 390 nm. 4-Nitroanilide appears in a time-dependent and a PMOD concentration-dependent manner.

PMOD galactosyltransferase activity can be determined by measuring the transfer of radiolabeled galactose from UDP-galactose to a GlcNAc-terminated oligosaccharide chain (Kolbinger, F. et al. (1998) J; Biol. Chem. 273:58-65). The sample is incubated with 14 μl of assay stock solution (180 mM sodium cacodylate, pH 6.5, 1 mg/ml bovine serum albumin, 0.26 mM UDP-galactose, 2 μl of UDP-[³H]galactose), 1 μl of MnCl₂ (500 mM), and 2.5 μl of GlcNAcμO-(CH₂)₈—CO₂Me (37 mg/ml in dimethyl sulfoxide) for 60 minutes at 37° C. The reaction is quenched by the addition of 1 ml of water and loaded on a C18 Sep-Pak cartridge (Waters), and the column is washed twice with 5 ml of water to remove unreacted UDP-[³H]galactose. The [³H]galactosylated GlcNAcμO-(CH₂)₈—CO₂Me remains bound to the column during the water washes and is eluted with 5 ml of methanol. Radioactivity in the eluted material is measured by liquid scintillation counting and is proportional to galactosyltransferase activity in the starting sample.

PMOD induction by heat or toxins may be demonstrated using primary cultures of human fibroblasts or human cell lines such as CCL-13, HEK293, or HEP G2 (ATCC). To heat induce PMOD expression, aliquots of cells are incubated at 42° C. for 15, 30, or 60 minutes. Control aliquots are incubated at 37° C. for the same time periods. To induce PMOD expression by toxins, aliquots of cells are treated with 100 μM arsenite or 20 mM azetidine-2-carboxylic acid for 0, 3, 6, or 12 hours. After exposure to heat, arsenite, or the amino acid analogue, samples of the treated cells are harvested and cell lysates prepared for analysis by western blot. Cells are lysed in lysis buffer containing 1% Nonidet P-40, 0.15 M NaCl, 50 mM Tris-HCl, 5 mM EDTA, 2 mM N-ethylmaleimide, 2 mM phenylmethylsulfonyl fluoride, 1 mg/ml leupeptin, and 1 mg/ml pepstatin. Twenty micrograms of the cell lysate is separated on an 8% SDS-PAGE gel and transferred to a membrane. After blocking with 5% nonfat dry milk/phosphate-buffered saline for 1 h, the membrane is incubated overnight at 4° C. or at room temperature for 2-4 hours with an appropriate dilution of anti-PMOD serum in 2% nonfat dry milk/phosphate-buffered saline. The membrane is then washed and incubated with a 1:1000 dilution of horseradish peroxidase-conjugated goat anti-rabbit IgG in 2% dry milk/phosphate-buffered saline. After washing with 0.1% Tween 20 in phosphate-buffered saline, the PMOD protein is detected and compared to controls using chemiluminescence.

PMOD lysyl hydroxylase activity is determined by measuring the production of hydroxy[¹⁴C]lysine from [¹⁴C]lysine. Radiolabeled protocollagen is incubated with PMOD in buffer containing ascorbic acid, iron sulfate, dithiothreitol, bovine serum albumin, and catalase. Following a 30 minute incubation, the reaction is stopped by the addition of acetone, and centrifuged. The sedimented material is dried, and the hydroxy[¹⁴C]lysine is converted to [¹⁴C]formaldehyde by oxidation with periodate, and then extracted into toluene. The amount of ¹⁴C extracted into toluene is quantified by scintillation counting, and is proportional to the activity of PMOD in the sample (Kivirikko, K., and Myllyla, R. (1982) Methods Enzymol. 82:245-304).

XVIII. Identification of PMOD Substrates

Phage display libraries can be used to identify optimal substrate sequences for PMOD. A random hexamer followed by a linker and a known antibody epitope is cloned as an N-terminal extension of gene III in a filamentous phage library. Gene III codes for a coat protein, and the epitope will be displayed on the surface of each phage particle. The library is incubated with PMOD under proteolytic conditions so that the epitope will be removed if the hexamer codes for a PMOD cleavage site. An antibody that recognizes the epitope is added along with immobilized protein A. Uncleaved phage, which still bear the epitope, are removed by centrifugation. Phage in the supernatant are then amplified and undergo several more rounds of screening. Individual phage clones are then isolated and sequenced. Reaction kinetics for these peptide substrates can be studied using an assay in Example XVII, and an optimal cleavage sequence can be derived (Ke, S. H. et al. (1997) J. Biol. Chem. 272:16603-16609).

To screen for in vivo PMOD substrates, this method can be expanded to screen a cDNA expression library displayed on the surface of phage particles (T7SELECT™ 10-3 Phage display vector, Novagen, Madison, Wis.) or yeast cells (pYD1 yeast display vector kit, Invitrogen, Carlsbad, Calif.). In this case, entire cDNAs are fused between Gene III and the appropriate epitope.

XIX. Identification of PMOD Inhibitors

Compounds to be tested are arrayed in the wells of a multi-well plate in varying concentrations along with an appropriate buffer and substrate, as described in the assays in Example XVII. PMOD activity is measured for each well and the ability of each compound to inhibit PMOD activity can be determined, as well as the dose-response kinetics. This assay could also be used to identify molecules which enhance PMOD activity.

In the alternative, phage display libraries can be used to screen for peptide PMOD inhibitors. Candidates are found among peptides which bind tightly to a protease. In this case, multi-well plate wells are coated with PMOD and incubated with a random peptide phage display library or a cyclic peptide library (Koivunen, E. et al. (1999) Nature Biotech 17:768-774). Unbound phage are washed away and selected phage amplified and rescreened for several more rounds. Candidates are tested for PMOD inhibitory activity using an assay described in Example XVII.

Various modifications and variations of the described compositions, methods, and systems of the invention will be apparent to those skilled in the art without departing from the scope and spirit of the invention. It will be appreciated that the invention provides novel and useful proteins, and their encoding polynucleotides, which can be used in the drug discovery process, as well as methods for using these compositions for the detection, diagnosis, and treatment of diseases and conditions. Although the invention has been described in connection with certain embodiments, it should be understood that the invention as claimed should not be unduly limited to such specific embodiments. Nor should the description of such embodiments be considered exhaustive or limit the invention to the precise forms disclosed. Furthermore, elements from one embodiment can be readily recombined with elements from one or more other embodiments. Such combinations can form a number of embodiments within the scope of the invention. It is intended that the scope of the invention be defined by the following claims and their equivalents.

TABLE 1

| | Polypeptide | | | Incyte | |
|---|---|---|---|---|---|
| Incyte | SEQ ID | Incyte | Polynucleotide | Polynucleotide | Incyte Full |
| Project ID | NO: | Polypeptide ID | SEQ ID NO: | ID | Length Clones |
| 7994355 | 1 | 7994355CD1 | 29 | 7994355CB1 | |
| 7475875 | 2 | 7475875CD1 | 30 | 7475875CB1 | |
| 71231882 | 3 | 71231882CD1 | 31 | 71231882CB1 | |
| 2875922 | 4 | 2875922CD1 | 32 | 2875922CB1 | 90129891CA2 |
| 8158136 | 5 | 8158136CD1 | 33 | 8158136CB1 | 90038744CA2 |
| 5969491 | 6 | 5969491CD1 | 34 | 5969491CB1 | |
| 7497367 | 7 | 7497367CD1 | 35 | 7497367CB1 | 90092215CA2 |
| 7632424 | 8 | 7632424CD1 | 36 | 7632424CB1 | |
| 1804436 | 9 | 1804436CD1 | 37 | 1804436CB1 | |
| 7486358 | 10 | 7486358CD1 | 38 | 7486358CB1 | |
| 7472344 | 11 | 7472344CD1 | 39 | 7472344CB1 | |
| 7192959 | 12 | 7192959CD1 | 40 | 7192959CB1 | |
| 6169565 | 13 | 6169565CD1 | 41 | 6169565CB1 | 6169565CA2 |
| 7494717 | 14 | 7494717CD1 | 42 | 7494717CB1 | |
| 7497510 | 15 | 7497510CD1 | 43 | 7497510CB1 | 90166158CA2, |
| | | | | | 90166182CA2, |
| | | | | | 90166190CA2, |
| | | | | | 90166282CA2, |
| | | | | | 90166290CA2, |
| | | | | | 90189432CA2, |
| | | | | | 90189440CA2, |
| | | | | | 90189464CA2, |
| | | | | | 90189480CA2, |
| | | | | | 90189516CA2, |
| | | | | | 90189532CA2, |
| | | | | | 90189548CA2, |
| | | | | | 90189596CA2, |
| | | | | | 90189833CA2, |
| | | | | | 90189849CA2, |
| | | | | | 90189857CA2, |
| | | | | | 90189865CA2, |
| | | | | | 90189881CA2, |
| | | | | | 90189933CA2, |
| | | | | | 90189941CA2, |
| | | | | | 90189949CA2, |
| | | | | | 90189957CA2, |
| | | | | | 90189981CA2, |
| | | | | | 90189989CA2, |
| | | | | | 90190189CA2 |
| 7498882 | 16 | 7498882CD1 | 44 | 7498882CB1 | |
| 5524205 | 17 | 5524205CD1 | 45 | 5524205CB1 | |
| 7102342 | 18 | 7102342CD1 | 46 | 7102342CB1 | |
| 4169939 | 19 | 4169939CD1 | 47 | 4169939CB1 | |
| 6539977 | 20 | 6539977CD1 | 48 | 6539977CB1 | 90188738CA2, |
| | | | | | 90188893CA2, |
| | | | | | 95003737CA2, |
| | | | | | 95003761CA2, |
| | | | | | 95003853CA2, |
| | | | | | 95003869CA2, |
| | | | | | 95003905CA2, |
| | | | | | 95003913CA2, |
| | | | | | 95003969CA2, |
| | | | | | 95004005CA2, |
| | | | | | 95004061CA2, |
| | | | | | 95004069CA2 |
| 7675588 | 21 | 7675588CD1 | 49 | 7675588CB1 | 4213559CA2, |
| | | | | | 90166613CA2 |
| 6244077 | 22 | 6244077CD1 | 50 | 6244077CB1 | 90110106CA2, |
| | | | | | 90110114CA2, |
| | | | | | 90110154CA2, |
| | | | | | 90110162CA2, |
| | | | | | 90110170CA2, |
| | | | | | 90110186CA2, |
| | | | | | 90110194CA2, |
| | | | | | 90110270CA2, |
| | | | | | 90110278CA2, |
| | | | | | 90110286CA2, |
| | | | | | 90110294CA2, |
| | | | | | 90110470CA2 |
| 7498404 | 23 | 7498404CD1 | 51 | 7498404CB1 | |
| 7391748 | 24 | 7391748CD1 | 52 | 7391748CB1 | |
| 7499780 | 25 | 7499780CD1 | 53 | 7499780CB1 | |
| 7499881 | 26 | 7499881CD1 | 54 | 7499881CB1 | |
| 7488579 | 27 | 7488579CD1 | 55 | 7488579CB1 | |
| 7510521 | 28 | 7510521CD1 | 56 | 7510521CB1 | |

TABLE 2

| Poly- | | | | |
|---|---|---|---|---|
| peptide | | GenBank ID NO: | | |
| SEQ | Incyte | or PROTEOME | Probability | |
| ID NO: | Polypeptide ID | ID NO: | Score | Annotation |
| 1 | 7994355CD1 | g7108521 | 1.3E−85 | [Arabidopsis thaliana] Ubiquitin-protein ligase 2 |
| | | | | Bates, P. W. and Vierstra, R. D. (1999) Plant J. 20 (2), 183-195 UPL1 and 2, two |
| | | | | 405 kDa ubiquitin-protein ligases from Arabidopsis thaliana related to the HECT- |
| | | | | domain protein family |
| 2 | 7475875CD1 | g4262617 | 1.8E−42 | [Caenorhabditis elegans] contains similarity to dual specificity phosphatase, |
| | | | | catalytic domain (Pfam: PF00782) |
| 3 | 71231882CD1 | g1545952 | 1.7E−36 | [Homo sapiens] herpesvirus associated ubiquitin-specific protease (HAUSP) |
| | | | | Everett, R. D., et al. (1997) A novel ubiquitin-specific protease is dynamically |
| | | | | associated with the PML nuclear domain and binds to a herpesvirus regulatory |
| | | | | protein. EMBO J. 16, 566-577 |
| 4 | 2875922CD1 | g14582773 | 0.0 | [Homo sapiens] sumo/sentrin-specific protease |
| 5 | 8158136CD1 | g11993492 | 1.1E−79 | [Arabidopsis thaliana] ubiquitin-specific protease 26 |
| | | | | Yan, N., et al. (2000) The Ubiquitin-Specific Protease Family from Arabidopsis. |
| | | | | AtUBP1 and 2 Are Required for the Resistance to the Amino Acid Analog |
| | | | | Canavanine. Plant Physiol. 124, 1828-1843 |
| 6 | 5969491CD1 | g1545952 | 1.6E−36 | [Arabidopsis thaliana] ubiquitin carboxyl-terminal hydrolase-like protein Everett |
| | | | | et al., supra |
| 7 | 7497367CD1 | g179936 | 1.7E−235 | Homo sapiens] carboxypeptidase N (EC 3.4.17.3) |
| | | | | Tan, F. et al. (1990) The deduced protein sequence of the human |
| | | | | carboxypeptidase N high molecular weight subunit reveals the presence of leucine- |
| | | | | rich tandem repeats J. Biol. Chem. 265: 13-19 |
| 8 | 7632424CD1 | g11071727 | 5.1E−216 | [Homo sapiens] putative sialoglycoprotease type 2 |
| 10 | 7486358CD1 | g3928491 | 1.4E−27 | [Mus musculus] testatin |
| | | | | Tohonen, V. et al. (1998) Proc. Natl. Acad. Sci. U.S.A. 95 (24), 14208-14213 |
| 11 | 7472344CD1 | g15559064 | 0.0 | [Mus musculus] SNAG1 |
| 12 | 7192959CD1 | g6456116 | 9.5E−70 | [Mus musculus] F-box protein FBX17 |
| 13 | 6169565CD1 | g804764 | 1.3E−10 | [Homo sapiens] neutral protease large subunit Johansen, T. et al. (1989) Members |
| | | | | of the RTVL-H family of human endogenous retrovirus-like elements are |
| | | | | expressed in placenta Gene 79: 259-267 |
| 14 | 7494717CD1 | g8489831 | 4.8E−31 | [Homo sapiens] ubiquitin-conjugating BIR-domain enzyme APOLLON Chen, Z. |
| | | | | et al. (1999) A human IAP-family gene, apollon, expressed in human brain cancer |
| | | | | cells Biochem. Biophys. Res. Commun. 264: 847-854 |
| 15 | 7497510CD1 | g450529 | 6.3E−34 | [Homo sapiens] Kunitz-type protease inhibitor, HKIB9 |
| 16 | 7498882CD1 | g17933077 | 0.0 | [Homo sapiens] cathepsin C |
| 17 | 5524205CD1 | g3170887 | 2.0E−30 | [Mus musculus] ubiquitin-protein ligase E3-alpha Kwon, Y. T. et al. (1998) The |
| | | | | mouse and human genes encoding the recognition component of the N-end rule |
| | | | | pathway Proc. Natl. Acad. Sci. U.S.A. 95: 7898-7903 |
| 18 | 7102342CD1 | g20070980 | 0.0 | [Homo sapiens]Similar to a disintegrin and metalloproteinase domain 18 |
| 18 | 7102342CD1 | g4585653 | 9.1E−134 | [Homo sapiens] tMDC III protein |
| 19 | 4169939CD1 | g13937848 | 8.5E−114 | [Homo sapiens] (BC007028) Similar to elastase 3, pancreatic (protease E) |
| 20 | 6539977CD1 | g13897995 | 2.1E−136 | [Homo sapiens] kallikrein 14 Hooper, J. D. et al. (2001) Identification and |
| | | | | characterization of klk14, a novel kallikrein serine protease gene located on |
| | | | | human chromosome 19q13.4 and expressed in prostate and skeletal muscle |
| | | | | Genomics 73: 117-122 |
| 21 | 7675588CD1 | g13591753 | 9.7E−22 | [Oryctolagus cuniculus] eppin Richardson, R. T. et al. (2001) Cloning and |
| | | | | sequencing of human Eppin: a novel family of protease inhibitors expressed in the |
| | | | | epididymis and testis. Gene 270: 93-102 |
| 22 | 6244077CD1 | g6648623 | 3.3E−76 | [Homo sapiens] DNAj homolog |
| 23 | 7498404CD1 | g3062806 | 3.4E−121 | [Homo sapiens] dolichol-phosphate-mannose synthase Tomita, S. et al. (1998) A |
| | | | | homologue of Saccharomyces cerevisiae Dpm1p is not sufficient for synthesis of |
| | | | | dolichol-phosphate-mannose in mammalian cells. J. Biol. Chem. 273: 9249-9254 |
| 24 | 7391748CD1 | g181182 | 1.8E−75 | [Homo sapiens] preprocathepsin G Salvesen, G. et al. (1987) Molecular cloning |
| | | | | of human cathepsin G: structural similarity to mast cell and cytotoxic T |
| | | | | lymphocyte proteinases. |
| | | | | Biochemistry 26: 2289-2293 |
| 25 | 7499780CD1 | g7546824 | 0.0 | [Homo sapiens] lysyl hydroxylase 3 Rautavuoma, K. et al. (2000) Complete exon- |
| | | | | intron organization of the gene for human lysyl hydroxylase 3 (LH3). Matrix |
| | | | | Biol. 19: 73-79 |
| 26 | 7499881CD1 | g5257133 | 0.0 | [Homo sapiens] protein O-mannosyl-transferase 1 |
| 27 | 7488579CD1 | g4336577 | 1.8E−130 | [Homo sapiens] putative mast cell mMCP-7-like II typtase Pallaoro, M. et al. |
| | | | | (1999) Characterization of genes encoding known and novel human mast cell |
| | | | | tryptases on chromosome 16p13.3. J. Biol. Chem. 274: 3355-3362 |
| 28 | 7510521CD1 | g1006657 | 4.5E−17 | [Homo sapiens] cathepsin C Paris, A. et al. (1995) Molecular cloning and |
| | | | | sequence analysis of human preprocathepsin C FEBS Lett. 369, 326-330 |
| 28 | 7510521CD1 | 334886\|CTSC | 3.9E−18 | [Homo sapiens][Hydrolase; Protease (other than |
| | | | | proteasomal)][Lysosome/vacuole; Cytoplasmic] Cathepsin C (dipeptidyl peptidase |
| | | | | I), a lysosomal aminopeptidase and member of the papain family of cysteine |
| | | | | proteinases, involved in activation of serine proteases in immune and |
| | | | | inflammatory cells, mutations are associated with Papillon-Lefevre syndrome |
| | | | | Hart, T. C. et al. |
| | | | | Mutations of the cathepsin C gene are responsible for Papillon-Lefevre syndrome. |
| | | | | J Med Genet 36, 881-7 (1999). |

TABLE 3

| | Incyte | Amino | | |
|---|---|---|---|---|
| SEQ | Poly- | Acid | | Analytical |
| ID | peptide | Resi- | | Methods |
| NO: | ID | dues | Signature Sequences, Domains and Motifs | and Databases |
| 1 | 7994355CD1 | 774 | HECT-domain (ubiquitin-transferase): D467-A774 | HMMER_PFAM |
| | | | Ank repeat: V26-I58, S59-K92, N93-I125 | HMMER_PFAM |
| | | | Ank repeat proteins; PF00023: L31-L46, G60-R69 | BLIMPS_PFAM |
| | | | HECT-domain (ubiquitin-transferase): PF00632: | BLIMPS_PFAM |
| | | | V586-G592, W680-P707, L735-C766 | |
| | | | PROTEIN LIGASE UBIQUITIN | BLAST_PRODOM |
| | | | CONJUGATION REPEAT | |
| | | | UBIQUITIN D20PROTEIN DNA | |
| | | | BINDING PROBABLE ONCOGENIC PD002225: P492-Y771 | |
| | | | HECT DOMAIN DM01690; P39940\|513-808: | BLAST_DOMO |
| | | | N475-L764; P51593\|9-306: N475-Y771; | |
| | | | A38919\|785-1082: N475-A774; | |
| | | | P53119\|615-909: N475-S768 | |
| | | | Potential Phosphorylation Sites: S7 S15 S159 | MOTIFS |
| | | | S176 S231 S250 S291 S338 S430 S669 S703 T96 | |
| | | | T128 T216 T689 Y397 | |
| | | | Potential Glycosylation Sites: N283 N722 | MOTIFS |
| 2 | 7475875CD1 | 703 | Transmembrane domain: V113-A129, | TMAP |
| | | | A141-V169 N-terminus is non-cytosolic | |
| | | | Tyrosine specific protein phosphatase | BLIMPS_BLOCKS |
| | | | BL00383: I137-G147, R170-C185 | |
| | | | Tyrosine specific protein phosphatases | PROFILESCAN |
| | | | signature and profiles: L116-P174 | |
| | | | Tyrosine specific protein phosphatases | MOTIFS |
| | | | active site: I137-L149 | |
| | | | Leucine zipper pattern: L333-L354 | MOTIFS |
| | | | Potential Phosphorylation Sites: D18S21 S201 | MOTIFS |
| | | | S341 S343 S448 S486 S496 S503·S594 T118 | |
| | | | T272 T493 T605 T679 T687 T695 | |
| | | | Potential Glycosylation Sites: N550 | MOTIFS |
| 3 | 71231882CD1 | 1256 | Ubiquitin carboxyl-terminal hydrolases family 1: S41-L72 | HMMER_PFAM |
| | | | Ubiquitin carboxyl-terminal hydrolase family 2: L285-K482 | HMMER_PFAM |
| | | | Ubiquitin carboxyl-terminal hydrolases family | BLIMPS_BLOCKS |
| | | | 2 proteins BL00972: G42-L59, M129-N138, I158- | |
| | | | C172, I288-D312, C446-Q467 | |
| | | | PROTEASE UBIQUITIN HYDROLASE | BLAST_PRODOM |
| | | | UBIQUITINSPECIFIC ENZYME | |
| | | | DEUBIQUITINATING CARBOXYLTERMINAL | |
| | | | THIOLESTERASE PROCESSING | |
| | | | CONJUGATION | |
| | | | UBIQUITIN CARBOXYL-TERMINAL HYDROLASES FAMILY 2; | BLAST_DOMO |
| | | | DM00659\|P50101\|209-458: N45-G299 | |
| 1 | 7994355CD1 | 774 | HECT-domain (ubiquitin-transferase): D467-A774 | HMMER_PFAM |
| | | | Ank repeat: V26-I58, S59-K92, N93-I125 | HMMER_PFAM |
| | | | Ank repeat proteins; PF00023: L31-L46, G60-R69 | BLIMPS_PFAM |
| | | | HECT-domain (ubiquitin-transferase): PF00632: | BLIMPS_PFAM |
| | | | V586-G592, W680-P707, L735-C766 | |
| | | | PROTEIN LIGASE UBIQUITIN CONJUGATION | BLAST_PRODOM |
| | | | REPEAT UBIQUITIN D20PROTEIN DNA | |
| | | | BINDING PROBABLE ONCOGENIC PD002225: P492-Y771 | |
| | | | HECT DOMAIN DM01690; P39940\|513-808: | BLAST_DOMO |
| | | | N475-L764; P51593\|9-306: N475-Y771; | |
| | | | A38919\|785-1082: N475-A774; | |
| | | | P53119\|615-909: N475-S768 | |
| | | | Potential Phosphorylation Sites: S7 S15 S159 | MOTIFS |
| | | | S176 S231 S250 S291 S338 S430 S669 S703 T96 | |
| | | | T128 T216 T689 Y397 | |
| | | | Potential Glycosylation Sites: N283 N722 | MOTIFS |
| 2 | 7475875CD1 | 703 | Transmembrane domain: V113-A129, A141-V169 | TMAP |
| | | | N-terminus is non-cytosolic | |
| | | | Tyrosine specific protein phosphatase BL00383: | BLIMPS_BLOCKS |
| | | | I137-G147, R170-C185 | |
| | | | Tyrosine specific protein phosphatases signature | PROFILESCAN |
| | | | and profiles: L116-P174 | |
| | | | Tyrosine specific protein phosphatases active | MOTIFS |
| | | | site: I137-L149 | |
| | | | Leucine zipper pattern: L333-L354 | MOTIFS |
| | | | Potential Phosphorylation Sites: D18S21 S201 | MOTIFS |
| | | | S341 S343 S448 S486 S496 S503 S594 T118 | |
| | | | T272 T493 T605 T679 T687 T695 | |
| | | | Potential Glycosylation Sites: N550 | MOTIFS |
| 3 | 71231882CD1 | 1256 | Ubiquitin carboxyl-terminal hydrolases family 1: S41-L72 | HMMER_PFAM |
| | | | Ubiquitin carboxyl-terminal hydrolase family 2: L285-K482 | HMMER_PFAM |
| | | | Ubiquitin carboxyl-terminal hydrolases family | BLIMPS_BLOCKS |
| | | | 2 proteins BL00972: G42-L59, M129-N138, I158- | |
| | | | C172, I288-D312, C446-Q467 | |
| | | | PROTEASE UBIQUITIN HYDROLASE | BLAST_PRODOM |
| | | | UBIQUITINSPECIFIC ENZYME | |
| | | | DEUBIQUITINATING CARBOXYLTERMINAL | |
| | | | THIOLESTERASE PROCESSING | |
| | | | CONJUGATION | |
| | | | UBIQUITIN CARBOXYL-TERMINAL HYDROLASES | BLAST_DOMO |
| | | | FAMILY 2; DM00659\|P50101\|209- | |
| | | | 458: N45-G299 | |
| | | | Ubiquitin carboxyl-terminal hydrolases family | MOTIFS |
| | | | 2 signature 2: Y289-Y306 | |
| | | | Potential Phosphorylation Sites: S113 S125 | MOTIFS |
| | | | S143 S153 S179 S196 S232 S280 S326 S337 S402 | |
| | | | S550 S554 S653 S729 S745 S868 S907 S971 S995 | |
| | | | S1122 S1220 S1233 S1254 T124 T407 T516 | |
| | | | T565 T608 T635 T651 T712 T737 T818 T857 T928 | |
| | | | T1017 T1058 T1161 T1200 T1205 Y265 | |
| | | | Y896 Y1104 | |
| | | | Potential Glycosylation Sites: N39 N177 N194 | MOTIFS |
| | | | N413 N441 N453 N525 N720 N977 | |
| 4 | 2875922CD1 | 755 | PROTEIN CHROMOSOME C17A5.07C I SMT4 | BLAST_PRODOM |
| | | | SIMILARITY A SMALL REGION OF; | |
| | | | PD009801: K602-L748 | |
| | | | Potential Phosphorylation Sites: S18 S93 S107 | MOTIFS |
| | | | S122 S126 S176 S300 S339 S363·S388 S405 | |
| | | | S416 S423 S430 S447 S465 S488 S492 S521 S538 | |
| | | | S655 S660 S733 T278 T390 T626 Y130 | |
| | | | Potential Glycosylation Sites: N88 N349 | MOTIFS |
| | | | N401 N709 | |
| 5 | 8158136CD1 | 1034 | Ubiquitin carboxyl-terminal hydrolases | HMMER_PFAM |
| | | | family 1: V89-C120 | |
| | | | Ubiquitin carboxyl-terminal hydrolase | HMMER_PFAM |
| | | | family 2: G332-L420 | |
| | | | Ubiquitin carboxyl-terminal hydrolases | BLIMPS_BLOCKS |
| | | | family 2 proteins BL00972: G90-W107, G177-S186, | |
| | | | Y336-D360, S363-L384 | |
| | | | PROTEASE UBIQUITIN HYDROLASE | BLAST_PRODOM |
| | | | UBIQUITINSPECIFIC ENZYME | |
| | | | DEUBIQUITINATING CARBOXYLTERMINAL | |
| | | | THIOLESTERASE PROCESSING | |
| | | | F30A10.10 PROTEIN; PD185574: E641-A879 | BLAST_PRODOM |
| | | | UBIQUITIN CARBOXYL-TERMINAL HYDROLASES FAMILY | BLAST_DOMO |
| | | | 2; DM00659\|P40818\|782-1103: Q200-I374, L94-N202 | |
| | | | Ubiquitin carboxyl-terminal hydrolases family | MOTIFS |
| | | | 2 signature 2: Y336-Y354 | |
| | | | Potential Phosphorylation Sites: S87 S158 S191 | MOTIFS |
| | | | S445 S461 S531 S597 S600 S633 S659 S686 | |
| | | | S878 S885 S887 S934 T17 T123 T143 T280 T398 | |
| | | | T549 T760 T788 T819 T889 T913 T953 | |
| | | | Y268 Y687 | |
| | | | Potential Glycosylation Sites: N278 N427 | MOTIFS |
| | | | N624 N883 N907 N951 N973 | |
| 6 | 5969491CD1 | 1236 | Ubiquitin carboxyl-terminal hydrolases | HMMER_PFAM |
| | | | family 1: S41-L72 | |
| | | | Ubiquitin carboxyl-terminal hydrolase | HMMER_PFAM |
| | | | family 2: L285-K482 | |
| | | | Ubiquitin carboxyl-terminal hydrolases | BLIMPS_BLOCKS |
| | | | family 2 proteins BL00972: G42-L59, M129-N138, I158- | |
| | | | C172, I288-D312, C446-Q467 | |
| | | | PROTEASE UBIQUITIN HYDROLASE | BLAST_PRODOM |
| | | | UBIQUITINSPECIFIC ENZYME | |
| | | | DEUBIQUITINATING CARBOXYLTERMINAL | |
| | | | THIOLESTERASE PROCESSING | |
| | | | CONJUGATION; PD017412: R181-E286; | |
| | | | (P-value = 1.1e−10) | |
| | | | UBIQUITIN CARBOXYL-TERMINAL | BLAST_DOMO |
| | | | HYDROLASES FAMILY 2; DM00659\|P50101\|209- | |
| | | | 458: N45-G299 | |
| | | | Ubiquitin carboxyl-terminal hydrolases family | MOTIFS |
| | | | 2 signature 2: Y289-Y306 | |
| | | | Potential Phosphorylation Sites: S113 S125 S143 | MOTIFS |
| | | | S153 S179 S196 S232 S280 S326 S337 S402 | |
| | | | S550 S554 S653 S729 S745 S868 S951 S975 S1102 | |
| | | | S1200 S1213 S1234 T124 T407 T516 T565 | |
| | | | T608 T635 T651 T712 T737 T818 T857 T908 T997 | |
| | | | T1038 T1141 T1180 T1185 Y265 Y896 | |
| | | | Y1084 | |
| | | | Potential Glycosylation Sites: N39 N177 N194 | MOTIFS |
| | | | N413 N441 N453 N525 N720 N957 | |
| 7 | 7497367CD1 | 545 | signal_cleavage: M1-P21 | SPSCAN |
| | | | Signal Peptide: M1-P21 | HMMER |
| | | | Leucine Rich Repeat: H170-T193, N266-P289, | HMMER_PFAM |
| | | | S122-A145, K362-Y385, C290-S313, R98-T121, | |
| | | | N74-P97, N314-E337, S194-G217, | |
| | | | S218-F241, A146-T169, E338-S361, C242-G265 | |
| | | | Leucine rich repeat C-terminal domain: N395-P446 | HMMER_PFAM |
| | | | Leucine rich repeat N-terminal domain: P21-P48 | HMMER_PFAM |
| | | | Leucine zipper pattern: L126-L147, L150-L171, | MOTIFS |
| | | | L174-L195, L270-L291, L342-L363 | |
| | | | Potential Phosphorylation Sites: S36 S218 S361 | MOTIFS |
| | | | S484 S507 S531 T62 T197 T303 T375 T414 | |
| | | | T457 | |
| | | | Potential Glycosylation Sites: N74 N111 N119 | MOTIFS |
| | | | N228 N266 N348 N359 N518 | |
| 8 | 7632424CD1 | 414 | Glycoprotease family: K37-R372 | HMMER_PFAM |
| | | | Glycoprotease family proteins BL01016: G325-R334, | BLIMPS_BLOCKS |
| | | | L355-A364, V39-V53, G76-H87, D108- | |
| | | | A152, L166-A178, L189-C216, C249-T260 | |
| | | | O-sialoglycoprotein endopeptidase (M22) metallo- | BLIMPS_PRINTS |
| | | | protease family signature PR00789: L40-V53, | |
| | | | I112-L132, Q133-A152, L166-A178, | |
| | | | L189-L210, L321-N330 | |
| | | | PROTEIN HYDROLASE METALLOPROTEASE | BLAST_PRODOM |
| | | | ZINC ENDOPEPTIDASE O- | |
| | | | SIALOGLYCOPROTEIN PUTATIVE | |
| | | | GLYCOPROTEASE INTERGENIC REGION | |
| | | | PD002367: I38-I368 | |
| | | | GLYCOPROTEASE FAMILY DM02236; \|P05852\|1- | BLAST_DOMO |
| | | | 276: V39-I305; \|P43764\|1-281: V39-I305; | |
| | | | \|P47292\|5-277: V39-I305; | |
| | | | \|P43122\|33-333: V39-R334 | |
| | | | Potential Phosphorylation Sites: S15 S45 S67 | MOTIFS |
| | | | S105 S209 S281 S404 T44 T115 T154 T158 T260 | |
| | | | T301 | |
| | | | Potential Glycosylation Sites: N342 | MOTIFS |
| 9 | 1804436CD1 | 611 | Serine proteases, V8 family, histidine | BLIMPS_BLOCKS |
| | | | proteins; BL00672: T531-D547 | |
| | | | V8 serine protease family signature; PR00839: | BLIMPS_PRINTS |
| | | | A370-I387, I530-F546, D547-G559 | |
| | | | Potential Phosphorylation Sites: S2 S8 S12 S45 | MOTIFS |
| | | | S62 S233 S284 S302 S307 S343 S503 S570 S576 | |
| | | | S607 T81 T97 T220 T248 T289 T325 T382 T516 | |
| | | | Y359 Y424 Y511 | |
| | | | Potential Glycosylation Sites: N35 N75 N101 N246 N340 N566 | MOTIFS |
| 10 | 7486358CD1 | 147 | signal_cleavage: M1-A28 | SPSCAN |
| | | | Signal Peptide: M1-V25, M1-A28 | HMMER |
| | | | Cystatin domain: P49-C142 | HMMER_PFAM |
| | | | CYSTEINE PROTEASES INHIBITORS; | BLAST_DOMO |
| | | | DM00182\|P01035\|1-110: L51-C142; | |
| | | | DM00182\|P01034\|34-143: L51-C142; | |
| | | | DM00182\|P28325\|29-139: L51-C142; | |
| | | | DM00182\|P01038\|29-138: L47-C142 | |
| | | | Potential Phosphorylation Sites: S76 T133 Y68 | MOTIFS |
| | | | Potential Glycosylation Sites: N117 N139 | MOTIFS |
| 11 | 7472344CD1 | 624 | PX domain: P273-P382 | HMMER_PFAM |
| | | | SH3 domain: L3-I59 | HMMER_PFAM |
| | | | Potential Phosphorylation Sites: S20 S31 S44 | MOTIFS |
| | | | S96 S150 S196 S238 S297 S350 S383 S384 S426 | |
| | | | S470 S549 T163 T197 T279 T341 T435 T448 | |
| | | | T577 T611 Y320 | |
| | | | Potential Glycosylation Sites: N446 | MOTIFS |
| 12 | 7192959CD1 | 283 | signal_cleavage: M1-L47 | SPSCAN |
| | | | F-box domain: L24-A72 | HMMER_PFAM |
| | | | Potential Phosphorylation Sites: S181 T78 T214 | MOTIFS |
| | | | Potential Glycosylation Sites: N141 N212 N273 | MOTIFS |
| 13 | 6169565CD1 | 142 | Signal_cleavage: M1-G38 | SPSCAN |
| | | | Signal Peptide: M1-G35 | HMMER |
| | | | Potential Phosphorylation Sites: S52 S106 S135 | MOTIFS |
| 14 | 7494717CD1 | 354 | Signal_cleavage: M1-G57 | SPSCAN |
| | | | Ubiquitin-conjugating enzyme: G93-R250 | HMMER_PFAM |
| | | | Ubiquitin-conjugating enzymes active site: F152-E219 | PROFILESCAN |
| | | | UBIQUITIN LIGASE ENZYME CONJUGATING | BLAST_PRODOM |
| | | | CARRIER; MULTIGENE FAMILY | |
| | | | PD000461: T86-P221 | |
| | | | UBIQUITIN-CONJUGATING ENZYMES DM00225; | BLAST_DOMO |
| | | | P35133\|1-146: R103-V253; P35128\|1- | |
| | | | 148: R103-P221; P25865\|2-149: | |
| | | | R103-R245; P23566\|2-149: R103-R245 | |
| | | | Potential Phosphorylation Sites: S4 S81 S89 S274 | MOTIFS |
| | | | S342 S351 T141 T174 T248 T296 T344 Y240 | |
| | | | Potential Glycosylation Sites: N172 | MOTIFS |
| 15 | 7497510CD1 | 89 | Signal_cleavage: M1-S21 | SPSCAN |
| | | | Signal Peptide: M1-E18, M1-S21, M1-L23, M1-A24 | HMMER |
| | | | Kunitz/Bovine pancreatic trypsin inhibitor: C36-C86 | HMMER_PFAM |
| | | | Pancreatic trypsin inhibitor (Kunitz) family | PROFILESCAN |
| | | | signature: P44-K87 | |
| | | | Basic protease (Kunitz-type) inhibitor family | BLIMPS_PRINTS |
| | | | signature PR00759: P33-T47, C61-G71, G71-C86 | |
| | | | INHIBITOR PROTEASE SERINE GLYCOPROTEIN | BLAST_PRODOM |
| | | | RECURSOR; SIGNAL FACTOR | |
| | | | REPEAT TISSUE PD000222: C36-C86 | |
| | | | ANIMAL KUNITZ-TYPE PROTEINASE INHIBITOR DM00114; | BLAST_DOMO |
| | | | S41399\|5-61: D30-K87; | |
| | | | P10646\|48-110: L31-C86; P10646\|112- | |
| | | | 175: L23-K87; P10280\|1-55: C36-C86 | |
| | | | Pancreatic trypsin inhibitor (Kunitz) family | MOTIFS |
| | | | signature: F64-C82 | |
| | | | Potential Phosphorylation Sites: T27 | MOTIFS |
| 16 | 7498882CD1 | 419 | Signal_cleavage: M1-G20 | SPSCAN |
| | | | Signal Peptide: M1-C24, M1-G20, M1-A28, M1-R23 | HMMER |
| | | | Papain family cysteine protease: G253-T414, L231-Q252 | HMMER_PFAM |
| | | | Eukaryotic thiol (cysteine) proteases cysteine | BLIMPS_BLOCKS |
| | | | proteins BL00139: N251-P259, N360-G369, Y378- | |
| | | | Y394 | |
| | | | Eukaryotic thiol (cysteine) proteases | PROFILESCAN |
| | | | active sites: D334-F395 | |
| | | | Papain cysteine protease (C1) family signature | BLIMPS_PRINTS |
| | | | PR00705: H361-D371, Y378-S384 | |
| | | | PRECURSOR C HYDROLASE SIGNAL | BLAST_PRODOM |
| | | | CATHEPSIN THIOL PROTEASE ZYMOGEN | |
| | | | DIPEPTIDYL PEPTIDASE I; PD013797: G20-H230 | |
| | | | PROTEASE PRECURSOR SIGNAL | BLAST_PRODOM |
| | | | CYSTEINE PROTEINASE HYDROLASE THIOL | |
| | | | ZYMOGEN CATHEPSIN GLYCOPROTEIN; | |
| | | | PD000158: N251-R399 | |
| | | | EUKARYOTIC THIOL (CYSTEINE) PROTEASES CYSTEINE | BLAST_DOMO |
| | | | DM00081\|P80067\|124-457: | |
| | | | Q252-P415, G124-G264; P53634\|124-458: | |
| | | | Q252-P415, G124-Y368; P09668\|25-332: N251- | |
| | | | P417, H155-G256; S52212\|34-335: G253-P415 | |
| | | | Eukaryotic thiol (cysteine) proteases | MOTIFS |
| | | | asparagine active site: Y378-I397 | |
| | | | Eukaryotic thiol (cysteine) proteases | MOTIFS |
| | | | histidine active site: T359-G369 | |
| | | | Potential Phosphorylation Sites: S48 S164 | MOTIFS |
| | | | S209 S374 T31 T73 T138 T144 T189 T197 T233 | |
| | | | T281 T401 Y296 | |
| | | | Potential Glycosylation Sites: N29 N53 N119 | MOTIFS |
| 17 | 5524205CD1 | 951 | LIGASE UBIQUITIN E3 COMPONENT | BLAST_PRODOM |
| | | | PROTEIN NENDRECOGNIZING N RECOGNIN | |
| | | | CONJUGATION; PD007850: I776-H935 | |
| | | | Potential Phosphorylation Sites: S25 S30 S78 | MOTIFS |
| | | | S116 S129 S164 S190 S266 S308 S341 S400 S404 | |
| | | | S440 S451 S454 S639 S643 S659 S734 S765 S786 | |
| | | | S860 T70 T82 T196 T224 T233 T270 T286 | |
| | | | T493 T520 T574 T660 T788 | |
| | | | Potential Glycosylation Sites: N5 N114 N452 | MOTIFS |
| | | | N477 N491 N882 | |
| 18 | 7102342CD1 | 668 | Signal_cleavage: M1-A16 | SPSCAN |
| | | | Signal Peptide: M1-A16, M1-P19, M1-G20, M1-Q22 | HMMER |
| | | | Non-cytosolic domain: M1-E574; Transmembrane | TMHMMER |
| | | | domain: N575-A597; Cytosolic domain: R598- | |
| | | | Reprolysin family propeptide: H63-S176 | HMMER_PFAM |
| | | | Reprolysin (M12B) family zinc metalloprotease: L186-P346 | HMMER_PFAM |
| | | | Disintegrin: A301-C365 | HMMER_PFAM |
| | | | Disintegrins signature: G287-D368 | PROFILESCAN |
| | | | Disintegrin signature PR00289: C326-R345, E356-D368 | BLIMPS_PRINTS |
| | | | EGF-like domain signature 2: C536-C547 | MOTIFS |
| | | | Potential Phosphorylation Sites: S42 S135 S160 | MOTIFS |
| | | | S173 S234 S311 S340 S361 S376 S417 S419 | |
| | | | S494 S564 S570 S616 S629 S648 T197 T210 T247 | |
| | | | T452 T483 T642 T652 Y275 | |
| | | | Potential Glycosylation Sites: N39 N125 N359 N492 | MOTIFS |
| 19 | 4169939CD1 | 206 | Signal_cleavage: M1-G17 | SPSCAN |
| | | | Signal Peptide: M2-G17, M2-G19, M1-P21, | HMMER |
| | | | M1-S23, M1-S25, M1-G17, M1-G19, M2-S25 | |
| | | | Trypsin: I36-I199 | HMMER_PFAM |
| | | | Serine proteases, trypsin family, histidine | BLIMPS_BLOCKS |
| | | | proteins BL00134: S147-G170, P186-I199 | |
| | | | Apple domain proteins BL00495: V171-W198, | BLIMPS_BLOCKS |
| | | | S147-T187, A69-T103, V139-S173, G177-S205 | |
| | | | Type I fibronectin domain proteins BL01253: | BLIMPS_BLOCKS |
| | | | R67-T103, N104-G142, R146-C159, V168-T202 | |
| | | | Serine proteases, trypsin family, active | PROFILESCAN |
| | | | sites: V134-C180 | |
| | | | Chymotrypsin serine protease family (S1) | BLIMPS_PRINTS |
| | | | signature PR00722: A55-A69, R146-N158 | |
| | | | PROTEASE SERINE PRECURSOR SIGNAL | BLAST_PRODOM |
| | | | HYDROLASE ZYMOGEN GLYCOPROTEIN | |
| | | | FAMILY MULTIGENE FACTOR; D000046: S68-I199, G19-G165 | |
| | | | TRYPSIN DM00018; P05805\|1-236: V29-I203; | BLAST_DOMO |
| | | | P08217\|28-265: A35-I203; P55091\|29-265: E33- | |
| | | | I203, R28-S40; P05208\|31-267: S23-I203 | |
| | | | Serine proteases, trypsin family, serine | MOTIFS |
| | | | active site: S147-N158 | |
| | | | Potential Phosphorylation Sites: S26 T133 | MOTIFS |
| | | | Potential Glycosylation Sites: N50 | MOTIFS |
| 20 | 6539977CD1 | 267 | Signal_cleavage: M1-A30 | SPSCAN |
| | | | Signal Peptide: P15-A30, M17-S34 | HMMER |
| | | | Trypsin: I41-I260 | HMMER_PFAM |
| | | | Kringle domain proteins; BL00021: C68-G85, | BLIMPS_BLOCKS |
| | | | R144-G165, D219-I260 | |
| | | | Serine proteases, trypsin family, histidine | BLIMPS_BLOCKS |
| | | | proteins BL00134: C68-C84, D214-G237, P247-I260 | |
| | | | Type I fibronectin domain proteins BL01253: | BLIMPS_BLOCKS |
| | | | C68-A81, S169-G207, K213-C226, Q229-T263 | |
| | | | Serine proteases, trypsin family, active | PROFILESCAN |
| | | | sites: A60-T104, I199-A242 | |
| | | | Chymotrypsin serine protease family (S1) | BLIMPS_PRINTS |
| | | | signature PR00722: G69-C84, T123-A137, K213-V225 | |
| | | | REPEAT PRECURSOR GLYCOPROTEIN PD00120: | BLIMPS_PRODOM |
| | | | G69-A81, D214-G222 | |
| | | | PROTEASE SERINE PRECURSOR SIGNAL | BLAST_PRODOM |
| | | | HYDROLASE ZYMOGEN GLYCOPROTEIN | |
| | | | FAMILY MULTIGENE FACTOR; PD000046: | |
| | | | P155-I260, I41-P136 | |
| | | | TRYPSIN DM00018; P32821\|24-242: K40-M264; | BLAST_DOMO |
| | | | P19799\|21-239: I41-M264; P06872\|24-242: | |
| | | | I41-M264; P06871\|23-242: K40-M264 | |
| | | | Serine proteases, trypsin family, serine | MOTIFS |
| | | | active site: D214-V225 | |
| | | | Serine proteases, trypsin family, histidine | MOTIFS |
| | | | active site: I79-C84 | |
| | | | Potential Phosphorylation Sites: S2 S34 | MOTIFS |
| | | | S158 S186 S258 T263 | |
| 21 | 7675588CD1 | 86 | Signal_cleavage: M1-A25 | SPSCAN |
| | | | Signal Peptide: M1-G27 | HMMER |
| | | | Non-cytosolic domain: M1-E86 | MOTIFS |
| | | | WAP-type (Whey Acidic Protein) ’four- | HMMER_PFAM |
| | | | disulfide core: K31-F72 | |
| | | | WAP-type ’four-disulfide core’ | PROFILESCAN |
| | | | domain signature: P32-D71 | |
| | | | 4-disulphide core signature PR00003C: | BLIMPS_PRINTS |
| | | | C54-F63, Q47-C54, S64-F42 | |
| | | | Potential Phosphorylation Sites: S76 | MOTIFS |
| 22 | 6244077CD1 | 232 | DnaJ domain: N3-G69 | HMMER_PFAM |
| | | | Nt-dnaJ domain proteins BL00636: D18-K34, F46-D66 | BLIMPS_BLOCKS |
| | | | dnaJ domains signatures and profile: R24-P86 | PROFILESCAN |
| | | | DnaJ protein family signature PR00625: | BLIMPS_PRINTS |
| | | | A14-D33, F46-D66, S184-I199 | |
| | | | DNAJ PROTEIN HOMOLOG HSJ1 HSJ1 | BLAST_PRODOM |
| | | | CHAPERONE NEURONE ALTERNATIVE | |
| | | | SPLICING TESTIS PD013370: Y91-K232 | |
| | | | PROTEIN CHAPERONE DNAJ HEAT SHOCK DNA | BLAST_PRODOM |
| | | | REPLICATION REPEAT ANTIGEN T | |
| | | | PD000231: N3-D66 | |
| | | | NT-DNAJ DOMAIN; DM00098\|S23509\|1-108: | BLAST_DOMO |
| | | | M1-G105; DM00098\|P25686\|1-108: M1-G105; | |
| | | | DM00098\|P30725\|1-102: M1-G106; | |
| | | | DM00098\|S34632\|1-99: Y4-G106 | |
| | | | Binding-protein-dependent transport systems | MOTIFS |
| | | | inner membrane comp. sign: F114-P142 | |
| | | | Nt-dnaJ domain signature: F46-Y65 | MOTIFS |
| | | | Potential Phosphorylation Sites: S15 S59 S63 | MOTIFS |
| | | | S72 S85 S121 S184 T92 T195 T196 | |
| 23 | 7498404CD1 | 237 | signal_cleavage: M1-S52 | SPSCAN |
| | | | Glycosyl transferase: S28-R195 | HMMER_PFAM |
| | | | Glycosyl transferases. PF00535: I61-T71, | BLIMPS_PFAM |
| | | | A109-D118; P < 3e−3 | |
| | | | SYNTHASE DOLICHOL-PHOSPHATE | BLAST_PRODOM |
| | | | MANNOSE MANNOSYLTRANSFERASE | |
| | | | DOLICHYLPHOSPHATE BETADMANNOSYL- | |
| | | | TRANSFERASE GLYCOSYL- | |
| | | | TRANSFERASE TRANSMEMBRANE | |
| | | | PD151196: L175-A235 | |
| | | | GLYCOSYL-TRANSFERASE | BLAST_PRODOM |
| | | | BIOSYNTHESIS GLYCOSYL SYNTHASE | |
| | | | TRANSMEMBRANE N-ACETYL- | |
| | | | GALACTOSAMINYLTRANSFERASE MEMBRANE | |
| | | | PD000196: S28-R147 | |
| | | | Potential Phosphorylation Sites: S9 S13 S21 | MOTIFS |
| | | | S50 S84 S207 T33 T200 | |
| | | | Potential Glycosylation Sites: N198 | MOTIFS |
| 24 | 7391748CD1 | 146 | Trypsin: M1-I129 | HMMER_PFAM |
| | | | Serine proteases, trypsin family, histidine | BLIMPS_BLOCKS |
| | | | proteins BL00134: A86-G109, P116-I129 | |
| | | | Type I fibronectin domain proteins BL01253: | BLIMPS_BLOCKS |
| | | | K85-C98, C98-T132, R7-M43 | |
| | | | Serine proteases, trypsin family, active | PROFILESCAN |
| | | | sites: L27-V114 | |
| | | | PROTEASE SERINE PRECURSOR SIGNAL | BLAST_PRODOM |
| | | | HYDROLASE ZYMOGEN GLYCOPROTEIN | |
| | | | FAMILY MULTIGENE FACTOR PD000046: N14-G113 | |
| | | | TRYPSIN; DM00018\|P08311\|21-241: M1-M133; | BLAST_DOMO |
| | | | DM00018\|P28293\|21-241: M1-M133; | |
| | | | DM00018\|P80219\|1-221: M1-M133; | |
| | | | DM00018\|P20718\|21-242: M1-M133 | |
| | | | ATP/GTP-binding site motif A (P-loop): G104-S111 | MOTIFS |
| | | | Serine proteases, trypsin family, serine | MOTIFS |
| | | | active site: A86-L97 | |
| | | | Potential Phosphorylation Sites: S6 S42 S135 | MOTIFS |
| | | | T47 T49 T132 | |
| 25 | 7499780CD1 | 696 | signal_cleavage: M1-A24 | SPSCAN |
| | | | Signal Peptide: M1-S23, M1-A24, M1-R27 | HMMER |
| | | | Lysyl hydrolase: Q293-P696, E37-G292 | HMMER_PFAM |
| | | | PROCOLLAGEN-LYSINE 2-OXOGLUTARATE | BLAST_PRODOM |
| | | | 5-DIOXYGENASE PRECURSOR LYSYL | |
| | | | HYDROXYLASE OXIDOREDUCTASE DIOXYGENASE | |
| | | | SIGNAL IRON; PD011980: L13- | |
| | | | D285; PD011578: E500-P696; PD009947: E294-K499 | |
| | | | LYSYL HYDROXYLASE CHAIN; DM07920\|P24802\|1- | BLAST_DOMO |
| | | | 729: H297-P696, L12-G291; | |
| | | | DM07920\|Q02809\|1-726: E294-P696, P15-E294 | |
| | | | Potential Phosphorylation Sites: S23 S25 | MOTIFS |
| | | | S121 S325 S391 S441 S443 S454 S477 S621 S660 | |
| | | | S692 T65 T159 T360 T429 T445 T476 Y64 Y346 | |
| | | | Potential Glycosylation Sites: N63 N506 | MOTIFS |
| 26 | 7499881CD1 | 630 | MIR domain: D339-H396, P275-V332, P201-P264 | HMMER_PFAM |
| | | | Dolichyl-phosphate-mannose-protein | HMMER_PFAM |
| | | | mannosyltransferase: M1-F172 | |
| | | | Intracellular domains: M1-E8, N58-S63, | TMHMMER |
| | | | D111-R148, R503-D514, Q565-H630; Transmembrane | |
| | | | domains: L9-T31, L35-F57, P64-I86, | |
| | | | V91-G110, A149-V171, I480-L502, A515-F537, Y547- | |
| | | | L564; Extracellular domains: Q32-R34, | |
| | | | K87-G90, F172-N479, F538-L546 | |
| | | | DETHIOBIOTIN SYNTHETASE PD02561: N479-R506, V332-T343 | BLIMPS_PRODOM |
| | | | DOLICHYLPHOSPHATEMANNOSE | BLAST_PRODOM |
| | | | PROTEIN MANNOSYLTRANSFERASE | |
| | | | GLYCOSYLTRANSFERASE GLYCOPROTEIN | |
| | | | TRANSMEMBRANE ENDOPLASMIC | |
| | | | RETICULUM MULTIGENE FAMILY; PD009956: | |
| | | | H144-H288; PD005044: M1-L108; | |
| | | | ROTATED ABDOMEN PROTEIN PD116484: Q402-G523 | BLAST_PRODOM |
| | | | LUMENAL DOMAIN; DM02305\|P31382\|36-704: | BLAST_DOMO |
| | | | H144-L560, M1-S115; DM02305\|P47190\|23- | |
| | | | 698: H144-L560, M1-S115; DM02305\|P46971\|28- | |
| | | | 693: H144-S574, Y3-L109; | |
| | | | DM02305\|P42934\|30-714: H144-L564, F11-V117 | |
| | | | Leucine zipper pattern: L539-L560 | MOTIFS |
| | | | Potential Phosphorylation Sites: S226 S336 S451 | MOTIFS |
| | | | S610 T211 T291 T598 T603 | |
| | | | Potential Glycosylation Sites: N318 N354 N422 | MOTIFS |
| 27 | 7488579CD1 | 242 | signal_cleavage: M1-A25 | SPSCAN |
| | | | Signal Peptides: M8-A23, M1-A23, M8-A25, M1-A25 | HMMER |
| | | | Trypsin: I38-T242 | HMMER_PFAM |
| | | | Type I fibronectin domain proteins BL01253: | BLIMPS_BLOCKS |
| | | | C66-A79, E136-N172, H224-C237 | |
| | | | Serine proteases, trypsin family, active | PROFILESCAN |
| | | | sites: H64-Q107, V212-T242 | |
| | | | Chymotrypsin serine protease family (S1) | BLIMPS_PRINTS |
| | | | signature PR00722: G67-C82, Q124-V138, H224- | |
| | | | REPEAT PRECURSOR GLYCOPROTEIN PD00120: | BLIMPS_PRODOM |
| | | | G67-A79, D128-L132, D225-G233 | |
| | | | PROTEASE SERINE PRECURSOR SIGNAL | BLAST_PRODOM |
| | | | HYDROLASE ZYMOGEN GLYCOPROTEIN | |
| | | | FAMILY MULTIGENE FACTOR PD000046: D106-K238, I38-G220 | |
| | | | TRYPSIN; DM00018\|P15157\|31-270: | BLAST_DOMO |
| | | | I38-T242; DM00018\|Q02844\|29-268: I38-T242; | |
| | | | DM00018\|P21845\|31-271: G37-T242; | |
| | | | DM00018\|P15944\|31-270: I38-G241 | |
| | | | Serine proteases, trypsin family, histidine | MOTIFS |
| | | | active site: L77-C82 | |
| | | | Serine proteases, trypsin family, serine | MOTIFS |
| | | | active site: D225-V236 | |
| | | | Potential Phosphorylation Sites: S54 T125 | MOTIFS |
| | | | Potential Glycosylation Sites: N139 | MOTIFS |
| 28 | 7510521CD1 | 48 | Signal_cleavage: M1-G20 | SPSCAN |
| | | | Signal Peptide: M1-G20 | HMMER |
| | | | Signal Peptide: M1-C24 | HMMER |
| | | | Signal Peptide: M1-A28 | HMMER |
| | | | Signal Peptide: M1-R23 | HMMER |
| | | | Plant lipid transfer proteins signature: M1-K48 | PROFILESCAN |
| | | | Potential Phosphorylation Sites: T31 | MOTIFS |
| | | | Potential Glycosylation Sites: N29 | MOTIFS |

TABLE 4

| Polynucleotide | |
|---|---|
| SEQ ID NO:/ | |
| Incyte ID/Sequence | |
| Length | Sequence Fragments |
| 29/7994355CB1/4384 | 1-677, 1-779, 432-679, 432-950, 432-1041, 432-4364, 863-1620, 930-1257, 1087-1751, 1104-1357, 1338-1984, |
| | 1386-1900, 1446-1630, 1659-1969, 1659-2021, 1661-2064, 1812-2430, 1839-2144, 1843-2450, 1847-2151, 1872- |
| | 2135, 1872-2348, 1976-2242, 1976-2562, 2002-2245, 2182-2636, 2212-2466, 2277-2797, 2277-3069, 2298-2770, |
| | 2376-2812, 2381-2672, 2408-2450, 2446-2937, 2476-3046, 2499-2718, 2499-2973, 2577-2886, 2666-3114, 2701- |
| | 3285, 2766-3485, 2963-3504, 3039-3266, 3039-3284, 3068-3538, 3083-3701, 3088-3331, 3095-3607, 3110-3779, |
| | 3120-3528, 3122-3690, 3128-3267, 3128-3382, 3128-3396, 3130-3556, 3130-3605, 3178-3451, 3180-3446, 3220- |
| | 3700, 3274-3514, 3274-3551, 3274-3560, 3308-3725, 3309-3491, 3311-3627, 3314-3957, 3357-3530, 3357-3540, |
| | 3383-3632, 3383-3653, 3389-3546, 3409-3709, 3419-3528, 3430-3527, 3440-3585, 3491-3721, 3491-3725, 3491- |
| | 4023, 3494-3646, 3494-3738, 3500-3990, 3572-3826, 3572-3962, 3579-4002, 3597-4006, 3625-3885, 3660-3922, |
| | 3665-4247, 3691-4287, 3701-4353, 3704-3936, 3704-4198, 3709-3925, 3733-4376, 3768-4307, 3774-4031, |
| | 3782-4324, 3799-3983, 3802-4000, 3834-4333, 3857-4127, 3859-4009, 3888-4383, 3900-4382, 3933-4181, 3933- |
| | 4352, 3933-4360, 3942-4200, 3944-4382, 4006-4265, 4025-4379, 4036-4333, 4041-4265, 4049-4275, 4049-4359, |
| | 4058-4384, 4070-4361, 4094-4343, 4094-4351, 4150-4352, 4150-4363, 4150-4366, 4150-4380, 4171-4384, 4193- |
| | 4384, 4196-4360, 4246-4367, 4249-4374, 4257-4384, 4311-4384 |
| 30/7475875CB1/4007 | 1-180, 25-740, 433-1078, 809-1509, 961-1177, 961-1575, 974-1232, 1327-1981, 1373-1926, 1420-1875, 1611-1902, |
| | 1620-1905, 1705-1850, 1705-1951, 1705-2022, 1705-2052, 1705-2067, 1705-2068, 1705-2249, 1705-2279, 1713- |
| | 2264, 1727-2068, 1727-2157, 1755-2385, 1774-2290, 1800-2021, 1800-2258, 1800-2266, 1800-2346, 1802-2269, |
| | 1885-2113, 1885-2409, 1885-2410, 1913-2316, 1920-2309, 1959-2047, 2011-2329, 2078-2312, 2159-2247, 2219- |
| | 2770, 2303-2955, 2381-2812, 2428-2642, 2430-2875, 2453-3077, 2456-2994, 2467-3100, 2565-3092, 2568-3216, |
| | 2572-3061, 2577-3035, 2603-3036, 2609-3045, 2611-3030, 2614-3212, 2616-3180, 2630-2899, 2632-3221, 2635- |
| | 3211, 2638-3159, 2641-3134, 2655-3148, 2656-2943, 2660-3272, 2663-3361, 2672-3180, 2672-3332, 2672-3337, |
| | 2674-3162, 2678-3188, 2681-2925, 2681-3192, 2684-3262, 2690-2976, 2703-3216, 2705-3376, 2711-3279, 2712- |
| | 3216, 2715-3238, 2715-3410, 2715-3461, 2722-3304, 2724-3273, 2730-2969, 2732-2844, 2732-2995, 2734-3290, |
| | 2736-3093, 2754-3452, 2754-3454, 2767-3422, 2781-3285, 2787-3338, 2790-3359, 2792-3313, 2794-3005, |
| | 2794-3066, 2794-3250, 2794-3276, 2797-3412, 2806-2970, 2811-3067, 2822-3373, 2833-3451, 2836-3326, 2836- |
| | 3714, 2854-3355, 2876-3419, 2879-3447, 2885-3153, 2885-3461, 2888-3497, 2922-3451, 2929-3488, 2944-3547, |
| | 3010-3447, 3015-3521, 3021-3564, 3036-3663, 3059-3650, 3063-3475, 3070-3213, 3074-3442, 3094-3667, 3094- |
| | 3668, 3100-3663, 3107-3556, 3116-3679, 3129-3432, 3136-3845, 3149-3721, 3173-3668, 3184-3679, 3186-3448, |
| | 3188-3703, 3231-3754, 3258-3783, 3266-3790, 3287-3948, 3319-3844, 3320-3811, 3328-3795, 3330-4004, 3331- |
| | 3960, 3341-3594, 3346-3555, 3346-3839, 3359-3914, 3388-3602, 3388-3627, 3388-3638, 3388-3664, 3390-4001, |
| | 3409-3983, 3420-4007, 3424-3937, 3425-4000, 3426-3790, 3438-3981, 3448-4005, 3463-4003, 3468-3896, 3473- |
| | 3711, 3493-3941, 3552-4007, 3554-4007, 3555-4007, 3560-4007, 3573-4007, 3581-4007, 3582-4007, 3591-4007, |
| | 3594-4007, 3604-4007, 3615-3992, 3620-4007, 3659-4007, 3679-4007, 3790-4007, 3796-4007, 3805-4007, 3810- |
| | 3951, 3907-4007 |
| 31/71231882CB1/4524 | 1-372, 6-295, 8-714, 20-270, 21-590, 21-620, 21-651, 37-651, 87-4524, 102-651, 114-677, 129-850, 129-851, 131- |
| | 726, 131-761, 131-776, 131-798, 132-779, 231-488, 306-677, 437-677, 566-1211, 757-798, 872-1236, 1045-1219, |
| | 1045-1332, 1045-1522, 1045-1558, 1045-1576, 1048-1274, 1053-1576, 1189-1839, 1431-1912, 1469-1772, 1485- |
| | 2017, 1744-2336, 1774-2519, 1904-2425, 1930-2569, 1955-2713, 2018-2497, 2072-2716, 2153-2694, 2172-2352, |
| | 2172-2386, 2172-2615, 2172-2655, 2172-2694, 2172-2724, 2172-2730, 2172-2742, 2255-2463, 2255-2911, 2271- |
| | 2488, 2370-2772, 2400-2771, 2491-2724, 2491-2805, 2498-2686, 2501-2687, 2637-3165, 2696-3192, 2718-3058, |
| | 2821-3239, 2959-3204, 3105-4037, 3168-3564, 3204-3835, 3208-3588, 3213-3594, 3253-3596, 3258-4032, 3267- |
| | 4032, 3286-3655, 3294-4032, 3295-4032, 3296-3753, 3297-4032, 3302-3930, 3344-4032, 3362-3591, 3375-3824, |
| | 3413-4032, 3488-3680, 3488-3812, 3488-4018, 3488-4079, 3510-4032, 3530-4117, 3555-4144, 3566-4503, 3593- |
| | 3840, 3593-3872, 3593-4200, 3594-3907, 3600-4508, 3606-4236, 3626-4347, 3626-4358, 3634-4141, 3648-4428, |
| | 3653-4364, 3656-4102, 3659-4323, 3661-4368, 3702-4524, 3709-3983, 3733-3919, 3758-3972, 3758-3997, 3874- |
| | 4220, 3876-4206, 3900-4441, 3911-4524 |
| 32/2875922CB1/3250 | 1-565, 9-482, 112-594, 190-821, 190-860, 256-469, 318-1062, 394-861, 473-668, 473-977, 638-1128, 682-943, 682- |
| | 1241, 1108-1735, 1141-1573, 1414-1719, 1414-1930, 1414-1933, 1414-1936, 1414-1937, 1414-1951, 1414-1985, |
| | 1414-2050, 1453-2185, 1458-1983, 1616-1894, 1616-2120, 1626-2140, 1663-2321, 1672-1977, 1689-2110, 1708- |
| | 2045, 1708-2071, 1708-2083, 1708-2088, 1728-2171, 1731-2368, 1753-2415, 1787-2376, 1813-2117, 1820-2568, |
| | 1841-2324, 1850-2403, 1858-2330, 1870-2511, 1881-2123, 1881-2482, 1895-2246, 1903-2180, 1923-2059, 1924- |
| | 2631, 1925-2466, 1977-2534, 1992-2521, 2035-2658, 2049-2687, 2059-2197, 2060-2197, 2063-2658, 2067-2618, |
| | 2087-2673, 2147-2742, 2157-2742, 2161-2673, 2176-2622, 2194-2584, 2214-2519, 2217-2673, 2226-2742, 2232- |
| | 2523, 2232-2716; 2233-2481, 2266-2631, 2296-2700, 2323-2750, 2333-2750, 2339-2749, 2365-2742, 2377-2879, |
| | 2444-2708, 2683-3152, 2688-3110, 2715-3250, 2737-3203, 2750-3196, 2783-3016, 2798-3075, 2800-3128, 2866- |
| | 3196, 2963-3195, 3039-3182, 3113-3250 |
| 33/8158136CB1/3834 | 1-523, 1-633, 1-638, 1-3802, 19-869, 26-302, 26-340, 26-603, 34-300, 34-528, 43-595, 61-947, 132-833, 207-447, |
| | 221-784, 419-916, 433-1161, 509-1021, 646-1308, 672-1213, 679-1111, 719-1106, 735-1120, 766-1264, 775-1408, |
| | 792-1292, 804-1351, 810-1309, 823-1510, 839-1163, 855-1430, 895-1147, 895-1399, 897-1201, 919-1168, 970- |
| | 1202, 983-1237, 990-1391, 1013-1655, 1041-1537, 1041-1541, 1065-1524, 1094-1593, 1100-1369, 1116-1368, 1116- |
| | 1654, 1116-1691, 1117-1642, 1127-1389, 1132-1771, 1184-1433, 1190-1406, 1217-1629, 1220-1448, 1237-1711, |
| | 1246-1491, 1252-1810, 1329-1738, 1368-2054, 1387-1933, 1391-1668, 1392-1956, 1437-1597, 1460-1688, 1461- |
| | 1609, 1490-2065, 1501-1799, 1523-1806, 1529-1784, 1556-1820, 1586-1841, 1686-2203, 1717-2475, 1722-1981, |
| | 1795-2397, 1798-2537, 1848-2107, 1855-2601, 1900-2578, 1908-2625, 1910-2273, 1911-2623, 1995-2606, 2002- |
| | 2705, 2019-2628, 2040-2775, 2061-2763, 2061-2804, 2061-2845, 2066-2694, 2083-2769, 2089-2678, 2091-2797, |
| | 2097-2827, 2102-2550, 2123-2527, 2133-2421, 2178-2619, 2179-2473, 2191-2804, 2207-2706, 2210-2921, |
| | 2215-2901, 2215-2938, 2247-2496, 2256-3023, 2261-2477, 2266-2865, 2267-2543, 2299-2604, 2325-2786, 2329- |
| | 2838, 2329-2968, 2336-3076, 2336-3162, 2336-3209, 2361-2624, 2366-2938, 2372-2875, 2405-3011, 2435-3071, |
| | 2449-2718, 2581-3450, 2636-3304, 2666-3445, 2741-3405, 2812-3481, 2816-3480, 2824-3480, 2828-3480, 2832- |
| | 3441, 2860-3478, 2863-3436, 2866-3483, 2888-3588, 2896-3450, 2907-3482, 2916-3490, 2922-3464, 2923-3467, |
| | 2926-3376, 2941-3482, 2954-3482, 2961-3479, 2966-3475, 2982-3490, 2997-3448, 3016-3804, 3034-3503, 3042- |
| | 3476, 3043-3487, 3047-3483, 3062-3488, 3064-3482, 3067-3483, 3087-3494, 3093-3483, 3099-3375, 3112-3487, |
| | 3118-3488, 3124-3323, 3137-3390, 3137-3558, 3144-3357, 3159-3489, 3176-3490, 3177-3444, 3178-3381, 3186- |
| | 3391, 3186-3416, 3187-3803, 3205-3470, 3222-3487, 3230-3804, 3232-3804, 3237-3495, 3257-3698, 3257-3798, |
| | 3268-3483, 3270-3488, 3281-3465, 3311-3490, 3320-3519, 3320-3616, 3331-3482, 3334-3481, 3334-3487, 3334- |
| | 3494, 3343-3805, 3357-3808, 3398-3483, 3400-3804, 3403-3833, 3412-3834, 3426-3804, 3430-3802 |
| 34/5969491CB1/4493 | 1-568, 1-598, 1-666, 65-4493, 80-621, 92-316, 92-655, 107-828, 107-829, 109-704, 109-739, 109-754, 109-776, 110- |
| | 757, 209-466, 284-655, 415-655, 544-1189, 735-776, 850-1211, 1023-1197, 1023-1310, 1023-1500, 1023-1536, |
| | 1023-1554, 1026-1252, 1032-1554, 1049-1498, 1136-1420, 1167-1407, 1167-1817, 1235-1472, 1263-1554, 1301- |
| | 1517, 1409-1890, 1447-1750, 1463-1995, 1722-2314, 1752-2497, 1882-2403, 1908-2547, 1933-2691, 1995-2475, |
| | 2051-2692, 2133-2672, 2150-2330, 2150-2364, 2150-2593, 2150-2633, 2150-2672, 2150-2701, 2150-2708, 2150- |
| | 2720, 2153-2888, 2233-2441, 2249-2466, 2298-2901, 2348-2750, 2378-2749, 2378-2803, 2392-2842, 2469-2702, |
| | 2469-2783, 2476-2664, 2479-2665, 2498-2803, 2538-2845, 2568-2827, 2581-3326, 2583-3077, 2587-3122, 2592- |
| | 2994, 2632-2952, 2636-3358, 2648-2910, 2650-2901, 2666-2940, 2677-2950, 2698-2914, 2716-3143, 2756-3243, |
| | 2793-3022, 2793-3195, 2793-3368, 2793-3457, 2793-3473, 2793-3475, 2793-3492, 2839-3074, 2849-3317, 2877- |
| | 3122, 2883-3411, 2907-3499, 2919-3490, 2943-3259, 2945-3502, 2948-3511, 2959-3266, 3043-3955, 3060-3516, |
| | 3086-3367, 3086-3482, 3094-3526, 3118-3425, 3122-3716, 3131-3512, 3139-3428, 3161-3520, 3171-3514, 3176- |
| | 3950, 3185-3950, 3204-3511, 3208-3573, 3211-3526, 3212-3950, 3213-3950, 3214-3671, 3215-3950, 3220-3848, |
| | 3262-3950, 3280-3509, 3296-3742, 3331-3950, 3346-3512, 3406-3597, 3406-3730, 3406-3936, 3406-3996, 3428- |
| | 3950, 3448-4035, 3473-4021, 3495-3685, 3504-4243, 3511-3758, 3511-3790, 3511-4118, 3512-3825, 3518-4427, |
| | 3525-4153, 3544-4276, 3545-4265, 3554-4059, 3566-4347, 3571-4282, 3574-3937, 3577-4158, 3579-4286, 3620- |
| | 4472, 3627-3901, 3651-3837, 3676-3890, 3676-3915, 3792-4138, 3799-4124, 3818-4360, 3830-4442 |
| 35/7497367CB1/2921 | 1-798, 33-671, 37-635, 48-194, 48-197, 50-191, 51-197, 59-194, 59-197, 95-197, 95-1824, 303-663, 332-434, 356- |
| | 396, 356-410, 526-1210, 552-1231, 1199-1347, 1199-1799, 1199-1864, 1199-1871, 1199-1968, 1199-1992, 1199- |
| | 2061, 1199-2064, 1199-2123, 1200-1968, 1452-1563, 1871-2287, 1871-2601, 1871-2762, 1874-2428, 1874-2467, |
| | 1874-2496, 2047-2707, 2051-2712, 2064-2585, 2109-2552, 2156-2687, 2193-2646, 2196-2752, 2291-2921, 2423- |
| | 2907, 2435-2921, 2756-2858, 2756-2862, 2759-2861, 2780-2820, 2780-2834, 2867-2921 |
| 36/7632424CB1/2572 | 1-714, 24-727, 33-612, 49-776, 49-811, 50-586, 60-720, 71-700, 85-885, 87-591, 87-618, 98-895, 122-664, 122-754, |
| | 123-606, 210-707, 251-928, 370-413, 370-565, 370-633, 370-737, 420-924, 420-1020, 453-704, 453-959, 468-937, |
| | 515-1213, 575-1277, 580-826, 580-1180, 581-1188, 599-1257, 780-1292, 788-1404, 809-961, 862-1416, 902-1548, |
| | 906-1144, 936-1683, 945-1613, 956-1108, 962-1108, 963-1108, 969-1108, 969-1167, 985-1108, 989-1534, 1021- |
| | 1736, 1025-1680, 1031-1736, 1063-1564, 1063-1719, 1084-1880, 1090-1714, 1106-1153, 1137-1768, 1150-1797, |
| | 1151-1790, 1195-1798, 1197-1797, 1201-1810, 1219-1850, 1228-1804, 1240-1594, 1241-1664, 1338-1624, 1387- |
| | 1999, 1417-2183, 1456-2180, 1497-1756, 1545-2118, 1642-2083, 1660-2052, 1665-2086, 1667-1717, 1670-2204, |
| | 1679-1967, 1693-1967, 1695-1972, 1695-2078, 1695-2083, 1700-2078, 1749-2063, 1754-2183, 1755-2063, 1774- |
| | 2018, 1774-2079, 1774-2088, 1810-2041, 1817-2074, 1826-2086, 1870-2075, 1897-2204, 1941-2183, 2101-2556, |
| | 2101-2572, 2108-2359, 2117-2572, 2264-2570, 2383-2572 |
| 37/1804436CB1/3758 | 1-426, 23-459, 66-703, 87-455, 159-805, 162-643, 162-677, 170-811, 455-1225, 497-1051, 540-1201, 588-878, 626- |
| | 1361, 637-1122, 670-1491, 731-1134, 774-1469, 782-1261, 792-1211, 819-1398, 863-1409, 893-1531, 911-1409, |
| | 991-1577, 998-1236, 1003-1138, 1012-1394, 1015-1630, 1079-1546, 1086-1673, 1087-1597, 1087-1599, 1088- |
| | 1602, 1153-1435, 1187-1522, 1199-1467, 1332-1948, 1358-3567, 1374-1951, 1397-1738, 1399-1988, 1411-1844, |
| | 1424-1723, 1438-1723, 1492-2157, 1572-2168, 1601-2175, 1608-1884, 1632-1902, 1635-1900, 1643-2079, 1693- |
| | 2337, 1695-2296, 1695-2318, 1695-2379, 1695-2406, 1796-2046, 1807-2082, 1807-2276, 1807-2280, 1807-2311, |
| | 1823-2331, 1846-2389, 1860-2393, 1941-2206, 2002-2224, 2024-2243, 2050-2227, 2284-2773, 2329-2906, 2392- |
| | 2881, 2445-2749, 2449-2792, 2449-2929, 2449-2935, 2449-2948, 2449-2969, 2449-3034, 2449-3043, 2449-3052, |
| | 2449-3061, 2449-3082, 2457-2746, 2503-2782, 2524-2776, 2670-3308, 2681-3222, 2685-2875, 2685-3230, 2697- |
| | 3334, 2704-2943, 2704-2946, 2704-2947, 2704-2949, 2713-3322, 2718-3329, 2736-3331, 2737-3282, 2749-3369, |
| | 2750-3407, 2753-3035, 2759-3156, 2763-3306, 2767-3369, 2771-3405, 2775-3027, 2775-3400, 2783-3287, 2785- |
| | 3034, 2803-3199, 2803-3456, 2809-3350, 2815-3158, 2820-3402, 2826-3078, 2826-3453, 2840-3357, 2854-3317, |
| | 2855-3369, 2857-3314, 2857-3318, 2858-3098, 2865-3102, 2865-3481, 2866-3149, 2878-3539, 2881-3551, 2901- |
| | 3439, 2906-3553, 2908-3347, 2918-3514, 2920-3555, 2923-3502, 2937-3094, 2944-3201, 2944-3213, 2947-3567, |
| | 2960-3562, 2975-3223, 2975-3248, 2975-3553, 2994-3207, 2999-3257, 3001-3270, 3004-3290, 3008-3233, 3011- |
| | 3464, 3014-3276, 3015-3252, 3026-3280, 3027-3281, 3035-3567, 3046-3274, 3046-3306, 3046-3314, 3046-3315, |
| | 3052-3567, 3057-3324, 3061-3567, 3063-3326, 3069-3330, 3072-3567, 3074-3567, 3076-3378, 3077-3563, 3090- |
| | 3360, 3092-3567, 3100-3559, 3102-3560, 3105-3564, 3108-3556, 3110-3489, 3113-3562, 3113-3567, 3117-3567, |
| | 3125-3560, 3130-3387, 3135-3559, 3138-3436, 3140-3394, 3141-3367, 3141-3559, 3144-3567, 3151-3559, |
| | 3155-3559, 3157-3562, 3157-3563, 3162-3554, 3169-3546, 3171-3560, 3171-3562, 3175-3559, 3176-3428, 3180- |
| | 3235, 3180-3559, 3181-3564, 3181-3693, 3181-3758, 3187-3410, 3197-3563, 3199-3566, 3205-3559, 3210-3559, |
| | 3218-3442, 3218-3445, 3218-3455, 3220-3559, 3240-3565, 3243-3510, 3246-3559, 3265-3559, 3266-3559, 3269- |
| | 3559, 3286-3507, 3286-3552, 3286-3559, 3286-3562, 3289-3560, 3290-3544, 3291-3559, 3294-3529, 3297-3566, |
| | 3335-3561, 3352-3567, 3359-3566, 3388-3567, 3420-3557 |
| 38/7486358CB1/1036 | 1-698, 1-832, 143-831, 148-831, 588-1030, 588-1036, 592-831 |
| 39/7472344CB1/3651 | 1-654, 179-734, 214-871, 515-980, 542-1000, 812-1067, 852-1392, 958-1474, 1002-1222, 1031-1598, 1037-1799, |
| | 1039-1738, 1062-1440, 1200-1824, 1241-1860, 1281-1824, 1675-1960, 1675-1986, 1675-2039, 1675-2167, 1675- |
| | 2184, 1675-2207, 1675-2299, 1675-2324, 1709-2324, 1748-2324, 2014-2637, 2204-2328, 2235-2328, 2550-2826, |
| | 2660-2889, 2689-2938, 2689-2952, 2692-2889, 2693-2923, 2693-2984, 2693-3189, 2694-3222, 2695-3173, 2697- |
| | 2990, 2697-3183, 2699-3248, 2701-3002, 2703-2896, 2763-3021, 2787-3021, 2834-3218, 2834-3238, 2837-3388, |
| | 2845-3232, 3005-3181, 3005-3317, 3011-3414, 3015-3409, 3074-3314, 3081-3321, 3081-3651, 3084-3345, 3084- |
| | 3601 |
| 40/7192959CB1/1989 | 1-517, 170-815, 170-870, 170-882, 172-564, 172-775, 172-810, 174-871, 197-769, 222-826, 430-889, 539-1122, 559- |
| | 802, 559-1127, 573-875, 621-1361, 640-1063, 666-904, 720-991, 963-1617, 991-1282, 991-1497, 1012-1622, 1069- |
| | 1618, 1145-1539, 1275-1507, 1275-1630, 1370-1589, 1370-1622, 1378-1619, 1424-1743, 1465-1716, 1645-1989, |
| | 1730-1989, 1751-1935, 1751-1989, 1766-1989, 1773-1989, 1780-1989, 1784-1989, 1785-1989, 1829-1989, 1840- |
| | 1989, 1845-1989, 1853-1989, 1880-1989, 1886-1989 |
| 41/6169565CB1/1629 | 1-747, 275-554, 275-957, 275-992, 275-1064, 275-1082, 282-1085, 643-1230, 690-1438, 694-1407, 733-1590, 842- |
| | 1629, 863-1629, 868-1628, 871-1587, 935-1629 |
| 42/7494717CB1/3166 | 1-237, 90-702, 100-922, 100-955, 104-842, 127-307, 137-222, 137-776, 137-784, 141-405, 144-307, 144-608, 150- |
| | 279, 150-613, 166-600, 171-756, 193-977, 391-696, 394-885, 424-887, 459-649, 496-1311, 506-916, 541-1014, 566- |
| | 864, 566-1031, 566-1077, 574-989, 629-1193, 637-1266, 638-1116, 639-1235, 648-1283, 673-866, 684-1028, 689- |
| | 946, 701-1248, 735-977, 748-1311, 759-1002, 819-1297, 824-1450, 842-1479, 853-1495, 853-1535, 857-1079, 878- |
| | 1118, 883-1567, 893-1393, 912-1320, 924-1531, 944-1549, 954-1235, 1018-1486, 1020-1264, 1032-1331, 1036- |
| | 1515, 1037-1467, 1053-1179, 1072-1560, 1151-1760, 1196-1484, 1217-1543, 1268-1486, 1280-1579, 1286-1750, |
| | 1295-1560, 1320-1641, 1379-1625, 1390-1645, 1398-1526, 1437-1756, 1444-1689, 1476-1736, 1501-1733, 1719- |
| | 1950, 1720-2098, 1798-2096, 1827-2175, 1879-2107, 1889-2144, 1937-2197, 1954-2196, 1958-2248, 1958-2490, |
| | 1974-2216, 2001-2257, 2003-2646, 2012-2208, 2045-2280, 2046-2248, 2113-2353, 21 14-2309, 2144-2409, 2204- |
| | 2454, 2304-2654, 2338-2537, 2340-2552, 2346-2583, 2357-2702, 2379-2596, 2425-2703, 2477-2731, 2482-2697, |
| | 2495-2740, 2503-2697, 2522-2782, 2630-3132, 2631-2844, 2634-3116, 2649-3126, 2665-2904, 2737-3020, 2737- |
| | 3143, 2737-3166, 2814-3027, 2818-3007, 2828-3074, 2861-2999, 2965-3109 |
| 43/7497510CB1/399 | 1-144, 69-338, 156-335, 192-399, 212-399 |
| 44/7498882CB1/1811 | 1-237, 15-181, 21-247, 22-466, 22-519, 23-197, 27-253, 27-289, 28-302, 28-366, 29-280, 34-315, 35-161, 35-278, |
| | 35-281, 37-248, 37-291, 48-200, 48-309, 48-421, 58-310, 59-359, 60-294, 60-871, 61-184, 61-273, 61-299, 61-304, |
| | 61-315, 61-328, 61-333, 61-334, 61-391, 61-657, 61-703, 62-251, 62-299, 62-315, 62-321, 62-344, 62-460, 63-319, |
| | 63-442, 63-592, 64-302, 65-274, 65-307, 65-308, 65-326, 65-328, 65-329, 65-342, 65-630, 66-283, 66-301, 66-305, |
| | 66-315, 66-322, 66-325, 66-326, 66-331, 66-343, 66-440, 66-634, 67-196, 67-248, 67-275, 67-292, 67-294, 67-302, |
| | 67-316, 67-381, 67-490, 68-300, 68-306, 68-645, 69-308, 69-460, 70-300, 70-321, 70-328, 70-331, 70-339, 70-354, |
| | 70-356, 70-423, 71-171, 71-673, 72-287, 73-351, 73-386, 76-364, 79-346, 80-334, 82-346, 83-346, 83-348, 85-329, |
| | 85-338, 85-360, 85-361, 85-364, 85-374, 85-526, 85-564, 86-284, 86-310, 86-322, 86-329, 86-352, 86-355, 86-361, |
| | 86-362, 86-365, 86-373, 86-381, 86-383, 86-386, 86-387, 86-432, 86-450, 86-549, 86-555, 86-574, 86-619, 86-664, |
| | 86-668, 86-669, 86-675, 86-680, 86-714, 87-446, 92-619, 96-380, 102-472, 103-308, 103-329, 103-684, 104-351, |
| | 104-1374, 107-290, 117-359, 117-401, 118-365, 129-381, 129-387, 135-388, 141-403, 156-431, 162-822, 165-632, |
| | 166-448, 172-576, 172-769, 173-386, 183-415, 183-601, 197-534, 210-621, 211-518, 224-403, 225-427, 243-528, |
| | 261-522, 267-553, 393-851, 412-841, 443-683, 447-699, 449-715, 449-742, 451-698, 453-721, 454-601, 459-778, |
| | 474-762, 487-744, 487-750, 490-801, 507-705, 507-764, 509-759, 522-827, 527-752, 527-785, 553-867, 565-838, |
| | 565-869, 582-869, 584-871, 595-843, 597-821, 597-856, 602-845, 602-871, 603-826, 608-706, 632-868, 656-853, |
| | 663-766, 674-829, 693-1238, 694-983, 868-1351, 868-1509, 870-962, 871-1143, 873-1154, 873-1292, 880-1109, |
| | 909-1 168, 909-1457, 913-1175, 915-1156, 915-1379, 916-11 16, 916-1143, 930-1176, 933-1232, 934-1 194, 935- |
| | 1191, 935-1368, 936-1181, 938-1204, 939-1177, 941-1265, 974-1195, 990-1264, 991-1257, 1005-1254, 1008-1252, |
| | 1020-1253, 1020-1363, 1024-1314, 1027-1315, 1027-1322, 1028-1270, 1039-1285, 1039-1329, 1043-1292, 1044- |
| | 1265, 1044-1351, 1046-1289, 1046-1303, 1048-1331, 1051-1714, 1055-1267, 1055-1361, 1079-1358, 1079-1584, |
| | 1086-1381, 1087-1276, 1087-1351, 1092-1598, 1099-1383, 1101-1391, 1107-1530, 1109-1370, 1112-1430, 1115- |
| | 1377, 1115-1576, 1117-1624, 1123-1317, 1125-1406, 1130-1438, 1131-1315, 1131-1318, 1132-1440, 1135-1403, |
| | 1136-1425, 1141-1391, 1143-1427, 1145-1775, 1146-1414, 1149-1407, 1149-1592, 1152-1393, 1152-1406, 1154- |
| | 1380, 1154-1402, 1154-1410, 1158-1772, 1161-1260, 1161-1413, 1161-1430, 1165-1390, 1166-1773, 1167-1740, |
| | 1175-1437, 1175-1463, 1176-1419, 1176-1449, 1176-1739, 1184-1729, 1188-1438, 1188-1456, 1195-1676, 1201- |
| | 1772, 1207-1470, 1210-1456, 1219-1414, 1219-1497, 1246-1773, 1249-1403, 1250-1552, 1255-1523, 1256-1775, |
| | 1265-1555, 1275-1711, 1289-1811, 1300-1787, 1304-1547, 1306-1775, 1312-1775, 1315-1787, 1319-1747, 1324- |
| | 1775, 1325-1431, 1327-1775, 1331-1787, 1332-1787, 1333-1781, 1336-1582, 1347-1593, 1352-1781, 1354-1782, |
| | 1355- |
| | 1783, 1357-1644, 1357-1787, 1358-1790, 1359-1781, 1360-1787, 1360-1790, 1361-1775, 1361-1794, 1364-1777, |
| | 1364-1783, 1365-1775, 1365-1781, 1367-1734, 1367-1774, 1368-1781, 1370-1749, 1371-1791, 1373-1782, 1374- |
| | 1781 |
| 45/5524205CB1/4407 | 1-981, 25-703, 144-868, 183-482, 286-913, 531-1263, 614-868, 615-1263, 617-915, 617-1000, 769-1034, 871-1014, |
| | 871-1186, 924-1568, 926-1167, 926-1243, 926-1372, 926-1568, 931-1514, 1004-1385, 1004-1595, 1008-1595, 1049- |
| | 1294, 1122-1654, 1155-1399, 1155-4378, 1160-1457, 1160-1501, 1400-1600, 1400-1737, 1409-1600, 1409-1737, |
| | 1601-1737, 1601-1823, 1627-1931, 1627-2195, 1627-2218, 1687-2376, 1772-2386, 1917-2373, 1939-2661, 2008- |
| | 2267, 2049-2394, 2094-2371, 2137-2635, 2138-2408, 2186-2427, 2285-2698, 2311-2499, 2317-2548, 2367-3019, |
| | 2396-3011, 2398-2699, 2398-3213, 2403-3014, 2460-3046, 2546-3044, 2555-2808, 2555-2817, 2566-3177, 2568- |
| | 3049, 2570-2825, 2665-2881, 2679-3099, 2834-3025, 2834-3282, 2924-3178, 2947-3553, 2996-3550, 3067-3325, |
| | 3098-3667, 3200-3704, 3285-3544, 3290-3605, 3291-3454, 3417-3750, 3429-3672, 3541-3830, 3541-3831, 3571- |
| | 3822, 3571-4122, 3572-3817, 3587-4067, 3671-3938, 3671-3946, 3683-3971, 3740-4007, 3765-4057, 3825-3892, |
| | 3836-4044, 3874-4060, 3883-4197, 3906-4150, 3915-4249, 3984-4224, 4008-4291, 4011-4247, 4012-4262, |
| | 4030-4321, 4147-4407, 4161-4374 |
| 46/7102342CB1/2177 | 1-745, 6-581, 13-543, 15-510, 21-759, 24-769, 44-772, 194-371, 194-531, 194-578, 194-580, 195-580, 198-268, 198- |
| | 579, 198-580, 199-580, 204-580, 209-580, 220-580, 255-578, 278-580, 279-580, 650-1213, 650-1252, 650-1310, |
| | 650-1328, 650-1355, 902-1145, 902-1453, 902-1508, 1185-1584, 1206-1722, 1219-1616, 1220-1735, 1227-1907, |
| | 1229-1657, 1272-1444, 1322-2121, 1342-2177, 1370-1666, 1384-2176, 1404-2177, 1414-2177, 1514-2161, 1542- |
| | 2177, 1598-2171, 1894-1961, 1909-1961, 1948-2000, 2051-2171, 2055-2171, 2112-2171 |
| 47/4169939CB1/703 | 1-603, 5-300, 109-703, 112-703, 129-417, 132-427 |
| 48/6539977CB1/1295 | 1-448, 1-450, 1-1295, 3-322, 150-455, 156-455, 449-933, 459-778, 459-842, 459-865, 475-721, 475-1050, 865- |
| | 1295, 1061-1112, 1061-1149, 1061-1186, 1061-1222, 1061-1259, 1097-1295, 1134-1295, 1171-1295, 1207-1295, |
| | 1244-1295 |
| 49/7675588CB1/575 | 1-273, 1-492, 1-566, 65-323, 79-566, 89-319, 196-328, 243-575, 262-575, 420-566 |
| 50/6244077CB1/1062 | 1-652, 50-1060, 490-970, 490-986, 490-1018, 490-1019, 490-1062, 493-1050, 514-1021, 624-1049, 647-735, 656- |
| | 1055, 667-1062, 696-1053, 744-1055, 770-852 |
| 51/7498404CB1/1029 | 1-599, 2-1029 |
| 52/7391748CB1/905 | 1-528, 30-256, 30-309, 246-475, 250-492, 261-697, 265-842, 317-626, 321-583, 325-780, 335-887, 336-618, 360- |
| | 558, 360-836, 374-606, 378-837, 386-553, 387-618, 388-865, 399-645, 400-658, 418-905, 422-905, 428-888, 429- |
| | 884, 430-701, 433-659, 433-660, 444-836, 445-889, 446-888, 449-713, 454-579, 456-752, 459-625, 459-884, 464- |
| | 888, 472-888, 480-710, 482-732 |
| 53/7499780CB1/2667 | 1-585, 66-747, 173-512, 173-650, 173-745, 203-450, 215-494, 215-498, 215-742, 215-803, 234-663, 266-841, 288- |
| | 573, 297-799, 373-1007, 389-924, 445-850, 449-950, 510-791, 512-837, 513-1071, 516-782, 518-652, 520-772, 520- |
| | 1123, 521-1054, 528-1044, 530-807, 530-1022, 530-1081, 532-703, 535-1022, 536-797, 539-655, 550-1138, 558- |
| | 1155, 570-860, 572-704, 572-814, 572-829, 589-1087, 591-909, 613-892, 618-822, 622-926, 635-1121, 666-927, |
| | 675-782, 784-1015, 784-1028, 801-992, 844-1144, 849-957, 849-1536, 872-1121, 921-1155, 933-1155, 964-1155, |
| | 1026-1130, 1155-1278, 1159-1758, 1159-1797, 1162-1698, 1165-1606, 1186-1686, 1196-1372, 1198-1677, 1210- |
| | 1516, 1210-1731, 1211-1775, 1215-1682, 1215-1793, 1232-1487, 1232-1509, 1260-1924, 1263-1519, 1267-1916, |
| | 1273-1502, 1277-1758, 1278-1943, 1282-1573, 1286-1555, 1286-1938, 1291-1925, 1296-1925, 1298-1576, 1300- |
| | 1553, 1322-1620, 1327-1570, 1329-1847, 1343-1602, 1349-1633, 1352-2038, 1362-1485, 1365-1962, 1371-1929, |
| | 1379-2064, 1381-1513, 1382-1641, 1382-1669, 1403-2073, 1405-2004, 1414-2054, 1423-1916, 1427-1937, |
| | 1435-1869, 1441-1695, 1453-1908, 1473-1704, 1475-1731, 1476-1978, 1476-2024, 1476-2036, 1476-2056, 1480- |
| | 2036, 1497-1719, 1497-1782, 1507-1708, 1515-2186, 1517-2215, 1518-2011, 1520-2072, 1534-1809, 1536-2234, |
| | 1537-1774, 1543-2102, 1547-2009, 1550-2012, 1551-2147, 1551-2168, 1552-2248, 1563-2005, 1565-1910, 1568- |
| | 2089, 1574-1832, 1574-2181, 1576-2289, 1578-2282, 1580-2218, 1583-1852, 1587-2126, 1596-2278, 1606-2264, |
| | 1608-2108, 1609-1912, 1611-2126, 1614-1767, 1620-2110, 1622-2173, 1624-1872, 1624-1917, 1624-2237, 1630- |
| | 2179, 1638-2211, 1641-1869, 1642-2291, 1649-2105, 1650-2277, 1653-1787, 1658-1927, 1659-1926, 1667-2182, |
| | 1667-2275, 1668-1907, 1668-1943, 1670-1938, 1675-2288, 1679-1952, 1679-2192, 1681-1974, 1682-2363, 1682- |
| | 2383, 1685-1955, 1685-2379, 1687-1955, 1719-2343, 1719-2363, 1721-2200, 1723-1983, 1723-2005, 1725-2312, |
| | 1726-2032, 1726-2279, 1727-2433, 1742-1980, 1764-1981, 1765-1833, 1765-2240, 1765-2355, 1766-2053, |
| | 1769-2347, 1773-1834, 1774-2278, 1775-2267, 1776-2078, 1778-2196, 1793-2390, 1799-2388, 1809-2081, 1809- |
| | 2358, 1821-2012, 1822-2088, 1822-2096, 1823-2416, 1824-2102, 1829-2113, 1835-2355, 1844-2510, 1850-2138, |
| | 1850-2170, 1861-2071, 1861-2174, 1870-2152, 1876-2514, 1880-2281, 1880-2297, 1881-2145, 1882-2151, 1884- |
| | 2489, 1885-2141, 1885-2468, 1888-2121, 1888-2451, 1893-2180, 1895-2416, 1896-2563, 1896-2584, 1898-2618, |
| | 1902-2121, 1902-2523, 1904-2113, 1909-2484, 1920-2128, 1920-2559, 1921-2578, 1923-2141, 1923-2550, 1929- |
| | 2576, 1933-2172, 1953-2244, 1959-2405, 1962-2226, 1963-2326, 1963-2646, 1975-2667, 1981-2635, 1982-2653, |
| | 1989-2258, 1989-2583, 1992-2234, 1992-2239, 1992-2248, 2003-2274, 2004-2583, 2005-2651, 2005-2653, 2006- |
| | 2240, 2007-2266, 2008-2298, 2009-2248, 2015-2347, 2021-2503, 2021-2667, 2022-2360, 2030-2480, 2040-2666, |
| | 2040-2667, 2041-2302, 2044-2309, 2044-2410, 2048-2355, 2050-2667, 2051-2172, 2051-2306, 2051-2320, |
| | 2051-2345, 2051-2355, 2056-2603, 2059-2221, 2060-2621, 2064-2311, 2064-2334, 2064-2365, 2070-2324, 2074- |
| | 2298, 2088-2661, 2089-2650, 2091-2372, 2091-2667, 2094-2399, 2098-2630, 2100-2347, 2104-2667, 2105-2667, |
| | 2108-2667, 2110-2656, 2111-2666, 2122-2556, 2132-2667, 2135-2666, 2141-2653, 2143-2667, 2156-2667, 2160- |
| | 2664, 2162-2424, 2172-2409, 2172-2663, 2172-2664, 2181-2667, 2187-2440, 2191-2667, 2194-2379, 2195-2667, |
| | 2196-2662, 2196-2667, 2197-2457, 2202-2661, 2204-2666, 2207-2662, 2208-2663, 221 1-2457, 2211-2660, 2211- |
| | 2664, 2212-2545, 2212-2667, 2216-2459, 2218-2510, 2218-2653, 2218-2667, 2221-2506, 2221-2663, 2223-2667, |
| | 2225-2663, 2227-2666, 2230-2665, 2231-2663, 2231-2667, 2243-2663, 2251-2639, 2251-2662, 2253-2664, 2254- |
| | 2664, 2260-2666, 2261-2663, 2263-2662, 2269-2662, 2271-2664, 2274-2664, 2277-2666, 2278-2664, 2286-2662, |
| | 2288-2664, 2290-2662, 2298-2547, 2305-2665, 2313-2663, 2317-2660, 2336-2573, 2336-2626, 2358-2505 |
| 54/7499881CB1/2977 | 1-258, 1-310, 33-767, 42-258, 42-610, 306-729, 372-941, 407-687, 440-794, 545-713, 795-1350, 1031-1646, 1062- |
| | 1309, 1088-1337, 1108-1603, 1137-1345, 1161-1432, 1200-1581, 1230-1793, 1239-1499, 1245-1793, 1288-1738, |
| | 1348-1793, 1368-1517, 1375-1656, 1380-2242, 1383-1629, 1393-1715, 1415-1631, 1433-1571, 1439-1744, 1461- |
| | 1671, 1469-1693, 1469-2019, 1478-1665, 1495-1707, 1517-1732, 1520-2009, 1528-1763, 1589-1862, 1606-1910, |
| | 1640-1928, 1679-1914, 1681-1929, 1682-2087, 1692-1862, 1727-2227, 1746-2533, 1790-2063, 1795-2120, 1806- |
| | 2059, 1809-2083, 1827-2533, 1836-2067, 1845-2098, 1870-2160, 1876-2169, 1904-2179, 1911-2533, 1923-2260, |
| | 1925-2288, 1940-2200, 1941-2214, 1945-2209, 1947-2533, 1956-2554, 1957-2215, 1957-2535, 1966-2088, 1972- |
| | 2533, 1975-2312, 1977-2217, 1977-2246, 1983-2254, 1997-2533, 1999-2291, 2033-2320, 2046-2499, 2062-2563, |
| | 2073-2687, 2074-2646, 2077-2499, 2079-2341, 2083-2329, 2090-2355, 2093-2420, 2103-2336, 2115-2386, 2118- |
| | 2533, 2120-2396, 2136-2636, 2179-2910, 2185-2506, 2192-2848, 2199-2895, 2200-2790, 2201-2714, 2214-2488, |
| | 2215-2458, 2228-2927, 2236-2732, 2262-2579, 2264-2520, 2285-2552, 2288-2541, 2292-2916, 2304-2964, 2311- |
| | 2952, 2338-2848, 2358-2598, 2362-2665, 2368-2977, 2372-2927, 2374-2956, 2376-2848, 2397-2655, 2402-2921, |
| | 2408-2952, 2415-2977, 2417-2905, 2422-2702, 2422-2952, 2424-2650, 2436-2617, 2443-2947, 2463-2759, 2469- |
| | 2690, 2474-2977, 2484-2727, 2491-2965, 2494-2748, 2494-2961, 2494-2965, 2496-2970, 2502-2971, 2509-2966, |
| | 2509-2972, 2510-2965, 2512-2971, 2513-2778, 2513-2951, 2513-2975, 2514-2965, 2515-2977, 2517-2971, 2518- |
| | 2764, 2518-2964, 2519-2822, 2519-2969, 2525-2805, 2526-2965, 2527-2780, 2527-2965, 2529-2965, 2533-2959, |
| | 2535-2965, 2544-2974, 2546-2809, 2548-2974, 2551-2803, 2551-2969, 2554-2965, 2555-2969, 2558-2971, 2559- |
| | 2965, 2560-2977, 2561-2962, 2561-2965, 2563-2965, 2564-2965, 2566-2965, 2567-2969, 2568-2965, 2573-2972, |
| | 2579-2977, 2598-2965, 2600-2958, 2604-2964, 2604-2965, 2604-2970, 2608-2965, 2609-2965, 2617-2969, 2618- |
| | 2965, 2640-2959, 2653-2917, 2653-2921, 2657-2965, 2659-2965, 2693-2888, 2699-2965, 2702-2965, 2702-2970, |
| | 2703-2965, 2773-2965, 2811-2959, 2858-2977 |
| 55/7488579CB1/729 | 1-729, 23-520, 27-331, 27-357, 27-361, 34-327, 50-337, 83-364, 117-503, 127-405, 130-410, 173-438, 192-481, 218- |
| | 503, 232-503 |
| 56/7510521CB1/1879 | 1-237, 15-181, 21-241, 21-1869, 23-197, 27-241, 30-241, 35-161, 37-241, 48-200, 61-96, 61-241, 62-241, 66-241, |
| | 67-196, 67-240, 70-241, 71-171, 80-231, 112-378, 118-375, 118-386, 268-364, 269-525, 270-808, 271-515, 294- |
| | 524, 300-565, 305-592, 312-804, 317-894, 317-925, 322-575, 328-911, 330-578, 335-649, 341-597, 341-888, 347- |
| | 688, 353-933, 356-604, 366-622, 366-795, 367-525, 367-769, 368-614, 377-687, 379-943, 397-637, 400-675, 401- |
| | 653, 403-669, 403-696, 403-987, 404-715, 405-652, 407-675, 413-732, 428-716, 428-1015, 430-772, 430-998, 433- |
| | 1043, 436-723, 436-1072, 439-1150, 441-698, 441-704, 444-755, 445-1009, 458-991, 459-918, 461-659, 461-718, |
| | 463-713, 465-995, 469-758, 469-1013, 472-1053, 476-781, 481-706, 481-739, 494-904, 496-655, 496-768, 499-764, |
| | 500-962, 504-852, 507-821, 519-743, 519-792, 519-823, 527-753, 528-816, 535-817, 536-823, 538-825, 545-1094, |
| | 549-797, 551-775, 551-810, 554-839, 556-799, 556-835, 557-780, 560-660, 562-807, 565-783, 567-919, 567-943, |
| | 567-970, 586-822, 588-1129, 594-963, 595-856, 595-1316, 596-877, 598-1196, 602-844, 610-807, 612-865, |
| | 612-873, 617-720, 617-1093, 619-923, 625-867, 625-875, 625-900, 626-890, 627-875, 628-783, 628-903, 628-917, |
| | 631-879, 647-886, 660-921, 660-1127, 660-1231, 661-930, 669-920, 669-943, 669-1208, 670-905, 671-907, 672- |
| | 1285, 673-944, 676-918, 679-925, 685-890, 685-897, 685-936, 688-947, 688-1270, 689-943, 689-960, 689-1257, |
| | 696-1013, 699-1333, 703-1168, 705-999, 705-1316, 706-957, 711-999, 711-1263, 720-1072, 724-950, 724-981, 724- |
| | 1295, 732-1009, 741-1026, 745-987, 745-996, 745-998, 755-990, 769-1041, 769-1042, 778-979, 778-1174, 779- |
| | 1007, 779-1017, 781-1036, 782-1351, 788-1329, 788-1467, 791-1026, 791-1035, 797-1028, 799-1291, 803-1005, |
| | 803-1350, 814-1071, 815-1507, 821-1083, 822-1101, 824-1082, 824-1507, 829-1012, 834-1108, 836-1138, 839- |
| | 1013, 839-1087, 839-1172, 845-1113, 851-1062, 852-1288, 867-1121, 867-1133, 867-1153, 867-1166, 867-1170, |
| | 869-1122, 872-1110, 872-1135, 873-1096, 874-1712, 876-1142, 876-1146, 876-1147, 876-1150, 877-1119, 886- |
| | 1448, 888-1242, 888-1514, 896-1352, |
| | 898-1161, 901-1110, 902-1286, 904-1149, 905-1155, 908-1177, 908-1334, 909-1258, 909-1355, 910-1134, 912- |
| | 1712, 914-1103, 914-1436, 924-1165, 925-1357, 926-1204, 927-1165, 928-1034, 935-1337, 937-1081, 937-1140, |
| | 937-1150, 937-1154, 941-1136, 944-1140, 946-1595, 954-1286, 957-1225, 957-1229, 958-1712, 959-1151, 959- |
| | 1240, 959-1378, 966-1195, 966-1201, 966-1235, 967-1712, 970-1269, 977-1131, 981-1537, 985-1401, 993-1632, |
| | 995-1254, 995-1543, 999-1261, 1000-1684, 1001-1242, 1001-1465, 1002-1202, 1002-1229, 1002-1248, 1003-1712, |
| | 1014-1545, 1016-1262, 1016-1306, 1019-1318, 1020-1280, 1020-1616, 1021-1277, 1022-1267, 1022-1273, 1024- |
| | 1290, 1025-1263, 1027-1351, 1060-1281, 1068-1356, 1076-1350, 1077-1343, 1077-1732, 1077-1784, 1078-1367, |
| | 1084-1352, 1086-1691, 1091-1271, 1091-1340, 1094-1338, 1106-1339, 1106-1449, 1110-1400, 1113-1401, 1113- |
| | 1408, 1114-1356, 1114-1385, 1125-1371, 1125-1415, 1125-1675, 1129-1378, 1130-1351, 1130-1437, 1132-1375, |
| | 1132-1389, 1133- |
| | 1635, 1134-1417, 1134-1717, 1137-1800, 1141-1286, 1141-1353, 1141-1412, 1141-1429, 1141-1431, 1147-1627, |
| | 1165-1444, 1165-1668, 1172-1467, 1173-1362, 1173-1437, 1173-1580, 1178-1684, 1185-1469, 1187-1477, 1193- |
| | 1616, 1195-1456, 1198-1487, 1198-1516, 1201-1463, 1201-1662, 1203-1710, 1209-1376, 1209-1398, 1209-1403, |
| | 1210-1356, 1210-1840, 1211-1489, 1211-1492, 1216-1524, 1217-1401, 1217-1404, 1217-1470, 1218-1526, 1221- |
| | 1489, 1222-1511, 1227-1477, 1229-1462, 1229-1513, 1230-1497, 1231-1861, 1232-1500, 1234-1869, 1235-1414, |
| | 1235-1493, 1235-1678, 1238-1479, 1238-1492, 1240-1466, 1240-1488, 1240-1496, 1247-1346, 1247-1499, 1247- |
| | 1516, 1251-1476, 1255-1527, 1259-1869, 1261-1523, 1261-1549, 1262-1505, 1262-1535, 1270-1823, 1274-1524, |
| | 1274-1542, 1275-1826, 1281-1762, 1283-1802, 1292-1869, 1293-1556, 1293-1825, 1296-1542, 1296-1570, 1301- |
| | 1639, 1301-1824, 1305-1500, 1305-1583, 1313-1540, 1313-1578, 1314-1829, 1318-1773, 1332-1828, 1335- |
| | 1489, 1336-1638, 1340-1865, 1340-1879, 1341-1609, 1341-1828, 1342-1861, 1351-1641, 1352-1869, 1358-1879, |
| | 1361-1797, 1362-1633, 1371-1689, 1375-1869, 1377-1869, 1383-1835, 1384-1630, 1386-1869, 1392-1861, 1398- |
| | 1861, 1401-1869, 1404-1868, 1405-1836, 1409-1869, 1410-1861, 1411-1517, 1411-1869, 1412-1869, 1413-1861, |
| | 1417-1869, 1418-1869, 1419-1867, 1420-1867, 1422-1668, 1424-1869, 1424-1879, 1428-1696, 1433-1679, 1433- |
| | 1879, 1438-1632, 1438-1867, 1440-1868, 1440-1869, 1441-1869, 1443-1596, 1443-1730, 1443-1869, 1444-1869, |
| | 1445-1867, 1446-1869, 1447-1861, 1447-1869, 1448-1869, 1450-1711, 1450-1863, 1450-1869, 1451-1861, 1451- |
| | 1867, 1451-1868, 1451-1869, 1452-1861, 1452-1879, 1453-1820, 1453-1869, 1454-1867, 1456-1835, 1456-1869, |
| | 1457-1759, 1457-1869, 1458-1709, 1459-1868, 1460-1867, 1462-1861, 1462-1867, 1463-1868, 1463-1869, 1464- |
| | 1867, 1465-1689, 1465-1869, 1466-1601, 1466-1712, 1466-1729, 1466-1850, 4466-1861, 1467-1869, 1471-1861 |
| | 1471-1867, 1475-1861, 1476-1854, 1477-1868, 1478-1763, 1480-1869, 1481-1834, 1481-1867, 1481-1868, 1483- |
| | 1861, 1483-1867, 1484-1741, 1486-1737, 1491-1857, 1491-1869, 1492-1879, 1493-1869, 1495-1678, 1496-1756, |
| | 1498-1869, 1500-1861, 1501-1869, 1502-1861, 1504-1869, 1505-1759, 1506-1731, 1506-1748, 1507-1869, 1513- |
| | 1784, 1524-1861, 1524-1879, 1527-1869, 1530-1755, 1531-1862, 1533-1809, 1535-1815, 1541-1710, 1543-1784, |
| | 1544-1795, 1552-1826, 1554-1869, 1555-1867, 1556-1861, 1556-1868, 1563-1861, 1564-1854, 1575-1850, 1577- |
| | 1879, 1586-1869, 1588-1869, 1589-1869, 1590-1868, 1592-1868, 1592-1869, 1593-1861, 1596-1868, 1596-1869, |
| | 1598-1861, 1599-1867, 1600-1851, 1600-1869, 1601-1869, 1602-1861, 1604-1879, 1605-1868, 1611-1869, 1612- |
| | 1861, 1613-1835, 1623-1868, 1623-1869, 1631-1869, 1641-1861, 1642-1869, 1643-1864, 1644-1868, 1645-1861, |
| | 1658-1868, 1665-1861, 1666-1861, 1669-1869, 1672-1866, 1674-1869, 1675-1879, 1686-1869, 1716-1879, 1735- |
| | 1824, 1739-1869, 1744-1879, 1745-1869, 1773-1861, 1795-1861 |

TABLE 5

| Polynucleotide SEQ | | Representative |
|---|---|---|
| ID NO: | Incyte Project ID: | Library |
| 29 | 7994355CB1 | ESOGTUT02 |
| 30 | 7475875CB1 | ENDCNOT03 |
| 31 | 71231882CB1 | BRAZNOT01 |
| 32 | 2875922CB1 | PLACFER06 |
| 33 | 8158136CB1 | LNODNON02 |
| 34 | 5969491CB1 | BEPINON01 |
| 35 | 7497367CB1 | LIVRTMR01 |
| 36 | 7632424CB1 | PROSNOT11 |
| 37 | 1804436CB1 | SINTBST01 |
| 39 | 7472344CB1 | OVARDIR01 |
| 40 | 7192959CB1 | BRAITUT08 |
| 41 | 6169565CB1 | UTRSTDT01 |
| 42 | 7494717CB1 | BRSTNOT02 |
| 44 | 7498882CB1 | DENDNOT01 |
| 45 | 5524205CB1 | BRAUTDR02 |
| 46 | 7102342CB1 | BRSTTUT02 |
| 47 | 4169939CB1 | PANCNOT19 |
| 48 | 6539977CB1 | BRAIFEN08 |
| 49 | 7675588CB1 | BRONDIT01 |
| 50 | 6244077CB1 | TESTNOT17 |
| 52 | 7391748CB1 | THP1NOT01 |
| 53 | 7499780CB1 | PITUNON01 |
| 54 | 7499881CB1 | CRBLNOT01 |
| 55 | 7488579CB1 | SINTTMR02 |
| 56 | 7510521CB1 | DENDNOT01 |

TABLE 6

| Library | Vector | Library Description |
|---|---|---|
| BEPINON01 | PSPORT | Normalized library was constructed from 5.12 million |
| | | independent clones from a bronchial epithelium library. RNA was |
| | | made from a bronchial epithelium primary cell line derived |
| | | from a 54-year-old Caucasian male. The normalization and |
| | | hybridization conditions were adapted from Soares et al., |
| | | PNAS (1994) 91: 9228, using a longer (24-hour) reannealing |
| | | hybridization period. |
| BRAIFEN08 | pINCY | This normalized fetal brain tissue library was constructed |
| | | from 400 thousand independent clones from a fetal brain tissue |
| | | library. Starting RNA was made from brain tissue removed |
| | | from a Caucasian male fetus who was stillborn with a |
| | | hypoplastic left heart at 23 weeks' gestation. The library |
| | | was normalized in 2 rounds using conditions adapted from Soares |
| | | et al., PNAS (1994) 91: 9228 and Bonaldo et al., Genome |
| | | Research (1996) 6: 791, except that a significantly longer (48 |
| | | hours/round) reannealing hybridization was used. |
| BRAITUT08 | pINCY | Library was constructed using RNA isolated from brain tumor |
| | | tissue removed from the left frontal lobe of a 47-year-old |
| | | Caucasian male during excision of cerebral meningeal tissue. |
| | | Pathology indicated grade 4 fibrillary astrocytoma with focal |
| | | tumoral radionecrosis. Patient history included cerebrovascular |
| | | disease, deficiency anemia, hyperlipidemia, epilepsy, and |
| | | tobacco use. Family history included cerebrovascular disease |
| | | and a malignant prostate neoplasm. |
| BRAUTDR02 | PCDNA2.1 | This random primed library was constructed using RNA isolated |
| | | from pooled amygdala and entorhinal cortex tissue |
| | | removed from a 55-year-old Caucasian female who died from |
| | | cholangiocarcinoma. Pathology indicated mild meningeal |
| | | fibrosis predominately over the convexities, scattered axonal |
| | | spheroids in the white matter of the cingulate cortex and the |
| | | thalamus, and a few scattered neurofibrillary tangles in the |
| | | entorhinal cortex and the periaqueductal gray region. Pathology |
| | | for the associated tumor tissue indicated well-differentiated |
| | | cholangiocarcinoma of the liver with residual or relapsed |
| | | tumor. Patient history included cholangiocarcinoma, post- |
| | | operative Budd-Chiari syndrome, biliary ascites, hydrothorax, |
| | | dehydration, malnutrition, oliguria and acute renal failure. |
| | | Previous surgeries included cholecystectomy and resection of |
| | | 85% of the liver. |
| BRAZNOT01 | pINCY | Library was constructed using RNA isolated from striatum, |
| | | globus pallidus and posterior putamen tissue removed from a |
| | | 45-year-old Caucasian female who died from a dissecting aortic |
| | | aneurysm and ischemic bowel disease. Pathology indicated |
| | | mild arteriosclerosis involving the cerebral cortical white |
| | | matter and basal ganglia. Grossly, there was mild meningeal |
| | | fibrosis and mild focal atherosclerotic plaque in the middle |
| | | cerebral artery, as well as vertebral arteries bilaterally. |
| | | Microscopically, the cerebral hemispheres, brain stem and |
| | | cerebellum reveal focal areas in the white matter that contain |
| | | blood vessels that were barrel-shaped, hyalinized, with |
| | | hemosiderin-laden macrophages in the Virchow-Robin space. In |
| | | addition, there were scattered neurofibrillary tangles within |
| | | the basolateral nuclei of the amygdala. Patient history included |
| | | mild atheromatosis of aorta and coronary arteries, bowel and |
| | | liver infarct due to aneurysm, physiologic fatty liver |
| | | associated with obesity, mild diffuse emphysema, thrombosis |
| | | of mesenteric and portal veins, cardiomegaly due to |
| | | hypertrophy of left ventricle, arterial hypertension, acute |
| | | pulmonary edema, splenomegaly, obesity (300 lb.), leiomyoma of |
| | | uterus, sleep apnea, and iron deficiency anemia. |
| BRONDIT01 | pINCY | Library was constructed using RNA isolated from right lower |
| | | lobe bronchial tissue removed from a pool of 3 asthmatic |
| | | Caucasian male and female donors, 22- to 51-years-old during |
| | | bronchial pinch biopsies. Patient history included atopy as |
| | | determined by positive skin tests to common aero-allergens. |
| BRSTNOT02 | PSPORT1 | Library was constructed using RNA isolated from diseased breast |
| | | tissue removed from a 55-year-old Caucasian female |
| | | during a unilateral extended simple mastectomy. Pathology |
| | | indicated proliferative fibrocysytic changes characterized by |
| | | apocrine metaplasia, sclerosing adenosis, cyst formation, and |
| | | ductal hyperplasia without atypia. Pathology for the |
| | | associated tumor tissue indicated an invasive grade 4 mammary |
| | | adenocarcinoma. Patient history included atrial tachycardia |
| | | and a benign neoplasm. Family history included cardiovascular |
| | | and cerebrovascular disease. |
| BRSTTUT02 | PSPORT1 | Library was constructed using RNA isolated from breast tumor |
| | | tissue removed from a 54-year-old Caucasian female during |
| | | a bilateral radical mastectomy with reconstruction. Pathology |
| | | indicated residual invasive grade 3 mammary ductal |
| | | adenocarcinoma. The remaining breast parenchyma exhibited |
| | | proliferative fibrocystic changes without atypia. One of 10 |
| | | axillary lymph nodes had metastatic tumor as a microscopic |
| | | intranodal focus. Patient history included kidney infection and |
| | | condyloma acuminatum. Family history included benign |
| | | hypertension, hyperlipidemia, and a malignant colon neoplasm. |
| CRBLNOT01 | PSPORT1 | Library was constructed using RNA isolated from the cerebellum |
| | | tissue of a 69-year-old Caucasian male who died from |
| | | chronic obstructive pulmonary disease. Patient history included |
| | | myocardial infarction, hypertension, and osteoarthritis. |
| DENDNOT01 | pINCY | Library was constructed using RNA isolated from untreated |
| | | dendritic cells from peripheral blood. |
| ENDCNOT03 | pINCY | Library was constructed using RNA isolated from dermal |
| | | microvascular endothelial cells removed from a neonatal |
| | | Caucasian male. |
| ESOGTUT02 | pINCY | Library was constructed using RNA isolated from esophageal |
| | | tumor tissue obtained from a 61-year-old Caucasian male |
| | | during a partial esophagectomy, proximal gastrectomy, |
| | | pyloromyotomy, and regional lymph node excision. Pathology |
| | | indicated an invasive grade 3 adenocarcinoma in the esophagus. |
| | | Family history included atherosclerotic coronary artery |
| | | disease, type II diabetes, chronic liver disease, primary |
| | | cardiomyopathy, benign hypertension, and cerebrovascular disease. |
| LIVRTMR01 | PCDNA2.1 | This random primed library was constructed using RNA isolated |
| | | from liver tissue removed from a 62-year-old Caucasian |
| | | female during partial hepatectomy and exploratory laparotomy. |
| | | Pathology for the matched tumor tissue indicated metastatic |
| | | intermediate grade neuroendocrine carcinoma, consistent with |
| | | islet cell tumor, forming nodules ranging in size, in the |
| | | lateral and medial left liver lobe. The pancreas showed |
| | | fibrosis, chronic inflammation and fat necrosis consistent with |
| | | pseudocyst. The gallbladder showed mild chronic cholecystitis. |
| | | Patient history included malignant neoplasm of the |
| | | pancreas tail, pulmonary embolism, hyperlipidemia, thrombophlebitis, |
| | | joint pain in multiple joints, type II diabetes, benign |
| | | hypertension, cerebrovascular disease, and normal delivery. |
| | | Previous surgeries included distal pancreatectomy, total |
| | | splenectomy, and partial hepatectomy. Family history included |
| | | pancreas cancer with secondary liver cancer, benign |
| | | hypertension, and hyperlipidemia. |
| LNODNON02 | pINCY | This normalized lymph node tissue library was constructed from |
| | | .56 million independent clones from a lymph node tissue |
| | | library. Starting RNA was made from lymph node tissue removed |
| | | from a 16-month-old Caucasian male who died from |
| | | head trauma. Serologies were negative. Patient history included |
| | | bronchitis. Patient medications included Dopamine, |
| | | Dobutamine, Vancomycin, Vasopressin, Proventil, and Atarax. |
| | | The library was normalized in two rounds using conditions |
| | | adapted from Soares et al., PNAS (1994) 91: 9228-9932 |
| | | and Bonaldo et al.. Genome Research 6 (1996): 791, except that a |
| | | significantly longer (48 hours/round) reannealing hybridization was used. |
| OVARDIR01 | PCDNA2.1 | This random primed library was constructed using RNA isolated |
| | | from right ovary tissue removed from a 45-year-old |
| | | Caucasian female during total abdominal hysterectomy, |
| | | bilateral salpingo-oophorectomy, vaginal suspension and fixation, |
| | | and incidental appendectomy. Pathology indicated stromal |
| | | hyperthecosis of the right and left ovaries. Pathology for the |
| | | matched tumor tissue indicated a dermoid cyst (benign |
| | | cystic teratoma) in the left ovary. Multiple (3) intramural |
| | | leiomyomata were identified. The cervix showed squamous |
| | | metaplasia. Patient history included metrorrhagia, female stress |
| | | incontinence, alopecia, depressive disorder, pneumonia, |
| | | normal delivery, and deficiency anemia. Family history included |
| | | benign hypertension, atherosclerotic coronary artery disease, |
| | | hyperlipidemia, and primary tuberculous complex. |
| PANCNOT19 | pINCY | Library was constructed using RNA isolated from pancreatic |
| | | tissue removed from an 8-year-old Black male who died from |
| | | anoxia. |
| PITUNON01 | pINCY | This normalized pituitary gland tissue library was constructed |
| | | from 6.92 million independent clones from a pituitary gland |
| | | tissue library. Starting RNA was made from pituitary gland |
| | | tissue removed from a 55-year-old male who died from chronic |
| | | obstructive pulmonary disease. Neuropathology indicated there |
| | | were no gross abnormalities, other than mild ventricular |
| | | enlargement. There was no apparent microscopic abnormality |
| | | in any of the neocortical areas examined, except for a |
| | | number of silver positive neurons with apical dendrite |
| | | staining, particularly in the frontal lobe. The significance of this was |
| | | undetermined. The only other microscopic abnormality was that |
| | | there was prominent silver staining with some swollen |
| | | axons in the CA3 region of the anterior and posterior hippocampus. |
| | | Microscopic sections of the cerebellum revealed mild |
| | | Bergmann's gliosis in the Purkinje cell layer. Patient history |
| | | included schizophrenia. The library was normalized in two |
| | | rounds using conditions adapted from Soares et al., PNAS (1994) |
| | | 91: 9228-9232 and Bonaldo et al., Genome Research |
| PLACFER06 | pINCY | This random primed library was constructed using RNA isolated |
| | | from placental tissue removed from a Caucasian fetus who |
| | | died after 16 weeks' gestation from fetal demise and |
| | | hydrocephalus. Patient history included umbilical cord wrapped |
| | | around the head (3 times) and the shoulders (1 time). |
| | | Serology was positive for anti-CMV. Family history included |
| | | multiple pregnancies and live births, and an abortion. |
| PROSNOT11 | pINCY | Library was constructed using RNA isolated from the prostate |
| | | tissue of a 28-year-old Caucasian male, who died from a self |
| | | inflicted gunshot wound. |
| SINTBST01 | pINCY | Library was constructed using RNA isolated from ileum tissue |
| | | obtained from an 18-year-old Caucasian female during |
| | | bowel anastomosis. Pathology indicated Crohn's disease of |
| | | the ileum, involving 15 cm of the small bowel. Family history |
| | | included cerebrovascular disease and atherosclerotic coronary |
| | | artery disease. |
| SINTTMR02 | PCDNA2.1 | This random primed library was constructed using RNA isolated |
| | | from small intestine tissue removed from a 59-year-old |
| | | male. Pathology for the matched tumor tissue indicated multiple |
| | | (9) carcinoid tumors, grade 1, in the small bowel. The |
| | | largest tumor was associated with a large mesenteric mass. |
| | | Multiple convoluted segments of bowel were adhered to the |
| | | tumor. A single (1 of 13) regional lymph node was positive |
| | | for malignancy. The peritoneal biopsy indicated focal fat |
| | | necrosis. |
| TESTNOT17 | pINCY | Library was constructed from testis tissue removed from a |
| | | 26-year-old Caucasian male who died from head trauma due to a |
| | | motor vehicle accident. Serologies were negative. Patient |
| | | history included a hernia at birth, tobacco use (1½ ppd), |
| | | marijuana use, and daily alcohol use (beer and hard liquor). |
| THP1NOT01 | pINCY | Library was constructed using RNA isolated from untreated |
| | | THP-1 cells. THP-1 is a human promonocyte line derived from |
| | | the peripheral blood of a 1-year-old Caucasian male with |
| | | acute monocytic leukemia (ref: Int. J. Cancer (1980) 26: 171). |
| UTRSTDT01 | pINCY | Library was constructed using RNA isolated from uterus |
| | | tissue removed from a 46-year-old Caucasian female who died |
| | | From cardiopulmonary arrest. Patient history included liver |
| | | and breast cancer. |

TABLE 7

| Program | Description | Reference | Parameter Threshold |
|---|---|---|---|
| ABI | A program that removes | Applied Biosystems, Foster City, CA. | |
| FACTURA | vector sequences and masks | | |
| | ambiguous bases in | | |
| | nucleic acid sequences. | | |
| ABI/ | A Fast Data Finder | Applied Biosystems, Foster City, CA; | Mismatch < 50% |
| PARACEL | useful in comparing and | Paracel Inc., Pasadena, CA. | |
| FDF | annotating amino acid or | | |
| | nucleic acid sequences. | | |
| ABI | A program that assembles | Applied Biosystems, Foster City, CA. | |
| AutoAssembler | nucleic acid sequences. | | |
| BLAST | A Basic Local Alignment | Altschul, S. F. et al. (1990) J. Mol. Biol. | ESTs: Probability value = 1.0E−8 |
| | Search Tool useful in | 215: 403-410; Altschul, S. F. et al. (1997) | or less; Full Length sequences: |
| | sequence similarity search | Nucleic Acids Res. 25: 3389-3402. | Probability value = 1.0E−10 or |
| | for amino acid and nucleic | | less |
| | acid sequences. BLAST | | |
| | includes five functions: | | |
| | blastp, blastn, blastx, | | |
| | tblastn, and tblastx. | | |
| FASTA | A Pearson and Lipman | Pearson, W. R. and D. J. Lipman (1988) Proc. | ESTs: fasta E value = 1.06E−6; |
| | algorithm that searches for | Natl. Acad Sci. USA 85: 2444-2448; Pearson, | Assembled ESTs: fasta |
| | similarity between a query | W.R. (1990) Methods Enzymol. 183: 63-98; | Identity = |
| | sequence and a group of | and Smith, T. F. and M. S. Waterman (1981) | 95% or greater and Match |
| | sequences of the same type. | Adv. Appl. Math. 2: 482-489. | length = 200 bases or greater; |
| | FASTA comprises as | | fastx E value = 1.0E−8 or less; |
| | least five functions: fasta, | | Full Length sequences: fastx |
| | tfasta, fastx, tfastx, and | | score = 100 or greater |
| | ssearch. | | |
| BLIMPS | A BLocks IMProved | Henikoff, S. and J. G. Henikoff (1991) | Probability value = 1.0E−3 or less |
| | Searcher that matches a | Nucleic Acids Res. 19: 6565-6572; Henikoff, | |
| | sequence against those | J. G. and S. Henikoff (1996) Methods | |
| | in BLOCKS, PRINTS, | Enzymol. 266: 88-105; and Attwood, T. K. et | |
| | DOMO, PRODOM, and PFAM | al. (1997) J. Chem. Inf. Comput. Sci. 37: 417- | |
| | databases to search | | |
| | for gene families, sequence | | |
| | homology, and structural | | |
| | fingerprint regions. | | |
| HMMER | An algorithm for searching | Krogh, A. et al. (1994) J. Mol. Biol. | PFAM, INCY, SMART or |
| | a query sequence against | 235: 1501-1531; Sonnhammer, E. L. L. et al. | TIGRFAM hits: Probability |
| | hidden Markov model | (1988) Nucleic Acids Res. 26: 320-322; | value = 1.0E−3 or less; Signal |
| | (HMM)-based databases of | Durbin, R. et al. (1998) Our World View, in | peptide hits: Score = 0 or greater |
| | protein family consensus | a Nutshell, Cambridge Univ. Press, pp. 1- | |
| | sequences, such as PFAM, | | |
| | INCY, SMART and TIGRFAM. | | |
| ProfileScan | An algorithm that searches | Gribskov, M. et al. (1988) CABIOS 4: 61-66; | Normalized quality score ≧ GCG |
| | for structural and | Gribskov, M. et al. (1989) Methods | specified “HIGH” value for that |
| | sequence motifs in protein | Enzymol. 183: 146-159; Bairoch, A. et al. | particular Prosite motif. |
| | sequences that match | (1997) Nucleic Acids Res. 25: 217-221. | Generally, score = 1.4-2.1. |
| | sequence patterns defined | | |
| | in Prosite. | | |
| Phred | A base-calling algorithm | Ewing, B. et al. (1998) Genome Res. 8: 175- | |
| | that examines automated | 185; Ewing, B. and P. Green (1998) Genome | |
| | sequencer traces with high | Res. 8: 186-194. | |
| | sensitivity and probability. | | |
| Phrap | A Phils Revised Assembly | Smith, T.F. and M. S. Waterman (1981) Adv. | Score = 120 or greater; Match |
| | Program including | Appl. Math. 2: 482-489; Smith, T. F. and | length = 56 or greater |
| | SWAT and CrossMatch, | M. S. Waterman (1981) J. Mol. Biol. 147: 195- | |
| | programs based on efficient | 197; and Green, P., University of | |
| | implementation of the | Washington, Seattle, WA. | |
| | Smith-Waterman algorithm, | | |
| | useful in searching | | |
| | sequence homology and | | |
| | assembling DNA sequences. | | |
| Consed | A graphical tool for | Gordon, D. et al. (1998) Genome Res. 8: 195- | |
| | viewing and editing Phrap | 202. | |
| | assemblies. | | |
| SPScan | A weight matrix analysis | Nielson, H. et al. (1997) Protein Engineering | Score = 3.5 or greater |
| | program that scans protein | 10: 1-6; Claverie, J. M. and S. Audic (1997) | |
| | sequences for the presence | CABIOS 12: 431-439. | |
| | of secretory signal | | |
| | peptides. | | |
| TMAP | A program that uses weight | Persson, B. and P. Argos (1994) J. Mol. Biol. | |
| | matrices to delineate | 237: 182-192; Persson, B. and P. Argos | |
| | transmembrane segments | (1996) Protein Sci. 5: 363-371. | |
| | on protein sequences and | | |
| | determine orientation. | | |
| TMHMMER | A program that uses a | Sonnhammer, E. L. et al. (1998) Proc. Sixth | |
| | hidden Markov model (HMM) | Intl. Conf. On Intelligent Systems for Mol. | |
| | to delineate transmembrane | Biol., Glasgow et al., eds., The Am. Assoc. | |
| | segments on protein | for Artificial Intelligence (AAAI) Press, | |
| | sequences and determine | Menlo Park, CA, and MIT Press, Cambridge, | |
| | orientation. | MA, pp. 175-182. | |
| Motifs | A program that searches | Bairoch, A. et al. (1997) Nucleic Acids Res. | |
| | amino acid sequences for | 25: 217-221; Wisconsin Package Program | |
| | patterns that matched | Manual, version 9, page M51-59, Genetics | |
| | those defined in Prosite. | Computer Group, Madison, WI. | |

TABLE 8

| SEQ | | | | | | EST | Al- | Al- | | Caucasian | African | Asian | Hispanic |
|---|---|---|---|---|---|---|---|---|---|---|---|---|---|
| ID | | | | EST | CB1 | Al- | lele | lele | Amino | Allele 1 | Allele 1 | Allele 1 | Allele 1 |
| NO: | PID | EST ID | SNP ID | SNP | SNP | lele | 1 | 2 | Acid | frequency | frequency | frequency | frequency |
| 56 | 7510521 | 064728H1 | SNP00006579 | 124 | 526 | T | C | T | noncoding | 0.82 | n/a | n/a | n/a |
| 56 | 7510521 | 073306H1 | SNP00105444 | 119 | 1259 | C | C | T | noncoding | n/a | n/a | n/a | n/a |
| 56 | 7510521 | 073566H1 | SNP00105444 | 51 | 1259 | C | C | T | noncoding | n/a | n/a | n/a | n/a |
| 56 | 7510521 | 073573H1 | SNP00105444 | 51 | 1259 | C | C | T | noncoding | n/a | n/a | n/a | n/a |
| 56 | 7510521 | 074430H1 | SNP00105444 | 119 | 1259 | C | C | T | noncoding | n/a | n/a | n/a | n/a |
| 56 | 7510521 | 075234H1 | SNP00105444 | 119 | 1259 | C | C | T | noncoding | n/a | n/a | n/a | n/a |
| 56 | 7510521 | 075593H1 | SNP00105444 | 43 | 1259 | C | C | T | noncoding | n/a | n/a | n/a | n/a |
| 56 | 7510521 | 076131H1 | SNP00105444 | 119 | 1259 | C | C | T | noncoding | n/a | n/a | n/a | n/a |
| 56 | 7510521 | 076800H1 | SNP00105444 | 119 | 1259 | C | C | T | noncoding | n/a | n/a | n/a | n/a |
| 56 | 7510521 | 076808H1 | SNP00105444 | 119 | 1259 | C | C | T | noncoding | n/a | n/a | n/a | n/a |
| 56 | 7510521 | 078964H1 | SNP00105444 | 119 | 1259 | C | C | T | noncoding | n/a | n/a | n/a | n/a |
| 56 | 7510521 | 1239677H1 | SNP00067426 | 12 | 97 | T | T | C | noncoding | 0.95 | 0.95 | n/d | 0.96 |
| 56 | 7510521 | 1362396H1 | SNP00067426 | 12 | 97 | T | T | C | noncoding | 0.95 | 0.95 | n/d | 0.96 |
| 56 | 7510521 | 1397195H1 | SNP00067426 | 37 | 97 | T | T | C | noncoding | 0.95 | 0.95 | n/d | 0.96 |
| 56 | 7510521 | 1424530H1 | SNP00105444 | 235 | 1259 | C | C | T | noncoding | n/a | n/a | n/a | n/a |
| 56 | 7510521 | 1452807H1 | SNP00105444 | 166 | 1259 | C | C | T | noncoding | n/a | n/a | n/a | n/a |
| 56 | 7510521 | 1453282H1 | SNP00067426 | 28 | 97 | T | T | C | noncoding | 0.95 | 0.95 | n/d | 0.96 |
| 56 | 7510521 | 1454803H1 | SNP00067426 | 28 | 97 | T | T | C | noncoding | 0.95 | 0.95 | n/d | 0.96 |
| 56 | 7510521 | 1514958H1 | SNP00067426 | 28 | 97 | T | T | C | noncoding | 0.95 | 0.95 | n/d | 0.96 |
| 56 | 7510521 | 156942R1 | SNP00105444 | 457 | 1259 | C | C | T | noncoding | n/a | n/a | n/a | n/a |
| 56 | 7510521 | 1636617H1 | SNP00105444 | 43 | 1259 | C | C | T | noncoding | n/a | n/a | n/a | n/a |
| 56 | 7510521 | 1637257H1 | SNP00105444 | 43 | 1259 | C | C | T | noncoding | n/a | n/a | n/a | n/a |
| 56 | 7510521 | 1806536H1 | SNP00105444 | 243 | 1259 | C | C | T | noncoding | n/a | n/a | n/a | n/a |
| 56 | 7510521 | 1806750H1 | SNP00006580 | 47 | 1241 | G | T | G | noncoding | n/a | n/a | n/a | n/a |
| 56 | 7510521 | 1880982H1 | SNP00105444 | 131 | 1259 | C | C | T | noncoding | n/a | n/a | n/a | n/a |
| 56 | 7510521 | 1900090H1 | SNP00006579 | 197 | 526 | C | C | T | noncoding | 0.82 | n/a | n/a | n/a |
| 56 | 7510521 | 1974452H1 | SNP00105444 | 148 | 1259 | C | C | T | noncoding | n/a | n/a | n/a | n/a |
| 56 | 7510521 | 1979551H1 | SNP00105444 | 95 | 1259 | C | C | T | noncoding | n/a | n/a | n/a | n/a |
| 56 | 7510521 | 1979551R6 | SNP00105444 | 95 | 1262 | C | C | T | noncoding | n/a | n/a | n/a | n/a |
| 56 | 7510521 | 2107073H1 | SNP00067426 | 8 | 97 | T | T | C | noncoding | 0.95 | 0.95 | n/d | 0.96 |
| 56 | 7510521 | 2116360H1 | SNP00067426 | 12 | 97 | T | T | C | noncoding | 0.95 | 0.95 | n/d | 0.96 |
| 56 | 7510521 | 2135075H1 | SNP00067426 | 70 | 97 | T | T | C | noncoding | 0.95 | 0.95 | n/d | 0.96 |
| 56 | 7510521 | 2137253H1 | SNP00067426 | 12 | 97 | T | T | C | noncoding | 0.95 | 0.95 | n/d | 0.96 |
| 56 | 7510521 | 2183517H1 | SNP00067426 | 13 | 97 | T | T | C | noncoding | 0.95 | 0.95 | n/d | 0.96 |
| 56 | 7510521 | 2211782H1 | SNP00067426 | 28 | 97 | T | T | C | noncoding | 0.95 | 0.95 | n/d | 0.96 |
| 56 | 7510521 | 2212924H1 | SNP00067426 | 28 | 97 | T | T | C | noncoding | 0.95 | 0.95 | n/d | 0.96 |
| 56 | 7510521 | 2303437H1 | SNP00105444 | 25 | 1259 | C | C | T | noncoding | n/a | n/a | n/a | n/a |
| 56 | 7510521 | 2311978H1 | SNP00105444 | 20 | 1259 | C | C | T | noncoding | n/a | n/a | n/a | n/a |
| 56 | 7510521 | 2369247H1 | SNP00006579 | 8 | 526 | C | C | T | noncoding | 0.82 | n/a | n/a | n/a |
| 56 | 7510521 | 2397207H1 | SNP00067426 | 28 | 97 | T | T | C | noncoding | 0.95 | 0.95 | n/d | 0.96 |
| 56 | 7510521 | 2398368H1 | SNP00006579 | 161 | 526 | C | C | T | noncoding | 0.82 | n/a | n/a | n/a |
| 56 | 7510521 | 2436959H1 | SNP00006579 | 130 | 526 | C | C | T | noncoding | 0.82 | n/a | n/a | n/a |
| 56 | 7510521 | 2437672H1 | SNP00067426 | 35 | 97 | T | T | C | noncoding | 0.95 | 0.95 | n/d | 0.96 |
| 56 | 7510521 | 2438148H1 | SNP00067426 | 32 | 97 | T | T | C | noncoding | 0.95 | 0.95 | n/d | 0.96 |
| 56 | 7510521 | 2454291H1 | SNP00006579 | 46 | 526 | T | C | T | noncoding | 0.82 | n/a | n/a | n/a |
| 56 | 7510521 | 2582571H1 | SNP00067426 | 35 | 97 | T | T | C | noncoding | 0.95 | 0.95 | n/d | 0.96 |
| 56 | 7510521 | 2591249H2 | SNP00105444 | 13 | 1259 | C | C | T | noncoding | n/a | n/a | n/a | n/a |
| 56 | 7510521 | 2591549H1 | SNP00105444 | 13 | 1259 | C | C | T | noncoding | n/a | n/a | n/a | n/a |
| 56 | 7510521 | 2620592H1 | SNP00067426 | 29 | 97 | C | T | C | noncoding | 0.95 | 0.95 | n/d | 0.96 |
| 56 | 7510521 | 2621494H1 | SNP00105444 | 169 | 1259 | C | C | T | noncoding | n/a | n/a | n/a | n/a |
| 56 | 7510521 | 2622226H1 | SNP00006580 | 226 | 1241 | T | T | G | noncoding | n/a | n/a | n/a | n/a |
| 56 | 7510521 | 2675269H1 | SNP00067426 | 27 | 97 | T | T | C | noncoding | 0.95 | 0.95 | n/d | 0.96 |
| 56 | 7510521 | 2686334H1 | SNP00105444 | 31 | 1259 | C | C | T | noncoding | n/a | n/a | n/a | n/a |
| 56 | 7510521 | 2722514H1 | SNP00067426 | 50 | 97 | T | T | C | noncoding | 0.95 | 0.95 | n/d | 0.96 |
| 56 | 7510521 | 2741742H1 | SNP00105444 | 238 | 1259 | C | C | T | noncoding | n/a | n/a | n/a | n/a |
| 56 | 7510521 | 2745948H1 | SNP00067426 | 15 | 97 | T | T | C | noncoding | 0.95 | 0.95 | n/d | 0.96 |
| 56 | 7510521 | 2767239H1 | SNP00067426 | 26 | 97 | T | T | C | noncoding | 0.95 | 0.95 | n/d | 0.96 |
| 56 | 7510521 | 2799215H1 | SNP00105444 | 13 | 1259 | C | C | T | noncoding | n/a | n/a | n/a | n/a |
| 56 | 7510521 | 2848443H1 | SNP00067426 | 30 | 97 | T | T | C | noncoding | 0.95 | 0.95 | n/d | 0.96 |
| 56 | 7510521 | 2862235H1 | SNP00105444 | 39 | 1259 | C | C | T | noncoding | n/a | n/a | n/a | n/a |
| 56 | 7510521 | 2884679H1 | SNP00067426 | 12 | 97 | T | T | C | noncoding | 0.95 | 0.95 | n/d | 0.96 |
| 56 | 7510521 | 2910110H1 | SNP00067426 | 16 | 97 | T | T | C | noncoding | 0.95 | 0.95 | n/d | 0.96 |
| 56 | 7510521 | 2913667H1 | SNP00067426 | 33 | 97 | T | T | C | noncoding | 0.95 | 0.95 | n/d | 0.96 |
| 56 | 7510521 | 2969030H1 | SNP00067426 | 10 | 97 | T | T | C | noncoding | 0.95 | 0.95 | n/d | 0.96 |
| 56 | 7510521 | 2994484H1 | SNP00006579 | 20 | 526 | C | C | T | noncoding | 0.82 | n/a | n/a | n/a |
| 56 | 7510521 | 3045590H1 | SNP00006579 | 28 | 526 | C | C | T | noncoding | 0.82 | n/a | n/a | n/a |
| 56 | 7510521 | 3095345H1 | SNP00006579 | 120 | 526 | T | C | T | noncoding | 0.82 | n/a | n/a | n/a |
| 56 | 7510521 | 3117031H1 | SNP00067426 | 26 | 97 | T | T | C | noncoding | 0.95 | 0.95 | n/d | 0.96 |
| 56 | 7510521 | 3138158H1 | SNP00105444 | 144 | 1259 | C | C | T | noncoding | n/a | n/a | n/a | n/a |
| 56 | 7510521 | 3220920H1 | SNP00105444 | 42 | 1259 | C | C | T | noncoding | n/a | n/a | n/a | n/a |
| 56 | 7510521 | 3236768H1 | SNP00105444 | 19 | 1259 | C | C | T | noncoding | n/a | n/a | n/a | n/a |
| 56 | 7510521 | 3240887H1 | SNP00105444 | 143 | 1259 | C | C | T | noncoding | n/a | n/a | n/a | n/a |
| 56 | 7510521 | 3283818H1 | SNP00067426 | 12 | 97 | T | T | C | noncoding | 0.95 | 0.95 | n/d | 0.96 |
| 56 | 7510521 | 3343887H1 | SNP00006579 | 159 | 526 | T | C | T | noncoding | 0.82 | n/a | n/a | n/a |
| 56 | 7510521 | 3345625H1 | SNP00105444 | 236 | 1259 | C | C | T | noncoding | n/a | n/a | n/a | n/a |
| 56 | 7510521 | 3377028H1 | SNP00105444 | 33 | 1259 | C | C | T | noncoding | n/a | n/a | n/a | n/a |
| 56 | 7510521 | 3402507H1 | SNP00067426 | 61 | 97 | T | T | C | noncoding | 0.95 | 0.95 | n/d | 0.96 |
| 56 | 7510521 | 3403476H1 | SNP00006579 | 121 | 526 | C | C | T | noncoding | 0.82 | n/a | n/a | n/a |
| 56 | 7510521 | 3405513H1 | SNP00067426 | 30 | 97 | T | T | C | noncoding | 0.95 | 0.95 | n/d | 0.96 |
| 56 | 7510521 | 3411758H1 | SNP00067426 | 33 | 97 | T | T | C | noncoding | 0.95 | 0.95 | n/d | 0.96 |
| 56 | 7510521 | 3422192H1 | SNP00067426 | 25 | 97 | C | T | C | noncoding | 0.95 | 0.95 | n/d | 0.96 |
| 56 | 7510521 | 3450266H1 | SNP00006579 | 205 | 526 | C | C | T | noncoding | 0.82 | n/a | n/a | n/a |
| 56 | 7510521 | 3455078H1 | SNP00006579 | 36 | 526 | T | C | T | noncoding | 0.82 | n/a | n/a | n/a |
| 56 | 7510521 | 3473335H1 | SNP00105444 | 126 | 1259 | C | C | T | noncoding | n/a | n/a | n/a | n/a |
| 56 | 7510521 | 3503020H1 | SNP00067426 | 61 | 97 | T | T | C | noncoding | 0.95 | 0.95 | n/d | 0.96 |
| 56 | 7510521 | 3510182H1 | SNP00067426 | 33 | 97 | C | T | C | noncoding | 0.95 | 0.95 | n/d | 0.96 |
| 56 | 7510521 | 3556979H1 | SNP00105444 | 144 | 1259 | C | C | T | noncoding | n/a | n/a | n/a | n/a |
| 56 | 7510521 | 3595275H1 | SNP00067426 | 10 | 97 | T | T | C | noncoding | 0.95 | 0.95 | n/d | 0.96 |
| 56 | 7510521 | 3600404H1 | SNP00105444 | 87 | 1259 | C | C | T | noncoding | n/a | n/a | n/a | n/a |
| 56 | 7510521 | 3617096H1 | SNP00067426 | 11 | 97 | T | T | C | noncoding | 0.95 | 0.95 | n/d | 0.96 |
| 56 | 7510521 | 3628252H1 | SNP00105444 | 132 | 1259 | C | C | T | noncoding | n/a | n/a | n/a | n/a |
| 56 | 7510521 | 3814147H1 | SNP00006580 | 172 | 1241 | T | T | G | noncoding | n/a | n/a | n/a | n/a |
| 56 | 7510521 | 3814616H1 | SNP00006579 | 81 | 526 | C | C | T | noncoding | 0.82 | n/a | n/a | n/a |
| 56 | 7510521 | 3833454H1 | SNP00105444 | 60 | 1259 | C | C | T | noncoding | n/a | n/a | n/a | n/a |
| 56 | 7510521 | 3836524H1 | SNP00006580 | 221 | 1241 | T | T | G | noncoding | n/a | n/a | n/a | n/a |
| 56 | 7510521 | 3837242H1 | SNP00006579 | 124 | 526 | C | C | T | noncoding | 0.82 | n/a | n/a | n/a |
| 56 | 7510521 | 3841051H1 | SNP00105444 | 62 | 1259 | C | C | T | noncoding | n/a | n/a | n/a | n/a |
| 56 | 7510521 | 3842154H1 | SNP00105444 | 72 | 1259 | C | C | T | noncoding | n/a | n/a | n/a | n/a |
| 56 | 7510521 | 3843327H1 | SNP00006579 | 126 | 526 | C | C | T | noncoding | 0.82 | n/a | n/a | n/a |
| 56 | 7510521 | 3843721H1 | SNP00105444 | 74 | 1259 | C | C | T | noncoding | n/a | n/a | n/a | n/a |
| 56 | 7510521 | 3844448H1 | SNP00006579 | 58 | 526 | C | C | T | noncoding | 0.82 | n/a | n/a | n/a |
| 56 | 7510521 | 3844674H1 | SNP00006579 | 99 | 526 | C | C | T | noncoding | 0.82 | n/a | n/a | n/a |
| 56 | 7510521 | 3845294H1 | SNP00105444 | 127 | 1259 | C | C | T | noncoding | n/a | n/a | n/a | n/a |
| 56 | 7510521 | 3845517H1 | SNP00067426 | 31 | 97 | T | T | C | noncoding | 0.95 | 0.95 | n/d | 0.96 |
| 56 | 7510521 | 3877879H1 | SNP00006579 | 122 | 526 | T | C | T | noncoding | 0.82 | n/a | n/a | n/a |
| 56 | 7510521 | 4044544H1 | SNP00105444 | 154 | 1259 | C | C | T | noncoding | n/a | n/a | n/a | n/a |
| 56 | 7510521 | 4054975H1 | SNP00006580 | 162 | 1241 | G | T | G | noncoding | n/a | n/a | n/a | n/a |
| 56 | 7510521 | 4083278H1 | SNP00006580 | 107 | 1241 | G | T | G | noncoding | n/a | n/a | n/a | n/a |
| 56 | 7510521 | 415116R6 | SNP00006579 | 97 | 526 | C | C | T | noncoding | 0.82 | n/a | n/a | n/a |
| 56 | 7510521 | 4209957H1 | SNP00006579 | 123 | 526 | C | C | T | noncoding | 0.82 | n/a | n/a | n/a |
| 56 | 7510521 | 4335846H1 | SNP00105444 | 48 | 1259 | C | C | T | noncoding | n/a | n/a | n/a | n/a |
| 56 | 7510521 | 4335847H1 | SNP00105444 | 46 | 1259 | C | C | T | noncoding | n/a | n/a | n/a | n/a |
| 56 | 7510521 | 4415058H1 | SNP00067426 | 30 | 97 | T | T | C | noncoding | 0.95 | 0.95 | n/d | 0.96 |
| 56 | 7510521 | 4424212H1 | SNP00006579 | 171 | 526 | C | C | T | noncoding | 0.82 | n/a | n/a | n/a |
| 56 | 7510521 | 4543723H1 | SNP00105444 | 57 | 1259 | C | C | T | noncoding | n/a | n/a | n/a | n/a |
| 56 | 7510521 | 4551410H1 | SNP00105444 | 23 | 1259 | C | C | T | noncoding | n/a | n/a | n/a | n/a |
| 56 | 7510521 | 4557539H1 | SNP00105444 | 18 | 1259 | C | C | T | noncoding | n/a | n/a | n/a | n/a |
| 56 | 7510521 | 4559026H1 | SNP00105444 | 18 | 1259 | C | C | T | noncoding | n/a | n/a | n/a | n/a |
| 56 | 7510521 | 4610804H1 | SNP00067426 | 12 | 97 | T | T | C | noncoding | 0.95 | 0.95 | n/d | 0.96 |
| 56 | 7510521 | 4637077H1 | SNP00067426 | 27 | 97 | T | T | C | noncoding | 0.95 | 0.95 | n/d | 0.96 |
| 56 | 7510521 | 4671661H1 | SNP00067426 | 30 | 97 | T | T | C | noncoding | 0.95 | 0.95 | n/d | 0.96 |
| 56 | 7510521 | 4705376H1 | SNP00067426 | 29 | 97 | T | T | C | noncoding | 0.95 | 0.95 | n/d | 0.96 |
| 56 | 7510521 | 4781251H1 | SNP00067426 | 11 | 97 | T | T | C | noncoding | 0.95 | 0.95 | n/d | 0.96 |
| 56 | 7510521 | 4794967H1 | SNP00006580 | 244 | 1241 | G | T | G | noncoding | n/a | n/a | n/a | n/a |
| 56 | 7510521 | 4894666H1 | SNP00006579 | 29 | 526 | C | C | T | noncoding | 0.82 | n/a | n/a | n/a |
| 56 | 7510521 | 4993290H1 | SNP00006579 | 257 | 526 | C | C | T | noncoding | 0.82 | n/a | n/a | n/a |
| 56 | 7510521 | 5038495H1 | SNP00006579 | 227 | 526 | C | C | T | noncoding | 0.82 | n/a | n/a | n/a |
| 56 | 7510521 | 5075161H1 | SNP00067426 | 32 | 97 | T | T | C | noncoding | 0.95 | 0.95 | n/d | 0.96 |
| 56 | 7510521 | 5111563H1 | SNP00006580 | 156 | 1241 | T | T | G | noncoding | n/a | n/a | n/a | n/a |
| 56 | 7510521 | 5115634H1 | SNP00105444 | 5 | 1259 | C | C | T | noncoding | n/a | n/a | n/a | n/a |
| 56 | 7510521 | 5187513H1 | SNP00105444 | 170 | 1259 | C | C | T | noncoding | n/a | n/a | n/a | n/a |
| 56 | 7510521 | 5274442H1 | SNP00105444 | 128 | 1259 | C | C | T | noncoding | n/a | n/a | n/a | n/a |
| 56 | 7510521 | 5313705H1 | SNP00105444 | 43 | 1259 | C | C | T | noncoding | n/a | n/a | n/a | n/a |
| 56 | 7510521 | 548863H1 | SNP00067426 | 30 | 97 | T | T | C | noncoding | 0.95 | 0.95 | n/d | 0.96 |
| 56 | 7510521 | 5551463H1 | SNP00105444 | 30 | 1259 | C | C | T | noncoding | n/a | n/a | n/a | n/a |
| 56 | 7510521 | 5610627H1 | SNP00105444 | 184 | 1259 | C | C | T | noncoding | n/a | n/a | n/a | n/a |
| 56 | 7510521 | 5636114H1 | SNP00006579 | 8 | 526 | C | C | T | noncoding | 0.82 | n/a | n/a | n/a |
| 56 | 7510521 | 5679473H1 | SNP00067426 | 31 | 97 | T | T | C | noncoding | 0.95 | 0.95 | n/d | 0.96 |
| 56 | 7510521 | 5685751H1 | SNP00067426 | 44 | 97 | T | T | C | noncoding | 0.95 | 0.95 | n/d | 0.96 |
| 56 | 7510521 | 5733518H1 | SNP00105444 | 38 | 1259 | C | C | T | noncoding | n/a | n/a | n/a | n/a |
| 56 | 7510521 | 5955250H1 | SNP00006579 | 21 | 526 | C | C | T | noncoding | 0.82 | n/a | n/a | n/a |
| 56 | 7510521 | 6028821H1 | SNP00105444 | 22 | 1259 | C | C | T | noncoding | n/a | n/a | n/a | n/a |
| 56 | 7510521 | 6096755H1 | SNP00105444 | 148 | 1259 | C | C | T | noncoding | n/a | n/a | n/a | n/a |
| 56 | 7510521 | 6109371H1 | SNP00006579 | 150 | 526 | C | C | T | noncoding | 0.82 | n/a | n/a | n/a |
| 56 | 7510521 | 6116936H1 | SNP00067426 | 32 | 97 | T | T | C | noncoding | 0.95 | 0.95 | n/d | 0.96 |
| 56 | 7510521 | 6325544H1 | SNP00006579 | 171 | 526 | C | C | T | noncoding | 0.82 | n/a | n/a | n/a |
| 56 | 7510521 | 6325576H1 | SNP00006579 | 177 | 526 | C | C | T | noncoding | 0.82 | n/a | n/a | n/a |
| 56 | 7510521 | 641917H1 | SNP00006579 | 67 | 526 | C | C | T | noncoding | 0.82 | n/a | n/a | n/a |
| 56 | 7510521 | 6458184H1 | SNP00006579 | 91 | 526 | C | C | T | noncoding | 0.82 | n/a | n/a | n/a |
| 56 | 7510521 | 652597H1 | SNP00067426 | 28 | 97 | T | T | C | noncoding | 0.95 | 0.95 | n/d | 0.96 |
| 56 | 7510521 | 6529484H1 | SNP00006579 | 255 | 526 | C | C | T | noncoding | 0.82 | n/a | n/a | n/a |
| 56 | 7510521 | 6532850H1 | SNP00105444 | 415 | 1259 | C | C | T | noncoding | n/a | n/a | n/a | n/a |
| 56 | 7510521 | 6869191H1 | SNP00105444 | 123 | 1259 | C | C | T | noncoding | n/a | n/a | n/a | n/a |
| 56 | 7510521 | 6916821H1 | SNP00006579 | 170 | 526 | C | C | T | noncoding | 0.82 | n/a | n/a | n/a |
| 56 | 7510521 | 6966242H1 | SNP00006579 | 338 | 526 | C | C | T | noncoding | 0.82 | n/a | n/a | n/a |
| 56 | 7510521 | 7185105H1 | SNP00105444 | 117 | 1259 | C | C | T | noncoding | n/a | n/a | n/a | n/a |
| 56 | 7510521 | 7218669H1 | SNP00105444 | 314 | 1259 | C | C | T | noncoding | n/a | n/a | n/a | n/a |
| 56 | 7510521 | 7332779H1 | SNP00105444 | 351 | 1224 | C | C | T | noncoding | n/a | n/a | n/a | n/a |
| 56 | 7510521 | 7365079H1 | SNP00067426 | 36 | 97 | T | T | C | noncoding | 0.95 | 0.95 | n/d | 0.96 |
| 56 | 7510521 | 7370713H1 | SNP00006580 | 557 | 1241 | T | T | G | noncoding | n/a | n/a | n/a | n/a |
| 56 | 7510521 | 7628538J1 | SNP00105444 | 459 | 1259 | C | C | T | noncoding | n/a | n/a | n/a | n/a |
| 56 | 7510521 | 7632152H1 | SNP00105444 | 561 | 1259 | C | C | T | noncoding | n/a | n/a | n/a | n/a |
| 56 | 7510521 | 766430H1 | SNP00105444 | 135 | 1259 | C | C | T | noncoding | n/a | n/a | n/a | n/a |
| 56 | 7510521 | 777571H1 | SNP00067426 | 12 | 97 | T | T | C | noncoding | 0.95 | 0.95 | n/d | 0.96 |
| 56 | 7510521 | 7936627H1 | SNP00006579 | 71 | 526 | C | C | T | noncoding | 0.82 | n/a | n/a | n/a |

1. An isolated polypeptide selected from the group consisting of:
a) a polypeptide comprising an amino acid sequence selected from the group consisting of SEQ ID NO:1-28,
b) a polypeptide comprising a naturally occurring amino acid sequence at least 90% identical to an amino acid sequence selected from the group consisting of SEQ ID NO:1, SEQ ID NO:3-7, SEQ ID NO:9-19, SEQ I) NO:21-26, and SEQ ID NO:28,
c) a polypeptide comprising a naturally occurring amino acid sequence at least 94% identical to an amino acid sequence selected from the group consisting of SEQ ID NO:2 and SEQ ID NO:20,
d) a polypeptide comprising a naturally occurring amino acid sequence at least 96% identical to an amino acid sequence of SEQ ID NO:8,
e) a polypeptide comprising a naturally occurring amino acid sequence at least 97% identical to an amino acid sequence of SEQ ID NO:27,
f) a biologically active fragment of a polypeptide having an amino acid sequence selected from the group consisting of SEQ ID NO:1-28, and
g) an immunogenic fragment of a polypeptide having an amino acid sequence selected from the group consisting of SEQ ID NO:1-28.

2. An isolated polypeptide of claim 1 comprising an amino acid sequence selected from the group consisting of SEQ I) NO:1-28.

3. An isolated polynucleotide encoding a polypeptide of claim 1.

4. An isolated polynucleotide encoding a polypeptide of claim 2.

5. An isolated polynucleotide of claim 4 comprising a polynucleotide sequence selected from the group consisting of SEQ ID NO:29-56.

6. A recombinant polynucleotide comprising a promoter sequence operably linked to a polynucleotide of claim 3.

7. A cell transformed with a recombinant polynucleotide of claim 6.

8. (CANCELED).

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. A method of claim 9, wherein the polypeptide comprises an amino acid sequence selected from the group consisting of SEQ ID NO:1-28.

11. An isolated antibody which specifically binds to a polypeptide of claim 1.

12. An isolated polynucleotide selected from the group consisting of:
a) a polynucleotide comprising a polynucleotide sequence selected from the group consisting of SEQ ID NO:29-56,
b) a polynucleotide comprising a naturally occurring polynucleotide sequence at least 90% identical to a polynucleotide sequence selected from the group consisting of SEQ ID NO:29-53 and SEQ ID NO:55-56,
c) a polynucleotide comprising a naturally occurring polynucleotide sequence at least 91% identical to a polynucleotide sequence of SEQ ID NO:54,
d) a polynucleotide complementary to a polynucleotide of a),
e) a polynucleotide complementary to a polynucleotide of b),
f) a polynucleotide complementary to a polynucleotide of c), and
g) an RNA equivalent of a)-f).

13. (CANCELED).

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. (CANCELED).

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. A composition of claim 17, wherein the polypeptide comprises an amino acid sequence selected from the group consisting of SEQ ID NO:1-28.

19. (CANCELED).

20. A method of screening a compound for effectiveness as an agonist of a polypeptide of claim 1, the method comprising:
a) exposing a sample comprising a polypeptide of claim 1 to a compound, and
b) detecting agonist activity in the sample.

21. (CANCELED).

22. (CANCELED).

23. A method of screening a compound for effectiveness as an antagonist of a polypeptide of claim 1, the method comprising:
a) exposing a sample comprising a polypeptide of claim 1 to a compound, and
b) detecting antagonist activity in the sample.

24. (CANCELED).

25. (CANCELED).

26. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. (CANCELED).

28. A method of screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

29. A method of assessing toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample is indicative of toxicity of the test compound.

30-111. (CANCELED).


SEQUENCE LISTING (56 sequences)

SEQ ID NO: 1 - protein, 774 aa, Homo sapiens
  misc_feature: Incyte ID No 7994355CD1
MADQHRSVSELLSNSKFDVNYAFGRVKRSLLHIAANCGSVECLVLLLKKGANPNYQDISGCTPLHLAARNGHGQRDTAQILLLRGAKYLPDKNGVTPLDLCVQGGYGETCEVLIQYHPRLFQTIIQMTQNEDLRENMLRQVLEHLSQQSESQYLKILTSLAEVATTNGHKLLSLSSNYDAQMKSLLRIVRMFCHVFRIGPSSPSNGIDMGYNGNKTPRSQVFKPLELLWHSLDEWLVLIATELMKNKRDSTEITSILLKQKGQDQDAASIPPFEPPGPGSYENLSTGTRESKPDALAGRQEASADCQDVISMTANRLSAVIQAFYMCCSCQMPPGMTSPRFIEFVCKHDEVLKCFVNRNPKIIFDHFHFLLECPELMSRFMHIIKAQPFKDRCEWFYEHLHSGQPDSDMVHRPVNENDILLVHRDSIFRSSCEVVSKANCAKLKQGIAVRFHGEEGMGQGVVREWFDILSNEIVNPDYALFTQSADGTTFQPNSNSYVNPDHLNYFRFAGQILGLALNHRQLVNIYFTRSFYKHILGIPVNYQDVASIDPEYAKNLQWILDNDISDLGLELTFSVETDVFGAMEEVPLKPGGGSILVTQNNKAEYVQLVTELRMTRAIQPQINAFLQGFHMFIPPSLIQLFDEYELELLLSGMPEIDVSDWIKNTEYTSGYEREDPVIQWFWEVVEDITQEERVLLLQFVTGSSRVPHGGFANIMGGSGLQNFTIAAVPYTPNLLPTSSTCINMLKLPEYPSKEILKDRLLVALHCGSYGYTMA

SEQ ID NO: 2 - protein, 703 aa, Homo sapiens
  misc_feature: Incyte ID No 7475875CD1
MACSMACGGRACKYENPARWSEQEQAIKGVYSSWVTDNILAMARPSSELLEKYHIIDQFLSHGIKTIINLQRPGEHASCGNPLEQESGFTYLPEAFMEAGIYFYNFGWKDYGVASLTTILDMVKVMTFALQEGKVAIHCHAGLGRTGVLIACYLVFATRMTADQAIIFVRAKRPNSIQTRGQLLCVREFTQFLTPLRNIFSCCDPKAHAVTLPQYLIRQRHLLHGYEARLLKHVPKIIHLVCKLLLDLAENRPVMMKDVSEGPGLSAEIEKTMSEMVTMQLDKELLRHDSDVSNPPNPTAVAADFDNRGMIFSNEQQFDPLWKRRNVECLQPLTHLKRRLSYSDSDLKRAENLLEQGETPQTVPAQILVGHKPRQQKLISHCYIPQSPEPDLHKEALVRSTLSFWSQSKFGGLEGLKDNGSPIFHGKIIPKEAQQSGAFSADVSGSHSPGEPVSPSFANVHKDPNPAHQQVSHCQCKTHGVGSPGSVRQNSRTPRSPLDCGSSPKAQFLVEHETQDSKDLSEAASHSALQSELSAEARRILAAKALANLNESVEKEELKRKVEMWQKELNSRDGAWERICGERDPFILCSLMWSWVEQLKEPVITKEDVDMLVDRRADAAEALFLLEKGQHQTILCVLHCIVNLQTIPVDVEEAFLAHAIKAFTKVNFDSENGPTVYNTLKKIFKHTLEEKRKMTKDGPKPGL

SEQ ID NO: 3 - protein, 1256 aa, Homo sapiens
  misc_feature: Incyte ID No 71231882CD1
MFGDLFEEEYSTVSNNQYGKGKKLKTKALEPPAPREFTNLSGIRNQGGTCYLNSLLQTLHFTPEFREALFSLGPEELGLFEDKDKPDAKVRIIPLQLQRLFAQLLLLDQEAASTADLTDSFGWTSNEEMRQHDVQELNRILFSALETSLVGTSGHDLIYRLYHGTIVNQIVCKECKNVSERQEDFLDLTVAVKNVSGLEDALWNMYVEEEVFDCDNLYHCGTCDRLVKAAKSAKLRKLPPFLTVSLLRFNFDFVKCERYKETSCYTFPLRINLKPFCEQSELDDLEYIYDLFSVIIHKGGCYGGHYHVYIKDVDHLGNWQFQEEKSKPDVNLKDLQSEEEIDHPLMILKAILLEEENNLIPVDQLGQKLLKKIGISWNKKYRKQHGPLRKFLQLHSQIFLLSSDESTVRLLKNSSLQAESDFQRNDQQIFKMLPPESPGLNNSISCPHWFDINDSKVQPIREKDIEQQFQGKESAYMLFYRKSQLQRPPEARANPRYGVPCHLLNEMDAANIELQTKRAECDSANNTFELHLHLGPQYHFFNGALHPVVSQTESVWDLTFDKRKTLGDLRQSIFQLLEFWEGDMVLSVAKLVPAGLHIYQSLGGDELTLCETEIADGEDIFVWNGVEVGGVHIQTGIDCEPLLLNVLHLDTSSDGEKCCQVIESPHVFPANAEVGTVLTALAIPAGVIFINSAGCPGGEGWTAIPKEDMRKTFREQGLRNGSSILIQDSHDDNSLLTKEEKWVTSMNEIDWLHVKNLCQLESEEKQVKISATVNTMVFDIRIKAIKELKLMKELADNSCLRPIDRNGKLLCPVPDSYTLKEAELKMGSSLGLCLGKAPSSSQLFLFFAMGSDVQPGTEMEIVVEETISVRDCLKLMLKKSGLQGDAWHLRKMDWCYEAGEPLCEENSARSQLITLGTGFSFQPCQDATLKELLICSGDTLLLIEGQLPPLGFLKVPIWWYQLQGPSGHWESHQDQTNCTSSWGRVWRATSSQGASGNEPAQVSLLYLGDIEISEDATLAELKSQAMTLPPFLEFGVPSPAHLRAWTVERKRPGRLLRTDRQPLREYKLGRRIEICLEPLQKGENLGPQDVLLRTQVRIPGERTYAPALDLVWNAAQGGTAGSLRQRVADFYRLPVEKIEIAKYFPEKFEWLPISSWNQQITKRKKKKKQDYLQGAPYYLKDGDTIGVKNLLIDDDDDFSTIRDDTGKEKQKQRALGRRKSQEALHEQSSYILSSAETPARPRAPETSLSIHVGSFR

SEQ ID NO: 4 - protein, 755 aa, Homo sapiens
  misc_feature: Incyte ID No 2875922CD1
MKKQRKILWRKGIHLAFSEKWNTGFGGFKKFYFHQHLCILKAKLGRPVTWNRQLRHFQGRKKALQIQKTWIKDEPLCAKTKFNVATQNVSTLSSKVKRKDAKHFISSSKTLLRLQAEKLLSSAKNSDHEYCREKNLLKAVTDFPSNSALGQANGHRPRTDPQPSDFPMKFNGESQSPGESGTIVVTLNNHKRKGFCYGCCQGPEHHRNGGPLIPKKFQLNQHRRIKLSPLMMYEKLSMIRFRYRILRSQHFRTKSKVCKLRKAQRSWVQKVTGDHQETRRENGEGGSCSPFPSPEPKDPSCRHQPYFPDMDSSAVVKGTNSHVPDCHTKGSSFLGKELSLDEAFPDQQNGSATNAWDQSSCSSPKWECTELIHDIPLPEHRSNTMFISETEREIMTLGQENQTSSVSDDRVKLSVSGADTSVSSVDGPVSQKAVQNENSYQMEEDGSLKQSILSSELLDHPYCKSPLEAPLVCSGLKLENQVGGGKNSQKASPVDDEQLSVCLSGFLDEVMKKYGSLVPLSEKEVLGRLKDVFNEDFSNRKPFINREITNYRARHQKCNFRIFYNKHMLDMDDLATLDGQNWLNDQVINMYGELIMDAVPDKVHFFNSFFHRQLVTKGYNGVKRWTKKVDLFKKSLLLIPIHLEVHWSLITVTLSNRIISFYDSQGIHFKFCVENIRKYLLTEAREKNRPEFLQGWQTAVTKCIPQQKNDSDCGVFVLQYCKCLALEQPFQFSQEDMPRVRKRIYKELCECRLMD

SEQ ID NO: 5 - protein, 1034 aa, Homo sapiens
  misc_feature: Incyte ID No 8158136CD1
MAPRLQLEKAAWRWAETVRPEEVSQEHIETAYRIWLEPCIRGVCRRNCKGNPNCLVGIGEHIWLGEIDENSFHNIDDPNCERRKKNSFVGLTNLGATCYVNTFLQVWFLNLELRQALYLCPSTCSDYMLGDGIQEEKDYEPQTICEHLQYLFALLQNSNRRYIDPSGFVKALGLDTGQQQDAQEFSKLFMSLLEDTLSKQKNPDVRNIVQQQFCGEYAYVTVCNQCGRESKLLSKFYELELNIQGHKQLTDCISEFLKEEKLEGDNRYFCENCQSKQNATRKIRLLSLPCTLNLQLMRFVFDRQTGHKKKLNTYIGFSEILDMEPYVEHKGGSYVYELSAVLIHRGVSAYSGHYIAHVKDPQSGEWYKFNDEDIEKMEGKKLQLGIEEDLAEPSKSQTRKPKCGKGTHCSRNAYMLVYRLQTQEKPNTTVQVPAFLQELVDRDNSKFEEWCIEMAEMRKQSVDKGKAKHEEVKELYQRLPAGAEPYEFVSLEWLQKWLDESTPTKPIDNHACLCSHDKLHPDKISIMKRISEYAADIFYSRYGGGPRLTVKALCKECVVERCRILRLKNQLNEDYKTVNNLLKAAVKGDGFWVGKSSLRSWRQLALEQLDEQDGDAEQSNGKMNGSTLNKDESKEERKEEEELNFNEDILCPHGELCISENERRLVSKEAWSKLQQYFPKAPEFPSYKECCSQCKILEREGEENEALHKMIANEQKTSLPNLFQDKNRPCLSNWPEDTDVLYIVSQFFVEEWRKFVRKPTRCSPVSSVGNSALLCPHGGLMFTFASMTKEDSKLIALIWPSEWQMIQKLFVVDHVIKITRIEVGDVNPSETQYISEPKLCPECREGLLCQQQRDLREYTQATIYVHKVVDNKKVMKDSAPELNVSSSETEEDKEEAKPDGEKDPDFNQSNGGTKRQKISHQNYIAYQKQVIRRSMRHRKVRGEKALLVSANQTLKELKIQIMHAFSVAPFDQNLSIDGKILSDDCATLGTLGVIPESVILLKADEPIADYAAMDDVMQVCMPEEGFKGTGLLGH

SEQ ID NO: 6 - protein, 1236 aa, Homo sapiens
  misc_feature: Incyte ID No 5969491CD1
MFGDLFEEEYSTVSNNQYGKGKKLKTKALEPPAPREFTNLSGIRNQGGTCYLNSLLQTLHFTPEFREALFSLGPEELGLFEDKDKPDAKVRIIPLQLQRLFAQLLLLDQEAASTADLTDSFGWTSNEEMRQHDVQELNRILFSALETSLVGTSGHDLIYRLYHGTIVNQIVCKECKNVSERQEDFLDLTVAVKNVSGLEDALWNMYVEEEVFDCDNLYHCGTCDRLVKAAKSAKLRKLPPFLTVSLLRFNFDFVKCERYKETSCYTFPLRINLKPFCEQSELDDLEYIYDLFSVIIHKGGCYGGHYHVYIKDVDHLGNWQFQEEKSKPDVNLKDLQSEEEIDHPLMILKAILLEEENNLIPVDQLGQKLLKKIGISWNKKYRKQHGPLRKFLQLHSQIFLLSSDESTVRLLKNSSLQAESDFQRNDQQIFKMLPPESPGLNNSISCPHWFDINDSKVQPIREKDIEQQFQGKESAYMLFYRKSQLQRPPEARANPRYGVPCHLLNEMDAANIELQTKRAECDSANNTFELHLHLGPQYHFFNGALHPVVSQTESVWDLTFDKRKTLGDLRQSIFQLLEFWEGDMVLSVAKLVPAGLHIYQSLGGDELTLCETEIADGEDIFVWNGVEVGGVHIQTGIDCEPLLLNVLHLDTSSDGEKCCQVIESPHVFPANAEVGTVLTALAIPAGVIFINSAGCPGGEGWTAIPKEDMRKTFREQGLRNGSSILIQDSHDDNSLLTKEEKWVTSMNEIDWLHVKNLCQLESEEKQVKISATVNTMVFDIRIKAIKELKLMKELADNSCLRPIDRNGKLLCPVPDSYTLKEAELKMGSSLGLCLGKAPSSSQLFLFFAMGSDVQPGTEMEIVVEETISVRDCLKLMLKKSGLQGDAWHLRKMDWCYEAGEPLCEEDATLKELLICSGDTLLLIEGQLPPLGFLKVPIWWYQLQGPSGHWESHQDQTNCTSSWGRVWRATSSQGASGNEPAQVSLLYLGDIEISEDATLAELKSQAMTLPPFLEFGVPSPAHLRAWTVERKRPGRLLRTDRQPLREYKLGRRIEICLEPLQKGENLGPQDVLLRTQVRIPGERTYAPALDLVWNAAQGGTAGSLRQRVADFYRLPVEKIEIAKYFPEKFEWLPISSWNQQITKRKKKKKQDYLQGAPYYLKDGDTIGVKNLLIDDDDDFSTIRDDTGKEKQKQRALGRRKSQEALHEQSSYILSSAETPARPRAPETSLSIHVGSFR

SEQ ID NO: 7 - protein, 545 aa, Homo sapiens [sequence omitted]

SEQ ID NO: 8 - protein, 414 aa, Homo sapiens
  misc_feature: Incyte ID No 7632424CD1
MLILTKTAGVFFKPSKRKVYEFLRSFNFHPGTLFLHKIVLGIETSCDDTAAAVVDETGNVLGEAIHSQTEVHLKTGGIVPPAAQQLHRENIQRIVQEALSASGVSPSDLSAIATTIKPGLALSLGVGLSFSLQLVGQLKKPFIPIHHMEAHALTIRLTNKVEFPFLVLLISGGHCLLALVQGVSDFLLLGKSLDIAPGDMLDKVARRLSLIKHPECSTMSGGKAIEHLAKQGNRFHFDIKPPLHHAKNCDFSFTGLQHVTDKIIMKKEKEEGIEKGQILSSAADIAATVQHTMACHLVKRTHRAILFCKQRDLLPQNNAVLVASGGVASNFYIRRALEILTNATQCTLLCPPPRLCTDNGIMIAWNGIERLRAGLGILHDIEGIRYEPKCPLGVDISKEVGEASIKVPQLKMEI

SEQ ID NO: 9 - protein, 611 aa, Homo sapiens
  misc_feature: Incyte ID No 1804436CD1
MSCKKQRSRKHSVNEKCNMKIEHYFSPVSKEQQNNCSTSLMRMESRGDPRATTNTQAQRFHSPKKNPEDQTMPQNRTIYVTLKVNHRRNQDMKLKLTHSENSSLYMALNTLQAVRKEIETHQGQEMLVRGTEGIKEYINLGMPLSCFPEGGQVVITFSQSKSKQKEDNHIFGRQDKASTECVKFYIHAIGIGKCKRRIVKCGKLHKKGRKLCVYAFKGETIKDALCKDGRFLSFLENDDWKLIENNDTILESTQPVDELEGRYFQVEVEKRMVPSAAASQNPESEKRNTCVLREQIVAQYPSLKRESEKIIENFKKKMKVKNGETLFELHRTTFGKVTKNSSSIKVVKLLVRLSDSVGYLFWDSATTGYATCFVFKGLFILTCRHVIDSIVGDGIEPSKWATIIGQCVRVTFGYEELKDKETNYFFVEPWFEIHNEELDYAVLKLKENGQQVPMELYNGITPVPLSGLIHIIGHPYGEKKQIDACAVIPQGQRAKKCQERVQSKKAESPEYVHMYTQRSFQKIVHNPDVITYDTEFFFGASGSPVFDSKGSLVAMHAAGFAYTYQNETRSIIEFGSTMESILLDIKQRHKPWYEEVFVNQQDVEMMSDEDL

SEQ ID NO: 10 - protein, 147 aa, Homo sapiens
  misc_feature: Incyte ID No 7486358CD1
MWSLPPSRALSCAPLLLLFSFQFLVTYAWRFQEEEEWNDQKQIAVYLPPTLEFAVYTFNKQSKDWYAYKLVPVLASWKEQGYDKMTFSMNLQLGRTMCGKFEDDIDNCPFQESPELNNTCTCFFTIGIEPWRTRFDLWNKTCSGGHS

SEQ ID NO: 11 - protein, 624 aa, Homo sapiens
  misc_feature: Incyte ID No 7472344CD1
MALRARALYDFRSENPGEISLREHEVLSLCSEQDIEGWLEGVNSRGDRGLFPASYVQVIRAPEPGPAGDGGPGAPARYANVPPGGFEPLPVAPPASFKPPPDAFQALLQPQQAPPPSTFQPPGAGFPYGGGALQPSPQQLYGGYQASQGSDDDWDDEWDDSSTVADEPGALGSGAYPDLDGSSSAGVGAAGRYRLSTRSDLSLGSRGGSVPPQHHPSGPKSSATVSRNLNRFSTFVKSGGEAFVLGEASGFVKDGDKLCVVLGPYGPEWQENPYPFQCTIDDPTKQTKFKGMKSYISYKLVPTHTQVPVHRRYKHFDWLYARLAEKFPVISVPHLPEKQATGRFEEDFISKRRKGLIWWMNHMASHPVLAQCDVFQHFLTCPSSTDEKAWKQGKRKAEKDEMVGANFFLTLSTPPAAALDLQEVESKIDGFKCFTKKMDDSALQLNHTANEFARKQVTGFKKEYQKVGQSFRGLSQAFELDQQAFSVGLNQAIAFTGDAYDAIGELFAEQPRQDLDPVMDLLALYQGHLANFPDIIHVQKGALTKVKESRRHVEEGKMEVQKADGIQDRCNTISFATLAEIHHFHQIRVRDFKSQMQHFLQQQIIFFQKVTQKLEEALHKYDSV

SEQ ID NO: 12 - protein, 283 aa, Homo sapiens
  misc_feature: Incyte ID No 7192959CD1
MGASVSRGRAARVPAPEPEPEEALDLSQLPPELLLVVLSHVPPRTLLGRCRQVCRGWRALVDGQALWLLILARDHGATGRALLHLARSCQSPARNARPCPLGRFCARRPIGRNLIRNPCGQEGLRKWMVQHGGDGWVVEENRTTVPGAPSQTCFVTSFSWCCKKQVLDLEEEGLWPELLDSGRIEICVSDWWGARHDSGCMYRLLVQLLDANQTVLDKFSAVPDPIPQWNNNACLHVTHVFSNIKMGVRFVSFEHRGQDTQFWAGHYGARVTNSSVIVRVRLS

SEQ ID NO: 13 - protein, 142 aa, Homo sapiens [sequence omitted]

SEQ ID NO: 14 - protein, 354 aa, Homo sapiens
  misc_feature: Incyte ID No 7494717CD1
MAESPTEEAATAGAGAAGPGASSVAGVVGVSGSGGGFGPPFLPDVWAAAAAAGGAGGPGSGLAPLPGLPPSAAAHGAALLSHWDPTLSSDWDGERTAPQCLLRIKRDIMSIYKEPPPGMFVVPDTVDMTKIHALITGPFDTPYEGGFFLFVFRCPPDYPIHPPRVKLMTTGNNTVRFNPNFYRNGKVCLSILGTWTGPAWSPAQSISSVLISIQSLMTENPYHNEPGFEQERHPGDSKNYNECIRHETIRVAVCDMMEGKCPCPEPLRGVMEKSFLEYYDFYEVACKDRLHLQGQTMQDPFGEKRGHFDYQSLLMRLGLIRQKVLERLHNENAEMDSDSSSSGTETDLHGSLRV

SEQ ID NO: 15 - protein, 89 aa, Homo sapiens
  misc_feature: Incyte ID No 7497510CD1
MQLQASLSFLLILTLCLELRSELARDTIKDLLPNVCAFPMEKGPCQTYMTRWFFNFETGECELFAYGGCGGNSNNFLRKEKCEKFCKFT

SEQ ID NO: 16 - protein, 419 aa, Homo sapiens
  misc_feature: Incyte ID No 7498882CD1
MGAGPSLLLAALLLLLSGDGAVRCDTPANCTYLDLLGTWVFQVGSSGSQRDVNCSVMGPQEKKVVVYLQKLDTAYDDLGNSGHFTIIYNQGFEIVLNDYKWFAFFKYKEEGSKVTTYCNETMTGWVHDVLGRNWACFTGKKVGTASENVYVNTAHLKNSQEKYSNRLYKYDHNFVKAINAIQKSWTATTYMEYETLTLGDMIRRSGGHSRKIPRPKPAPLTAEIQQKILHLPTSWDWRNVHGINFVSPVRNQGCEGGFPYLIAGKYAQDFGLVEEACFPYTGTDSPCKMKEDCFRYYSSEYHYVGGFYGGCNEALMKLELVHHGPMAVAFEVYDDFLHYKKGIYHHTGLRDPFNPFELTNHAVLLVGYGTDSASGMDYWIVKNSWGTGWGENGYFRIRRGTDECAIESIAVAATPIPKL

SEQ ID NO: 17 - protein, 951 aa, Homo sapiens
  misc_feature: Incyte ID No 5524205CD1
MDHQNLSEHVLCMVLYLIELGLENSAEEESDEEASVGGPERCHDSWFPGSNLVSNMRHFINYVRVRVPETAPEVKRDSPASTSSDNLGSLQNSGTAQVFSLVAERRKKFQEIINRSSSEANQVVRPKTSSKWSAPGSAPQLTTAILEIKESILSLLIKLHHKLSGKQNSYYPPWLDDIEILIQPEIPKYSHGDGITAVERILLKAASQSRMNKRIIEEICRKVTPPVPPKKVTAAEKKTLDKEERRQKARERQQKLLAEFASRQKSFMETAMDVDSPENDIPMEITTAEPQVSEAVYDCVICGQSGPSSEDRPTGLVVLLQASSVLGQCRDNVEPKKLPISEEEQIYPWDTCAAVHDVRLSLLQRYFKDSSCLLAVSIGWEGGVYVQTCGHTLHIDCHKSYMESLRNDQVLQGFSVDKGEFTCPLCRQFANSVLPCYPGSNVENNPWQRPSNKSIQDLIKEVEELQGRPGAFPSETNLSKEMESVMKDIKNTTQKKYRDYSKTPGSPDNDFLFMYSVARTNLELELIHRGGNLCSGGASTAGKRSCLNQLFHVLALHMRLYSIDSEYNPWRKLTQLEEMNPQLGYEEQQPEVPILYHDVTSLLLIQILMMPQPLRKDHFTCIVKVLFTLLYTQALAALSVKCSEEDRSAWKHAGALKKSTCDAEKSYEVLLSFVISELFKGKLYHEEGTQECAMVNPIAWSPESMEKCLQDFCLPFLRITSLLQHHLFGEDLPSCQEEEEFSVLASCLGLLPTFYQTEHPFISASCLDWPVPAFDIITQWCFEIKSFTERHAEQGKALLIQESKWKLPHLLQLPENYNTIFQYYHRKTCSVCTKVPKDPAVCLVCGTFVCLKGLCCKQQSYCECVLHSQNCGAGTGIFLLINASVIIIIRGHRFCLWGSVYLDAHGEEDRDLRRGKPLYICKERYKVLEQQWISHTFDHINKRWGPHYNGL

SEQ ID NO: 18 - protein, 668 aa, Homo sapiens
  misc_feature: Incyte ID No 7102342CD1
MFRLWLLLAGLCGLLASRPGFQNSLLQIVIPEKIQTNTNDSSEIEYEQISYIIPIDEKLYTVHLKQRYFLADNFMIYLYNQGSMNTYSSDIQTQCYYQGNIEGYPDSMVTLSTCSGLRGILQFENVSYGIEPLESAVEFQHVLYKLKNEDNDIAIFIDRSLKEQPMDDNIFISEKSEPAVPDLFPLYLEMHIVVDKTLYDYWGSDSMIVTNKVIEIVGLANSMFTQFKVTIVLSSLELWSDENKISTVGEADELLQKFLEWKQSYLNLRPHDIAYLLIYMDYPRYLGAVFPGTMCITRYSAGVALQCGPASCCDFRTCVLKDGAKCYKGLCCKDCQILQSGVECRPKAHPECDIAENCNGSSPECGPDITLINGLSCKNNKFICYDGDCHDLDARCESVFGKGSRNAPFACYEEIQSQSDRFGNCGRDRNNKYVFCGWRNLICGRLVCTYPTRKPFHQENGDVIYAFVRDSVCITVDYKLPRTVPDPLAVKNGSQCDIGRVCVNRECVESRIIKASAHVCSQQCSGHGVCDSRNKCHCSPGYKPPNCQIRSKGFSIFPEEDMGSIMERASGKTENTWLLGFLIALPILIVTTAIVLARKQLKKWFAKEEEFPSSESKSEGSTQTYASQSSSEGSTQTYASQTRSESSSQADTSKSKSQDSTQTQSSSN

SEQ ID NO: 19 - protein, 206 aa, Homo sapiens
  misc_feature: Incyte ID No 4169939CD1
MMLRLLSSLLLVAVASGYGPPSSHSSSRVVHGEDAIPINSEELFVHPLWNRSCVACGNDIALIKLSRSAQLGDAVQLASLPPAGDILPNKTPCYITGWGRLYTNGPLPDKLQQARLPVVDYKHCSRWNWWGSTVKKTMVCAGGYIRSGCNGDSGGPLNCPTEDGGWQVHGVTSFVSGFGCNFIWKPTVFTRVSAFIDWIEETIASH

SEQ ID NO: 20 - protein, 267 aa, Homo sapiens [sequence omitted]

SEQ ID NO: 21 - protein, 86 aa, Homo sapiens
  misc_feature: Incyte ID No 7675588CD1
MGLSGLLPILVPFILLGDIQEPGHAEGILGKPCPKIKVECEVEEIDQCTKPRDCPENMKCCPFSRGKKCLDFRKVSLTLYHKEELE

SEQ ID NO: 22 - protein, 232 aa, Homo sapiens
  misc_feature: Incyte ID No 6244077CD1
MANYYEVLGVQASASPEDIKKAYRKLALRWHPDKNPDNKEEAEKKFKLVSEAYEVLSDSKKRSLYDRAGCDSWRAGGGASTPYHSPFDTGYTFRNPEDIFREFFGGLDPFSFEFWDSPFNSDRGGRGHGLRGAFSAGFGEFPAFMEAFSSFNMLGCSGGSHTTFSSTSFGGSSSGSSGFKSVMSSTEMINGHKVTTKRIVENGQERVEVEEDGQLKSVTVNGKEQLKWMDSK

SEQ ID NO: 23 - protein, 237 aa, Homo sapiens
  misc_feature: Incyte ID No 7498404CD1
MASLEVSRSPRRSRRELEVRSPRQNKYSVLLPTYNERENLPLIVWLLVKSFSESGINYEIIIIDDGSPDGTRDVAEQLEKIYGSDRILLRPREKKLGLGTAYIHGMKHATGNYIIIMDADLSHHPKFIPEFIRKQKEGNFDIVSGTRYKGNGGVYGWDLKRKIIRLYRKEVLEKLIEKCVSKGYVFQMEMIVRARQLNYTIGEVPISFVDRVYGESKLGGNEIVSFLKGLLTLFATT

SEQ ID NO: 24 - protein, 146 aa, Homo sapiens
  misc_feature: Incyte ID No 7391748CD1
MLLQLSRRVRRNRNVNPVALPRAQEGLRPGTLCTVAGWGRVSMRRGTDTLREVQLRVQRDRQCLRIFGSYDPRRQICVGDRRERKAAFKGDSGGPLLCNNVAHGIVSYGKSSGVPPEVFTRVSSFLPWIRTTMRSFKLLDQMETPL

SEQ ID NO: 25 - protein, 696 aa, Homo sapiens
  misc_feature: Incyte ID No 7499780CD1
MTSSGPGPRFLLLLPLLLPPAASASDRPRGRDPVNPEKLLVITVATAETEGYLRFLRSAEFFNYTVRTLGLGEEWRGGDVARTVGGGQKVRWLKKEMEKYADREDMIIMFVDSYDVILAGSPTELLKKFVQSGSRLLFSAESFCWPEWGLAEQYPEVGTGKRFLNSGGFIGFATTIHQIVRQWKYKDDDDDQLFYTRLYLDPGLREKLSLNLDHKSRIFQNLNGALDEVVLKFDRNRVRIRNVAYDTLPIVVHGNGPTKLQLNYLGNYVPNGWTPEGGCGFCNQDRRTLPGGQEVFHEPHIADSWPQLQDHFSAVKLVGPEEALSPGEARDMAMDLCRQDPECEFYFSLDADAVLTNLQTLRILIEENRKVIAPMLSRHGKLWSNFWGALSPDEYYARSEDYVELVQRKRVGVWNVPYISQAYVIRGDTLRMELPQRDVFSGSDTDPDMAFCKSFRDKGIFLHLSNQHEFGRLLATSRYDTEHLHPDLWQIFDNPVDWKEQYIHENYSRALEGEGIVEQPCPDVYWFPLLSEQMCDELVAEMEHYGQWSGGRHEDSRLAGGYENVPTVDIHMKQVGYEDQWLQLLRTYVGPMTESLFPGYHTKARAVMNFVVRYRPDEQPSLRPHHDSSTFTLNVALNHKGLDYEGGGCRFLRYDCVISSPRKGWALLHPGRLTHYHEGLPTTWGTRYIMVSFVDP

SEQ ID NO: 26 - protein, 630 aa, Homo sapiens
  misc_feature: Incyte ID No 7499881CD1
MAYQIVLELHFSHCAAMGAALLMLIENALITQSRLMLLESVLIFFNLLAVLSYLKFFNCQKHSPFSLSWWFWLTLTGVACSCAVGIKYMGVFTYVLVLGVAAVHAWHLLGDQTLSNVGADVQCCMRPACMGQMRMSQGVCVFCHLLARAVALLVIPVVLYLLFFYVHLILVFRSGPHDQIMSSAFQASLEGGLARITQGQPLEVAFGSQVTLRNVFGKPVPCWLHSHQDTYPMIYENGRGSSHQQQVTCYPFKDVNNWWIVKDPRRHQLVVSSPPRPVRHGDMVQLVHGMTTRSLNTHDVAAPLSPHSQEVSCYIDYNISMPAQNLWRLEIVNRGSDTDVWKTILSEVRFVHVNTSAVLKLSGAHLPDWGYRQLEIVGEKLSRGYHGSTVWNVEEHRYGASQEQRERERELHSPAQVDVSRNLSFMARFSELQWRMLALRSDDSEHKYSSSPLEWVTLDTNIAYWLHPRTSAQIHLLGNIVIWVSGSLALAIYALLSLWYLLRRRRNVHDLPQDAWLRWVLAGALCAGGWAVNYLPFFLMEKTLFLYHYLPALTFQILLLPVVLQHISDHLCRSQLQRSIFSALVVAWYSSACHVSNTLRPLTYGDKSLSPHELKALRWKDSWDILIRKH

SEQ ID NO: 27 - protein, 242 aa, Homo sapiens
  misc_feature: Incyte ID No 7488579CD1
MLLLAPQMLNLLLLALPVLASRAYAAPAPGQALQRVGIVGGQEAPRSKWPWQVSLRVHGPYWMHFCGGSLIHPQWVLTAAHCVGPDVKDLAALRVQLREQHLYYQDQLLPVSRIIVHPQFYIIQTGADIALLELEEPVNISSHIHTVTLPPASETFPPGMPCWVTGWGDVDNNVHLPPPYPLKEVEVPVVENHLCNAEYHTGLHTGHSFQIVRDDMLCAGSENHDSCQGDSGGPLVCKVNGT

SEQ ID NO: 28 - protein, 48 aa, Homo sapiens
  misc_feature: Incyte ID No 7510521CD1
MGAGPSLLLAALLLLLSGDGAVRCDTPANCTYLDLLGTWVFQDHKKKK

SEQ ID NO: 29 - DNA, 4384 bp, Homo sapiens
  misc_feature: Incyte ID No 7994355CB1
agaggcggcggcgggtgtctgtaggtggtcggtccggcagcagcccggcccccggacgcaggacgtggccccaggcagccctcgcagctcagtgctctagccggggcaagcccgcgtctccgcctgctggacgggcccaggcgagatgtagggctctgggcgcggaggccgccggtggggcggctgatcgcggaggatcgcggagggcgcgccgaggatggagagagcgatggagcaactcaaccgcctgacgcgctcgctgcgccgcgcgcgcaccgtggagttgcccgaggagatgaggtctcactatgttccccaggctggtcttgaactcctgggctcaagtgatccgcaccctgctccccccaaagtgttgggattacagatgtgagccaccgcgcctggcctggggataatcttcttgaaaaaatgataatgaaactgctgtttatacattaatgccaatggttatggctgatcaacacaggtctgtttctgaactactatcaaattcaaaatttgatgtcaattatgcattcggacgtgtgaaaagaagcttgcttcacattgcagcaaattgtggatcggtggaatgcttggttttgctgttaaagaaaggagcaaatcctaactatcaagatatttcaggctgtacaccccttcatttggcagcaagaaatggtcatggtcagagagatacagcacagatcctactattacgaggagccaaatatctgccagataaaaatggagtaactcctctggatttatgtgtacagggtggatatggagagacttgtgaagtattaattcaatatcacccgaggctttttcagactattattcaaatgacacagaatgaagacctccgagaaaacatgttacggcaagttctggagcatttgtctcagcaaagtgaaagccagtacctaaagattctaacaagccttgctgaagttgctacaacaaatggtcataaactgcttagcctctctagcaattatgatgctcaaatgaagagccttttaaggattgtgagaatgttttgtcacgtctttcgaattggtccatcctcccccagtaatggaattgatatgggctacaatgggaataaaactccaagaagccaggtgttcaagcctctggaattgctttggcactcgttagatgaatggctagttttaatagccacagaattgatgaaaaacaaaagagactcaacagagatcacttctattttactgaaacaaaaaggccaagatcaagatgctgcttccattcctccatttgaacctccaggacctgggagctatgaaaatctgtccactggcacaagggaatctaaaccagatgctcttgcagggagacaggaagccagtgcagattgtcaggatgttatttctatgacagctaaccggctaagtgctgtcattcaagctttttacatgtgctgttcttgtcagatgcctccgggaatgacttcacctcgtttcattgaatttgtctgcaaacatgatgaagttttaaaatgctttgttaatagaaatcccaaaattatatttgaccactttcactttctccttgaatgtcctgagttgatgtcaagattcatgcatatcataaaagcacagccttttaaagatcgctgtgaatggttctatgaacatttgcattcaggacagccagattcagatatggtgcacaggccagtgaatgaaaatgatatcctgctggttcacagagattctatttttaggagtagctgtgaagttgtgtcaaaagcaaattgtgcaaagctaaagcaagggattgctgtacggttccatggagaagaaggcatgggtcaaggtgttgtgcgtgagtggtttgatattctgtccaatgagatagtcaatcctgattatgcattgtttacccagtcagctgatggaacaacttttcagcctaatagcaactcttatgtaaatcctgatcacttgaactattttcggtttgctgggcagatcttgggattagcgttgaaccacaggcagctggtcaatatttacttcacacgatccttctacaagcacattcttggtattcctgtaaattaccaagatgtggcatccattgatccagaatatgcgaaaaatttgcaatggattttagataatgatataagtgatctgggtctagaactaactttttctgttgagactgatgtgtttggagcaatggaagaggtgcctttgaaacctgggggtgggagtattcttgtgacacaaaataataaagcggagtacgtccagcttgttactgaacttcgaatgacaagagccattcagcctcagatcaatgcttttttacagggctttcatatgttcattccaccctccctcatacagctttttgatgaatatgaattggagctactgctttctggcatgccagaaattgatgtgagtgattggataaaaaatacagaatacacaagtggctatgaaagagaagatccagttattcagtggttctgggaagttgtagaagacattactcaagaggagagagttcttctcttacagtttgttacgggcagttccagggtcccacatggtgggtttgctaatatcatgggtggaagtggattgcaaaactttacaatcgctgctgtgccatatactccaaatcttttaccaacttcaagcacatgcatcaacatgctcaagttacctgaatacccaagtaaagaaatactcaaggacagacttcttgtggcactacattgtggcagctatggttacacaatggcataatgaagtctggaaaactcctctgactactgatgcacaattcagaatggcagaagtaatttgggaaaatgtcaacaaaaaagcagcctaaatgcaacccataggcagggctgatgcttccaatttataaaggatcatcaggttttctgtttctctcttttcccttttatgttttctctgtttgtgatacaattagaaaatataaaatcacagtagattttattttttaaaatgctaactgaaagtaatagagactgtcctttttcataattaattttatccaagattgtattaaggcaaaatctgattctacattccacctctgctatgtaactgtcttgttaaaagggtgttttctcctaatttctgatatattatatgaggtcatccagctggtgtgttcttttgcatgtaaactgccatttatattttagaaaactattgtatagaatggatttagattgtctataaagccacaaatacgtattttgccacagtgtattctatattgcaatgatttttttagcattttaatattttaatatatattgtaaaatttagactgatgatactaacagttgatgaaatgacatataatttatatatgaaagcttacgctatattgtatgaattatttgcatctttcagtggccagttttccatatgtatatattatggtctcaatgtttttcttacgcctcattttaatttataatgaaggtaaaattaaaatgtattttaccacgtttcttttcattacttttatctgtgagctctgacacatctgaaaaagtaatctgatgtgcaaattataatttaaatatgttaatttttttgcttcttaaatttgcttttcatcattaaaatgtcaagttcaagtgatatgtgcctaatatcacttggatgttggtgggtttttgaatttttgggtggttaatcagttttattttgaaaagacgtacttgaatagttacagcatatgtttgaacaggaagtaggaacatgcatacacgaagaaatgctaacggaaggatttgttatgtttaggatcttcccttggaaactaaaaatagaatattaatgacattactgtttgtagaatgacatatgcagattttctcataagcagtcattgtgtttgccagtaatgtttgagagacatgtaagttgaaagttttgctaaattataaagctcctttaattcgttggttttgattctcttattctcttgtcttttctaaatgttaacaaaatatatcttaacagattacatgaaatttaggaattatttaaaagttaccattagctctaaaattaagattcggatgctttatttatagtaactgaagctaataatgttttatgttttgattttttgaaatttaattgtagaagtcactgccttctgagttttcaaatagataaccacctttaatattacactgcttataatactaatgtttacagatatgtttctgtttataaccatataatacattggctttgtcatattagttttttttgcaagtagttatgtaaaagagatagataataaaatattaaataactgagaaaaaaaaaaaaaaaaaaaa

SEQ ID NO: 30 - DNA, 4007 bp, Homo sapiens
  misc_feature: Incyte ID No 7475875CB1
atggcatgttccatggcgtgtggcggtagagcttgcaagtatgagaacccagcccgctggagtgagcaggagcaagccattaagggggtttactcatcctgggtcactgataatatactggccatggcccgcccatcctctgagctcctggagaagtaccacatcattgatcagttcctcagccatggcataaaaacaataatcaacctccagcgccctggtgagcatgctagctgtgggaaccctctggaacaagaaagtggcttcacataccttcctgaggctttcatggaggctggcatttacttctacaatttcggatggaaggattatggtgtagcgtctcttactactatcctagatatggtgaaggtgatgacatttgccttacaggaaggaaaagtagctatccattgtcatgcagggcttggtcgaacaggtgttttaatagcctgttacttagtttttgcaacgagaatgactgctgaccaagcaattatatttgtgcgggcaaagcgacccaattccatacaaaccagaggacagctcctctgtgtaagggaatttactcagtttctaactcctctccgcaatatattctcttgctgtgatcccaaagcacatgctgtcaccttacctcaatatctaattcgccagcgtcatctgcttcatggttatgaggcacgacttctgaaacacgtgccaaaaattatccacctagtttgcaaattgctgctggacttagcggagaacaggccagtgatgatgaaggatgtgtccgaaggacctggtctctctgctgaaatagaaaagacaatgtctgagatggtcaccatgcagctggataaagagttactgaggcatgacagtgatgtgtccaacccgcctaaccccactgcagtggcagcagattttgacaatcgaggcatgattttctccaatgagcaacagtttgaccctctttggaaaaggcggaatgttgagtgccttcaacccctgactcatctgaaaaggcggctcagctacagtgactcagatttaaagagggccgagaacctcctggagcaaggggagactccacagacagtgcctgcccagatcttggttggccacaagcccaggcagcagaagctcataagccattgttacatcccacagtctccagaaccagacttacacaaggaagccttggttcgcagcacactttctttctggagtcagtcaaagtttggaggcctggaaggactcaaagataatgggtcaccaattttccatggaaagatcattccaaaggaagcacagcagagtggagctttctctgcagatgtttcaggctcacacagccctggggagccagtttcacccagctttgcaaatgtccataaggatccaaaccctgctcaccagcaagtgtctcactgtcagtgtaaaactcatggtgttgggagccctggctctgtcaggcagaacagcaggacaccccgaagccctctggactgtggctccagtcccaaagcacagttcttggttgaacatgaaacccaggacagtaaagatctgtctgaagcagcttcacactctgcattacagtctgaattgagtgctgaggcaagaagaatactggcggccaaagccctagcaaatttaaatgaatctgtagaaaaggaggaactaaaaaggaaggtagaaatgtggcagaaagagcttaattcccgagatggagcttgggaaagaatatgtggcgagagggaccctttcatcctatgcagcttgatgtggtcttgggtggagcaactgaaggagcctgtaatcaccaaagaggatgtggacatgttggttgacaggcgagcagatgccgcagaagcactttttttattagagaagggacagcaccagactattctctgcgtgttgcactgcatagtgaacctgcagacaattcccgtggatgtggaggaagctttccttgcccatgccattaaggcattcactaaggttaattttgattctgaaaatggaccaacagtttacaacaccctgaagaaaatatttaagcacacgctggaagaaaaaagaaaaatgacaaaagatggccctaagcctggcctctagctttcactcgtggtgaatatttcagacctaaagatccagatagtatctctgttcatatgtgaataagttgaagattgtggggctactttttctcatagcactttattttgaatgttgttagtttgtgctgagaatggtcgtccgtatttgaaccaattatttattttaaaatatatttaagctacatttttgttttgaaaaattgccataaatttggtgccactttcttttatttatttgactgagttaatattattgtattaacattttaagtatatggtgtttacattcttatttctttggcattttggaaataatcataacttgtctttccaaaataaccattttcttgatggaactcttcctagagtttttaccaaatagctaactttagtagtaaaacctcattgtgtatccattcccccacagatgaactaagaaagtcaccaagtgtcttaagctgttttatatttgttacgaagaaggctattgctacaatatttttaaaggtttcttttttaactttgaaattttttgtttttccttttctttttataaatgtaacagagggtttcaaagcatattatttttcagagagatttagttttactttaatggagtgactgtgaagtggttgggattttttgcttgtagaaagtagacttgctctttgtcagatttccaaacaaccttgccagccttggctgtcaaaaggaggcaggagcagttctcaacacaccaagccttattcccactcccttgggttgctgctgagccaaatagcatctttacagaggaagtgggatcagaggcaggaagtgtggaaagttgctaagaagcagggcttgcctctgtcctcccggggactccacagggatattcgtgcagggcaggggctctgtgccagccctgctctctcagatgccacagccactctgcagaggtgactcttggagctggaggaagtcaaaactgggccactgtttgtactgatggtgtattagcatgagcagcgtggccctggccccacactcccaaatctgccactccatagacccacttgcctcaaggctttatatttggctgctttcttacaatgagaattaagatttttaaactgaagttgaccatacaggttgcattagccctaactggcttcatgtaagaagggtgactgcctaaactagttccttgtaagctgaaccatcaattatcagttgaagccatacttttatttaaattaatatacgtagataccagaggccaagccacagagaggataatagttcttcccaataaaggtgatattaatcagactaatttcgaactaaagaagttactgcttaaagacggaatttcaggggaagcaagactcatttagaacaaatgaaatttctccagtcctacatttctgaattgacttctagcacatcaaaaatatttcagtcattatcagtctcattaactgaaatgccaaatgctaaatgcagtgttctttcacactgttttaattttcttgggaaattgagtccagtggatgttaatggagtgggttgcccatccctgaaatgtcttattttcaagtgcctggcctgggaaagaaggggaagaaacaattgcattatatccaaagatacactataaaaatagagtttttaccaaaaaaagatgtttgttctcatctcagtaggcctcatttgggcaagtgacccacaggtcttttggcgagtttgctatttgcctgttgaaatacttgtttcaacttagagaacagttatgatgtgaccatagcatggcacaactaaaaatctaagcctgaaacctgaaaaaagagatatgacaagggaaattaatcaggctatacataagtattgtatttatttgaataaaaataa

SEQ ID NO: 31 - DNA, 4524 bp, Homo sapiens
  misc_feature: Incyte ID No 71231882CB1
ccgccctcgccaacatggcggcgcccagttggggcgggttcgttcgcttcgcgttttggccagggcgggggtctgggctttaggcaggtagtatttagtttcacaatgtttggggacctgtttgaagaggagtattccactgtgtctaataatcagtatggaaaagggaagaaattaaagactaaagctttggagccacctgctcctagagaattcaccaatttaagcggaatcagaaatcagggtggaacctgttacctcaattcccttcttcagactcttcatttcacacctgaattcagagaagctctattttctcttggcccagaagagcttggtttgtttgaagataaggataaacccgatgcaaaggttcgaatcatccctttacagttacagcgcttgtttgctcagcttctgctcttagaccaggaagctgcatccacagcagacctcactgacagctttgggtggaccagtaatgaggaaatgaggcaacatgatgtgcaggaactgaatcgaatcctcttcagcgctttggaaacttctttagttgggacctccggtcatgacctcatctatcgtctgtaccatggaaccattgttaaccagattgtttgtaaagaatgtaagaacgttagcgagaggcaggaagacttcttagatctaacagtagcagtcaaaaatgtatccggtttggaagatgctctctggaacatgtatgtagaagaggaagtttttgattgtgacaacttgtaccactgtggaacttgtgacaggctggttaaagcagcaaagtcggccaaattacgtaagctgcctccttttcttactgtttcattactaagatttaattttgattttgtgaaatgcgaacgctacaaggaaactagctgttatacattccctctccggattaatctcaagcccttttgtgaacagagtgaattggatgacttagaatatatatatgacctcttctcagttattatacacaaaggtggctgctacggaggccattaccatgtatatattaaagatgttgatcatttgggaaactggcagtttcaagaggaaaaaagtaaaccagatgtgaatctgaaagatctccagagtgaagaagagattgatcatccactgatgattctaaaagcaatcttattagaggaggagaataatctaattcctgttgatcagctgggccagaaacttttgaaaaagataggaatatcttggaacaagaagtacagaaaacagcatggaccattgcggaagttcttacagctccattctcagatatttctactcagttcagatgaaagtacagttcgtctcttgaagaatagttctctccaggctgagtctgatttccaaaggaatgaccagcaaattttcaagatgcttcctccagaatccccaggtttaaacaatagcatctcctgtccccactggtttgatataaatgattctaaagtccagccaatcagggaaaaggatattgaacagcaatttcagggtaaagaaagtgcctacatgttgttttatcggaaatcccagttgcagagaccccctgaagctcgagctaatccaagatatggggttccatgtcatttactgaatgaaatggatgcagctaacattgaactgcaaaccaaaagggcagaatgtgattctgcaaacaatacttttgaattgcatcttcacctgggccctcagtatcatttcttcaatggggctctgcacccagtagtctctcaaacagaaagcgtgtgggatttgacctttgataaaagaaaaactttaggagatctccggcagtcaatatttcagctgttagaattttgggaaggagacatggttcttagtgttgcaaagcttgtaccagcaggacttcacatttaccagtcacttggcggggatgaactgacactgtgtgaaactgaaattgctgatggggaagacatctttgtgtggaatggggtggaggttggtggagtccacattcaaactggtattgactgcgaacctctacttttaaatgttcttcatctagacacaagcagtgatggagaaaagtgttgtcaggtgatagaatctccacatgtctttccagctaatgcagaagtgggcactgtcctcacagccttagcaatcccagcaggtgtcatcttcatcaacagtgctggatgtccaggtggggagggttggacggccatccccaaggaagacatgaggaagacgttcagggagcaagggctcagaaatggaagctcaattttaattcaggattctcatgatgataacagcttgttgaccaaggaagagaaatgggtcactagtatgaatgagattgactggctccacgttaaaaatttatgccagttagaatctgaagagaagcaagttaaaatatcagcaactgttaacacaatggtgtttgatattcgaattaaagccataaaggaattaaaattaatgaaggaactagctgacaacagctgtttgagacctattgatagaaatgggaagcttctttgtccagtgccggacagctatactttgaaggaagcagaattgaagatgggaagttcattgggactgtgtcttggaaaagcaccaagttcgtctcagttgttcctgttttttgcaatggggagtgacgttcaacctgggacagaaatggaaatcgtagtagaagaaacaatatctgtgagagattgtttaaagttaatgctgaagaaatctggcctacaaggagatgcctggcatttacgaaaaatggattggtgctatgaagctggagagcctttatgtgaagaaaattcagccagaagccaactcattacccttggaactggcttctcgttccagccttgccaggatgcaacactgaaagaacttctgatatgttctggagatactttgcttttaattgaaggacaacttcctcctctgggtttcctgaaggtgcccatctggtggtaccagcttcagggtccctcaggacactgggagagtcatcaggaccagaccaactgtacttcgtcttggggcagagtttggagagccacttccagccaaggtgcttctgggaacgagcctgcgcaagtttctctcctctacttgggagacatagagatctcagaagatgccacgctggcggagctgaagtctcaggccatgaccttgcctcctttcctggagttcggtgtcccgtccccagcccacctcagagcctggacggtggagaggaagcgcccaggcaggcttttacgaactgaccggcagccactcagggaatataaactaggacggagaattgagatctgcttagagccccttcagaaaggcgaaaacttgggcccccaggacgtgctgctgaggacacaggtgcgcatccctggtgagaggacctatgcccctgccctggacctggtgtggaacgcggcccagggtgggactgccggctccctgaggcagagagttgccgatttctatcgtcttcccgtggagaagattgaaattgccaaatactttcccgaaaagttcgagtggcttccgatatctagctggaaccaacaaataaccaagaggaaaaagaaaaaaaaacaagattatttgcaaggggcaccgtattacttgaaagacggagatactattggtgttaagaatctcctgattgacgacgatgatgatttcagtacaatcagagatgacactggaaaagaaaagcagaaacaacgggccctggggagaaggaaaagccaagaagccctccatgagcagagcagctacatcctctccagtgcagagacgcctgcccggccccgagccccggaaacttctctctccatccacgtggggagcttcagataaccgcgccgctgcacggctctactcccgatgaactctccggctgatgccacaaacgtgggtttcctgggcatggggactggctgcctggcgcctccaatcccaaatcctctgcttcctttgagcacagggacggctcctctgaggcctggccagtgcatgtagtcacttagctctgcaacacgtggcagccacgggggctggtgcagctctggatgtcgcccacccagctgccagtaggtgctgggctctctcacacagcacccggccccagctgcctttttttttcttttaaccagaaaatgcacaacgtgtgcgtgaaccgcaggtatggaggcagcggcatgccgttgctccgctgtgggaggtgtgtggggtcaggccagccactttcctccgtgttcagatgactctcgttcgccctgaccggcttctcacagtgtctcaggccactgcgccaccgcgctggtgctgagcagaagcgggcagaagtggggtctgctttcaggacttcatttcccccactcgttccggccccgcatgctccacgtctgccctttggtctgagttaaaactgcgatgctgaaaagtgcgagctctttccacgaggaggagccacacagggtggcctccgagggtgagtcgctctgc

SEQ ID NO: 32 - DNA, 3250 bp, Homo sapiens
  misc_feature: Incyte ID No 2875922CB1
cggcggctgtggccgcggcgaacaggccggacgcctcgtcgctggcggggcttcccttggagctgacgaaggcggggtccgcggtccaggctgctgccgcgacggggccggcggcggggcagctgccaggaacgagggtagcagctgcatccagatctcattatgcatcagaaaaatgaaaaaacagaggaaaattctatggaggaaaggaatccacttagccttttctgagaaatggaatactgggtttggaggctttaagaagttttattttcaccaacacttgtgcattctgaaagctaagctgggaaggccagttacttggaatagacagttgagacatttccagggtagaaagaaagctcttcaaatccagaaaacgtggatcaaggatgaacccctttgtgctaagaccaagttcaatgtggctactcaaaatgttagtactttgtcctctaaagtgaaaagaaaggacgctaaacacttcatttcctcctcaaagactctcctgagactccaagcagagaagctgttgtcatcagcaaagaattctgaccatgaatactgcagagagaaaaatctcttgaaggcagttactgactttccatcaaatagtgctttaggtcaggccaatggtcacagacctaggacagacccacaaccttctgactttcccatgaagttcaatggggagagccaaagtccaggtgagagtggcacgattgtggtcaccttgaacaaccataagagaaagggcttttgttacggctgctgccaagggccggagcaccacaggaatgggggacccttgattccaaaaaagttccaacttaaccaacatagaaggataaaattatctcctcttatgatgtatgagaaattatccatgattagatttcggtacaggattctcagatcccagcacttcagaaccaaaagcaaggtttgcaagctaagaaaagcccagcgaagctgggtacagaaagtcactggggaccatcaagagacccgtagggagaacggtgagggtggcagttgcagcccatttccttccccagaacctaaagacccttcttgtcggcatcagccgtactttccagatatggacagcagtgctgtggtgaaggggacgaactctcatgtgcctgattgccacactaaaggaagctctttcttgggcaaggagcttagtttagacgaagcattccctgaccaacagaatggcagtgccacaaacgcctgggaccagtcatcctgttcttctcctaagtgggagtgtacagagctgattcatgacatccccttaccagaacatcgttctaataccatgttcatttcagaaactgaaagagaaattatgactctgggtcaggaaaatcagacaagttctgtcagtgatgacagagtaaaactgtcagtgtctggagcagatacatctgtgagtagcgtagatgggcctgtgtcccaaaaggctgttcaaaatgagaactcataccagatggaggaggatggatctctcaagcagagcattcttagttctgagttgctggaccacccttactgtaaaagtccactggaggctcccttggtgtgcagtggactcaaactagaaaatcaagtaggaggtggaaagaacagtcagaaagcctctccagtggatgatgaacagctgtcagtctgtctttctggattcctagatgaggttatgaagaagtatggcagtttggttccactcagtgaaaaagaagtccttggaagattaaaagatgtctttaatgaagacttttctaatagaaaaccatttatcaatagggaaataacaaactatcgggccagacatcaaaaatgtaacttccgtatcttctataataaacacatgctggatatggacgacctggcgactctggatggtcagaactggctgaatgaccaggtcattaatatgtatggtgagctgataatggatgcagtcccagacaaagttcacttcttcaacagcttttttcatagacagctggtaaccaaaggatataatggagtaaaaagatggactaaaaaggtggatttgtttaaaaagagtcttctgttgattcctattcacctggaagtccactggtctctcattactgtgacactctctaatcgaattatttcattttatgattcccaaggcattcattttaagttttgtgtagagaatataagaaagtatttgctgactgaagccagagaaaaaaatagacctgaatttcttcagggttggcagactgctgttacgaagtgtattccacaacagaaaaacgacagtgactgtggagtctttgtgctccagtactgcaagtgcctcgccttagagcagcctttccagttttcacaagaagacatgccccgagtgcggaagaggatttacaaggagctatgtgagtgccggctcatggactgaaactcagcagggactctgggaagtctgaccaagttggagcagatggtttgttacttgaatctccaaacacttagttgaatttttacagatatttcagatcagtggtgttgggccactattgttacctcaaatttattttttgcccttattcatttctccagctaccatgtactattgtttaatgttcagtttggtttcatttttaattttatggttctgtgcgtcccccatatttaatatttattattcaaacgcatgcatatagacagagcatgcagtgaagagtattaaaaaaaaaagcttagtagatttggtgcagcttttgaaacttagttagacgtgaactgaatacaggtttcaaatttactcccagaacctaaaaatgcaagatgtttttgatacaacataactctgagaatagtaagtgttccctggggcattaagggtagctgggggtggttttgacaaatccagtcctgttttactttaccagcggcaactttcaccaacttcccctctccaagtgagtcttagagagtgcagtccattccttttgaagggtgagatggaagtggtcgtaaactgactggtgtcttctgtttctggaggcacacttgtaagcacagtggctgctttgggaggagtaaggtgtgagaaaaagcaaccttggaggccagtaacaatgacagatttcaatcgtggttttaggaattataatacgtggcatacatctcataaaggcttttgctgggatattgaattccctgaatttttctgttttcgacctgttaaaaaaatcttaacatc

SEQ ID NO: 33 - DNA, 3834 bp, Homo sapiens
  misc_feature: Incyte ID No 8158136CB1
gcggccggtcgtgcgggtcgggcgcgggcgggcgcggcggcagtggcgcgcacaggtgattgactggccagctgcctgaaggagcgccaggtcctccttgctggcaggtggcgaagcccattggggcggcggtgcagaccgcggcggcggctgcggcggtctggctcgggaggcgttcctggggccaaggccatggccccgcggctgcagctggagaaggcggcctggcgctgggcggagacggtgcggcccgaggaggtgtcgcaggagcacatcgagaccgcttaccgcatctggctggagccctgcattcgcggcgtgtgcagacgaaactgcaaaggaaatccgaattgcttggttggtattggtgagcatatttggttaggagaaatagatgaaaatagttttcataacatcgatgatcccaactgtgagaggagaaaaaagaactcatttgtgggcctgactaaccttggagccacttgttatgtcaacacatttcttcaagtgtggtttctcaacttggagcttcggcaggcactctacttatgtccaagcacttgtagtgactacatgctgggagacggcatccaagaagaaaaagattatgagcctcaaacaatttgtgagcatctccagtacttgtttgccttgttgcaaaacagtaataggcgatacattgatccatcaggatttgttaaagccttgggcctggacactggacaacagcaggatgctcaagaattttcaaagctctttatgtctctattggaagatactttgtctaaacaaaagaatccagatgtgcgcaatattgttcaacagcagttctgtggagaatatgcctatgtaactgtttgcaaccagtgtggcagagagtctaagcttttgtcaaaattttatgagctggagttaaatatccaaggccacaaacagttaacagattgtatctcggaatttttgaaggaagaaaaattagaaggagacaatcgctatttttgcgagaactgtcaaagcaaacagaatgcaacaagaaagattcgacttcttagccttccttgcactctgaacttgcagctaatgcgttttgtctttgacaggcaaactggacataagaaaaagctgaatacctacattggcttctcagaaattttggatatggagccttatgtggaacataaaggtgggtcctacgtgtatgaactcagcgcagtcctcatacacagaggagtgagtgcttattctggccactacatcgcccacgtgaaagatccacagtctggtgaatggtataagtttaatgatgaagacatagaaaagatggaggggaagaaattacaactagggattgaggaagatctagcagaaccttctaagtctcagacacgtaaacccaagtgtggcaaaggaactcattgctctcgaaatgcatatatgttggtttatagactgcaaactcaagaaaagcccaacactactgttcaagttccagcctttcttcaagagctggtagatcgggataattccaaatttgaggagtggtgtattgaaatggctgagatgcgtaagcaaagtgtggataaaggaaaagcaaaacacgaagaggttaaggagctgtaccaaaggttacctgctggagctgagccctatgagtttgtctctctggaatggctgcaaaagtggttggatgaatcaacacctaccaaacctattgataatcacgcttgcctgtgttcccatgacaagcttcacccggataaaatatcaattatgaagaggatatctgaatatgcagctgacattttctatagtagatatggaggaggtccaagactaactgtgaaagccctgtgtaaggaatgtgtagtagaacgttgtcgcatattgcgtctgaagaaccaactaaatgaagattataaaactgttaataatctgctgaaagcagcagtaaagggcgatggattttgggtggggaagtcctccttgcggagttggcgccagctagctcttgaacagctggatgagcaagatggtgatgcagaacaaagcaacggaaagatgaacggtagcaccttaaataaagatgaatcaaaggaagaaagaaaagaagaggaggaattaaattttaatgaagatattctgtgtccacatggtgagttatgcatatctgaaaatgaaagaaggcttgtttctaaagaggcttggagcaaactgcagcagtactttccaaaggctcctgagtttccaagttacaaagagtgctgttcacagtgcaagattttagaaagagaaggggaagaaaatgaagccttacataagatgattgcaaacgagcaaaagacttctctcccaaatttgttccaggataaaaacagaccgtgtctcagtaactggccagaggatacggatgtcctctacatcgtgtctcagttctttgtagaagagtggcggaaatttgttagaaagcctacaagatgcagccctgtgtcatcagttgggaacagtgctcttttgtgtccccacgggggcctcatgtttacatttgcttccatgaccaaagaagattctaaacttatagctctcatatggcccagtgagtggcaaatgatacaaaagctctttgttgtggatcatgtaattaaaatcacgagaattgaagtgggagatgtaaacccttcagaaacacagtatatttctgagcccaaactctgtccagaatgcagagaaggcttattgtgtcagcagcagagggacctgcgtgaatacactcaagccaccatctatgtccataaagttgtggataataaaaaggtgatgaaggattcggctccggaactgaatgtgagtagttctgaaacagaggaggacaaggaagaagctaaaccagatggagaaaaagatccagattttaatcaaagcaatggtggaacaaagcggcaaaagatatcccatcaaaattatatagcctatcaaaagcaagttattcgccgaagtatgcgacatagaaaagttcgtggtgagaaagcacttctcgtttctgctaatcagacgttaaaagaattgaaaattcagatcatgcatgcattttcagttgctccttttgaccagaatttgtcaattgatggaaagattttaagtgatgactgtgccaccctaggcacccttggcgtcattcctgaatctgtcattttattgaaggctgatgaaccaattgcagattatgctgcaatggatgatgtcatgcaagtttgtatgccagaagaagggtttaaaggtactggtcttcttggacattaatctttgaatacttgctgactgctaagaaatgaccagaggggaagaggagtttgacatgttagggcattaaagcaaaggtggatttaagaattaaaccattacatgccccttccaaaaggcagaaatccattcaaacgtgactgtcccaaatgccttatgtcaaataaagcagattgcactgatggacatcagacttgaaggaaatgtttccaattttatatttaaggggggtggtgggtgggagggggcaagtaaagacggaacaagtttagtagcagtaatagtaaatcatgtttacatatgagatttatagtcgtgggaggggaataaagttctgttatatttccttgctcgagtttcataccagatgcgttggtccataaaggattgtatcaagtagatgggacaacattctgctctgaacgaaaagtaattttagagacataacctgcttaccaatgcctgtctttgattcatattctactttcaataaagcatgaaagtgaaaaaaaaaaaaaaaaaaaaaaaaaaaaaaaa

SEQ ID NO: 34 - DNA, 4493 bp, Homo sapiens
  misc_feature: Incyte ID No 5969491CB1
gcgcccagttggggcgggttcgttcgcttcgcgtttggccaggcgggggtctgggctttaggcaggtagtatttagtttcacaatgtttggggacctgtttgaagaggagtattccactgtgtctaataatcagtatggaaaagggaagaaattaaagactaaagctttggagccacctgctcctagagaattcaccaatttaagcggaatcagaaatcagggtggaacctgttacctcaattcccttcttcagactcttcatttcacacctgaattcagagaagctctattttctcttggcccagaagagcttggtttgtttgaagataaggataaacccgatgcaaaggttcgaatcatccctttacagttacagcgcttgtttgctcagcttctgctcttagaccaggaagctgcatccacagcagacctcactgacagctttgggtggaccagtaatgaggaaatgaggcaacatgatgtgcaggaactgaatcgaatcctcttcagcgctttggaaacttctttagttgggacctccggtcatgacctcatctatcgtctgtaccatggaaccattgttaaccagattgtttgtaaagaatgtaagaacgttagcgagaggcaggaagacttcttagatctaacagtagcagtcaaaaatgtatccggtttggaagatgctctctggaacatgtatgtagaagaggaagtttttgattgtgacaacttgtaccactgtggaacttgtgacaggctggttaaagcagcaaagtcggccaaattacgtaagctgcctccttttcttactgtttcattactaagatttaattttgattttgtgaaatgcgaacgctacaaggaaactagctgttatacattccctctccggattaatctcaagcccttttgtgaacagagtgaattggatgacttagaatatatatatgacctcttctcagttattatacacaaaggtggctgctacggaggccattaccatgtatatattaaagatgttgatcatttgggaaactggcagtttcaagaggaaaaaagtaaaccagatgtgaatctgaaagatctccagagtgaagaagagattgatcatccactgatgattctaaaagcaatcttattagaggaggagaataatctaattcctgttgatcagctgggccagaaacttttgaaaaagataggaatatcttggaacaagaagtacagaaaacagcatggaccattgcggaagttcttacagctccattctcagatatttctactcagttcagatgaaagtacagttcgtctcttgaagaatagttctctccaggctgagtctgatttccaaaggaatgaccagcaaattttcaagatgcttcctccagaatccccaggtttaaacaatagcatctcctgtccccactggtttgatataaatgattctaaagtccagccaatcagggaaaaggatattgaacagcaatttcagggtaaagaaagtgcctacatgttgttttatcggaaatcccagttgcagagaccccctgaagctcgagctaatccaagatatggggttccatgtcatttactgaatgaaatggatgcagctaacattgaactgcaaaccaaaagggcagaatgtgattctgcaaacaatacttttgaattgcatcttcacctgggccctcagtatcatttcttcaatggggctctgcacccagtagtctctcaaacagaaagcgtgtgggatttgacctttgataaaagaaaaactttaggagatctccggcagtcaatatttcagctgttagaattttgggaaggagacatggttcttagtgttgcaaagcttgtaccagcaggacttcacatttaccagtcacttggcggggatgaactgacactgtgtgaaactgaaattgctgatggggaagacatctttgtgtggaatggggtggaggttggtggagtccacattcaaactggtattgactgcgaacctctacttttaaatgttcttcatctagacacaagcagtgatggagaaaagtgttgtcaggtgatagaatctccacatgtctttccagctaatgcagaagtgggcactgtcctcacagccttagcaatcccagcaggtgtcatcttcatcaacagtgctggatgtccaggtggggagggttggacggccatccccaaggaagacatgaggaagacgttcagggagcaagggctcagaaatggaagctcaattttaattcaggattctcatgatgataacagcttgttgaccaaggaagagaaatgggtcactagtatgaatgagattgactggctccacgttaaaaatttatgccagttagaatctgaagagaagcaagttaaaatatcagcaactgttaacacaatggtgtttgatattcgaattaaagccataaaggaattaaaattaatgaaggaactagctgacaacagctgtttgagacctattgatagaaatgggaagcttctttgtccagtgccggacagctatactttgaaggaagcagaattgaagatgggaagttcattgggactgtgtcttggaaaagcaccaagttcgtctcagttgttcctgttttttgcaatggggagtgacgttcaacctgggacagaaatggaaatcgtagtagaagaaacaatatctgtgagagattgtttaaagttaatgctgaagaaatctggcctacaaggagatgcctggcatttacgaaaaatggattggtgctatgaagctggagagcctttatgtgaagaagatgcaacactgaaagaacttctgatatgttctggagatactttgcttttaattgaaggacaacttcctcctctgggtttcctgaaggtgcccatctggtggtaccagcttcagggtccctcaggacactgggagagtcatcaggaccagaccaactgtacttcgtcttggggcagagtttggagagccacttccagccaaggtgcttctgggaacgagcctgcgcaagtttctctcctctacttgggagacatagagatctcagaagatgccacgctggcggagctgaagtctcaggccatgaccttgcctcctttcctggagttcggtgtcccgtccccagcccacctcagagcctggacggtggagaggaagcgcccaggcaggcttttacgaactgaccggcagccactcagggaatataaactaggacggagaattgagatctgcttagagccccttcagaaaggcgaaaacttgggcccccaggacgtgctgctgaggacacaggtgcgcatccctggtgagaggacctatgcccctgccctggacctggtgtggaacgcggcccagggtgggactgccggctccctgaggcagagagttgccgatttctatcgtcttcccgtggagaagattgaaattgccaaatactttcccgaaaagttcgagtggcttccgatatctagctggaaccaacaaataaccaagaggaaaaagaaaaaaaaacaagattatttgcaaggggcaccgtattacttgaaagacggagatactattggtgttaagaatctcctgattgacgacgatgatgatttcagtacaatcagagatgacactggaaaagaaaagcagaaacaacgggccctggggagaaggaaaagccaagaagccctccatgagcagagcagctacatcctctccagtgcagagacgcctgcccggccccgagccccggaaacttctctctccatccacgtggggagcttcagataaccgcgccgctgcacggctctactcccgatgaactctccggctgatgccacaaacgtgggtttcctgggcatggggactggctgcctggcgcctccaatcccaaatcctctgcttcctttgagcacagggacggctcctctgaggcctggccagtgcatgtagtcacttagctctgcaacacgtggcagccacgggggctggtgcagctctggatgtcgcccacccagctgccagtaggtgctgggctctctcacacagcacccggccccagctgcctttttttttcttttaaccagaaaatgcacaacgtgtgcgtgaaccgcaggtatggaggcagcggcatgccgttgctccgctgtgggaggtgtgtggggtcaggccagccactttcctccgtgttcagatgactctcgttcgccctgaccggcttctcacagtgtctcaggccactgcgccaccgcgctggtgctgagcagaagcgggcagaagtggggtctgctttcaggacttcatttcccccactcgttccggcccccgcatgctccacgtctgccctttggtctgagttaaaactgcgatgctgaaaagtgcgagctctttccacgaggaggagccacacagggtggcctccgagggtgagtcgctctgctaagcaagggcagccgctgcacgtcagcccgcaggccaagggtccagctt

SEQ ID NO: 35 - DNA, 2921 bp, Homo sapiens
  misc_feature: Incyte ID No 7497367CB1
ctggggctggattgagctgaccacaggccacaccagactcctctctgctcctgaggaagacagggcagcccggcgccacccgctcggccctcacgaagatgctccctggagcctggctgctctggacctccctcctgctcctggccaggcctgcccagccctgtcccatgggttgtgactgcttcgtccaggaggtgttctgctcagatgaggagcttgccaccgtcccgctggacatcccgccatatacgaaaaacatcatctttgtggagacctcgttcaccacattggaaaccagagcttttggcagtaaccccaacttgaccaaggtggtcttcctcaacactcagctctgccagtttaggccggatgcctttggggggctgcccaggctggaggacctggaggtcacaggcagtagcttcttgaacctcagcaccaacatcttctccaacctgacctcgctgggcaagctcaccctcaacttcaacatgctggaggctctgcccgagggtcttttccagcacctggctgccctggagtccctccacctgcaggggaaccagctccaggccctgcccaggaggctcttccagcctctgacccatctgaagacactcaacctggcccagaacctcctggcccagctcccggaggagctgttccacccactcaccagcctgcagaccctgaagctgagcaacaacgcgctctctggtctcccccagggtgtgtttggcaaactgggcagcctgcaggagctcttcctggacagcaacaacatctcggagctgccccctcaggtgttctcccagctcttctgcctagagaggctgtggctgcaacgcaacgccatcacgcacctgccgctctccatctttgcctccctgggtaatctgacctttctgagcttgcagtggaacatgcttcgggtcctgcctgccggcctctttgcccacaccccatgcctggttggcctgtctctgacccataaccagctggagactgtcgctgagggcacctttgcccacctgtccaacctgcgttccctcatgctctcatacaatgccattacccacctcccagctggcatcttcagagacctggaggagttggtcaaactctacctgggcagcaacaaccttacggcgctgcacccagccctcttccagaacctgtccaagctggagctgctcagcctctccaagaaccagctgaccacacttccggagggcatcttcgacaccaactacaacctgttcaacctggccctgcacggtaacccctggcagtgcgactgccacctggcctacctcttcaactggctgcagcagtacaccgatcggctcctgaacatccagacctactgcgctggccctgcctacctcaaaggccaggtggtgcccgccttgaatgagaagcagctggtgtgtcccgtcacccgggaccacttgggcttccaggtcacgtggccggacgaaagcaaggcagggggcagctgggatctggctgtgcaggaaagggcagcccggagccagtgcacctacagcaaccccgagggcaccgtggtgctcgcctgtgaccaggcccagtgtcgctggctgaacgtccagctctctcctcggcagggctccctgggactgcagtacaatgctagtcaggagtgggacctgaggtcgagctgcggttctctgcggctcaccgtgtctatcgaggctcgggcagcagggccctagtagcagcgcatacaggagctggggaagggggcctctggggcctgaccaggcgacaggtaggggcggaggggagctgagtctccgaagccttggcttttcacatgcaagggacagggttacatccccaaggtgagggggtggagtctggtctgctccactaaccagggtctcctcctcctcttccttcatcgcttctcctggagtgtgcggcctaacaaggccatccttatgctttgcaaagcaccctcaaaagctgcaccacagcctggagaataaaatatcctcagccctgatgcctccccattatgtaacacccaaccgctctcacctacaccctgaggtctattcactgcatcccagtgatacaaagtggaggccactgccttctgacatctggctcaaaagcccagtgtctgtttccatttatttccctggaatttcatttaaaattggtatagagaaaaaaaggatgtgacagaagcagagatgaccagaaagcacaggggcagggttctgactggcgtgtgggagaccctgtggccggcacccacctccacacgaggactaagctctgatttttttatcttgcccaaattcctacctaaggggtctagggagtcgcgccttacaaatcataaattctcatcagatgggttttatttgaccctgtatatcatgacttatttttaatctgactatggcataacattacaagacgaggcaaaaatatttaacccccaaatatatttctttgccctaccttgaacttgccctgcagagtctcttgtgaggagaatccacatcctataaagaagcccctttcccctttgttttccttcctttctttccagtccaggagatcatcaactaagagccaggcaccccttttaagtcgataagaaacagtttacaacctgctctctctctctctgaagtctgctgagagcttcccctgcacaataaaacttggcctccacaatcctttatcttaacctgaacattcctttccattgatcccaggtcttcctcaacactcagctctgccagtttaggccggatgcctttggggggctgcccaggctggaggacctggaggtcacaggcagtagcttcttgaacctccaggtcctccagtttaggccggatgcctttggggggctgcccaggctggaggacctggaggtc

SEQ ID NO: 36 - DNA, 2572 bp, Homo sapiens
  misc_feature: Incyte ID No 7632424CB1
tagtgacctatagaacactattagtcgcatgccgcgtcgtaagctcggtgcagcgataagggcagtcgacagtctttagtagggaaaggagacaagtgctagctactgccgcccaagtggaaggaattatctatagagtaagtatgctaatcttgactaagactgcaggagttttttttaaaccatcaaaaaggaaagtttatgaatttttaagaagttttaattttcatcctggaacactatttcttcataaaatagtattgggaattgaaactagttgtgatgatacagcagctgctgtggtggatgaaactggaaatgtgttgggagaagcaatacattcccaaactgaagttcatttaaaaacaggtgggattgttcctccagcagctcaacagcttcacagagaaaatattcaacgaatagtacaagaagctctttctgccagtggagtctctccaagtgacctctcagcaattgcaactaccataaaaccaggacttgctttaagcctgggagtgggcttatcatttagcttacagctggtaggacagttaaaaaagccattcattcccattcatcatatggaggctcatgcacttactattaggttgaccaataaagtagaatttccttttttagttcttttgatttctggaggtcactgtctgttggcattagttcaaggagtttcagattttctgcttcttggaaagtctttggacatagcaccaggtgacatgcttgacaaggtggcaagaagactttctttaataaaacatccagagtgctccaccatgagtggtgggaaagccatagaacatttggccaaacaaggaaatagatttcattttgacatcaaacctcccttgcatcatgctaaaaattgtgatttttcttttactggacttcaacacgttactgataaaataataatgaaaaaggaaaaagaggaaggtattgagaaggggcaaatcctgtcttcagcagcagacattgctgccacagtacagcacacaatggcatgtcatcttgtgaaaagaacacatcgggctattctgttttgtaagcagagagacttgttacctcaaaataatgcagtactggttgcatctggtggtgtcgcaagtaacttctatatccgcagagctctggaaattttaacaaacgcaacacagtgcactttgttgtgtcctcctcccagactatgcactgataatggcattatgattgcatggaatggtattgaaagactacgtgctggcttgggcattttacatgacatagaaggcatccgctatgaaccaaaatgtcctcttggagtagacatatcaaaagaagttggagaagcttccataaaagtaccacaattaaaaatggagatatgatttctgctgttcaaaaaagtccctaaagggtctcactctctgacctcagctggagtacagtagccagatcacaactcactgcaaccctgacttcctgaactcaagaaatcctcctgccttagcctcttgaatagccgggactacaggtgtgcatgtccatgcccagccaactttatttctattttttgtagagacaggctcttgccatgttgcccgggctggtcctgaactgctgaattcaagtgatcctcccaccttggcctccagaagtgctgggattatgggtgtgagccaccatgcctagccaaaatgtttcttaaggtatacattttgggtcttagaagacttatacatttgtaatatttattactaaatatctcaaagtattacaataaatgttaccatgtgagctactttgaatcaggcttcttgcacaccaatttaaaaatgttaactcttgatatatacactagttataccactcatgtcagtcaataaattttaaggtttaagtgcaggcctttgtttacagaaatcctaattttttgaaaccataactctgacctgacactaaattcctgtagacatgctaaggaaaatctgcttagtatcgagatcaagaacttccattcaaaaagattattcagttatgttatttgcatattaccattgttaaaaataaaaaaatttttaaaagatggctcaggaatcctttcattctaaaatgttttattagccttctgcaggctctgggttggctttctggacatgtttcccaagttggttcaaacacactattcttggtcttgatacatttgtgatatctgatgtaattgtagatgaaaaagaaaaaatattatgggaatccttggtcttttttgttgctttgaagagtcccatgaagttagttacaggaggtaataaagtcaaagaatcttttggttgaattttaggttttaattttcttcggtgctccttccctaaaaattagccacaattaagtgtctctcctgagagccctaggtatagtctcattacttctacttctatgtagttctattttctttacataactaaaacatactaagagaacagatcagtacttccagacatttagatgtcaggggccaaaaatttgtggggaccactatatggtttgctagcttgttatgtcaagttagcac

SEQ ID NO: 37 - DNA, 3758 bp, Homo sapiens
  misc_feature: Incyte ID No 1804436CB1
ggagctctggagcctttgcttcctcaaatacgagcgggaactgcgttgagcgctggattccaggccgagtgctggcgaggcgcgcagttctctgctgtttaaaaagtatccctgtgctttgaagatactgctataatttgaaaatttgaaattagtgtttcagctgaaccatccgttcatcttcaagccatcatgagctgtaagaagcagaggtcacggaagcactcagtcaatgaaaaatgtaatatgaaaatcgagcactatttttctccggtctctaaagagcaacagaataattgcagtacttctctaatgaggatggagtctagaggagacccaagagccacaactaatacccaggctcaaagattccattcacctaagaaaaatccagaagaccagaccatgccccaaaataggacaatatatgttaccttgaaggtaaaccacaggagaaaccaagatatgaaacttaagctcacacatagtgagaatagtagcttatatatggctctcaacactctccaggctgtcagaaaagagatagaaactcaccaaggccaagaaatgcttgtgcgtggcacagaaggaatcaaagagtacataaaccttggaatgcccctcagttgtttccctgaaggtggccaggtggtcattacattttcccaaagtaaaagtaagcagaaggaagataaccacatatttggcaggcaggacaaagcatcgactgaatgtgtcaaattttacattcatgcaattggaattgggaagtgtaaaagaaggattgttaaatgtgggaagcttcacaaaaaggggcgcaaactctgtgtttatgctttcaaaggagaaaccatcaaggatgcactgtgcaaggatggcagatttctttcctttctggagaatgatgattggaaactcattgaaaacaatgacaccattttagaaagcacccagccagttgatgaattagaaggcagatactttcaggttgaggttgagaaaagaatggtccccagtgcagcagcttctcagaatcctgagtcagagaaaagaaacacctgtgtgttgagagaacaaatcgtggctcagtaccccagtttgaaaagagaaagtgaaaaaatcattgaaaacttcaagaaaaaaatgaaagtaaaaaatggggaaacattatttgaattgcatagaacaacgtttgggaaagtaacaaaaaattcttcttcgattaaagtagtgaaacttcttgtacgtctcagtgactcagttgggtacttattctgggacagtgcaactacgggttacgccacctgctttgtttttaaaggattgttcattttaacttgtcggcatgtaatagatagcattgtgggagacggaatagagccaagtaagtgggcaaccataattggtcaatgtgtaagggtgacatttggttatgaagagctaaaagacaaggaaacaaactacttttttgttgaaccttggtttgagatacataatgaagagcttgactatgctgtcctgaaactgaaggaaaatggacaacaagtacctatggaactatataatggaattactcctgtgccacttagtgggttgatacatattattggccatccatatggagaaaaaaagcagattgatgcttgtgctgtgatccctcagggtcagcgagcaaagaaatgtcaggaacgtgttcagtctaaaaaagcagaaagtccagagtatgtccatatgtatactcaaagaagtttccagaaaatagttcacaaccctgatgtgattacctatgacactgaatttttctttggggcttccggctcccctgtgtttgattcaaaaggttcattggtggccatgcatgctgctggctttgcttatacttaccaaaatgagactcgtagtatcattgagtttggctctaccatggaatccatcctccttgatattaagcaaagacataaaccatggtatgaagaagtatttgtaaatcagcaggatgtagaaatgatgagtgatgaggacttgtgagaattcagtctactggatttaagggaatggcttatggagttgttatttcataggcattgaaaatggttttctaaactccaaaatggtcatcttatcaataataataatattgaccatttcctatctgccaggcatttttctaagcacatgaagaaattagtcctaacaacactatgagatggactataacttgcccaaattttttttttttttttgagactgagtctcactctgtcgcctgggctggagtacagtggtgcgatctcagctcactgcaacttccacctcccaggttcaagcgattcttatgcctcagtctcctgagcagctgggattacaggcaaacgccaccacacccagctaaaatttttttttttttttgtatttttagtagagacagggtttcaccatgttggtcaggcgggtctcgaactcctgacctcgtgatccacctgcctcggccttccaaagtgctgggattacaagtttgagccactgcacctggctaacttgccctattttaaagtcaagcaatgggaagaataacaagattatatagtaatcagtttcatgacactaaaagtcatatagtcatagggttttttcatctttcatatctttgcctaaattcatttgctacagtgcaggaaccaaaacttgttcatctcatgattccctacatctgacataaggaaagtaagtgctcagaaaaatgtgcaggtcaataagttgcaaaagttggggctgcaattaatgctaacataagagctaaatgcttgattagaaatgatctcaaaaccttttagaatttccaaaatcttcatattactgaaactgtcggaatatatgggtcctgaaattcagaagatgatagtcactcttcccatatttataggctattaaggcaagggatatcttaaacatcatattactttatttagatttctactactccaattattaatgttatgtatttctcattgttttacttcttcatggtattatgaagactatatagatgattcaaccaagcctgcaaatctccctcttgtggaattccactggacccaatctgttttccatttccattgcaatactactaaagccatacaatatcaagcaccctccctctaggtccagggactatcacagaagaagcaggcatgtaagattttaaggactggtttcgaggggtcgagtgtaggaaaacagcctgttgcattgtaagagtgatgtcatcttgaagagcagctggcatgatgactgctgtttgactcctgcataccaagatattctgcagcaatgtctttaaacagtgccggtagtacagataacccctcataaagatgcttatctaacctccccagtgttcaggtgtttcacaagaaagtctgagatatgactagctacacgttttgccaaaaatgcttgttatataaagggtacttttgggagggtgagtgccgccatttagtggctgctagaaacattgcttctgtttgtaagttcctattaaatgtttctttctgagaaaaaaaagtatatgttttaaaattgacaaggtgcagtcaatttctaggaacaggtgaccactttttcagagatgaagtgtttatattaattaaggagcacttggtttctgtatctaataatagaactgacttagaagtagcagtaggtgatctccctctaaagtccgggggttctgcgcctgggatctgccacgagct

SEQ ID NO: 38 - DNA, 1036 bp, Homo sapiens
  misc_feature: Incyte ID No 7486358CB1
cgggcggcggagccccggcgccagagcatcgggttgctgatacttaggcaggtgctgtggagccttcctatgagtccccatgcagccatgccttctgttcactcacagcacttttactcctggcagctcctccttttagtccttgacccatctggttttgtttccccaccccactggcatgtccagggatcaccttccactcacacctcttgtacttctattcttatttcagggactgatcctgggacacccgaggaagtgggtgctctcctctagcccactgaggcactagggagcagtggtggctggacctcatgccctgctcttgcccactggggtcccttatctttctaacccacccccatggttgctgttacttctccctgaacaagagatgattgaaacagctttgtctggaggaggcgaagccacatttgggagggacagattataatgccagagatgaggcctggagggaggagtttccacctgagctcaagagtttaagggccagaactgtgtgcagtaggctcctgtgggagtccaggctgggaaggctggaggttaggagtgcttcctgtgccctgcgccatgtggagtctgccgccgagcagggctctgtcctgtgcgccactgctgcttctcttcagcttccagttcctggttacctatgcttggcgtttccaagaggaagaggagtggaatgaccaaaaacaaattgctgtttatctccctcccaccctggagtttgccgtgtacacattcaacaagcagagcaaggactggtatgcctacaagctggtgcctgtcctggcttcctggaaggagcagggttatgataagatgacattctccatgaatctgcaactgggcagaaccatgtgtgggaaatttgaagatgacattgacaactgcccttttcaagagagcccagagctgaacaatacctgcacctgcttcttcaccattggaatagagccctggaggacacggtttgacctctggaacaagacgtgctcaggcgggcattcctgag

SEQ ID NO: 39 - DNA, 3651 bp, Homo sapiens
  misc_feature: Incyte ID No 7472344CB1
ggaggcgctgcgagcggagccgcgcggagggcgcgaccggctggtccgggcagcgggggtttgccgccttcggggctccagtccgcgcgccagtgctcgatgcagtaccgcgggcccctcaggtgggcctcggctcgggacgccgggagtcgggaccgccagtcggggcgccgggaccatggcgctgcgcgcccgggcgctgtacgacttcaggtcggagaaccccggagagatctcgctgcgagagcacgaggtgctgagcctgtgcagcgagcaggacatcgagggctggctcgagggggtcaacagccgcggcgaccgcggcctcttcccggcctcctatgtgcaggtgatccgcgcccccgagcctggcccggcgggagacggcggcccgggcgccccggcccgctacgccaatgtgccccccgggggcttcgagcccctgcctgtcgcgccccccgcctccttcaagccgccgcctgacgccttccaggcgctgctgcagccacagcaggcgccgcctccgagcaccttccagccgcccggcgcgggcttcccgtacggcgggggcgccctgcagccgtcgcctcagcagctctacggcggctaccaggccagccaaggcagcgatgatgactgggacgacgagtgggacgacagctccacggtggcggacgagccgggcgctctgggcagcggagcatacccggacctcgacggctcgtcttcggcgggtgtgggcgcagccggccgctaccgcctgtccacgcgctccgacctgtccctgggctcccgcggcggctcggtccccccgcagcaccacccgtcggggcccaagagctcggccaccgtgagccgcaacctcaatcgcttctccaccttcgtcaagtccggcggggaggccttcgtgctgggggaggcgtcaggcttcgtgaaggacggggacaagctgtgcgtggtgctggggccctatggccccgagtggcaggagaacccctacccgttccagtgcaccatcgacgaccccaccaagcagaccaagttcaagggcatgaagagctacatctcctacaagctggtgcccacgcacacgcaggtgccggtgcatcggcgctacaagcacttcgactggctgtacgcgcgcctggcggagaagttcccggtcatctccgtgccccacctgcccgagaagcaggccaccggccgcttcgaggaggacttcatctctaagcgcaggaagggcctgatctggtggatgaaccacatggccagccacccagtgctggcgcagtgcgacgtcttccagcacttcctgacgtgccccagcagcaccgacgagaaagcctggaagcagggcaagaggaaggccgagaaggacgagatggtgggcgccaacttcttcctgacccttagcacgccccccgccgctgcccttgacctgcaggaggtggagagcaagatagacggcttcaagtgcttcaccaagaagatggacgacagcgcgctgcagctcaaccacacggccaacgagttcgcgcgcaagcaggtgaccggcttcaaaaaggagtatcagaaggtgggccagtccttccgcggcctcagccaggcctttgagctggaccagcaggccttctcggtgggcctgaaccaggctatcgccttcaccggagatgcctatgacgccattggcgagctcttcgcggagcagcccaggcaggacctggatcccgtcatggacctattagcgctgtatcaggggcatctggctaacttcccggacatcatccacgttcagaaaggagctcttaccaaagtcaaggagagtaggcgacacgtggaggaagggaagatggaggtgcagaaggctgacggcattcaggatcgctgtaacactatttcttttgccactttggctgaaattcaccacttccatcaaattcgagtgagagactttaaatcacagatgcagcatttcttacaacaacaaataatatttttccaaaaagttacccagaagttggaagaagctcttcacaaatatgatagtgtttaatgactggacgttggattatggactttttcagttcaaggataatttctacagcagaataaaaactgctgtcaaagagctattgccagctatcagtggtggtacaaggacggttttgtgttcatctgaaacccagctgaatttataattatgtaggaaataaacagttaatatggttatataatagaaacagtaccacacattgtaactaaattatactatgtatgcctacactaccattgtaacttttggaataatgattatactatttgccttattgctttttgaagtatgggtattttagtgcatactttgtagacctcaaaacccatgaagggtctcaaagaagctggctggatacaagcctgctgtggatgcctttttactctcatagattgggattacctaaattcaacctattctctgtttacaaactccaactagagcagctatgcgaccttgtgcctttagactcttggtttttcatttctccccgtcccttccccacctttttaaagtaagccacagcttttctgattgaaagagtgaaaggccagtgcatataatgacaaactgatgataaccttatattggcagtagggggtggggggggcggtggggtgggacgatcagctgtcatcaatttgcacagcaagtattatctcctgataagatgctggtgaatgcaggggagtgagattcattgctcatctttggatatgaagtctgttagggaagaaacagtgccactattcccttagatgcaacagtagcatagcctcttcacccaggcgtcccaaaagcttggcgtgaagatttcagcaaacatgtcttacaacatgaggaggaggagtctaaatcagtcaggggataaaagtatcgaatcattgacaacacacacttggctttagttcttaggagggttttgtttttgtttttgtttttaggttgaagattttcttttaatattcagttttttgaaaaaaaatggatctacactgttaactgattgagactccactgtgattcacttgtttacttaaaaacttttcagggatgtctgtaaatttcagtgttaatatgtcatgaaaagtggtgtggattgatctaaggagggaccagaaataatttttgctattccaaatactgaaggaaaaagataattgatttatactatgttttaaaaaaaaaaaaggtattgatgagccccccccccccaggacatttaaccttaaaatttattttaaatgtattcttttattattataagggaaatacagatggctgataaataccaaaaagattcaaaagcagcttaatttaaaaagcacaaagagattctggcttacagtgccccaatctcaatgtttttatagttgctgagctaactaatgtgattattgagtttacagatttaaaaattgtcactgttagagtatctactgtttttatgaagtcaaacttatgctgcctcagaaatccctgggtactgaaatggtaacatggaagtgaagaggtcactttgaaatattggtgagtcacaaagattaaagaaaaggatcagtttgcagatactcagaaaaggttattgaaataattac

SEQ ID NO: 40 - DNA, 1989 bp, Homo sapiens
  misc_feature: Incyte ID No 7192959CB1
ggggtctccggatatcggggctatcaaggacggtctgggggatgtgggacttgcgtacggggcccagggatggtgaagacggggctgcggctggcagaatcagggcaggcagaggctgcgcgccgcggaggctgctggtccctgacgccttgacccttcctctcccgcagccacagccgggcctggcggggggaccatgggcgcctcggtctccaggggccgggccgcccgggtccccgcgccggagccggaacccgaagaggcgctggacctgagccaactacccccagagctgcttctggtggtgctgagccacgtccccccgcgcacgctgctcgggcgctgccgccaagtgtgccggggctggcgagccctggtggacggccaggccctgtggctgctgatcctggcccgcgaccacggcgccaccggccgcgcgctgctgcacctcgcccgcagctgccagtctcccgcccgtaacgccaggccttgccccctgggccgcttctgcgcgcgcagacccatcggacgcaaccttattcgcaacccctgcggccaagaaggcctccgaaagtggatggtgcaacacggtggggacggctgggtggtggaggaaaacaggacaaccgtgcctggggccccttctcagacgtgcttcgtgacttcattcagctggtgttgcaagaagcaggtcttggacctagaggaggagggtctgtggccagaactgctggatagtggcaggattgagatttgtgtctctgactggtggggagcccgacacgacagcggctgtatgtacagactcctcgtccaacttctagacgccaaccagactgttctagataaattctctgctgtgcctgatcccatcccgcagtggaacaacaatgcctgccttcacgtcacccacgtgttctccaacatcaagatgggcgtccgctttgtgtctttcgaacaccggggccaggacacacagttctgggctggccactatggagcccgtgtgaccaactccagtgtgatcgtgcgagtccgtctgtcctagtccagcactacccttcttgcaagacagcctgactgtgccttccagggcctgggaccattggctgggacccctcattaaccaaccaagcacttgtacctccctggcatactgggaattcctgggtccaatcaaaggccctgacgggccctgtcttcaggttctagaaactaccagaagaagcttcttccatcttatctacctactgcagctgctgctttgcgggggggccctactgtactgaggggaaacccacaagtgagcatgggggaatgcccatcctggagaagaattcttgccctcccctctcttctcctcccaaccaaaaccctccgatccagccccaggcccctctgtaccatcccctcccctccgcaccactgacctcttgtctcctattgtttctgccacagggacttccttggtcccttcagggaagccctcaactcatctctgaacttagaatctcatccttagggctcagagagtcccagccctaagggtgtgaacctcacctcctggggcttccgagctacagggctgggaccaggtcatgcagctgaaagtctacgttaaaaaaaaaaagtttgggccaggcacagtggctcatgcctgtaatcccagcactatgggaggctgaggcaggcggatcacctgaggtcaggagttcaagaccagcctggccaacatagtgaaactgtgtctgtgaaggcaggcattgttgttccactgcgggatgggatcaggcacagcagagaatttatctagaacagtctggttggcgtctagaagttggacgaggagtctgtacatacagccgctgtcgtgtcgggctccccaccagtcagagacacaaatctcaatcctgccactatccagcagttctggccacagaccctcctcctctaggtccaagacctgcttcttgcaacaccagctgaatg

SEQ ID NO: 41 - DNA, 1629 bp, Homo sapiens
  misc_feature: Incyte ID No 6169565CB1
cgggcgctagggcgctggcaatgtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccaggttttcccagtcacgacgttgtaaaacgacggccagtgaattgaatttaggtgacactatagaagactcaaattactccttcctcccctctaggtttcctgatttgtaacctttctaaactcggtataaagggggacattaaagtaaagtgccttatattcttttgcaatactgcctggcctcaggccaagggcaaccgttggcctgaaaacggcacattcgaccaccaaattttaagggatttagataacttcatttatcgaaatggcaaatggcaagaagttccttcatttacctttgctcacgtccttccctctgccaagattgttccccacacctctaatttgctccttaatctctgttgttggcataaagggcattcagaaaactcccctccaaacactccccttatattgttcctttcgggacgtcaccctcatacattgcttcctgttaatcccacattgccccatgcctcttctaagtagagacctgctgcacaaattgcgtggcttcctccacctttgggctcttggccaaagccatccctacttatttttatgccaggaacccaaattctccctacctgaagttaaggagccaacacctgaccttagcattataactcaaacaaatcctattgtttggtcaacacaaattctgcagtcgtggcggcccaccactccccattaaaatttcattaaaggatccttctcattagatcacagtccaacagtattctgtcagccctgagaggcttcggggactcaagcccatcatctccactgtagaactgccttcttgcctcttctttccttcatctttctattccaatacttgctgccaaaacccgggatgagtcttggggctgagaactcttgcaacccaggaagcagtgggccccgacagctcatccctggctaattcctggatcctgagggttctctggcttcccgcctagtccctcaactcttcctcttttttaaaagtgatttgcatgaggtaattgagtgacaggagaggacttcgaggctgtggccagggctacccctggatgggttctcaaaactcccggacccagaattttgcctccaaccgctggatctgggcaatttactttctgtttctccccaacggctccagtttgagaggtcctttgctggttccttctaaaacatccaacaccagacactaattagtaaggtttgcccccacctgtctctcctgtgctacccaggagactcaggtccgcagtcccggtctcctggggaccattaggaccatcagagaccttgggagggtgacgttcagggatgcctgacctttcccctcagtctacctcctcttgagaggatctcccgtctctgccctttttctggggatgcctagtctaacggcagacaccctggcctttccatcgc

SEQ ID NO: 42 - DNA, 3166 bp, Homo sapiens
  misc_feature: Incyte ID No 7494717CB1
tcttgcagagcgcctctcgctggttggggcgggggtgggcggagccagcaccgtctgggctgtggaagcggaggggtggggacactctggcccggttctcggtggtgcgggagcgggcgggagcagcggccgctctggtcggcggacgtgctgccgagtagtcccggaagcgaagcagcgatggcggagagtccgactgaggaggcggcaacggcgggcgccggggcggcgggccccggggcgagcagcgttgctggtgttgttggcgttagcggcagcggcggcgggttcgggccgcctttcctgccggatgtgtgggcggcggcggcggcagcgggcggggccgggggcccggggagcggcctggctccgctgcccgggctcccgccctcagccgctgcccacggggccgcgctgcttagccactgggaccccacgctcagctccgactgggacggcgagcgcaccgcgccgcagtgtctactccggatcaagcgggatatcatgtccatttataaggagcctcctccaggaatgttcgttgtacctgatactgttgacatgactaagattcatgcattgatcacaggcccatttgacactccttatgaagggggtttcttcctgttcgtgtttcggtgtccgcccgactatcccatccacccacctcgggtcaaactgatgacaacgggcaataacacagtgaggtttaaccccaacttctaccgcaatgggaaagtctgcttgagtattctaggtacatggactggacctgcctggagcccagcccagagcatctcctcagtgctcatctctatccagtccctgatgactgagaacccctatcacaatgagcccggctttgaacaggagagacatccaggagacagcaaaaactataatgaatgtatccggcacgagaccatcagagttgcagtctgtgacatgatggaaggaaagtgtccctgtcctgaacccctacgaggggtgatggagaagtcctttctggagtattacgacttctacgaggtggcctgcaaagatcgcctgcaccttcaaggccaaactatgcaggacccttttggagagaagcggggccactttgactaccagtccctcttgatgcgcctgggactgatacgtcagaaagtgctggagaggctccataatgagaatgcagaaatggactctgatagcagttcatctgggacagagacagaccttcatgggagcctgagggtttagaccctgctcccatctccccttcccccactcaagagtcccagcagaatcccttccccccaccccagggatggagaggcactgtgtatctccctccagactcgaagtcatcctgcaagatggcaagaaccaagcaagctccgatcccagggtgtgggagtgggggcctgttcccggtctgacctccttggcactggagcatctggggcttcgttcatccattcatcccgtatcaggggccaaggtacctttacaggagcacctagagcgagggcctttggcaaaaacaaaacaaccaacacacctctccacagggccagctccttagggataagtggaagatggaaattgcaattccaagagggagtgtgcccaaatgatttatggggatacctggaagggagcttggggtgggggctgtctgtgacacttaagcagtctgggtggttgtctatttgtctgtcttcagtcttgaagcagggcttcccaatgcccttttcctccctgccttccttcccccattatttcccacaggccagcataattttgtttttcctaatttatagtcactgttctagacagaccaaagagaaggaacagtggtggagtctaggctgctgatcagtaagctttacctagcacctgagcacctttctcccctcccctctttcctcacccttttctagatgtaagacagaaagtaaatgtgactgggacttaaccaaggtcttggtaaagcctgcatggcaccgtaagaagctgaaaatactgtttgttcccgcaatcactgatttgaaaagttcccaacacaggcagctgctgtgtatatgggattagagccactacatagaatagtctcttacagattttcataaatactagtcacaataagggtatttttcttgggggtggagtaagggggagactgatgctagtccttgttgtattttgttgggctgtccttgtgtattttcaccccagcctgtagtcctcctcacttcaaccccagggatttttggggagcaagggtagccaatggcagagggggttggggctgggactctggaggctcctccccttctttctcttccttccgcctcccccgtgcccccagctgctcttgtcactgtctctgatgggtatttgcctggctttgttgcttctctatctgtatttagctgcagtgatcctttagctggttggctcagaaaaaaaaaaatgtgctttaggtgccctgtaatcctgggcatcaagggaatccatccttcccctttttgatatgttctccccgtacttccagatttattgttatggctcccagtgggtattggcgattcttgtgatgcagggcctcagtcagtgtccagccatgcataagggagaggatagtgtgtacctgccctgccctctgctatgaaggtctctgccttgtggatcatgggactccccttggaggatctgtgcaaaggggggctgggcacaaaggagaatgtcctatttgggagggcaggaagcaaaggaactggacagggattggtgggcttggggaacggaagtttatcttggatacccttgaagaggctgggtctcttcacatgaagatcgaaaagggaccctgcttccaatttccctcttccattcctcgagctactccagggcttagaagaatgctcttggtctgtgggtccagtgttgtctgtcatccatttaagtgttcccactttcaagtgacaatcctctccttggccctgccatagggcagagcatgtctggcatagcagcctgacttttatgccctaatcttgagttgaggaaatatatgcacaggagtcaaagagatgtctttatatctgactgtatataaatgaagtttttttgttttttttgttttcctttttggtgcataaagtttgttttggcagaa

SEQ ID NO: 43 - DNA, 399 bp, Homo sapiens
  misc_feature: Incyte ID No 7497510CB1
ctcctccccttggtcccttttctccctaaaatggcaaccccaggctgagccactggctgagtggcaccatgcagcttcaggcctctctctcgtttctcctgattctcactctctgcctagagcttcgatcagaactagcacgagacactatcaaggatctcctcccaaatgtatgcgcttttcctatggaaaagggcccttgtcaaacctacatgacgcgatggtttttcaactttgaaactggtgaatgtgagttatttgcttacggaggctgcggaggcaacagcaacaactttttgaggaaagaaaaatgtgagaaattctgcaagttcacctgattttctaacaagaacacagccctccatggattcgggattgctctgagggccatagaaggca

SEQ ID NO: 44 - DNA, 1811 bp, Homo sapiens
  misc_feature: Incyte ID No 7498882CB1
ggtttccgggccggcgtactatttcaaggcgcgcgcctcgtggtggactcaccgctagcccgcagcgctcggcttcctggtaattcttcacctcttttctcagctccctgcagcatgggtgctgggccctccttgctgctcgccgccctcctgctgcttctctccggcgacggcgccgtgcgctgcgacacacctgccaactgcacctatcttgacctgctgggcacctgggtcttccaggtgggctccagcggttcccagcgcgatgtcaactgctcggttatgggaccacaagaaaaaaaagtagtggtgtaccttcagaagctggatacagcatatgatgaccttggcaattctggccatttcaccatcatttacaaccaaggctttgagattgtgttgaatgactacaagtggtttgccttttttaagtataaagaagagggcagcaaggtgaccacttactgcaacgagacaatgactgggtgggtgcatgatgtgttgggccggaactgggcttgtttcaccggaaagaaggtgggaactgcctctgagaatgtgtatgtcaacacagcacaccttaagaattctcaggaaaagtattctaataggctctacaagtatgatcacaactttgtgaaagctatcaatgccattcagaagtcttggactgcaactacatacatggaatatgagactcttaccctgggagatatgattaggagaagtggtggccacagtcgaaaaatcccaaggcccaaacctgcaccactgactgctgaaatacagcaaaagattttgcatttgccaacatcttgggactggagaaatgttcatggtatcaattttgtcagtcctgttcgaaaccaaggctgtgaaggcggcttcccataccttattgcaggaaagtacgcccaagattttgggctggtggaagaagcttgcttcccctacacaggcactgattctccatgcaaaatgaaggaagactgctttcgttattactcctctgagtaccactatgtaggaggtttctatggaggctgcaatgaagccctgatgaagcttgagttggtccatcatgggcccatggcagttgcttttgaagtatatgatgacttcctccactacaaaaaggggatctaccaccacactggtctaagagaccctttcaacccctttgagctgactaatcatgctgttctgcttgtgggctatggcactgactcagcctctgggatggattactggattgttaaaaacagctggggcaccggctggggtgagaatggctacttccggatccgcagaggaactgatgagtgtgcaattgagagcatagcagtggcagccacaccaattcctaaattgtagggtatgccttccagtatttcataatgatctgcatcagttgtaaaggggaattggtatattcacagactgtagactttcagcagcaatctcagaagcttacaaatagatttccatgaagatatttgtcttcagaattaaaactgcccttaattttaatatacctttcaatcggccactggccatttttttctaagtattcaattaagtgggaattttctggaagatggtcagctatgaagtaatagagtttgcttaatcatttgtaattcaaacatgctatattttttaaaatcaatgtgaaaacatagacttatttttaaattgtaccaatcacaagaaaataatggcaataattatcaaaacttttaaaatagatgctcatatttttaaaataaagttttaaaaataactgcaaaaaaaaaaaaaaggggggggcg

SEQ ID NO: 45 - DNA, 4407 bp, Homo sapiens
  misc_feature: Incyte ID No 5524205CB1
cagtgtgctggaaagtggacagatatactgcatttttaaaacagagtggaaaatttcctggaaatccctggcctccatataagaaaaggacatcactccatcctagctataaaggtcttatgagactttgcactgtaaaactttacacattgtgctattcactctgctttacaagattttgatggatcatcaaaatctgtcagaacatgtactctgcatggttttatatctgattgaattaggacttgaaaattctgctgaagaagaatcagatgaagaggcatcagtgggtggaccagaacgttgtcatgacagttggtttcctggcagtaacttagtgtcaaacatgcgacactttataaactatgttagagtaagagttccagagactgctcctgaagtaaagagagactcacctgcaagtactagctctgataacttgggttctttacaaaattctggtacagctcaagttttcagtttagtagcagaacgtagaaagaaatttcaggaaatcatcaatcgcagtagcagtgaagcaaatcaggtggttcgtcccaaaacttcaagtaaatggtctgctcctggttcagctccacagttaactacagccattttggaaattaaagaaagcatattgtctttgctaattaaacttcaccacaaactctcaggaaaacaaaactcctactatcctccttggcttgatgacatagaaattttaatccaaccagaaattcctaaatacagtcatggagatggtataactgccgtggaaagaattttactaaaagctgcatcgcaaagtagaatgaacaaacgcatcattgaagagatatgtagaaaagtgacccctcctgtaccacctaaaaaagtcactgcagcagagaagaaaacattggacaaagaagaaaggcgacagaaggctagagagaggcagcagaaattgcttgcggagtttgcttcacgacagaaaagctttatggaaactgcaatggatgttgattctcctgagaatgatattcctatggagatcaccacggcagaaccacaggtttccgaggcagtatatgactgtgttatttgtggacagagtggcccctcctctgaagatcgacctactggattagttgtactgttacaagcatcctcagttttggggcagtgccgtgacaatgttgagccaaaaaagttgccgatcagtgaagaggagcagatttacccttgggatacctgtgcagccgttcatgatgtgaggctttcattattacagcgttattttaaggatagttcatgtctcttggcagtatcaattggctgggaaggaggtgtttatgtacaaacctgtggtcacacattacatatagattgtcataaatcttacatggaatcattacggaatgaccaggttcttcagggcttctcggtggacaaaggagaattcacgtgtccactctgtaggcagtttgctaacagtgttcttccatgttatcctggaagcaatgtggaaaataacccttggcaacgtcctagcaacaaaagcatacaagatctcataaaggaagtggaggagctgcagggacgaccgggagctttcccatcagaaacaaatttaagtaaagaaatggaatctgtaatgaaagatataaaaaataccactcagaagaaatatagagactatagcaagaccccgggctcaccagacaatgattttctctttatgtactctgttgctagaaccaatttagaacttgaattgattcatcgaggaggcaatttgtgttcaggtggtgcaagcacagctggcaaaaggtcttgtttaaatcagctgtttcatgtattagccttgcacatgcggctttatagcattgactctgagtataatccctggagaaagctcacccagttagaagagatgaatccacagctgggatatgaagaacaacagcctgaggttccaattctttatcatgatgtaacatcccttttgctcatccagatcttaatgatgccacaacccttacgcaaagaccactttacctgcattgtgaaggtactttttaccctactgtacacacaggctcttgcagcactctcagttaaatgcagcgaagaagataggtcagcctggaaacacgcgggagctctcaaaaagagtacatgtgatgcagaaaagtcttacgaagtattactgagctttgtgataagtgaactatttaaaggaaagttataccatgaagaaggaactcaggaatgtgcaatggttaaccctattgcttggtctcctgaatccatggaaaaatgcttacaggacttctgcttaccttttctcagaatcaccagccttcttcagcaccacctttttggggaagatttacctagctgccaggaagaagaagaattttcagttcttgccagctgcctgggacttctgccaacgttttaccaaacagaacatccattcatcagtgcctcctgtctggattggccagttccagcatttgatattataactcagtggtgttttgagataaaatcatttactgaaagacatgcagaacaaggaaaggccttgcttatccaagagtcaaaatggaaattaccacacctactacagttgcctgagaattataacaccatttttcagtactaccacagaaaaacctgtagtgtctgcaccaaggttcctaaagatcctgctgtttgccttgtgtgtggtacttttgtatgcctgaaaggactttgctgcaagcaacaaagttactgtgaatgtgtactgcactctcagaactgtggtgcaggaacaggtattttccttttgatcaatgcatcggtaattatcatcattcgaggtcaccgcttctgcctctggggttccgtgtatttggatgctcatggagaggaagaccgggatcttaggcgaggcaaacctctctacatttgtaaggaaagatacaaagttcttgagcaacagtggatttctcatacttttgatcacatcaataaaagatggggtccacattacaatgggctgtgactctccacctcagcattgcatcgtatcatcattttcgctacgaatttatttttcaacaataagctttaacttaatttgggggattaacacttttgctgagggagaaaaagaaaacatacattatgaagcctttccaaaattaggtgcttggtaatcacgttaatggtataattttttttttttaatatctggagaacattaataacaagttaaattattctttagtggtcattttttaagtgcacaattaataagaagcacaacttgttcacaaactcattcagaaatgattctcccaacaatgcatatcagctattcattgatacttagagtgggtgtgatttatttgacattttactgcttctttctgtctgtgtgttttaatttgcatctgccaagcataatgcatcttttttcctctgccattcttgtgttgattggagaatttttctgtatgtaattagaaaaaaatgtaaaacatgatttatgtgaaatactgtatagtaaaagttggtctaatagtagaactttaaaattttttcttattgtgaggaatctgttaaaagtttaaagctttgctgaaaactgaattcattctcaggaatttcataaatcttctccccaggtaaataattgaaatagctgtaaaataagtagatagctgctgttaatataatacagtacattttggggggcatatgtgtggttggggggtccttaaaaatcaaaatttgccatttcagttggatgaattactagaggtaataacaaatcttactataaaatcaagaggtttaagaacatacactgggcagatgttgattccgtgcatgcccaccttttattaccaaacaaggttttgtttatatgattgtattagaaatgctcagacttccccagaaatgaaccataaattttggaacttcctttcagctcaagaggttcagctatattgtatttgtgcagtgtaatcactactatttctgctcggtttcctaaaaggaaaaaaaaggcgcagtggtgatgaccctcatgaatgagccacgcttctgcattcttcttagaaactgctgtgaaaaacaatttatgtttgcagggtttaaaaatcagtaaaaatgggaatgattgagctaaaacccactctatgagaaggaagattactgaaaagcatgtgacatattgctacaaagattttttttcctaaatgattcagtaattgaatgattatttaatatatagtgctatcaagcaatccctggtactttggacttccatggcttgttatataaaattacatttttacatgtaaaaataaactaaacaaatctaatgataaaatataaacataatgtcagatccatgttctatac

SEQ ID NO: 46 - DNA, 2177 bp, Homo sapiens
  misc_feature: Incyte ID No 7102342CB1
agcggaattcccgacttcccaacggcttcccgctggcagccccgaagccgcaccatgttccgcctctggttgctgctggccgggctctgcggcctcctggcgtcaagacccggttttcaaaattcacttctacagatcgtaattccagagaaaatccaaacaaatacaaatgacagttcagaaatagaatatgaacaaatatcctatattattccaatagatgagaaactgtacactgtgcaccttaaacaaagatattttttagcagataattttatgatctatttgtacaatcaaggatctatgaatacttattcttcagatattcagactcaatgctactatcaaggaaatattgaaggatatccagattccatggtcacactcagcacgtgctctggactaagaggaatactgcaatttgaaaatgtttcttatggaattgagcctctggaatctgcagttgaatttcagcatgttctttacaaattaaagaatgaagacaatgatattgcaatttttattgacagaagcctgaaagaacaaccaatggatgacaacatttttataagtgaaaaatcagaaccagctgttccagatttatttcctctttatctagaaatgcatattgtggtggacaaaactttgtatgattactggggctctgatagcatgatagtaacaaataaagtcatcgaaattgttggccttgcaaattcaatgttcacccaatttaaagttactattgtgctgtcatcattggagttatggtcagatgaaaataagatttctacagttggtgaggcagatgaattattgcaaaaatttttagaatggaaacaatcttatcttaacctaaggcctcatgatattgcatatctactaatttatatggattatcctcgttatttgggagcagtgtttcctggaacaatgtgtattactcgttattctgcaggagttgcattgcaatgtggacctgcaagctgttgtgattttcgaacttgtgtactgaaagacggagcaaaatgttataaaggactgtgctgcaaagactgtcaaattttacaatcaggcgttgaatgtaggccgaaagcacatcctgaatgtgacatcgctgaaaattgtaatggaagctcaccagaatgtggtcctgacataactttaatcaatggactttcatgcaaaaataataagtttatttgttatgacggagactgccatgatctcgatgcacgttgtgagagtgtatttggaaaaggttcaagaaatgctccatttgcctgctatgaagaaatacaatctcaatcagacagatttgggaactgtggtagggatagaaataacaaatatgtgttctgtggatggaggaatcttatatgtggaagattagtttgtacctaccctactcgaaagcctttccatcaagaaaatggtgatgtgatttatgctttcgtacgagattctgtatgcataactgtagactacaaattgcctcgaacagttccagatccactggctgtcaaaaatggctctcagtgtgatattgggagggtttgtgtaaatcgtgaatgtgtagaatcaaggataattaaggcttcagcacatgtttgttcacaacagtgttctggacatggagtgtgtgattccagaaacaagtgccattgttcgccaggctataagcctccaaactgccaaatacgttccaaaggattttccatatttcctgaggaagatatgggttcaatcatggaaagagcatctgggaagactgaaaacacctggcttctaggtttcctcattgctcttcctattctcattgtaacaaccgcaatagttttggcaaggaaacagttgaaaaagtggttcgccaaggaagaggaattcccaagtagcgaatctaaatcggaaggtagcacacagacatatgccagccaatccagctcagaaggcagcactcagacatatgccagccaaaccagatcagaaagcagcagtcaagctgatactagcaaatccaaatcacaggacagtacccaaacacaaagcagtagtaactagtgattccttcagaaggcaacggataacatcgagagtctcgctaagaaatgaaaattctgtctttccttccgtggtcacagctgaaagaaacaataaattgagtgtggatctaaaaa

SEQ ID NO: 47 - DNA, 703 bp, Homo sapiens
  misc_feature: Incyte ID No 4169939CB1
atgatgctccggctgctcagttccctcctccttgtggccgttgcctcaggctatggcccaccttcctctcactcttccagccgcgttgtccatggtgaggatgcgatccccatcaactctgaggagctgtttgtgcatccactctggaaccgctcgtgtgtggcctgtggcaatgacatcgccctcatcaagctctcacgcagcgcccagctgggagatgccgtccagctcgcctcactccctcccgctggtgacatccttcccaacaagacaccctgctacatcaccggctggggccgtctctataccaatgggccactcccagacaagctgcagcaggcccggctgcccgtggtggactataagcactgctccaggtggaactggtggggttccaccgtgaagaaaaccatggtgtgtgctggagggtacatccgctccggctgcaacggtgactctggaggacccctcaactgccccacagaggatggtggctggcaggtccacggtgtgaccagctttgtttctggctttggctgcaacttcatctggaagcccacggtgttcactcgagtctccgccttcatcgactggattgaggagaccatagcaagccactagaaccaaggcccagctggcagtgctgatcgatcccacatcctgaataaagaataaagatctctcagaaaaaaaaaaaaaaaaa

SEQ ID NO: 48 - DNA, 1295 bp, Homo sapiens
  misc_feature: Incyte ID No 6539977CB1
ggggaatgccagttctggcaccaaccttcctgctccctgctggggcctctgctcccccatctctcaggagtcgaaagtgagaaagcaagacatcaaggagggacctgtgccctgctccacatcctcccacctgccgcccgcagagcctgcaggccccgcccccctcgtctctggtccctacctctctgctgtgtcttcatgtccctgagggtcttgggctctgggacctggccctcagcccctaaaatgttcctcctgctgacagcacttcaagtcctggctatagccatgacacggagccaagaggatgagaacaagataattggtggctatacgtgcacccggagctcccagccgtggcaggcggccctgctggcgggtcccaggcgccgcttcctctgcggaggcgccctgctttcaggccagtgggtcatcactgctgctcactgcggccgcccgatccttcaggttgccctgggcaagcacaacctgaggaggtgggaggccacccagcaggtgctgcgcgtggttcgtcaggtgacgcaccccaactacaactcccggacccacgacaacgacctcatgctgctgcagctacagcagcccgcacggatcgggagggcagtcaggcccattgaggtcacccaggcctgtgccagccccgggacctcctgccgagtgtcaggctggggaactatatccagccccatcgccaggtaccccgcctctctgcaatgcgtgaacatcaacatctccccggatgaggtgtgccagaaggcctatcctagaaccatcacgcctggcatggtctgtgcaggagttccccagggcgggaaggactcttgtcagggtgactctgggggacccctggtgtgcagaggacagctccagggcctcgtgtcttggggaatggagcgctgcgccctgcctggctaccccggtgtctacaccaacctgtgcaagtacagaagctggattgaggaaacgatgcgggacaaatgatggtcttcacggtgggatggacctcgtcagctgcccaggccctcctctctctactcaggacccaggagtccaggccccagcccctcctccctcagacccaggagtccaggcccccagcccctcctccctcagacccgggagtccaggcccccagcccctcctccctcagacccaggagtccaggccccagcccctcctccctcagacccgggagtccaggcccccagcccctcctccctcagacccaggagtccaggccccagtccctcctccctcagacccaggagtccagg

SEQ ID NO: 49 - DNA, 575 bp, Homo sapiens
  misc_feature: Incyte ID No 7675588CB1
ccgggaggtctgggtgttggctggtccattccaggacagctacatctttggcagacctggtgctgacagaaccagctctgatcaggaggccagtacttcctaaaatgggactctcaggacttctgccaatcctggtaccattcatccttttgggggacatccaggaacctgggcacgctgaaggcatccttggcaagccgtgtcccaaaatcaaagtggaatgcgaagtggaagaaatagaccagtgtaccaaacccagagattgcccagaaaacatgaagtgttgcccgttcagccgtggaaagaaatgtttagacttcagaaaggtcagccttactttataccataaggaggagcttgaataacctccaggatttggctcataatccaggcctctctccacgtgtgcctgattgatgctccaaattggcttccacgggccaaaccttggctgttccagaaactgaaccccaggaattgcttacacactttcttccagcgtagcatctcttcaaacacaatgctcttccccttgaccacttctcagtatgaaactctatgtcttcgggtcgttg

SEQ ID NO: 50 - DNA, 1062 bp, Homo sapiens
  misc_feature: Incyte ID No 6244077CB1
ataaatttagagagacgtatggccctcgagcanngaattcggcacgaggcctccagtccctgagaattggtactacgaaaaggtgaactcctgggcagaatcttgcctagagcttgcggagtccagccaggcccctgctgaagggccccagaccaccggccacttctcccccgtccatctgaccagctgggcccctgcgcccacctggcctccacgttccctctcctctcacccacacccctggccatggctaactactacgaagtgctgggcgtgcaggccagcgcttccccggaggacatcaagaaagcctaccgcaagctggcccttcgttggcaccccgacaagaaccctgacaataaggaggaggcggagaagaagttcaagctggtgtctgaggcctatgaggttctgtctgactccaagaaacgctccctgtatgaccgtgctggctgtgacagctggcgggctggtggcggggccagcacgccctaccacagccccttcgacaccggctacaccttccgtaaccctgaggacatcttccgggagtttttcggtggcctggaccctttctcctttgagttctgggacagcccattcaatagtgaccgtggtggccggggccatggcctgaggggggccttctcggcaggctttggagaatttccggccttcatggaggccttctcatccttcaacatgctgggctgcagcgggggcagccacaccaccttctcatccacctccttcgggggctccagttctggcagctcggggttcaagtcggtgatgtcgtccaccgagatgatcaatggccacaaggtcaccaccaagcgcatcgtggagaacgggcaggagcgcgtggaggtggaggaagacgggcagctcaagtcggtgactgtgaacggcaaggagcagctcaaatggatggacagcaagtaggcgctggccacccggccctgccttcccaccaccaccaccgtgcatggggcagcaaacacgtggggccgcagacatagcctgatggttaataaatgtgccaagtgagttcatggcaaa

SEQ ID NO: 51 - DNA, 1029 bp, Homo sapiens
  misc_feature: Incyte ID No 7498404CB1
ggcttccggcatctggctcagttccgccatggcctccttggaagtcagtcgtagtcctcgcaggtctcggcgggagctggaagtgcgcagtccacgacagaacaaatattcggtgcttttacctacctacaacgagcgcgagaacctgccgctcatcgtgtggctgctggtgaaaagcttctccgagagtggaatcaactatgaaattataatcatagatgatggaagcccagatggaacaagggatgttgctgaacagttggagaagatctatgggtcagacagaattcttctaagaccacgagagaaaaagttgggactaggaactgcatatattcatggaatgaaacatgccacaggaaactacatcattattatggatgctgatctctcacaccatccaaaatttattcctgaatttattaggaagcaaaaggagggtaattttgatattgtctctggaactcgctacaaaggaaatggaggtgtatatggctgggatttgaaaagaaaaataatcagattataccgaaaagaagttctagagaaattaatagaaaaatgtgtttctaaaggctacgtcttccagatggagatgattgttcgggcaagacagttgaattatactattggcgaggttccaatatcatttgtggatcgtgtttatggtgaatccaagttgggaggaaatgaaatagtatctttcttgaaaggattattgactctttttgctactacataaaagaaagatactcatttatagttacgttcatttcaggttaaacatgaaagaagcctggttactgatttgtataaaatgtactcttaaagtataaaatataaggtaaggtaaatttcatgcatctttttatgaagaccacctattttatatttcaaattaaataattttaaagttgctggcctaatgagcaatgttctcaattttcgttttcattttgctgtattgagacctataaataaatgtatatttttttttgcaaaaaaaaaaaaaaaaaaaaaaaaaaaaaa

SEQ ID NO: 52 - DNA, 905 bp, Homo sapiens
  misc_feature: Incyte ID No 7391748CB1
tggggctcaaaatataaactcaggctatttatcaacttaatctggggaagcaaacctgaaggcaagtaccaccctgtcatccctagctcagagctgctgagaaagaggatacagctgagccccagggccctcccatcccctcgattctggttagctgcagtcttgccctccccgtgctgtctgcctaccctgcagagctggtggaccatagctcctgcagcccagacctacctcttgcttttgcagcaatataaatgtcaccctgggcgcccacaatatccagagacgggaaaacacccagcaacacatcactgcgcgcagagccatccgccaccctcaatataatcagcggaccatccagaatgacatcatgttattgcagctgagcagaagagtcagacggaatcgaaacgtgaacccagtggctctgcctagagcccaggagggactgagacccgggacgctgtgcactgtggccggctggggcagggtcagcatgaggaggggaacagatacactccgagaggtgcagctgagagtgcagagggataggcagtgcctccgcatcttcggttcctacgacccccgaaggcagatttgtgtgggggaccggcgggaacggaaggctgccttcaagggggattccggaggccccctgctgtgtaacaatgtggcccacggcatcgtctcctatggaaagtcgtcaggggttcctccagaagtcttcaccagggtctcaagtttcctgccctggataaggacaacaatgagaagcttcaaactgctggatcagatggagacccccctgtgactgactcttcttctcggggacacaggccagctccacagtgttgccagagccttaataaacgtccacagagtataaataaaaaaaaaaaaaaaaa

SEQ ID NO: 53 - DNA, 2667 bp, Homo sapiens
  misc_feature: Incyte ID No 7499780CB1
agtcctgtctcccgcccgccggccgagccgcgcccgtgccccgcctcccgtgcgcccgggacaatcctcgccttgtctgtggcgccggcatctggagctttctgtagcctccggatacgcctttttttcagggcgtagccccagccaagctgctccccgcggcggccgcacaagcagcccgagcgccccctttccagagctcccctccggagctgggatccaggcgcgtagcggagatcccaggatcctgggtgctgtctgggcccgctccccaccatgacctcctcggggcctggaccccggttcctgctgctgctgccgctgctgctgccccctgcggcctcagcctccgaccggccccggggccgagacccggtcaacccagagaagctgctggtgatcactgtggccacagctgaaaccgaggggtacctgcgtttcctgcgctctgcggagttcttcaactacactgtgcggaccctgggcctgggagaggagtggcgagggggtgatgtggctcgaacagttggtggaggacagaaggtccggtggttaaagaaggaaatggagaaatacgctgaccgggaggatatgatcatcatgtttgtggatagctacgacgtgattctggccggcagccccacagagctgctgaagaagttcgtccagagtggcagccgcctgctcttctctgcagagagcttctgctggcccgagtgggggctggcggagcagtaccctgaggtgggcacggggaagcgcttcctcaattctggtggattcatcggttttgccaccaccatccaccaaatcgtgcgccagtggaagtacaaggatgatgacgacgaccagctgttctacacacggctctacctggacccaggactgagggagaaactcagccttaatctggatcataagtctcggatctttcagaacctcaacggggctttagatgaagtggttttaaagtttgatcggaaccgtgtgcgtatccggaacgtggcctacgacacgctccccattgtggtccatggaaacggtcccactaagctgcagctcaactacctgggaaactacgtccccaatggctggactcctgagggaggctgtggcttctgcaaccaggaccggaggacactcccgggggggcaggaggtcttccatgaaccccacatcgctgactcctggccgcagctccaggaccacttctcagctgtgaagctcgtggggccggaggaggctctgagcccaggcgaggccagggacatggccatggacctgtgtcggcaggaccccgagtgtgagttctacttcagcctggacgccgacgctgtcctcaccaacctgcagaccctgcgtatcctcattgaggagaacaggaaggtgatcgcccccatgctgtcccgccacggcaagctgtggtccaacttctggggcgccctgagccccgatgagtactacgcccgctccgaggactacgtggagctggtgcagcggaagcgagtgggtgtgtggaatgtaccatacatctcccaggcctatgtgatccggggtgataccctgcggatggagctgccccagagggatgtgttctcgggcagtgacacagacccggacatggccttctgtaagagctttcgagacaagggcatcttcctccatctgagcaatcagcatgaatttggccggctcctggccacttccagatacgacacggagcacctgcaccccgacctctggcagatcttcgacaaccccgtcgactggaaggagcagtacatccacgagaactacagccgggccctggaaggggaaggaatcgtggagcagccatgcccggacgtgtactggttcccactgctgtcagaacaaatgtgtgatgagctggtggcagagatggagcactacggccagtggtcaggcggccggcatgaggattcaaggctggctggaggctacgagaatgtgcccaccgtggacatccacatgaagcaggtggggtacgaggaccagtggctgcagctgctgcggacgtatgtgggccccatgaccgagagcctgtttcccggttaccacaccaaggcgcgggcggtgatgaactttgtggttcgctaccggccagacgagcagccgtctctgcggccacaccacgactcatccaccttcaccctcaacgttgccctcaaccacaagggcctggactatgagggaggtggctgccgcttcctgcgctacgactgtgtgatctcctccccgaggaagggctgggcactcctgcaccccggccgcctcacccactaccacgaggggctgccaacgacctggggcacacgctacatcatggtgtcctttgtcgacccctgacactcaaccactctgccaaacctgccctgccattgtgcctttttagggggcctggcccccgtcctgggagttgggggatgggtctctctgtctccccacttcctgagttcatgttccgcgtgcctgaactgaatatgtcaccttgctcccaagacacggccctctcaggaagctcccggagtccccgcctctctcctccgcccacaggggttcgtgggcacagggcttctggggactccccgcgtgataaattattaatgttccgcagtctcactctgaataaaggacagtttgtaaaaa

SEQ ID NO: 54 - DNA, 2977 bp, Homo sapiens
  misc_feature: Incyte ID No 7499881CB1
gtgcggagtgccgagcggcctcacccccaaccgtcggcccagtcggacggttccgaggcgttgccgggagccgggcgcggctctgtgtggactcggagaaacgcggggcgtctgcctgagcccgcttttctacaagatgtggggatttttgaagcgccctgtagtggtgacggctgacatcaacttgagccttgtggccctgactgggatggggttactgagccggctgtggcgactcacctacccgcgggctgtggtgttatttaggaggattcgatggcaattttttgtggaacagaattggagcagaatacagtagcaacgtgcctgtgtggtccctgcgcctgctgccagcactcgcgggggccttgtcggtccccatggcctaccagatagtgttggagctccacttttctcattgtgccgccatgggagctgctctgttgatgcttatcgagaatgctctcatcactcagtcaaggctaatgcttttggaatcagtgttaatatttttcaatctattggccgtgttgtcctacctgaagttcttcaactgccaaaagcacagccctttttctctgagctggtggttctggctaacactgacaggggtcgcttgttcctgtgcagtgggcatcaagtacatgggtgtgttcacgtacgtgctcgtgctgggtgttgcagctgtccatgcctggcacctgcttggagaccagactttgtccaatgtaggtgctgatgtccagtgctgcatgaggccggcctgtatggggcagatgcggatgtcacagggggtctgtgtgttctgtcacttgctcgcccgagcagtggctttgctggtcatcccggtcgtcctgtacttactgttcttctacgtccacttgattctagtcttccgctctgggccccacgaccaaatcatgtccagtgccttccaggccagcttagagggaggactagctcggatcacccagggtcagccactggaggtggcctttgggtcccaggtcactctgaggaacgtctttgggaaacctgtgccctgctggcttcattcccaccaggacacctaccccatgatatatgagaacggccgaggcagctcccaccagcaacaggtgacctgttaccccttcaaagacgtcaataactggtggattgtaaaggatcccaggaggcaccagctggtggtgagcagccctccgagacctgtgaggcacggggacatggtgcagctggtccacggcatgaccacccgctccctgaacacgcatgatgttgcagcccccctgagcccccattcacaggaggtctcctgctacattgactataacatctccatgcccgcccagaacctctggagactggaaattgtgaacagaggatctgacacagacgtctggaagaccatcctctcagaggtccgctttgtgcacgtgaacacttccgctgtcttaaagctgagcggggctcacctccctgactgggggtatcggcaactggagatcgtcggggagaagctgtcccggggctaccacgggagcacggtgtggaacgtggaggagcaccgatacggcgcgagccaggagcagagggagcgggaacgggagctgcactcacctgcgcaggtggacgtcagcaggaacctcagcttcatggcgagattctcggagctgcagtggaggatgctggcgctgagaagtgatgactcggaacacaagtacagctccagcccactggagtgggtcaccctggacaccaatattgcctactggctgcaccccaggaccagcgctcagatccacctacttggaaacatagtgatctgggtttcgggcagcctcgctctggccatctacgccctgctgtccttgtggtacctgctccgacggcgaagaaatgtccatgacctccctcaggatgcctggctgcgctgggtgctggctggggcgctgtgtgccggtggctgggcagtgaactacctcccgttcttcctgatggagaagacactcttcctctaccactacctgcccgcactcaccttccaaatccttctgctccctgtggtcctgcagcacatcagcgaccacctgtgcaggtcccagctccagaggagcatcttcagcgccctggtggtggcctggtactcctccgcgtgccacgtgtccaacacgctgcgcccactcacctacggggacaagtcactctcgccacatgaactcaaggcccttcgctggaaagacagctgggacatcttgatccgaaaacactagaacaagagtgtggcaaagaacacccgtgctggggtcgggacgaggttgaagggtcttggtcaatgtacgtaatgagcagggtgggccccacgctgggaggacacgggctgggctgagcagggcctctagtggaacacatgggggtctcattgaaaagctctctgatgagcacctccttttgtgcaaagttaattttttctcgacaataaagatattccgtgtcttcacccctgaactaagacacagggagtatttcagaggccagcgtaggagtcatcgacaacgaaaagccgagaacccagggccagcagtggagcctcagcagaccagggcctggtccttgctaattgctgcagggtggagtttgatctggcagacccgatcctccttcatgaacacccagcaacctgagcaagtcccggccctgccctcagcgagcccggcaggcgtcccgggacagctcagtgttggagggccacctgaaccacgagccagggctggggcttgcatgtcattgtctatgacagcgtcaagactggcccttggcaccgtgctgtgtggaaaccctcccctctgagactccactgagacgtggctgagtgaaatcttcctcgtcagtggtcaaggtgtgtcatccatacagctccatgcctttgtcttttttaaatgtaattaaaaaaggaaccaactggaaaaaaaaaaaa

SEQ ID NO: 55 - DNA, 729 bp, Homo sapiens
  misc_feature: Incyte ID No 7488579CB1
atgctcctccttgctccccagatgctgaatctgctgctgctggcgctgcccgtcctggcgagccgcgcctacgcggcccctgccccaggccaggccctgcagcgagtgggcatcgttgggggtcaggaggcccccaggagcaagtggccctggcaggtgagcctgagagtccacggcccatactggatgcacttctgcgggggctccctcatccacccccagtgggtgctgaccgcagcgcactgcgtgggaccggacgtcaaggatctggccgccctcagggtgcaactgcgggagcagcacctctactaccaggaccagctgctgccggtcagcaggatcatcgtgcacccacagttctacatcatccagaccggggcggacatcgccctgctggagctggaggagcccgtgaacatctccagccacatccacacggtcacgctgccccctgcctcggagaccttccccccggggatgccgtgctgggtcactggctggggcgacgtggacaataatgtgcacctgccgccgccatacccgctgaaggaggtggaagtccccgtagtggaaaaccacctttgcaacgcggaatatcacaccggcctccatacgggccacagctttcaaatcgtccgcgatgacatgctgtgtgcggggagcgaaaatcacgactcctgccagggtgactctggagggcccctggtctgcaaggtgaatggcacctaa

SEQ ID NO: 56 - DNA, 1879 bp, Homo sapiens
  misc_feature: Incyte ID No 7510521CB1
ggtttccgggccggcgtactatttcaaggcgcgcgcctcgtggtggactcaccgctagcccgcagcgctcggcttcctggtaattcttcacctcttttctcagctccctgcagcatgggtgctgggccctccttgctgctcgccgccctcctgctgcttctctccggcgacggcgccgtgcgctgcgacacacctgccaactgcacctatcttgacctgctgggcacctgggtcttccaggaccacaagaaaaaaaagtagtggtgtaccttcagaagctggatacagcatatgatgaccttggcaattctggccatttcaccatcatttacaaccaaggctttgagattgtgttgaatgactacaagtggtttgccttttttaagtataaagaagagggcagcaaggtgaccacttactgcaacgagacaatgactgggtgggtgcatgatgtgttgggccggaactgggcttgtttcaccggaaagaaggtgggaactgcctctgagaatgtgtatgtcaacacagcacaccttaagaattctcaggaaaagtattctaataggctctacaagtatgatcacaactttgtgaaagctatcaatgccattcagaagtcttggactgcaactacatacatggaatatgagactcttaccctgggagatatgattaggagaagtggtggccacagtcgaaaaatcccaaggcccaaacctgcaccactgactgctgaaatacagcaaaagattttgcatttgccaacatcttgggactggagaaatgttcatggtatcaattttgtcagtcctgttcgaaaccaagcatcctgtggcagctgctactcatttgcttctatgggtatgctagaagcgagaatccgtatactaaccaacaattctcagaccccaatcctaagccctcaggaggttgtgtcttgtagccagtatgctcaaggctgtgaaggcggcttcccataccttattgcaggaaagtacgcccaagattttgggctggtggaagaagcttgcttcccctacacaggcactgattctccatgcaaaatgaaggaagactgctttcgttattactcctctgagtaccactatgtaggaggtttctatggaggctgcaatgaagccctgatgaagcttgagttggtccatcatgggcccatggcagttgcttttgaagtatatgatgacttcctccactacaaaaaggggatctaccaccacactggtctaagagaccctttcaacccctttgagctgactaatcatgctgttctgcttgtgggctatggcactgactcagcctctgggatggattactggattgttaaaaacagctggggcaccggctggggtgagaatggctacttccggatccgcagaggaactgatgagtgtgcaattgagagcatagcagtggcagccacaccaattcctaaattgtagggtatgccttccagtatttcataatgatctgcatcagttgtaaaggggaattggtatattcacagactgtagactttcagcagcaatctcagaagcttacaaatagatttccatgaagatatttgtcttcagaattaaaactgcccttaattttaatatacctttcaatcggccactggccatttttttctaagtattcaattaagtgggaattttctggaagatggtcagctatgaagtaatagagtttgcttaatcatttgtaattcaaacatgctatattttttaaaatcaatgtgaaaacatagacttatttttaaattgtaccaatcacaagaaaataatggcaataattatcaaaacttttaaaatagatgctcatatttttaaaataaagttttaaaaataaaaaaaaaaaa